SEQUENCE LISTING (298 sequences)

SEQ ID NO: 1 - protein, 288 aa, Homo sapiens
  REGION 1..288: note = Description of sequence: Nucleotide binding domain of the ABCA1 gene product of UniProtKB - O95477
RILDGGGQNDILEIKELTKIYRRKRKPAVDRICVGIPPGECFGLLGVNGAGKSSTFKMLTGDTTVTRGDAFLNKNSILSNIHEVHQNMGYCPQFDAITELLTGREHVEFFALLRGVPEKEVGKVGEWAIRKLGLVKYGEKYAGNYSGGNKRKLSTAMALIGGPPVVFLDEPTTGMDPKARRFLWNCALSVVKEGRSVVLTSHSMEECEALCTRMAIMVNGRFRCLGSVQHLKNRFGDGYTIVVRIAGSNPDLKPVQDFFGLAFPGSVLKEKHRNMLQYQLPSSLSSLA

SEQ ID NO: 2 - protein, 257 aa, Homo sapiens
  REGION 1..257: note = Description of sequence: Nucleotide binding domain of ABCB1 gene product from UniProtKB - P08183
EGLMPNTLEGNVTFGEVVFNYPTRPDIPVLQGLSLEVKKGQTLALVGSSGCGKSTVVQLLERFYDPLAGKVLLDGKEIKRLNVQWLRAHLGIVSQEPILFDCSIAENIAYGDNSRVVSQEEIVRAAKEANIHAFIESLPNKYSTKVGDKGTQLSGGQKQRIAIARALVRQPHILLLDEATSALDTESEKVVQEALDKAREGRTCIVIAHRLSTIQNADLIVVFQNGRVKEHGTHQQLLAQKGIYFSMVSVQAGTKRQ

SEQ ID NO: 3 - protein, 389 aa, Homo sapiens
  REGION 1..389: note = Description of sequence: Nucleotide binding domain of ABCC1 gene product from UniProtKB - P33527
RRPVKDGGGTNSITVRNATFTWARSDPPTLNGITFSIPEGALVAVVGQVGCGKSSLLSALLAEMDKVEGHVAIKGSVAYVPQQAWIQNDSLRENILFGCQLEEPYYRSVIQACALLPDLEILPSGDRTEIGEKGVNLSGGQKQRVSLARAVYSNADIYLFDDPLSAVDAHVGKHIFENVIGPKGMLKNKTRILVTHSMSYLPQVDVIIVMSGGKISEMGSYQELLARDGAFAEFLRTYASTEQEQDAEENGVTGVSGPGKEAKQMENGMLVTDSAGKQLQRQLSSSSSYSGDISRHHNSTAELQKAEAKKEETWKLMEADKAQTGQVKLSVYWDYMKAIGLFISFLSIFLFMCNHVSALASNYWLSLWTDDPIVNGTQEHTKVRLSVYG

SEQ ID NO: 4 - protein, 280 aa, Homo sapiens
  REGION 1..280: note = Description of sequence: Nucleotide binding domain of he ABCD1 gene product from UniProtKB/Swiss-Prot: P33897
GPLKIRGQVVDVEQGIICENIPIVTPSGEVVVASLNIRVEEGMHLLITGPNGCGKSSLFRILGGLWPTYGGVLYKPPPQRMFYIPQRPYMSVGSLRDQVIYPDSVEDMQRKGYSEQDLEAILDVVHLHHILQREGGWEAMCDWKDVLSGGEKQRIGMARMFYHRPKYALLDECTSAVSIDVEGKIFQAAKDAGIALLSITHRPSLWKYHTHLLQFDGEGGWKFEKLDSAARLSLTEEKQRLEQQLAGIPKMQRRLQELCQILGEAVAPAHVPAPSPQGPG

SEQ ID NO: 5 - protein, 293 aa, Homo sapiens
  REGION 1..293: note = Description of sequence: Nucleotide binding domain of ABCE1 gene product from UniProtKB/Swiss-Prot: P61221
GALSIVNLPSNLEKETTHRYCANAFKLHRLPIPRPGEVLGLVGTNGIGKSTALKILAGKQKPNLGKYDDPPDWQEILTYFRGSELQNYFTKILEDDLKAIIKPQYVDQIPKAAKGTVGSILDRKDETKTQAIVCQQLDLTHLKERNVEDLSGGELQRFACAVVCIQKADIFMFDEPSSYLDVKQRLKAAITIRSLINPDRYIIVVEHDLSVLDYLSDFICCLYGVPSAYGVVTMPFSVREGINIFLDGYVPTENLRFRDASLVFKVAETANEEEVKKMCMYKYPGMKKKMGEF

SEQ ID NO: 6 - protein, 228 aa, Homo sapiens
  REGION 1..228: note = Description of sequence: Nucleotide binding domain of the ABCF1 gene product from UniProtKB - Q8NE71
PPLSPPVLGLHGVTFGYQGQKPLFKNLDFGIDMDSRICIVGPNGVGKSTLLLLLTGKLTPTHGEMRKNHRLKIGFFNQQYAEQLRMEETPTEYLQRGFNLPYQDARKCLGRFGLESHAHTIQICKLSGGQKARVVFAELACREPDVLILDEPTNNLDIESIDALGEAINEYKGAVIVVSHDARLITETNCQLWVVEEQSVSQIDGDFEDYKREVLEALGEVMVSRPRE

SEQ ID NO: 7 - protein, 269 aa, Homo sapiens
  REGION 1..269: note = Description of sequence: Nucleotide binding domain of the ABCG2 gene product from UniProtKB/Swiss-Prot: P45844
RFSSLPRRAAVNIEFRDLSYSVPEGPWWRKKGYKTLLKGISGKFNSGELVAIMGPSGAGKSTLMNILAGYRETGMKGAVLINGLPRDLRCFRKVSCYIMQDDMLLPHLTVQEAMMVSAHLKLQEKDEGRREMVKEILTALGLLSCANTRTGSLSGGQRKRLAIALELVNNPPVMFFDEPTSGLDSASCFQVVSLMKGLAQGGRSIICTIHQPSAKLFELFDQLYVLSQGQCVYRGKVCNLVPYLRDLGLNCPTYHNPADFVMEVASGEY

SEQ ID NO: 8 - protein, 748 aa, Homo sapiens
  REGION 1..748: note = Description of sequence: TAP1 NCBI NP_000584.3
MASSRCPAPRGCRCLPGASLAWLGTVLLLLADWVLLRTALPRIFSLLVPTALPLLRVWAVGLSRWAVLWLGACGVLRATVGSKSENAGAQGWLAALKPLAAALGLALPGLALFRELISWGAPGSADSTRLLHWGSHPTAFVVSYAAALPAAALWHKLGSLWVPGGQGGSGNPVRRLLGCLGSETRRLSLFLVLVVLSSLGEMAIPFFTGRLTDWILQDGSADTFTRNLTLMSILTIASAVLEFVGDGIYNNTMGHVHSHLQGEVFGAVLRQETEFFQQNQTGNIMSRVTEDTSTLSDSLSENLSLFLWYLVRGLCLLGIMLWGSVSLTMVTLITLPLLFLLPKKVGKWYQLLEVQVRESLAKSSQVAIEALSAMPTVRSFANEEGEAQKFREKLQEIKTLNQKEAVAYAVNSWTTSISGMLLKVGILYIGGQLVTSGAVSSGNLVTFVLYQMQFTQAVEVLLSIYPRVQKAVGSSEKIFEYLDRTPRCPPSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPCVLILDDATSALDANSQLQVEQLLYESPERYSRSVLLITQHLSLVEQADHILFLEGGAIREGGTHQQLMEKKGCYWAMVQAPADAPE

SEQ ID NO: 9 - protein, 257 aa, Homo sapiens
  REGION 1..257: note = Description of sequence: A nucleotide binding domain polypeptide of wild type TAP1
GLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPCVLILDDATSALDANSQLQVEQLLYESPERYSRSVLLITQHLSLVEQADHILFLEGGAIREGGTHQQLMEKKGCYWAMVQAPADAPE

SEQ ID NO: 10 - protein, 5 aa, synthetic construct
  REGION 1..5: note = Description of sequence: helical sub-domain
LDGGQ

SEQ ID NO: 11 - protein, 6 aa, synthetic construct
  REGION 1..6: note = Description of sequence: TAP1 C-Terminal sequence
PADAPE

SEQ ID NO: 12 - protein, 251 aa, Homo sapiens
  REGION 1..251: note = Description of sequence: A nucleotide binding domain polypeptide of wild type TAP1 with PADAPE deleted
GLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPCVLILDDATSALDANSQLQVEQLLYESPERYSRSVLLITQHLSLVEQADHILFLEGGAIREGGTHQQLMEKKGCYWAMVQA

SEQ ID NO: 13 - protein, 257 aa, synthetic construct
  REGION 1..257: note = Description of sequence: A nucleotide binding domain polypeptide based on TAP1
GLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAPADAPE

SEQ ID NO: 14 - protein, 251 aa, synthetic construct
  REGION 1..251: note = Description of sequence: A nucleotide binding domain polypeptide based on TAP1 with PADAPE deleted
  VARIANT 177: note = X is an amino acid other than aspartic acid or glutamic acid
GLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDXATSALDAGNQLRVQRLLYESPERYSRSVLLITQHLSLVEQADHILFLEGGAIREGGTHQQLMEKKGSYWAMVQA

SEQ ID NO: 15 - protein, 260 aa, synthetic construct
  REGION 1..260: note = Description of sequence: A nucleotide binding domain polypeptide based on TAP1 with D668N substitution
PPSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAPADAPE

SEQ ID NO: 16 - protein, 260 aa, synthetic construct
  REGION 1..260: note = Description of sequence: A nucleotide binding domain polypeptide based on TAP1 with D668 substitution
  VARIANT 180: note = X is an amino acid other than aspartic acid or glutamic acid
PPSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDXATSALDAGNQLRVQRLLYESPERYSRSVLLITQHLSLVEQADHILFLEGGAIREGGTHQQLMEKKGSYWAMVQAPADAPE

SEQ ID NO: 17 - protein, 10 aa, synthetic construct
  REGION 1..10: note = Description of sequence: Coupling helix aa sequence of TAP1
ETEFFQQNQT

SEQ ID NO: 18 - protein, 8 aa, synthetic construct
  REGION 1..8: note = Description of sequence: Coupling helix aa sequence of TAP2
GLQTVRSF

SEQ ID NO: 19 - protein, 289 aa, synthetic construct
  REGION 1..289: note = Description of sequence: A nucleotide binding domain polypeptide based on TAP1 with an N-terminal TAP1 and TAP2 cap.
ETEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDDATSALDAGNQLRVQRLLYESPERYSRSVLLITQHLSLVEQADHILFLEGGAIREGGTHQQLMEKKGSYWAMVQAPADAPE

SEQ ID NO: 20 - protein, 289 aa, synthetic construct
  REGION 1..289: note = Description of sequence: A nucleotide binding domain polypeptide based on TAP1 with an N-terminal TAP1 and TAP2 cap and substitution at D668
  VARIANT 209: note = X is an amino acid other than aspartic acid or glutamic acid
ETEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDXATSALDAGNQLRVQRLLYESPERYSRSVLLITQHLSLVEQADHILFLEGGAIREGGTHQQLMEKKGSYWAMVQAPADAPE

SEQ ID NO: 21 - protein, 686 aa, Homo sapiens
  REGION 1..686: note = Description of sequence: Tap2 UniProtKB - Q03519
MRLPDLRPWTSLLLVDAALLWLLQGPLGTLLPQGLPGLWLEGTLRLGGLWGLLKLRGLLGFVGTLLLPLCLATPLTVSLRALVAGASRAPPARVASAPWSWLLVGYGAAGLSWSLWAVLSPPGAQEKEQDQVNNKVLMWRLLKLSRPDLPLLVAAFFFLVLAVLGETLIPHYSGRVIDILGGDFDPHAFASAIFFMCLFSFGSSLSAGCRGGCFTYTMSRINLRIREQLFSSLLRQDLGFFQETKTGELNSRLSSDTTLMSNWLPLNANVLLRSLVKVVGLYGFMLSISPRLTLLSLLHMPFTIAAEKVYNTRHQEVLREIQDAVARAGQVVREAVGGLQTVRSFGAEEHEVCRYKEALEQCRQLYWRRDLERALYLLVRRVLHLGVQMLMLSCGLQQMQDGELTQGSLLSFMIYQESVGSYVQTLVYIYGDMLSNVGAAEKVFSYMDRQPNLPSPGTLAPTTLQGVVKFQDVSFAYPNRPDRPVLKGLTFTLRPGEVTALVGPNGSGKSTVAALLQNLYQPTGGQVLLDEKPISQYEHCYLHSQVVSVGQEPVLFSGSVRNNIAYGLQSCEDDKVMAAAQAAHADDFIQEMEHGIYTDVGEKGSQLAAGQKQRLAIARALVRDPRVLILDEATSALDVQCEQALQDWNSRGDRTVLVIAHRLQTVQRAHQILVLQEGKLQKLAQL

SEQ ID NO: 22 - protein, 226 aa, Homo sapiens
  REGION 1..226: note = Description of sequence: A nucleotide binding domain polypeptide of wild type TAP2
GTLAPTTLQGVVKFQDVSFAYPNRPDRPVLKGLTFTLRPGEVTALVGPNGSGKSTVAALLQNLYQPTGGQVLLDEKPISQYEHCYLHSQVVSVGQEPVLFSGSVRNNIAYGLQSCEDDKVMAAAQAAHADDFIQEMEHGIYTDVGEKGSQLAAGQKQRLAIARALVRDPRVLILDEATSALDVQCEQALQDWNSRGDRTVLVIAHRLQTVQRAHQILVLQEGKLQK

SEQ ID NO: 23 - protein, 7 aa, synthetic construct
  REGION 1..7: note = Description of sequence: TAP1 alpha helix 9
CYWAMVQ

SEQ ID NO: 24 - protein, 7 aa, synthetic construct
  REGION 1..7: note = Description of sequence: TAP1 alpha helix 8
THQQLME

SEQ ID NO: 25 - protein, 10 aa, synthetic construct
  REGION 1..10: note = Description of sequence: human TAP1 D-helix aa sequence
NSQLQVEQLL

SEQ ID NO: 26 - protein, 10 aa, synthetic construct
  REGION 1..10: note = Description of sequence: rat TAP1 D-helix aa sequence
GNQLRVQRLL

SEQ ID NO: 27 - protein, 1480 aa, Homo sapiens
  REGION 1..1480: note = Description of sequence: NCBI Accession ABD72215.1
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 28 - protein, 200 aa, Homo sapiens
  REGION 1..200: note = Description of sequence: A nucleotide binding domain polypeptide of the CFTR
VLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPD

SEQ ID NO: 29 - protein, 229 aa, Homo sapiens
  REGION 1..229: note = Description of sequence: A nucleotide binding domain polypeptide of the CFTR
  VARIANT 2: note = X is leucine or the residue may be deleted (absent)
  VARIANT 229: note = X is glycine or the residue may be deleted (absent)
SXTTTEVVMENVTAFWEEGGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMX

SEQ ID NO: 30 - protein, 4 aa, synthetic construct
  REGION 1..4: note = Description of sequence: Linker sequence
GGGS

SEQ ID NO: 31 - protein, 8 aa, synthetic construct
  REGION 1..8: note = Description of sequence: Linker sequence
GGGSGGGS

SEQ ID NO: 32 - protein, 12 aa, synthetic construct
  REGION 1..12: note = Description of sequence: Linker sequence
GGGSGGGSGGGS

SEQ ID NO: 33 - protein, 16 aa, synthetic construct
  REGION 1..16: note = Description of sequence: Linker sequence
GGGSGGGSGGGSGGGS

SEQ ID NO: 34 - protein, 20 aa, synthetic construct
  REGION 1..20: note = Description of sequence: Linker sequence
GGGSGGGSGGGSGGGSGGGS

SEQ ID NO: 35 - protein, 24 aa, synthetic construct
  REGION 1..24: note = Description of sequence: Linker sequence
GGGSGGGSGGGSGGGSGGGSGGGS

SEQ ID NO: 36 - protein, 24 aa, synthetic construct
  REGION 1..24: note = Description of sequence: Linker sequence
GGGSGGGSGGGSGGGSGGGSGGGS

SEQ ID NO: 37 - protein, 24 aa, synthetic construct
  REGION 1..24: note = Description of sequence: Linker sequence
GGGSGGGSGGGSGGGSGGGSGGGS

SEQ ID NO: 38 - protein, 28 aa, synthetic construct
  REGION 1..28: note = Description of sequence: Linker sequence
GGGSGGGSGGGSGGGSGGGSGGGSGGGS

SEQ ID NO: 39 - protein, 32 aa, synthetic construct
  REGION 1..32: note = Description of sequence: Linker sequence
GGGSGGGSGGGSGGGSGGGSGGGSGGGSGGGS

SEQ ID NO: 40 - protein, 5 aa, synthetic construct
  REGION 1..5: note = Description of sequence: Linker sequence
GGGGS

SEQ ID NO: 41 - protein, 10 aa, synthetic construct
  REGION 1..10: note = Description of sequence: Linker sequence
GGGGSGGGGS

SEQ ID NO: 42 - protein, 15 aa, synthetic construct
  REGION 1..15: note = Description of sequence: Linker sequence
GGGGSGGGGSGGGGS

SEQ ID NO: 43 - protein, 20 aa, synthetic construct
  REGION 1..20: note = Description of sequence: Linker sequence
GGGGSGGGGSGGGGSGGGGS

SEQ ID NO: 44 - protein, 25 aa, synthetic construct
  REGION 1..25: note = Description of sequence: Linker sequence
GGGGSGGGGSGGGGSGGGGSGGGGS

SEQ ID NO: 45 - protein, 30 aa, synthetic construct
  REGION 1..30: note = Description of sequence: Linker sequence
GGGGSGGGGSGGGGSGGGGSGGGGSGGGGS

SEQ ID NO: 46 - protein, 35 aa, synthetic construct
  REGION 1..35: note = Description of sequence: Linker sequence
GGGGSGGGGSGGGGSGGGGSGGGGSGGGGSGGGGS

SEQ ID NO: 47 - protein, 40 aa, synthetic construct
  REGION 1..40: note = Description of sequence: Linker sequence
GGGGSGGGGSGGGGSGGGGSGGGGSGGGGSGGGGSGGGGS

SEQ ID NO: 48 - protein, 45 aa, synthetic construct
  REGION 1..45: note = Description of sequence: Linker sequence
GGGGSGGGGSGGGGSGGGGSGGGGSGGGGSGGGGSGGGGSGGGGS

SEQ ID NO: 49 - protein, 50 aa, synthetic construct
  REGION 1..50: note = Description of sequence: Linker sequence
GGGGSGGGGSGGGGSGGGGSGGGGSGGGGSGGGGSGGGGSGGGGSGGGGS

SEQ ID NO: 50 - protein, 100 aa, synthetic construct
  REGION 1..100: note = Description of sequence: Linker
  REGION 1..100: note = Repeat unit EAAAK is repeated between 1 to 20 times
  VARIANT 6..10: note = This position may be absent; its presence requires the concurrent presence of each of the other positions in this feature
  VARIANT 11..15: note = This position may be absent; its presence requires the concurrent presence of each of the other positions in this feature
  VARIANT 16..20: note = This position may be absent; its presence requires the concurrent presence of each of the other positions in this feature
  VARIANT 21..25: note = This position may be absent; its presence requires the concurrent presence of each of the other positions in this feature
  VARIANT 26..30: note = This position may be absent; its presence requires the concurrent presence of each of the other positions in this feature
  VARIANT 31..35: note = This position may be absent; its presence requires the concurrent presence of each of the other positions in this feature
  VARIANT 36..40: note = This position may be absent; its presence requires the concurrent presence of each of the other positions in this feature
  VARIANT 41..45: note = This position may be absent; its presence requires the concurrent presence of each of the other positions in this feature
  VARIANT 46..50: note = This position may be absent; its presence requires the concurrent presence of each of the other positions in this feature
  VARIANT 51..55: note = This position may be absent; its presence requires the concurrent presence of each of the other positions in this feature
  VARIANT 56..60: note = This position may be absent; its presence requires the concurrent presence of each of the other positions in this feature
  VARIANT 61..65: note = This position may be absent; its presence requires the concurrent presence of each of the other positions in this feature
  VARIANT 66..70: note = This position may be absent; its presence requires the concurrent presence of each of the other positions in this feature
  VARIANT 71..75: note = This position may be absent; its presence requires the concurrent presence of each of the other positions in this feature
  VARIANT 76..80: note = This position may be absent; its presence requires the concurrent presence of each of the other positions in this feature
  VARIANT 81..85: note = This position may be absent; its presence requires the concurrent presence of each of the other positions in this feature
  VARIANT 86..90: note = This position may be absent; its presence requires the concurrent presence of each of the other positions in this feature
  VARIANT 91..95: note = This position may be absent; its presence requires the concurrent presence of each of the other positions in this feature
  VARIANT 96..100: note = This position may be absent; its presence requires the concurrent presence of each of the other positions in this feature
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 51 - protein, 40 aa, synthetic construct
  REGION 1..40: note = Description of sequence: Linker
  REGION 1..40: note = Repeat unit KP is repeated between 2 to 20 times
  VARIANT 5..6: note = This position may be absent; its presence requires the concurrent presence of both of the positions in this feature
  VARIANT 7..8: note = This position may be absent; its presence requires the concurrent presence of both of the positions in this feature
  VARIANT 9..10: note = This position may be absent; its presence requires the concurrent presence of both of the positions in this feature
  VARIANT 11..12: note = This position may be absent; its presence requires the concurrent presence of both of the positions in this feature
  VARIANT 13..14: note = This position may be absent; its presence requires the concurrent presence of both of the positions in this feature
  VARIANT 15..16: note = This position may be absent; its presence requires the concurrent presence of both of the positions in this feature
  VARIANT 17..18: note = This position may be absent; its presence requires the concurrent presence of both of the positions in this feature
  VARIANT 19..20: note = This position may be absent; its presence requires the concurrent presence of both of the positions in this feature
  VARIANT 21..22: note = This position may be absent; its presence requires the concurrent presence of both of the positions in this feature
  VARIANT 23..24: note = This position may be absent; its presence requires the concurrent presence of both of the positions in this feature
  VARIANT 25..26: note = This position may be absent; its presence requires the concurrent presence of both of the positions in this feature
  VARIANT 27..28: note = This position may be absent; its presence requires the concurrent presence of both of the positions in this feature
  VARIANT 29..30: note = This position may be absent; its presence requires the concurrent presence of both of the positions in this feature
  VARIANT 31..32: note = This position may be absent; its presence requires the concurrent presence of both of the positions in this feature
  VARIANT 33..34: note = This position may be absent; its presence requires the concurrent presence of both of the positions in this feature
  VARIANT 35..36: note = This position may be absent; its presence requires the concurrent presence of both of the positions in this feature
  VARIANT 37..38: note = This position may be absent; its presence requires the concurrent presence of both of the positions in this feature
  VARIANT 39..40: note = This position may be absent; its presence requires the concurrent presence of both of the positions in this feature
KPKPKPKPKPKPKPKPKPKPKPKPKPKPKPKPKPKPKPKP

SEQ ID NO: 52 - protein, 40 aa, synthetic construct
  REGION 1..40: note = Description of sequence: Linker
  REGION 1..40: note = Repeat unit EP is repeated between 2 to 20 times
  VARIANT 5..6: note = This position may be absent; its presence requires the concurrent presence of both of the positions in this feature
  VARIANT 7..8: note = This position may be absent; its presence requires the concurrent presence of both of the positions in this feature
  VARIANT 9..10: note = This position may be absent; its presence requires the concurrent presence of both of the positions in this feature
  VARIANT 11..12: note = This position may be absent; its presence requires the concurrent presence of both of the positions in this feature
  VARIANT 13..14: note = This position may be absent; its presence requires the concurrent presence of both of the positions in this feature
  VARIANT 15..16: note = This position may be absent; its presence requires the concurrent presence of both of the positions in this feature
  VARIANT 17..18: note = This position may be absent; its presence requires the concurrent presence of both of the positions in this feature
  VARIANT 19..20: note = This position may be absent; its presence requires the concurrent presence of both of the positions in this feature
  VARIANT 21..22: note = This position may be absent; its presence requires the concurrent presence of both of the positions in this feature
  VARIANT 23..24: note = This position may be absent; its presence requires the concurrent presence of both of the positions in this feature
  VARIANT 25..26: note = This position may be absent; its presence requires the concurrent presence of both of the positions in this feature
  VARIANT 27..28: note = This position may be absent; its presence requires the concurrent presence of both of the positions in this feature
  VARIANT 29..30: note = This position may be absent; its presence requires the concurrent presence of both of the positions in this feature
  VARIANT 31..32: note = This position may be absent; its presence requires the concurrent presence of both of the positions in this feature
  VARIANT 33..34: note = This position may be absent; its presence requires the concurrent presence of both of the positions in this feature
  VARIANT 35..36: note = This position may be absent; its presence requires the concurrent presence of both of the positions in this feature
  VARIANT 37..38: note = This position may be absent; its presence requires the concurrent presence of both of the positions in this feature
  VARIANT 39..40: note = This position may be absent; its presence requires the concurrent presence of both of the positions in this feature
EPEPEPEPEPEPEPEPEPEPEPEPEPEPEPEPEPEPEPEP

SEQ ID NO: 53 - protein, 60 aa, synthetic construct
  REGION 1..60: note = Description of sequence: Linker
  REGION 1..60: note = Repeat unit TPR is repeated between 2 to 20 times
  VARIANT 7..9: note = This position may be absent; its presence requires the concurrent presence of each of the other positions in this feature
  VARIANT 10..12: note = This position may be absent; its presence requires the concurrent presence of each of the other positions in this feature
  VARIANT 13..15: note = This position may be absent; its presence requires the concurrent presence of each of the other positions in this feature
  VARIANT 16..18: note = This position may be absent; its presence requires the concurrent presence of each of the other positions in this feature
  VARIANT 19..21: note = This position may be absent; its presence requires the concurrent presence of each of the other positions in this feature
  VARIANT 22..24: note = This position may be absent; its presence requires the concurrent presence of each of the other positions in this feature
  VARIANT 25..27: note = This position may be absent; its presence requires the concurrent presence of each of the other positions in this feature
  VARIANT 28..30: note = This position may be absent; its presence requires the concurrent presence of each of the other positions in this feature
  VARIANT 31..33: note = This position may be absent; its presence requires the concurrent presence of each of the other positions in this feature
  VARIANT 34..36: note = This position may be absent; its presence requires the concurrent presence of each of the other positions in this feature
  VARIANT 37..39: note = This position may be absent; its presence requires the concurrent presence of each of the other positions in this feature
  VARIANT 40..42: note = This position may be absent; its presence requires the concurrent presence of each of the other positions in this feature
  VARIANT 43..45: note = This position may be absent; its presence requires the concurrent presence of each of the other positions in this feature
  VARIANT 46..48: note = This position may be absent; its presence requires the concurrent presence of each of the other positions in this feature
  VARIANT 49..51: note = This position may be absent; its presence requires the concurrent presence of each of the other positions in this feature
  VARIANT 52..54: note = This position may be absent; its presence requires the concurrent presence of each of the other positions in this feature
  VARIANT 55..57: note = This position may be absent; its presence requires the concurrent presence of each of the other positions in this feature
  VARIANT 58..60: note = This position may be absent; its presence requires the concurrent presence of each of the other positions in this feature
TPRTPRTPRTPRTPRTPRTPRTPRTPRTPRTPRTPRTPRTPRTPRTPRTPRTPRTPRTPR

SEQ ID NO: 54 - protein, 40 aa, synthetic construct
  REGION 1..40: note = Description of sequence: Linker
  REGION 1..40: note = Repeat unit AP is repeated between 2 to 20 times
  VARIANT 5..6: note = This position may be absent; its presence requires the concurrent presence of both of the positions in this feature
  VARIANT 7..8: note = This position may be absent; its presence requires the concurrent presence of both of the positions in this feature
  VARIANT 9..10: note = This position may be absent; its presence requires the concurrent presence of both of the positions in this feature
  VARIANT 11..12: note = This position may be absent; its presence requires the concurrent presence of both of the positions in this feature
  VARIANT 13..14: note = This position may be absent; its presence requires the concurrent presence of both of the positions in this feature
  VARIANT 15..16: note = This position may be absent; its presence requires the concurrent presence of both of the positions in this feature
  VARIANT 17..18: note = This position may be absent; its presence requires the concurrent presence of both of the positions in this feature
  VARIANT 19..20: note = This position may be absent; its presence requires the concurrent presence of both of the positions in this feature
  VARIANT 21..22: note = This position may be absent; its presence requires the concurrent presence of both of the positions in this feature
  VARIANT 23..24: note = This position may be absent; its presence requires the concurrent presence of both of the positions in this feature
  VARIANT 25..26: note = This position may be absent; its presence requires the concurrent presence of both of the positions in this feature
  VARIANT 27..28: note = This position may be absent; its presence requires the concurrent presence of both of the positions in this feature
  VARIANT 29..30: note = This position may be absent; its presence requires the concurrent presence of both of the positions in this feature
  VARIANT 31..32: note = This position may be absent; its presence requires the concurrent presence of both of the positions in this feature
  VARIANT 33..34: note = This position may be absent; its presence requires the concurrent presence of both of the positions in this feature
  VARIANT 35..36: note = This position may be absent; its presence requires the concurrent presence of both of the positions in this feature
  VARIANT 37..38: note = This position may be absent; its presence requires the concurrent presence of both of the positions in this feature
  VARIANT 39..40: note = This position may be absent; its presence requires the concurrent presence of both of the positions in this feature
APAPAPAPAPAPAPAPAPAPAPAPAPAPAPAPAPAPAPAP

SEQ ID NO: 55 - protein, 4 aa, synthetic construct
  REGION 1..4: note = Description of sequence: Linker sequence
APAP

SEQ ID NO: 56 - protein, 6 aa, synthetic construct
  REGION 1..6: note = Description of sequence: Linker sequence
APAPAP

SEQ ID NO: 57 - protein, 8 aa, synthetic construct
  REGION 1..8: note = Description of sequence: Linker sequence
APAPAPAP

SEQ ID NO: 58 - protein, 10 aa, synthetic construct
  REGION 1..10: note = Description of sequence: Linker sequence
APAPAPAPAP

SEQ ID NO: 59 - protein, 227 aa, Homo sapiens
  REGION 1..227: note = Description of sequence: WT Homo sapiens IgG1 Fc Sequence
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 217 aa, synthetic construct
  REGION 1..217: note = Description of sequence: Non-dimerizing (monomeric) modified IgG1 heavy chain constant regions aa sequences
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTSPPSRDELTKNQVSLRCHVKGFYPSDIAVEWESNGQPENNYKTTKPVLDSDGSFFLYSKLTVDKSRWQQGNVFSCSVMHEALHNHYTQKSLSLSPGK

SEQ ID NO: 61 - protein, 217 aa, synthetic construct
  REGION 1..217: note = Description of sequence: Non-dimerizing (monomeric) modified IgG1 heavy chain constant regions aa sequences with disulfide bonds between cysteins substitued for L242 and K334
  DISULFID 12..104: note = Cysteine-cysteine disulfide bond
APELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTSPPSRDELTKNQVSLRCHVKGFYPSDIAVEWESNGQPENNYKTTKPVLDSDGSFFLYSKLTVDKSRWQQGNVFSCSVMHEALHNHYTQKSLSLSPGK

SEQ ID NO: 62 - protein, 217 aa, synthetic construct
  REGION 1..217: note = Description of sequence: Non-dimerizing (monomeric) modified IgG1 heavy chain constant regions aa sequences with disulfide bonds between cysteins substitued for L242 and K334 and/or between P343 and A431
  DISULFID 12..104: note = Cysteine-cysteine disulfide bond
  DISULFID 113..201: note = Cysteine-cysteine disulfide bond
APELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQCREPQVYTSPPSRDELTKNQVSLRCHVKGFYPSDIAVEWESNGQPENNYKTTKPVLDSDGSFFLYSKLTVDKSRWQQGNVFSCSVMHECLHNHYTQKSLSLSPGK

SEQ ID NO: 63 - protein, 482 aa, synthetic construct
  REGION 1..482: note = Description of sequence: scFc (wild type L234 L235; including Fc hinge
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 325 aa, Homo sapiens
  REGION 1..325: note = Description of sequence: Homo sapiens IgG2 Fc GenBank AAN76044
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 65 - protein, 246 aa, Homo sapiens
  REGION 1..246: note = Description of sequence: Homo sapiens IgG3 Fc GenBank AAW65947
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 223 aa, Homo sapiens
  REGION 1..223: note = Description of sequence: Homo sapiens IgG4 Fc Segment
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 67 - protein, 106 aa, Homo sapiens
  REGION 1..106: note = Description of sequence: An Ig kappa light chain constant region sequence
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 106 aa, Homo sapiens
  REGION 1..106: note = Description of sequence: An Ig lambda light chain constant region sequence
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 69 - protein, 112 aa, Homo sapiens
  REGION 1..112: note = Description of sequence: An Ig heavy chain CH1 constant region sequence
FTVRETASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 70 - protein, 226 aa, Homo sapiens
  REGION 1..226: note = Description of sequence: hIgG1 Fc with LALA substitutions
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 227 aa, Homo sapiens
  REGION 1..227: note = Description of sequence: hIgG1 Fc with LALA and P331S substitutions
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 253 aa, synthetic construct
  REGION 1..253: note = Description of sequence: A scFv anti-human CD3
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVTTYADSVKGRFTISVDKSKNTAYLQMNSLRAEDTAVYYCARSGYYGDSDWYFDVWGQGTLVTVSS

SEQ ID NO: 73 - protein, 253 aa, synthetic construct
  REGION 1..253: note = Description of sequence: A scFv anti-human CD3
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 74 - protein, 115 aa, synthetic construct
  REGION 1..115: note = Description of sequence: An anti-human CD3 VHH
QVQLVESGGGLVQPGGSLRLSCAASGSIFSANTMGWYRQAPGKQRELVAGMNTSGSTVYGDSVKGRFTISRDNAKNIAYLQMNSLIPEDTAVYYCTLVQRGPNYWGQGTQVTVSS

SEQ ID NO: 75 - protein, 240 aa, synthetic construct
  REGION 1..240: note = Description of sequence: An anti-human CD3 scFv (vH-vL)
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 76 - protein, 240 aa, synthetic construct
  REGION 1..240: note = Description of sequence: An anti-human CD3 scFv (vH-vL) with disulfide linkage
  DISULFID 43..233: note = Cysteine-cysteine disulfide bond
DVQLVQSGAEVKKPGASVKVSCKASGYTFTRYTMHWVRQAPGCGLEWIGYINPSRGYTNYADSVKGRFTITTDKSTSTAYMELSSLRSEDTATYYCARYYDDHYCLDYWGQGTTVTVSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGCGTKVEIK

SEQ ID NO: 77 - protein, 240 aa, synthetic construct
  REGION 1..240: note = Description of sequence: An anti-human CD3 scFv (vH-vL) with disulfide linkage
  DISULFID 43..234: note = Cysteine-cysteine disulfide bond
DVQLVQSGAEVKKPGASVKVSCKASGYTFTRYTMHWVRQAPGCGLEWIGYINPSRGYTNYADSVKGRFTITTDKSTSTAYMELSSLRSEDTATYYCARYYDDHYCLDYWGQGTTVTVSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGCTKVEIK

SEQ ID NO: 78 - protein, 240 aa, synthetic construct
  REGION 1..240: note = Description of sequence: An anti-human CD3 scFv (vH-vL) with disulfide linkage
  DISULFID 44..233: note = Cysteine-cysteine disulfide bond
DVQLVQSGAEVKKPGASVKVSCKASGYTFTRYTMHWVRQAPGQCLEWIGYINPSRGYTNYADSVKGRFTITTDKSTSTAYMELSSLRSEDTATYYCARYYDDHYCLDYWGQGTTVTVSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGCGTKVEIK

SEQ ID NO: 79 - protein, 240 aa, synthetic construct
  REGION 1..240: note = Description of sequence: An anti-human CD3 scFv (vH-vL) with disulfide linkage
  DISULFID 44..234: note = Cysteine-cysteine disulfide bond
DVQLVQSGAEVKKPGASVKVSCKASGYTFTRYTMHWVRQAPGQCLEWIGYINPSRGYTNYADSVKGRFTITTDKSTSTAYMELSSLRSEDTATYYCARYYDDHYCLDYWGQGTTVTVSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGCTKVEIK

SEQ ID NO: 80 - protein, 119 aa, synthetic construct
  REGION 1..119: note = Description of sequence: Anti-CD16 VHH
EVQLVESGGGLVQPGESLTLSCVVAGSIFSFAMSWYRQAPGKERELVARIGSDDRVTYADSVKGRFTISRDNIKRTAGLQMNSLKPEDTAVYYCNAQTDLRDWTVREYWGQGTQVTVSS

SEQ ID NO: 81 - protein, 122 aa, synthetic construct
  REGION 1..122: note = Description of sequence: Anti-CD16 VHH
EVQLVESGGELVQPGGSLRLSCAASGLTFSSYNMGWFRRAPGKEREFVASITWSGRDTFYADSVKGRFTISRDNAKNTVYLQMSSLKPEDTAVYYCAANPWPVAAPRSGTYWGQGTQVTVSS

SEQ ID NO: 82 - protein, 265 aa, synthetic construct
  REGION 1..265: note = Description of sequence: Single-chain interferon (IFN) gamma (monomeric engagement)
MQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIDELIQVMAEFSTEEQQEGPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRG

SEQ ID NO: 83 - protein, 486 aa, synthetic construct
  REGION 1..486: note = Description of sequence: Single-chain TNF (scTNF)
SSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGSGGGSGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGSGGGSGGGSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 84-86 - skipped (3 entries)

SEQ ID NO: 87 - DNA, 24 bp, synthetic construct
  misc_feature 1..24: note = Description of sequence: Oligonucleotide IMT504
  source 1..24: mol_type = other DNA
tcatcattttgtcattttgtcatt

SEQ ID NO: 88 - protein, 240 aa, synthetic construct
  REGION 1..240: note = Description of sequence: An anti-mesothelin scFv polypeptide related to the murine-derived SS1 antibody
  VARIANT 121: note = May be either valine or glycine
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSGXGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 89 - protein, 244 aa, synthetic construct
  REGION 1..244: note = Description of sequence: An anti-human mesothelin scFv (vH-vL) polypeptide related to the monoclonal antibody YP218
  VARIANT 121: note = May be either valine or glycine
QEQLVESGGGLVQPGASLTLTCTASGIDFSRYYMCWVRQAPGKGLEGIACIYIGGSGSTYYASWAKGRFTISKASSTTVTLQMTSLTAADTATYFCARGTNLNYIFRLWGPGTLVTVSSGXGGSGGGGSGGGGSDVVMTQTPASVSEPVGGTVTIKCQASQRISSYLSWYQQKPGQRPKLLIFGASTLASGVPSRFKGSGSGTEYTLTISDLECADAATYYCQSYAYFDSNNWHAFGGGTEVVV

SEQ ID NO: 90 - protein, 244 aa, synthetic construct
  REGION 1..244: note = Description of sequence: An anti-human mesothelin scFv (vL-vH) polypeptide related to the monoclonal antibody YP218
  VARIANT 112: note = May be either valine or glycine
DVVMTQTPASVSEPVGGTVTIKCQASQRISSYLSWYQQKPGQRPKLLIFGASTLASGVPSRFKGSGSGTEYTLTISDLECADAATYYCQSYAYFDSNNWHAFGGGTEVVVGXGGSGGGGSGGGGSQEQLVESGGGLVQPGASLTLTCTASGIDFSRYYMCWVRQAPGKGLEGIACIYIGGSGSTYYASWAKGRFTISKASSTTVTLQMTSLTAADTATYFCARGTNLNYIFRLWGPGTLVTVSS

SEQ ID NO: 91 - protein, 239 aa, synthetic construct
  REGION 1..239: note = Description of sequence: An anti-human mesothelin scFv (vH-vL) polypeptide related to the monoclonal antibody 15B6
  VARIANT 116: note = May be either valine or glycine
EVQLQQSGPVLVKPGASVKISCKASGYSFTGYYMHWVRQSNGKSLEWIGRINPYTGVPSYKHNFKDKASLTVDKSSSTAYMELHSLTSEDSAVYYCARELGGYWGQGTTLTVSSGXGGSGGGGSGGGGSQAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVLG

SEQ ID NO: 92 - protein, 239 aa, synthetic construct
  REGION 1..239: note = Description of sequence: An anti-human mesothelin scFv (vL-vH) polypeptide related to the monoclonal antibody 15B6
  VARIANT 112: note = May be either valine or glycine
QAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGGTKLTVLGGXGGSGGGGSGGGGSEVQLQQSGPVLVKPGASVKISCKASGYSFTGYYMHWVRQSNGKSLEWIGRINPYTGVPSYKHNFKDKASLTVDKSSSTAYMELHSLTSEDSAVYYCARELGGYWGQGTTLTVSS

SEQ ID NO: 93 - protein, 239 aa, synthetic construct
  REGION 1..239: note = Description of sequence: An anti-human mesothelin scFv (vL-vH) polypeptide related to the monoclonal antibody 15B6 with a disulfide linkage
  DISULFID 102..168: note = Cysteine-cysteine disulfide bond
QAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGCGTKLTVLGGGGGSGGGGSGGGGSEVQLQQSGPVLVKPGASVKISCKASGYSFTGYYMHWVRQSNGCSLEWIGRINPYTGVPSYKHNFKDKASLTVDKSSSTAYMELHSLTSEDSAVYYCARELGGYWGQGTTLTVSS

SEQ ID NO: 94 - protein, 239 aa, synthetic construct
  REGION 1..239: note = Description of sequence: An anti-human mesothelin scFv (vL-vH) polypeptide related to the monoclonal antibody 15B6 with a disulfide linkage
  DISULFID 102..169: note = Cysteine-cysteine disulfide bond
QAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGCGTKLTVLGGGGGSGGGGSGGGGSEVQLQQSGPVLVKPGASVKISCKASGYSFTGYYMHWVRQSNGKCLEWIGRINPYTGVPSYKHNFKDKASLTVDKSSSTAYMELHSLTSEDSAVYYCARELGGYWGQGTTLTVSS

SEQ ID NO: 95 - protein, 239 aa, synthetic construct
  REGION 1..239: note = Description of sequence: An anti-human mesothelin scFv (vL-vH) polypeptide related to the monoclonal antibody 15B6 with a disulfide linkage
  DISULFID 103..168: note = Cysteine-cysteine disulfide bond
QAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGCTKLTVLGGGGGSGGGGSGGGGSEVQLQQSGPVLVKPGASVKISCKASGYSFTGYYMHWVRQSNGCSLEWIGRINPYTGVPSYKHNFKDKASLTVDKSSSTAYMELHSLTSEDSAVYYCARELGGYWGQGTTLTVSS

SEQ ID NO: 96 - protein, 238 aa, synthetic construct
  REGION 1..238: note = Description of sequence: An anti-human mesothelin scFv (vL-vH) polypeptide related to the monoclonal antibody 15B6 with a disulfide linkage
  DISULFID 103..169: note = Cysteine-cysteine disulfide bond
QAVVTQESALTTSPGETVTLTCRSSTGAVTTGNYPNWVQEKPDHLFTGLIAGTNNRAPGVPARFSGSLIGDKAALTITGAQTEDEAIYFCALWFSSHWVFGGCTKLTVLGGGGGSGGGGSGGGGSEVQLQQSGPVLVKPGASVKISCKASGYSFTGYYMHWVRQSNGKCLEWIGRINPYTGVPSYKHNFKDKASLTVDKSSSTAYMELHSLTSEDSAVYYCARELGGYWGQGTTLTVS

SEQ ID NO: 97 - protein, 113 aa, synthetic construct
  REGION 1..113: note = Description of sequence: An anti-human mesothelin (VHH) polypeptide related to the monoclonal antibody SD1
QVQLVQSGGGLVQPGGSLRLSCAASDFDFAAYEMSWVRQAPGQGLEWVAIISHDGIDKYYTDSVKGRFTISRDNSKNTLYLQMNTLRAEDTATYYCLRLGAVGQGTLVTVSSS

SEQ ID NO: 98 - protein, 252 aa, synthetic construct
  REGION 1..252: note = Description of sequence: An anti-human EpCAM scFv (vH-vL) polypeptide related to the monoclonal antibody MT201
  VARIANT 129: note = May be either valine or glycine
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDMGWGSGWRPYYYYGMDVWGQGTTVTVSSGXGGSGGGGSGGGGSELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIKRTV

SEQ ID NO: 99 - protein, 252 aa, synthetic construct
  REGION 1..252: note = Description of sequence: An anti-human EpCAM scFv (vL-vH) polypeptide related to the monoclonal antibody MT201
  VARIANT 112: note = May be either valine or glycine
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIKRTVGXGGSGGGGSGGGGSEVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDMGWGSGWRPYYYYGMDVWGQGTTVTVSS

SEQ ID NO: 100 - protein, 242 aa, synthetic construct
  REGION 1..242: note = Description of sequence: An anti-human CTLA-4 scFv (vL-vH) polypeptide related to Ipilimumab
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 101 - protein, 242 aa, synthetic construct
  REGION 1..242: note = Description of sequence: An anti-human CTLA-4 scFv (vL-vH) polypeptide related to Ipilimumab with a disulfide linkage
  DISULFID 101..167: note = Cysteine-cysteine disulfide bond
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGCGTKVEIKRGGGGSGGGGSGGGGSQVQLVESGGGVVQPGRSLRLSCAASGFTFSSYTMHWVRQAPGCGLEWVTFISYDGNNKYYADSVKGRFTISRDNSKNTLYLQMNSLRAEDTAIYYCARTGWLGPFDYWGQGTLVTVSS

SEQ ID NO: 102 - protein, 242 aa, synthetic construct
  REGION 1..242: note = Description of sequence: An anti-human CTLA-4 scFv (vL-vH) polypeptide related to Ipilimumab with a disulfide linkage
  DISULFID 101..168: note = Cysteine-cysteine disulfide bond
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGCGTKVEIKRGGGGSGGGGSGGGGSQVQLVESGGGVVQPGRSLRLSCAASGFTFSSYTMHWVRQAPGKCLEWVTFISYDGNNKYYADSVKGRFTISRDNSKNTLYLQMNSLRAEDTAIYYCARTGWLGPFDYWGQGTLVTVSS

SEQ ID NO: 103 - protein, 242 aa, synthetic construct
  REGION 1..242: note = Description of sequence: An anti-human CTLA-4 scFv (vL-vH) polypeptide related to Ipilimumab with a disulfide linkage
  DISULFID 102..167: note = Cysteine-cysteine disulfide bond
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQCTKVEIKRGGGGSGGGGSGGGGSQVQLVESGGGVVQPGRSLRLSCAASGFTFSSYTMHWVRQAPGCGLEWVTFISYDGNNKYYADSVKGRFTISRDNSKNTLYLQMNSLRAEDTAIYYCARTGWLGPFDYWGQGTLVTVSS

SEQ ID NO: 104 - protein, 242 aa, synthetic construct
  REGION 1..242: note = Description of sequence: An anti-human CTLA-4 scFv (vL-vH) polypeptide related to Ipilimumab with a disulfide linkage
  DISULFID 102..168: note = Cysteine-cysteine disulfide bond
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQCTKVEIKRGGGGSGGGGSGGGGSQVQLVESGGGVVQPGRSLRLSCAASGFTFSSYTMHWVRQAPGKCLEWVTFISYDGNNKYYADSVKGRFTISRDNSKNTLYLQMNSLRAEDTAIYYCARTGWLGPFDYWGQGTLVTVSS

SEQ ID NO: 105 - protein, 130 aa, Homo sapiens
  REGION 1..130: note = Description of sequence: An IL-2 polypeptide
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIIS

SEQ ID NO: 106 - protein, 201 aa, synthetic construct
  REGION 1..201: note = Description of sequence: a CD80 amino acid sequence
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTK

SEQ ID NO: 107 - protein, 222 aa, synthetic construct
  REGION 1..222: note = Description of sequence: CD86 (IgV-IgC)
LKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 108 - protein, 109 aa, synthetic construct
  REGION 1..109: note = Description of sequence: CD86 (IGV)
LKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLA

SEQ ID NO: 109 - protein, 518 aa, synthetic construct
  REGION 1..518: note = Description of sequence: Single-chain IL12
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 110 - protein, 306 aa, synthetic construct
  REGION 1..306: note = Description of sequence: IL12 component p40
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 111 - protein, 197 aa, synthetic construct
  REGION 1..197: note = Description of sequence: IL12 component p35
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 112 - protein, 146 aa, synthetic construct
  REGION 1..146: note = Description of sequence: a CD40L polypeptide
GDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 113 - protein, 468 aa, synthetic construct
  REGION 1..468: note = Description of sequence: a single-chain CD40L (sequence trimer)
GDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSGGGGSGGGGSGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSGGGGSGGGGSGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 114 - protein, 205 aa, synthetic construct
  REGION 1..205: note = Description of sequence: a 4-1BBL amino acid sequence
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 115 - protein, 166 aa, synthetic construct
  REGION 1..166: note = Description of sequence: a 4-1BBL amino acid sequence
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 116 - protein, 645 aa, synthetic construct
  REGION 1..645: note = Description of sequence: single chain 4-1BBL (trimer)
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 117 - protein, 247 aa, synthetic construct
  REGION 1..247: note = Description of sequence: anti-CD28 scFv
DIELTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIKRGGGGSGGGGSGGGGSQVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTTVTVSS

SEQ ID NO: 118 - protein, 261 aa, synthetic construct
  REGION 1..261: note = Description of sequence: A nucleotide binding domain polypeptide based on TAP1
MPPSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAPADAPE

SEQ ID NO: 119 - protein, 290 aa, synthetic construct
  REGION 1..290: note = Description of sequence: A nucleotide binding domain polypeptide based on TAP1
METEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAPADAPE

SEQ ID NO: 120 - protein, 302 aa, synthetic construct
  REGION 1..302: note = Description of sequence: Construct 24
MRGVPHIVMVDAYKRYKGGGGSGGGGSPPSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDDATSALDAGNQLRVQRLLYESPERYSRSVLLITQHLSLVEQADHILFLEGGAIREGGTHQQLMEKKGSYWAMVQAPADAPEENLYFQGHHHHHHHH

SEQ ID NO: 121 - protein, 310 aa, synthetic construct
  REGION 1..310: note = Description of sequence: Construct 103
METEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAPADAPEGGGGSENLYFQGHHHHHHHH

SEQ ID NO: 122 - protein, 298 aa, synthetic construct
  REGION 1..298: note = Description of sequence: Construct 167
METEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAPADAPEHHHHHHHH

SEQ ID NO: 123 - protein, 292 aa, synthetic construct
  REGION 1..292: note = Description of sequence: Construct 175
METEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 124 - protein, 290 aa, synthetic construct
  REGION 1..290: note = Description of sequence: Construct 176
METEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVHHHHHHHH

SEQ ID NO: 125 - protein, 288 aa, synthetic construct
  REGION 1..288: note = Description of sequence: Construct 177
METEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAHHHHHHHH

SEQ ID NO: 126 - protein, 286 aa, synthetic construct
  REGION 1..286: note = Description of sequence: Construct 178
METEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYHHHHHHHH

SEQ ID NO: 127 - protein, 284 aa, synthetic construct
  REGION 1..284: note = Description of sequence: Construct 179
METEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGHHHHHHHH

SEQ ID NO: 128 - protein, 310 aa, synthetic construct
  REGION 1..310: note = Description of sequence: Construct 180
  DISULFID 41..204: note = cystine-cystine bond
METEFFQQNQTGGGGSGLQTVRSFGGGGGSGGSGLLTPLHCEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPCVLILDNATSALDAGNQLRVQRLLYESPERYSRSVLLITQHLSLVEQADHILFLEGGAIREGGTHQQLMEKKGSYWAMVQAPADAPEGGGGSENLYFQGHHHHHHHH

SEQ ID NO: 129 - protein, 310 aa, synthetic construct
  REGION 1..310: note = Description of sequence: Construct 181
  DISULFID 203..235: note = cystine-cystine bond
METEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPCVLILDNATSALDAGNQLRVQRLLYESPERYCRSVLLITQHLSLVEQADHILFLEGGAIREGGTHQQLMEKKGSYWAMVQAPADAPEGGGGSENLYFQGHHHHHHHH

SEQ ID NO: 130 - protein, 310 aa, synthetic construct
  REGION 1..310: note = Description of sequence: Construct 182
  DISULFID 35..115: note = cystine-cystine bond
METEFFQQNQTGGGGSGLQTVRSFGGGGGSGGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAPADAPEGGGGSENLYFQGHHHHHHHH

SEQ ID NO: 131 - protein, 310 aa, synthetic construct
  REGION 1..310: note = Description of sequence: Construct 183
  DISULFID 36..115: note = cystine-cystine bond
METEFFQQNQTGGGGSGLQTVRSFGGGGGSGGSGLCTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAPADAPEGGGGSENLYFQGHHHHHHHH

SEQ ID NO: 132 - protein, 310 aa, synthetic construct
  REGION 1..310: note = Description of sequence: Construct 184
  DISULFID 134..178: note = cystine-cystine bond
METEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFCRSLQENIAYGLTQKPTMEEITAAAVKSGAHSFISGLPQGYDTECDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAPADAPEGGGGSENLYFQGHHHHHHHH

SEQ ID NO: 133 - protein, 310 aa, synthetic construct
  REGION 1..310: note = Description of sequence: Construct 185
  DISULFID 93..111: note = cystine-cystine bond
METEFFQQNQTGGGGSGLQTVRSFGGGGGSGGSGLLTPLHLEGLVQFQDVSFAYPNRPDVLVLQGLTFTLRPGEVTALVGPNGSGKSTVAALCQNLYQPTGGQLLLDGKPCPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAPADAPEGGGGSENLYFQGHHHHHHHH

SEQ ID NO: 134 - protein, 310 aa, synthetic construct
  REGION 1..310: note = Description of sequence: Construct 186
  DISULFID 95..111: note = cystine-cystine bond
METEFFQQNQTGGGGSGLQTVRSFGGGGGSGGSGLLTPLHLEGLVQFQDVSFAYPNRPDVLVLQGLTFTLRPGEVTALVGPNGSGKSTVAALLQCLYQPTGGQLLLDGKPCPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAPADAPEGGGGSENLYFQGHHHHHHHH

SEQ ID NO: 135 - protein, 274 aa, synthetic construct
  REGION 1..274: note = Description of sequence: Construct 211
METEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGHHHHHHHH

SEQ ID NO: 136 - protein, 274 aa, synthetic construct
  REGION 1..274: note = Description of sequence: Construct 222
  DISULFID 35..115: note = cystine-cystine bond
METEFFQQNQTGGGGSGLQTVRSFGGGGGSGGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGHHHHHHHH

SEQ ID NO: 137 - protein, 274 aa, synthetic construct
  REGION 1..274: note = Description of sequence: Construct 223
METEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIRAGGHHHHHHHH

SEQ ID NO: 138 - protein, 274 aa, synthetic construct
  REGION 1..274: note = Description of sequence: Construct 224
  DISULFID 35..115: note = cystine-cystine bond
METEFFQQNQTGGGGSGLQTVRSFGGGGGSGGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIRAGGHHHHHHHH

SEQ ID NO: 139 - protein, 292 aa, synthetic construct
  REGION 1..292: note = Description of sequence: Construct 232
METEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 140 - protein, 292 aa, synthetic construct
  REGION 1..292: note = Description of sequence: Construct 233
  DISULFID 35..115: note = cystine-cystine bond
METEFFQQNQTGGGGSGLQTVRSFGGGGGSGGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 141 - protein, 292 aa, synthetic construct
  REGION 1..292: note = Description of sequence: Construct 234
  DISULFID 35..115: note = cystine-cystine bond
METEFFQQNQTGGGGSGLQTVRSFGGGGGSGGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 142 - protein, 290 aa, synthetic construct
  REGION 1..290: note = Description of sequence: Construct 268
  DISULFID 33..113: note = cystine-cystine bond
METEFFQQNQTGGGGSGLQTVRSFGGGGGSGGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 143 - protein, 288 aa, synthetic construct
  REGION 1..288: note = Description of sequence: Construct 269
  DISULFID 31..111: note = cystine-cystine bond
METEFFQQNQTGGGGSGLQTVRSFGGGGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 144 - protein, 294 aa, synthetic construct
  REGION 1..294: note = Description of sequence: Construct 270
  DISULFID 37..117: note = cystine-cystine bond
METEFFQQNQTGGGGSGLQTVRSFGGGGGSGGGGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 145 - protein, 296 aa, synthetic construct
  REGION 1..296: note = Description of sequence: Construct 271
  DISULFID 39..119: note = cystine-cystine bond
METEFFQQNQTGGGGSGLQTVRSFGGGGGSGGGGSGGGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 146 - protein, 292 aa, synthetic construct
  REGION 1..292: note = Description of sequence: Construct 272
  DISULFID 35..115: note = cystine-cystine bond
METEFFQQNQTGGGGSGLQTVRSFGGGGGSGGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAICEGGTHQQLMEKKGCYWAMVQAHHHHHHHH

SEQ ID NO: 147 - protein, 292 aa, synthetic construct
  REGION 1..292: note = Description of sequence: Construct 273
  DISULFID 35..115: note = cystine-cystine bond
  DISULFID 264..277: note = cystine-cystine bond
METEFFQQNQTGGGGSGLQTVRSFGGGGGSGGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIRCGGTHQQLMEKKGCYWAMVQAHHHHHHHH

SEQ ID NO: 148 - protein, 310 aa, synthetic construct
  REGION 1..310: note = Description of sequence: Construct 288
  DISULFID 35..115: note = cystine-cystine bond
METEFFQQNQTGGGGSGLQTVRSFGGGGGSGGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQEPQVFGRSLQENIAYGLTQKPTMEEITAAAVKSGAHSFISGLPQGYDTEVDEAGSQLSGGQRQAVALARALIRKPSVLILDQATSALDAGNQLRVQRLLYESPERYSRSVLLITQHLSLVEQADHILFLEGGAIREGGTHQQLMEKKGSYWAMVQAPADAPEGGGGSENLYFQGHHHHHHHH

SEQ ID NO: 149 - protein, 310 aa, synthetic construct
  REGION 1..310: note = Description of sequence: Construct 289
  DISULFID 35..115: note = cystine-cystine bond
  DISULFID 122..204: note = cystine-cystine bond
METEFFQQNQTGGGGSGLQTVRSFGGGGGSGGSGCLTPLHLEGLVQFQDVSFAYPNRPDVLVLQGLTFTLRPGEVTALVGPNGSGKSTVAALLQNLYQPTGGQLLLDGKPLPQYCHRYLHRCVAAVGQEPQVFGRSLQENIAYGLTQKPTMEEITAAAVKSGAHSFISGLPQGYDTEVDEAGSQLSGGQRQAVALARALIRKPCVLILDNATSALDAGNQLRVQRLLYESPERYSRSVLLITQHLSLVEQADHILFLEGGAIREGGTHQQLMEKKGSYWAMVQAPADAPEGGGGSENLYFQGHHHHHHHH

SEQ ID NO: 150 - protein, 310 aa, synthetic construct
  REGION 1..310: note = Description of sequence: Construct 290
  DISULFID 35..115: note = cystine-cystine bond
  DISULFID 204..236: note = cystine-cystine bond
METEFFQQNQTGGGGSGLQTVRSFGGGGGSGGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQEPQVFGRSLQENIAYGLTQKPTMEEITAAAVKSGAHSFISGLPQGYDTEVDEAGSQLSGGQRQAVALARALIRKPCVLILDNATSALDAGNQLRVQRLLYESPERYSCSVLLITQHLSLVEQADHILFLEGGAIREGGTHQQLMEKKGSYWAMVQAPADAPEGGGGSENLYFQGHHHHHHHH

SEQ ID NO: 151 - protein, 292 aa, synthetic construct
  REGION 1..292: note = Description of sequence: Construct 294
  DISULFID 35..115: note = cystine-cystine bond
METEFFQQNQTGGGGSGLQTVRSFGGGGGSGGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVGEKGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 152 - protein, 292 aa, synthetic construct
  REGION 1..292: note = Description of sequence: Construct 296
  DISULFID 35..115: note = cystine-cystine bond
METEFFQQNQTGGGGSGGGGVRSFGGGGGSGGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 153 - protein, 294 aa, synthetic construct
  REGION 1..294: note = Description of sequence: Construct 297
  DISULFID 37..117: note = cystine-cystine bond
METEFFQQNQTGGGGSGGGLQTVRSFGGGGGSGGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 154 - protein, 287 aa, synthetic construct
  REGION 1..287: note = Description of sequence: Construct 302
  DISULFID 30..110: note = cystine-cystine bond
METEFFQQNQTGGGGSGLQTVRSFGGGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 155 - protein, 285 aa, synthetic construct
  REGION 1..285: note = Description of sequence: Construct 303
  DISULFID 28..108: note = cystine-cystine bond
METEFFQQNQTGGGGSGLQTVRSFGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 156 - protein, 289 aa, synthetic construct
  REGION 1..289: note = Description of sequence: Construct 304
  DISULFID 32..112: note = cystine-cystine bond
METEFFQQNQTGGGLQTVRSFGGGGGSGGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 157 - protein, 289 aa, synthetic construct; identical to SEQ ID NO: 156
  REGION 1..289: note = Description of sequence: Construct 305
  DISULFID 33..113: note = cystine-cystine bond

SEQ ID NO: 158 - protein, 291 aa, synthetic construct
  REGION 1..291: note = Description of sequence: Construct 306
  DISULFID 34..114: note = cystine-cystine bond
METEFFQQNQTGGGGGLQTVRSFGGGGGSGGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 159 - protein, 292 aa, synthetic construct
  REGION 1..292: note = Description of sequence: Construct 307
  DISULFID 2..22: note = cystine-cystine bond
  DISULFID 35..115: note = cystine-cystine bond
MCTEFFQQNQTGGGGSGLQTVCSFGGGGGSGGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 160 - protein, 292 aa, synthetic construct
  REGION 1..292: note = Description of sequence: Construct 308
  DISULFID 4..55: note = cystine-cystine bond
  DISULFID 35..115: note = cystine-cystine bond
METCFFQQNQTGGGGSGLQTVRSFGGGGGSGGSGCLTPLHLEGLVQFQDVSFAYCNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 161 - protein, 292 aa, synthetic construct; identical to SEQ ID NO: 160
  REGION 1..292: note = Description of sequence: Construct 309
  DISULFID 35..115: note = cystine-cystine bond
  DISULFID 122..204: note = cystine-cystine bond
  DISULFID 263..277: note = cystine-cystine bond

SEQ ID NO: 162 - protein, 298 aa, synthetic construct
  REGION 1..298: note = Description of sequence: Construct 341
  DISULFID 4..55: note = cystine-cystine bond
  DISULFID 35..115: note = cystine-cystine bond
METCFFQQNQTGGGGSGLQTVRSFGGGGGSGGSGCLTPLHLEGLVQFQDVSFAYCNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAPADAPEHHHHHHHH

SEQ ID NO: 163 - protein, 292 aa, synthetic construct
  REGION 1..292: note = Description of sequence: Construct 342
  DISULFID 4..55: note = cystine-cystine bond
METCFFQQNQTGGGGSGLQTVRSFGGGGGSGGSGLLTPLHLEGLVQFQDVSFAYCNRPDVLVLQGLTFTLRPGEVTALVGPNGSGKSTVAALLQNLYQPTGGQLLLDGKPLPQYEHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 164 - protein, 297 aa, synthetic construct
  REGION 1..297: note = Description of sequence: Construct 343
  DISULFID 4..54: note = cystine-cystine bond
  DISULFID 34..114: note = cystine-cystine bond
METCFFQQNQTGAPAGLQTVRSFGGGGGSGGSGCLTPLHLEGLVQFQDVSFAYCNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAPADAPEHHHHHHHH

SEQ ID NO: 165 - protein, 297 aa, synthetic construct
  REGION 1..297: note = Description of sequence: Construct 344
  DISULFID 4..54: note = cystine-cystine bond
  DISULFID 34..114: note = cystine-cystine bond
METCFFQQNQTGPAPGLQTVRSFGGGGGSGGSGCLTPLHLEGLVQFQDVSFAYCNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAPADAPEHHHHHHHH

SEQ ID NO: 166 - protein, 285 aa, synthetic construct
  REGION 1..285: note = Description of sequence: Construct 352
  DISULFID 28..108: note = cystine-cystine bond
METEFFQQNQTGGGGSGLQTVRSFGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDQATSALDAGNQLRVQRLLYESPERYSRSVLLITQHLSLVEQADHILFLEGGAIREGGTHQQLMEKKGSYWAMVQAHHHHHHHH

SEQ ID NO: 167 - protein, 285 aa, synthetic construct
  REGION 1..285: note = Description of sequence: Construct 353
  DISULFID 25..105: note = cystine-cystine bond
METEFFQQNQTGGGGSGLQTVRSFGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDAATSALDAGNQLRVQRLLYESPERYSRSVLLITQHLSLVEQADHILFLEGGAIREGGTHQQLMEKKGSYWAMVQAHHHHHHHH

SEQ ID NO: 168 - protein, 285 aa, synthetic construct
  REGION 1..285: note = Description of sequence: Construct 354
  DISULFID 28..108: note = cystine-cystine bond
METEFFQQNQTGGGGSGLQTVRSFGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDQATSALDAGNQLRVQRLLYESPERYSRSVLLITHHLSLVEQADHILFLEGGAIREGGTHQQLMEKKGSYWAMVQAHHHHHHHH

SEQ ID NO: 169 - protein, 282 aa, synthetic construct
  REGION 1..282: note = Description of sequence: Construct 448
  DISULFID 28..108: note = cystine-cystine bond
METEFFQQNQTGGGLQTVRSFGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 170 - protein, 291 aa, synthetic construct
  REGION 1..291: note = Description of sequence: Construct 449
  DISULFID 28..108: note = cystine-cystine bond
METEFFQQNQTGGGGSGLQTVRSFGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAPADAPEHHHHHHHH

SEQ ID NO: 171 - protein, 270 aa, synthetic construct
  REGION 1..270: note = Description of sequence: Construct 450
  DISULFID 13..93: note = cystine-cystine bond
MGLQTVRSFGSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 172 - protein, 261 aa, synthetic construct
  REGION 1..261: note = Description of sequence: Construct 451
  DISULFID 4..84: note = cystine-cystine bond
MSGCLTPLHLEGLVQFQDVSFAYPNRPDVLVLQGLTFTLRPGEVTALVGPNGSGKSTVAALLQNLYQPTGGQLLLDGKPLPQYCHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 173 - protein, 250 aa, synthetic construct
  REGION 1..250: note = Description of sequence: Construct 452
MLVQFQDVSFAYPNRPDVLVLQGLTFTLRPGEVTALVGPNGSGKSTVAALLQNLYQPTGGQLLLDGKPLPQYEHRYLHRQVAAVGQRPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAHHHHHHHH

SEQ ID NO: 174 - protein, 239 aa, synthetic construct
  REGION 1..239: note = Description of sequence: An anti-CD3 VH-CH1 amino acid sequence
MEPWPLLLLFSLCSAGLVLGQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEP

SEQ ID NO: 175 - protein, 233 aa, synthetic construct
  REGION 1..233: note = Description of sequence: An anti-CD3 VL-Ck amino acid sequence
MEPWPLLLLFSLCSAGLVLGDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 176 - protein, 242 aa, synthetic construct
  REGION 1..242: note = Description of sequence: An anti-CD3 VH-CH1
MEPWPLLLLFSLCSAGLVLGQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYELVSVVTVPSSSLGTQTYICNVNHKPSNTKVDKKVEPKSC

SEQ ID NO: 177 - protein, 233 aa, synthetic construct
  REGION 1..233: note = Description of sequence: An anti-CD3 VL-Ck comprising MD13 sequence variations
MEPWPLLLLFSLCSAGLVLGDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLLSSLTLSKADYEKHKVYACEVTHQGLSSPVTKSFNRGEC

SEQ ID NO: 178 - protein, 260 aa, synthetic construct
  REGION 1..260: note = Description of sequence: An anti-mesothelin scFv aa sequence
MEPWPLLLLFSLCSAGLVLGQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSGVGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 179 - protein, 267 aa, synthetic construct
  REGION 1..267: note = Description of sequence: An anti-mesothelin scFv aa sequence
MEPWPLLLLFSLCSAGLVLGQOQLEESGGGLVKPEGSLTLTCKASGFDLGFYFYACWVRQAPGKGLEWIACIYTAGSGSTYYASWAKGRFTISKASSTTVTLQMTSLAAADTATYFCARSTANTRSTYLNLWGPGTLVTVSSGVGGSGGGGSGGGGSDVVMTQTPASVSEPVGGTVTIKCQASQRISSYLSWYOQKPGQRPKLLIFGASTLASGVPSRFKGSGSGTEYTLTISDLECADAATYYCOSYAYFDSNNWHAFGGGTEVVV

SEQ ID NO: 180 - protein, 119 aa, synthetic construct
  REGION 1..119: note = Description of sequence: An anti-BCMA VHH
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSS

SEQ ID NO: 181 - protein, 260 aa, synthetic construct
  REGION 1..260: note = Description of sequence: A nucleotide binding domain fragment from human TAP 1 modified with the rat D-helix aa sequence
PPSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDDATSALDAGNQLRVQRLLYESPERYSRSVLLITQHLSLVEQADHILFLEGGAIREGGTHQQLMEKKGSYWAMVQAPADAPE

SEQ ID NO: 182 - protein, 289 aa, synthetic construct
  REGION 1..289: note = Description of sequence: A nucleotide binding domain polypeptide based on TAP1 with a D668N substitution
ETEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAPADAPE

SEQ ID NO: 183 - protein, 556 aa, synthetic construct
  REGION 1..556: note = Description of sequence: Construct 156
MHHHHHHHHEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGSGGGGSQVQLVESGGGLVQPGGSLRLSCAASGSIFSANTMGWYRQAPGKQRELVAGMNTSGSTVYGDSVKGRFTISRDNAKNIAYLQMNSLIPEDTAVYYCTLVQRGPNYWGQGTQVTVSSGGGGSGGGGSETEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAPADAPEGGGGS

SEQ ID NO: 184 - protein, 556 aa, synthetic construct
  REGION 1..556: note = Description of sequence: Construct 157
MHHHHHHHHEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGSGGGGSETEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAPADAPEGGGGSGGGGSGGGGSQVQLVESGGGLVQPGGSLRLSCAASGSIFSANTMGWYRQAPGKQRELVAGMNTSGSTVYGDSVKGRFTISRDNAKNIAYLQMNSLIPEDTAVYYCTLVQRGPNYWGQGTQVTVSS

SEQ ID NO: 185 - protein, 556 aa, synthetic construct
  REGION 1..556: note = Description of sequence: Construct 158
MHHHHHHHHETEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAPADAPEGGGGSGGGGSGGGGSQVQLVESGGGLVQPGGSLRLSCAASGSIFSANTMGWYRQAPGKQRELVAGMNTSGSTVYGDSVKGRFTISRDNAKNIAYLQMNSLIPEDTAVYYCTLVQRGPNYWGQGTQVTVSSGGGGSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSS

SEQ ID NO: 186 - protein, 556 aa, synthetic construct
  REGION 1..556: note = Description of sequence: Construct 159
METEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDNATSALDAGNQLRVQRLLYESPERYSRSVLLITQHLSLVEQADHILFLEGGAIREGGTHQQLMEKKGSYWAMVQAPADAPEGGGGSGGGGSGGGGSQVQLVESGGGLVQPGGSLRLSCAASGSIFSANTMGWYRQAPGKQRELVAGMNTSGSTVYGDSVKGRFTISRDNAKNIAYLQMNSLIPEDTAVYYCTLVQRGPNYWGQGTQVTVSSGGGGSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSHHHHHHHH

SEQ ID NO: 187 - protein, 476 aa, synthetic construct
  REGION 1..476: note = Description of sequence: Construct No: 533; Description: AchRss-hIgG1 Fc(wt L234, L235; hole)-(G4S)2-SS1(anti-MSLN scFv)
  REGION 1..226: note = wt L234 L235; hole
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 188 - protein, 512 aa, synthetic construct
  REGION 1..512: note = Description of sequence: Construct No: 571; Description: AchRss-CH1-G4S-CH2-(NL)-TAP1 NBD (modified D-helix; L493C:E573C; S542A; C662S; D668Q; C735S; del PADAPE)-(G4S)2-hIgG1 Fc(wt L234, L235; knob)
  REGION 1..24: note = CAP sequence: AchRss-CH1-G4S-CH2-(NL)
  REGION 25..276: note = modified D-helix; L493C:E573C; S542A; C662S; D668Q; C735S; del PADAPE
  REGION 277..286: note = Linker
  REGION 287..512: note = wt L234 L235; knob
ETEFFQQNQTGGGGSGLQTVRSFGSGCLTPLHLEGLVQFQDVSFAYPNRPDVLVLQGLTFTLRPGEVTALVGPNGAGKSTVAALLQNLYQPTGGQLLLDGKPLPQYCHRYLHRQVAAVGQEPQVFGRSLQENIAYGLTQKPTMEEITAAAVKSGAHSFISGLPQGYDTEVDEAGSQLSGGQRQAVALARALIRKPSVLILDQATSALDAGNQLRVQRLLYESPERYSRSVLLITQHLSLVEQADHILFLEGGAIREGGTHQQLMEKKGSYWAMVQAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 189 - protein, 512 aa, synthetic construct
  REGION 1..512: note = Description of sequence: Construct No: 647; Description: AchRss-571TAP(D667A)-(G4S)2-hIgG1 Fc(wt L234, L235; knob)
  REGION 1..24: note = CAP sequence: AchRss
  REGION 25..276: note = TAP sequence: D667A
  REGION 277..286: note = Linker
  REGION 287..512: note = wt L234 L235; knob
ETEFFQQNQTGGGGSGLQTVRSFGSGCLTPLHLEGLVQFQDVSFAYPNRPDVLVLQGLTFTLRPGEVTALVGPNGAGKSTVAALLQNLYQPTGGQLLLDGKPLPQYCHRYLHRQVAAVGQEPQVFGRSLQENIAYGLTQKPTMEEITAAAVKSGAHSFISGLPQGYDTEVDEAGSQLSGGQRQAVALARALIRKPSVLILAQATSALDAGNQLRVQRLLYESPERYSRSVLLITQHLSLVEQADHILFLEGGAIREGGTHQQLMEKKGSYWAMVQAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 190 - protein, 512 aa, synthetic construct
  REGION 1..512: note = Description of sequence: Construct No: 648; Description: AchRss-571TAP(Q586A)-(G4S)2-hIgG1 Fc(wt L234, L235; knob)
  REGION 1..24: note = CAP sequence: AchRss
  REGION 25..276: note = TAP Sequence: Q586A
  REGION 277..286: note = Linker
  REGION 287..512: note = wt L234 L235; knob
ETEFFQQNQTGGGGSGLQTVRSFGSGCLTPLHLEGLVQFQDVSFAYPNRPDVLVLQGLTFTLRPGEVTALVGPNGAGKSTVAALLQNLYQPTGGQLLLDGKPLPQYCHRYLHRQVAAVGAEPQVFGRSLQENIAYGLTQKPTMEEITAAAVKSGAHSFISGLPQGYDTEVDEAGSQLSGGQRQAVALARALIRKPSVLILDQATSALDAGNQLRVQRLLYESPERYSRSVLLITQHLSLVEQADHILFLEGGAIREGGTHQQLMEKKGSYWAMVQAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 191 - protein, 20 aa, synthetic construct
  REGION 1..20: note = Description of sequence: acetylcholine receptor leader sequence
MEPWPLLLLFSLCSAGLVLG

SEQ ID NO: 192 - protein, 748 aa, synthetic construct
  REGION 1..748: note = Description of sequence: Construct No: 436; Description: AchRss-CH1-G3S-CH2-(NL)-TAP1 NBD (modified D-helix; L493C:E573C; Q580C:C662; E587R; D668Q; C735S; del PADAPE)-(G4S)2-scFc(G4S)6
  REGION 1..24: note = CAP Sequence: AchRss-CH1-G3S-CH2-(NL)
  REGION 25..276: note = modified D-helix; L493C:E573C; Q580C:C662; E587R; D668Q; C735S; del PADAPE
  REGION 277..286: note = Linker
  REGION 287..748: note = G4S
ETEFFQQNQTGGGGSGLQTVRSFGSGCLTPLHLEGLVQFQDVSFAYPNRPDVLVLQGLTFTLRPGEVTALVGPNGSGKSTVAALLQNLYQPTGGQLLLDGKPLPQYCHRYLHRCVAAVGQRPQVFGRSLQENIAYGLTQKPTMEEITAAAVKSGAHSFISGLPQGYDTEVDEAGSQLSGGQRQAVALARALIRKPCVLILDQATSALDAGNQLRVQRLLYESPERYSRSVLLITQHLSLVEQADHILFLEGGAIREGGTHQQLMEKKGSYWAMVQAGGGGSGGGGSAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 193 - protein, 226 aa, synthetic construct
  REGION 1..226: note = Description of sequence: Construct No: 476; Description: AchRss-hIgG1 Fc(wt L234, L235; hole)
  REGION 1..226: note = wt L234 L235; hole
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 194 - protein, 792 aa, synthetic construct
  REGION 1..792: note = Description of sequence: Construct No: 586; Description: hIgG1 Fc(L234A, L235A; knob)-(G4S)3- sincle chain (sc) trimer of 41BBL (three 41BBL sequence separated by (G4S)5 linkers) ( sct41BBL )
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGGGGSGGGGSGGGGSGGGGSGGGGSDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGGGGSGGGGSGGGGSGGGGSGGGGSDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 195 - protein, 792 aa, synthetic construct
  REGION 1..792: note = Description of sequence: Construct No: 587; Description: AchRss-hIgG1 Fc(L234A, L235A; hole)-(G4S)3-sct41BBL
  REGION 1..226: note = wt L234 L235; hole
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGGGGSGGGGSGGGGSGGGGSGGGGSDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGGGGSGGGGSGGGGSGGGGSGGGGSDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 196 - protein, 512 aa, synthetic construct
  REGION 1..512: note = Description of sequence: Construct No: 590; Description: AchRss-CH1-G4S-CH2-(NL)-TAP1 NBD (modified D-helix; L493C:E573C; S542A; C662S; D668Q; C735S; del PADAPE)-(G4S)2-hIgG1 Fc( L234A, L235A; knob)
  REGION 1..24: note = CAP Sequence: AchRss-hIgG1
  REGION 25..276: note = modified D-helix; L493C:E573C; S542A; C662S; D668Q; C735S; del PADAPE
  REGION 277..286: note = Linker
  REGION 287..512: note = wt L234 L235; knob
ETEFFQQNQTGGGGSGLQTVRSFGSGCLTPLHLEGLVQFQDVSFAYPNRPDVLVLQGLTFTLRPGEVTALVGPNGAGKSTVAALLQNLYQPTGGQLLLDGKPLPQYCHRYLHRQVAAVGQEPQVFGRSLQENIAYGLTQKPTMEEITAAAVKSGAHSFISGLPQGYDTEVDEAGSQLSGGQRQAVALARALIRKPSVLILDQATSALDAGNQLRVQRLLYESPERYSRSVLLITQHLSLVEQADHILFLEGGAIREGGTHQQLMEKKGSYWAMVQAGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 197 - protein, 226 aa, synthetic construct
  REGION 1..226: note = Description of sequence: Construct No: 473; Description: AchRss-hIgG1 Fc(L234A, L235A; knob)
  REGION 1..226: note = wt L234 L235; knob
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 198 - protein, 10 aa, synthetic construct
  REGION 1..10: note = Description of sequence: Linker
EAAAKEAAAK

SEQ ID NO: 199 - protein, 15 aa, synthetic construct
  REGION 1..15: note = Description of sequence: Linker
EAAAKEAAAKEAAAK

SEQ ID NO: 200 - protein, 20 aa, synthetic construct
  REGION 1..20: note = Description of sequence: Linker
EAAAKEAAAKEAAAKEAAAK

SEQ ID NO: 201 - protein, 25 aa, synthetic construct
  REGION 1..25: note = Description of sequence: Linker
EAAAKEAAAKEAAAKEAAAKEAAAK

SEQ ID NO: 202 - protein, 30 aa, synthetic construct
  REGION 1..30: note = Description of sequence: Linker
EAAAKEAAAKEAAAKEAAAKEAAAKEAAAK

SEQ ID NO: 203 - protein, 35 aa, synthetic construct
  REGION 1..35: note = Description of sequence: Linker
EAAAKEAAAKEAAAKEAAAKEAAAKEAAAKEAAAK

SEQ ID NO: 204 - protein, 40 aa, synthetic construct
  REGION 1..40: note = Description of sequence: Linker
EAAAKEAAAKEAAAKEAAAKEAAAKEAAAKEAAAKEAAAK

SEQ ID NO: 205 - protein, 45 aa, synthetic construct
  REGION 1..45: note = Description of sequence: Linker
EAAAKEAAAKEAAAKEAAAKEAAAKEAAAKEAAAKEAAAKEAAAK

SEQ ID NO: 206 - protein, 50 aa, synthetic construct
  REGION 1..50: note = Description of sequence: Linker
EAAAKEAAAKEAAAKEAAAKEAAAKEAAAKEAAAKEAAAKEAAAKEAAAK

SEQ ID NO: 207 - protein, 55 aa, synthetic construct
  REGION 1..55: note = Description of sequence: Linker
EAAAKEAAAKEAAAKEAAAKEAAAKEAAAKEAAAKEAAAKEAAAKEAAAKEAAAK

SEQ ID NO: 208 - protein, 60 aa, synthetic construct
  REGION 1..60: note = Description of sequence: Linker
EAAAKEAAAKEAAAKEAAAKEAAAKEAAAKEAAAKEAAAKEAAAKEAAAKEAAAKEAAAK

SEQ ID NO: 209 - protein, 65 aa, synthetic construct
  REGION 1..65: note = Description of sequence: Linker
EAAAKEAAAKEAAAKEAAAKEAAAKEAAAKEAAAKEAAAKEAAAKEAAAKEAAAKEAAAKEAAAK

SEQ ID NO: 210 - protein, 70 aa, synthetic construct
  REGION 1..70: note = Description of sequence: Linker
EAAAKEAAAKEAAAKEAAAKEAAAKEAAAKEAAAKEAAAKEAAAKEAAAKEAAAKEAAAKEAAAKEAAAK

SEQ ID NO: 211 - protein, 75 aa, synthetic construct
  REGION 1..75: note = Description of sequence: Linker
EAAAKEAAAKEAAAKEAAAKEAAAKEAAAKEAAAKEAAAKEAAAKEAAAKEAAAKEAAAKEAAAKEAAAKEAAAK

SEQ ID NO: 212 - protein, 80 aa, synthetic construct
  REGION 1..80: note = Description of sequence: Linker
EAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 213 - protein, 85 aa, synthetic construct
  REGION 1..85: note = Description of sequence: Linker
EAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 214 - protein, 90 aa, synthetic construct
  REGION 1..90: note = Description of sequence: Linker
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 215 - protein, 95 aa, synthetic construct
  REGION 1..95: note = Description of sequence: Linker
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 216 - protein, 100 aa, synthetic construct; identical to SEQ ID NO: 50
  REGION 1..100: note = Description of sequence: Linker

SEQ ID NO: 217 - protein, 4 aa, synthetic construct
  REGION 1..4: note = Description of sequence: Linker
KPKP

SEQ ID NO: 218 - protein, 6 aa, synthetic construct
  REGION 1..6: note = Description of sequence: Linker
KPKPKP

SEQ ID NO: 219 - protein, 8 aa, synthetic construct
  REGION 1..8: note = Description of sequence: Linker
KPKPKPKP

SEQ ID NO: 220 - protein, 10 aa, synthetic construct
  REGION 1..10: note = Description of sequence: Linker
KPKPKPKPKP

SEQ ID NO: 221 - protein, 12 aa, synthetic construct
  REGION 1..12: note = Description of sequence: Linker
KPKPKPKPKPKP

SEQ ID NO: 222 - protein, 14 aa, synthetic construct
  REGION 1..14: note = Description of sequence: Linker
KPKPKPKPKPKPKP

SEQ ID NO: 223 - protein, 16 aa, synthetic construct
  REGION 1..16: note = Description of sequence: Linker
KPKPKPKPKPKPKPKP

SEQ ID NO: 224 - protein, 18 aa, synthetic construct
  REGION 1..18: note = Description of sequence: Linker
KPKPKPKPKPKPKPKPKP

SEQ ID NO: 225 - protein, 20 aa, synthetic construct
  REGION 1..20: note = Description of sequence: Linker
KPKPKPKPKPKPKPKPKPKP

SEQ ID NO: 226 - protein, 22 aa, synthetic construct
  REGION 1..22: note = Description of sequence: Linker
KPKPKPKPKPKPKPKPKPKPKP

SEQ ID NO: 227 - protein, 24 aa, synthetic construct
  REGION 1..24: note = Description of sequence: Linker
KPKPKPKPKPKPKPKPKPKPKPKP

SEQ ID NO: 228 - protein, 26 aa, synthetic construct
  REGION 1..26: note = Description of sequence: Linker
KPKPKPKPKPKPKPKPKPKPKPKPKP

SEQ ID NO: 229 - protein, 28 aa, synthetic construct
  REGION 1..28: note = Description of sequence: Linker
KPKPKPKPKPKPKPKPKPKPKPKPKPKP

SEQ ID NO: 230 - protein, 30 aa, synthetic construct
  REGION 1..30: note = Description of sequence: Linker
KPKPKPKPKPKPKPKPKPKPKPKPKPKPKP

SEQ ID NO: 231 - protein, 32 aa, synthetic construct
  REGION 1..32: note = Description of sequence: Linker
KPKPKPKPKPKPKPKPKPKPKPKPKPKPKPKP

SEQ ID NO: 232 - protein, 34 aa, synthetic construct
  REGION 1..34: note = Description of sequence: Linker
KPKPKPKPKPKPKPKPKPKPKPKPKPKPKPKPKP

SEQ ID NO: 233 - protein, 36 aa, synthetic construct
  REGION 1..36: note = Description of sequence: Linker
KPKPKPKPKPKPKPKPKPKPKPKPKPKPKPKPKPKP

SEQ ID NO: 234 - protein, 38 aa, synthetic construct
  REGION 1..38: note = Description of sequence: Linker
KPKPKPKPKPKPKPKPKPKPKPKPKPKPKPKPKPKPKP

SEQ ID NO: 235 - protein, 40 aa, synthetic construct; identical to SEQ ID NO: 51
  REGION 1..40: note = Description of sequence: Linker

SEQ ID NO: 236 - protein, 4 aa, synthetic construct
  REGION 1..4: note = Description of sequence: Linker
EPEP

SEQ ID NO: 237 - protein, 6 aa, synthetic construct
  REGION 1..6: note = Description of sequence: Linker
EPEPEP

SEQ ID NO: 238 - protein, 8 aa, synthetic construct
  REGION 1..8: note = Description of sequence: Linker
EPEPEPEP

SEQ ID NO: 239 - protein, 10 aa, synthetic construct
  REGION 1..10: note = Description of sequence: Linker
EPEPEPEPEP

SEQ ID NO: 240 - protein, 12 aa, synthetic construct
  REGION 1..12: note = Description of sequence: Linker
EPEPEPEPEPEP

SEQ ID NO: 241 - protein, 14 aa, synthetic construct
  REGION 1..14: note = Description of sequence: Linker
EPEPEPEPEPEPEP

SEQ ID NO: 242 - protein, 16 aa, synthetic construct
  REGION 1..16: note = Description of sequence: Linker
EPEPEPEPEPEPEPEP

SEQ ID NO: 243 - protein, 18 aa, synthetic construct
  REGION 1..18: note = Description of sequence: Linker
EPEPEPEPEPEPEPEPEP

SEQ ID NO: 244 - protein, 20 aa, synthetic construct
  REGION 1..20: note = Description of sequence: Linker
EPEPEPEPEPEPEPEPEPEP

SEQ ID NO: 245 - protein, 22 aa, synthetic construct
  REGION 1..22: note = Description of sequence: Linker
EPEPEPEPEPEPEPEPEPEPEP

SEQ ID NO: 246 - protein, 24 aa, synthetic construct
  REGION 1..24: note = Description of sequence: Linker
EPEPEPEPEPEPEPEPEPEPEPEP

SEQ ID NO: 247 - protein, 26 aa, synthetic construct
  REGION 1..26: note = Description of sequence: Linker
EPEPEPEPEPEPEPEPEPEPEPEPEP

SEQ ID NO: 248 - protein, 28 aa, synthetic construct
  REGION 1..28: note = Description of sequence: Linker
EPEPEPEPEPEPEPEPEPEPEPEPEPEP

SEQ ID NO: 249 - protein, 30 aa, synthetic construct
  REGION 1..30: note = Description of sequence: Linker
EPEPEPEPEPEPEPEPEPEPEPEPEPEPEP

SEQ ID NO: 250 - protein, 32 aa, synthetic construct
  REGION 1..32: note = Description of sequence: Linker
EPEPEPEPEPEPEPEPEPEPEPEPEPEPEPEP

SEQ ID NO: 251 - protein, 34 aa, synthetic construct
  REGION 1..34: note = Description of sequence: Linker
EPEPEPEPEPEPEPEPEPEPEPEPEPEPEPEPEP

SEQ ID NO: 252 - protein, 36 aa, synthetic construct
  REGION 1..36: note = Description of sequence: Linker
EPEPEPEPEPEPEPEPEPEPEPEPEPEPEPEPEPEP

SEQ ID NO: 253 - protein, 38 aa, synthetic construct
  REGION 1..38: note = Description of sequence: Linker
EPEPEPEPEPEPEPEPEPEPEPEPEPEPEPEPEPEPEP

SEQ ID NO: 254 - protein, 40 aa, synthetic construct; identical to SEQ ID NO: 52
  REGION 1..40: note = Description of sequence: Linker

SEQ ID NO: 255 - protein, 42 aa, synthetic construct
  REGION 1..42: note = Description of sequence: Linker
EPEPEPEPEPEPEPEPEPEPEPEPEPEPEPEPEPEPEPEPEP

SEQ ID NO: 256 - protein, 6 aa, synthetic construct
  REGION 1..6: note = Description of sequence: Linker
TPRTPR

SEQ ID NO: 257 - protein, 9 aa, synthetic construct
  REGION 1..9: note = Description of sequence: Linker
TPRTPRTPR

SEQ ID NO: 258 - protein, 12 aa, synthetic construct
  REGION 1..12: note = Description of sequence: Linker
TPRTPRTPRTPR

SEQ ID NO: 259 - protein, 15 aa, synthetic construct
  REGION 1..15: note = Description of sequence: Linker
TPRTPRTPRTPRTPR

SEQ ID NO: 260 - protein, 18 aa, synthetic construct
  REGION 1..18: note = Description of sequence: Linker
TPRTPRTPRTPRTPRTPR

SEQ ID NO: 261 - protein, 21 aa, synthetic construct
  REGION 1..21: note = Description of sequence: Linker
TPRTPRTPRTPRTPRTPRTPR

SEQ ID NO: 262 - protein, 24 aa, synthetic construct
  REGION 1..24: note = Description of sequence: Linker
TPRTPRTPRTPRTPRTPRTPRTPR

SEQ ID NO: 263 - protein, 27 aa, synthetic construct
  REGION 1..27: note = Description of sequence: Linker
TPRTPRTPRTPRTPRTPRTPRTPRTPR

SEQ ID NO: 264 - protein, 30 aa, synthetic construct
  REGION 1..30: note = Description of sequence: Linker
TPRTPRTPRTPRTPRTPRTPRTPRTPRTPR

SEQ ID NO: 265 - protein, 33 aa, synthetic construct
  REGION 1..33: note = Description of sequence: Linker
TPRTPRTPRTPRTPRTPRTPRTPRTPRTPRTPR

SEQ ID NO: 266 - protein, 36 aa, synthetic construct
  REGION 1..36: note = Description of sequence: Linker
TPRTPRTPRTPRTPRTPRTPRTPRTPRTPRTPRTPR

SEQ ID NO: 267 - protein, 39 aa, synthetic construct
  REGION 1..39: note = Description of sequence: Linker
TPRTPRTPRTPRTPRTPRTPRTPRTPRTPRTPRTPRTPR

SEQ ID NO: 268 - protein, 42 aa, synthetic construct
  REGION 1..42: note = Description of sequence: Linker
TPRTPRTPRTPRTPRTPRTPRTPRTPRTPRTPRTPRTPRTPR

SEQ ID NO: 269 - protein, 45 aa, synthetic construct
  REGION 1..45: note = Description of sequence: Linker
TPRTPRTPRTPRTPRTPRTPRTPRTPRTPRTPRTPRTPRTPRTPR

SEQ ID NO: 270 - protein, 48 aa, synthetic construct
  REGION 1..48: note = Description of sequence: Linker
TPRTPRTPRTPRTPRTPRTPRTPRTPRTPRTPRTPRTPRTPRTPRTPR

SEQ ID NO: 271 - protein, 51 aa, synthetic construct
  REGION 1..51: note = Description of sequence: Linker
TPRTPRTPRTPRTPRTPRTPRTPRTPRTPRTPRTPRTPRTPRTPRTPRTPR

SEQ ID NO: 272 - protein, 54 aa, synthetic construct
  REGION 1..54: note = Description of sequence: Linker
TPRTPRTPRTPRTPRTPRTPRTPRTPRTPRTPRTPRTPRTPRTPRTPRTPRTPR

SEQ ID NO: 273 - protein, 57 aa, synthetic construct
  REGION 1..57: note = Description of sequence: Linker
TPRTPRTPRTPRTPRTPRTPRTPRTPRTPRTPRTPRTPRTPRTPRTPRTPRTPRTPR

SEQ ID NO: 274 - protein, 60 aa, synthetic construct; identical to SEQ ID NO: 53
  REGION 1..60: note = Description of sequence: Linker

SEQ ID NO: 275 - protein, 22 aa, synthetic construct
  REGION 1..22: note = Description of sequence: Linker
APAPAPAPAPAPAPAPAPAPAP

SEQ ID NO: 276 - protein, 24 aa, synthetic construct
  REGION 1..24: note = Description of sequence: Linker
APAPAPAPAPAPAPAPAPAPAPAP

SEQ ID NO: 277 - protein, 26 aa, synthetic construct
  REGION 1..26: note = Description of sequence: Linker
APAPAPAPAPAPAPAPAPAPAPAPAP

SEQ ID NO: 278 - protein, 28 aa, synthetic construct
  REGION 1..28: note = Description of sequence: Linker
APAPAPAPAPAPAPAPAPAPAPAPAPAP

SEQ ID NO: 279 - protein, 30 aa, synthetic construct
  REGION 1..30: note = Description of sequence: Linker
APAPAPAPAPAPAPAPAPAPAPAPAPAPAP

SEQ ID NO: 280 - protein, 32 aa, synthetic construct
  REGION 1..32: note = Description of sequence: Linker
APAPAPAPAPAPAPAPAPAPAPAPAPAPAPAP

SEQ ID NO: 281 - protein, 34 aa, synthetic construct
  REGION 1..34: note = Description of sequence: Linker
APAPAPAPAPAPAPAPAPAPAPAPAPAPAPAPAP

SEQ ID NO: 282 - protein, 36 aa, synthetic construct
  REGION 1..36: note = Description of sequence: Linker
APAPAPAPAPAPAPAPAPAPAPAPAPAPAPAPAPAP

SEQ ID NO: 283 - protein, 38 aa, synthetic construct
  REGION 1..38: note = Description of sequence: Linker
APAPAPAPAPAPAPAPAPAPAPAPAPAPAPAPAPAPAP

SEQ ID NO: 284 - protein, 40 aa, synthetic construct; identical to SEQ ID NO: 54
  REGION 1..40: note = Description of sequence: Linker

SEQ ID NO: 285 - protein, 651 aa, synthetic construct
  REGION 1..651: note = Description of sequence: cognate binding partner, ABCG5
MGDLSSLTPGGSMGLQVNRGSQSSLEGAPATAPEPHSLGILHASYSVSHRVRPWWDITSCRQQWTRQILKDVSLYVESGQIMCILGSSGSGKTTLLDAMSGRLGRAGTFLGEVYVNGRALRREQFQDCFSYVLQSDTLLSSLTVRETLHYTALLAIRRGNPGSFQKKVEAVMAELSLSHVADRLIGNYSLGGISTGERRRVSIAAQLLQDPKVMLFDEPTTGLDCMTANQIVVLLVELARRNRIVVLTIHQPRSELFQLFDKIAILSFGELIFCGTPAEMLDFFNDCGYPCPEHSNPFDFYMDLTSVDTQSKEREIETSKRVQMIESAYKKSAICHKTLKNIERMKHLKTLPMVPFKTKDSPGVFSKLGVLLRRVTRNLVRNKLAVITRLLQNLIMGLFLLFFVLRVRSNVLKGAIQDRVGLLYQFVGATPYTGMLNAVNLFPVLRAVSDQESQDGLYQKWQMMLAYALHVLPFSVVATMIFSSVCYWTLGLHPEVARFGYFSAALLAPHLIGEFLTLVLLGIVQNPNIVNSVVALLSIAGVLVGSGFLRNIQEMPIPFKIISYFTFQKYCSEILVVNEFYGLNFTCGSSNVSVTTNPMCAFTQGIQFIEKTCPGATSRFTMNFLILYSFIPALVILGIVVFKIRDHLISR

SEQ ID NO: 286 - protein, 242 aa, synthetic construct
  REGION 1..242: note = Description of sequence: Nucleotide binding domain for ABCG5
RPWWDITSCRQQWTRQILKDVSLYVESGQIMCILGSSGSGKTTLLDAMSGRLGRAGTFLGEVYVNGRALRREQFQDCFSYVLQSDTLLSSLTVRETLHYTALLAIRRGNPGSFQKKVEAVMAELSLSHVADRLIGNYSLGGISTGERRRVSIAAQLLQDPKVMLFDEPTTGLDCMTANQIVVLLVELARRNRIVVLTIHQPRSELFQLFDKIAILSFGELIFCGTPAEMLDFFNDCGYPCPE

SEQ ID NO: 287 - protein, 165 aa, synthetic construct
  REGION 1..165: note = Description of sequence: Nucleotide binding domain for ABCG5
GSSGSGKTTLLDAMSGRLGRAGTFLGEVYVNGRALRREQFQDCFSYVLQSDTLLSSLTVRETLHYTALLAIRRGNPGSFQKKVEAVMAELSLSHVADRLIGNYSLGGISTGERRRVSIAAQLLQDPKVMLFDEPTTGLDCMTANQIVVLLVELARRNRIVVLTIH

SEQ ID NO: 288 - protein, 673 aa, synthetic construct
  REGION 1..673: note = Description of sequence: cognate binding partner, ABCG8
MAGKAAEERGLPKGATPQDTSGLQDRLFSSESDNSLYFTYSGQPNTLEVRDLNYQVDLASQVPWFEQLAQFKMPWTSPSCQNSCELGIQNLSFKVRSGQMLAIIGSSGCGRASLLDVITGRGHGGKIKSGQIWINGQPSSPQLVRKCVAHVRQHNQLLPNLTVRETLAFIAQMRLPRTFSQAQRDKRVEDVIAELRLRQCADTRVGNMYVRGLSGGERRRVSIGVQLLWNPGILILDEPTSGLDSFTAHNLVKTLSRLAKGNRLVLISLHQPRSDIFRLFDLVLLMTSGTPIYLGAAQHMVQYFTAIGYPCPRYSNPADFYVDLTSIDRRSREQELATREKAQSLAALFLEKVRDLDDFLWKAETKDLDEDTCVESSVTPLDTNCLPSPTKMPGAVQQFTTLIRRQISNDFRDLPTLLIHGAEACLMSMTIGFLYFGHGSIQLSFMDTAALLFMIGALIPFNVILDVISKCYSERAMLYYELEDGLYTTGPYFFAKILGELPEHCAYIIIYGMPTYWLANLRPGLQPFLLHFLLVWLVVFCCRIMALAAAALLPTFHMASFFSNALYNSFYLAGGFMINLSSLWTVPAWISKVSFLRWCFEGLMKIQFSRRTYKMPLGNLTIAVSGDKILSVMELDSYPLYAIYLIVIGLSGGFMVLYYVSLRFIKQKPSQDW

SEQ ID NO: 289 - protein, 267 aa, synthetic construct
  REGION 1..267: note = Description of sequence: Nucleotide binding domain for ABCG8
LEVRDLNYQVDLASQVPWFEQLAQFKMPWTSPSCQNSCELGIQNLSFKVRSGQMLAIIGSSGCGRASLLDVITGRGHGGKIKSGQIWINGQPSSPQLVRKCVAHVRQHNQLLPNLTVRETLAFIAQMRLPRTFSQAQRDKRVEDVIAELRLRQCADTRVGNMYVRGLSGGERRRVSIGVQLLWNPGILILDEPTSGLDSFTAHNLVKTLSRLAKGNRLVLISLHQPRSDIFRLFDLVLLMTSGTPIYLGAAQHMVQYFTAIGYPCPR

SEQ ID NO: 290 - protein, 200 aa, synthetic construct
  REGION 1..200: note = Description of sequence: Nucleotide binding domain for ABCG8
LAIIGSSGCGRASLLDVITGRGHGGKIKSGQIWINGQPSSPQLVRKCVAHVRQHNQLLPNLTVRETLAFIAQMRLPRTFSQAQRDKRVEDVIAELRLRQCADTRVGNMYVRGLSGGERRRVSIGVQLLWNPGILILDEPTSGLDSFTAHNLVKTLSRLAKGNRLVLISLHQPRSDIFRLFDLVLLMTSGTPIYLGAAQHM

SEQ ID NO: 291 - protein, 250 aa, Homo sapiens
  REGION 1..250: note = Description of sequence: A homodimerizing TAP1 polypeptide
GLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPCVLILDDATSALDANSQLQVEQLLYESPERYSRSVLLITQHLSLVEQADHILFLEGGAIREGGTHQQLMEKKGCYWAMVQ

SEQ ID NO: 292 - protein, 250 aa, synthetic construct
  REGION 1..250: note = Description of sequence: A homodimerizing TAP1 polypeptide
  VARIANT 177: note = X is any amino acid other than aspartic acid or glutamic acid
GLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDXATSALDAGNQLRVQRLLYESPERYSRSVLLITQHLSLVEQADHILFLEGGAIREGGTHQQLMEKKGSYWAMVQ

SEQ ID NO: 293 - protein, 282 aa, Homo sapiens
  REGION 1..282: note = Description of sequence: homodimerizing human TAP1
ETEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDDATSALDAGNQLRVQRLLYESPERYSRSVLLITQHLSLVEQADHILFLEGGAIREGGTHQQLMEKKGSYWAMVQ

SEQ ID NO: 294 - protein, 240 aa, Homo sapiens
  REGION 1..240: note = Description of sequence: NBD amino acid sequence from human TAP1
  VARIANT 209: note = X is any amino acid other than aspartic acid or glutamic acid
ETEFFQQNQTGGGGSGLQTVRSFGGGGGSGGSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPSVLILDXATSALDAGNQLRVQRLLYESPERYSRSVLLI

SEQ ID NO: 295 - protein, 242 aa, synthetic construct
  REGION 1..242: note = Description of sequence: hydrolysis deficient ABCG5 NBD
  VARIANT 167: note = X is any amino acid other than aspartic acid or glutamic acid
RPWWDITSCRQQWTRQILKDVSLYVESGQIMCILGSSGSGKTTLLDAMSGRLGRAGTFLGEVYVNGRALRREQFQDCFSYVLQSDTLLSSLTVRETLHYTALLAIRRGNPGSFQKKVEAVMAELSLSHVADRLIGNYSLGGISTGERRRVSIAAQLLQDPKVMLFDXPTTGLDCMTANQIVVLLVELARRNRIVVLTIHQPRSELFQLFDKIAILSFGELIFCGTPAEMLDFFNDCGYPCPE

SEQ ID NO: 296 - protein, 165 aa, synthetic construct
  REGION 1..165: note = Description of sequence: hydrolysis deficient ABCG5 NBD
  VARIANT 133: note = X is any amino acid other than aspartic acid or glutamic acid
GSSGSGKTTLLDAMSGRLGRAGTFLGEVYVNGRALRREQFQDCFSYVLQSDTLLSSLTVRETLHYTALLAIRRGNPGSFQKKVEAVMAELSLSHVADRLIGNYSLGGISTGERRRVSIAAQLLQDPKVMLFDXPTTGLDCMTANQIVVLLVELARRNRIVVLTIH

SEQ ID NO: 297 - protein, 267 aa, synthetic construct
  REGION 1..267: note = Description of sequence: hydrolysis deficient ABCG8 NBD
  VARIANT 192: note = X is any amino acid other than aspartic acid, glutamic acid or asparagine
LEVRDLNYQVDLASQVPWFEQLAQFKMPWTSPSCQNSCELGIQNLSFKVRSGQMLAIIGSSGCGRASLLDVITGRGHGGKIKSGQIWINGQPSSPQLVRKCVAHVRQHNQLLPNLTVRETLAFIAQMRLPRTFSQAQRDKRVEDVIAELRLRQCADTRVGNMYVRGLSGGERRRVSIGVQLLWNPGILILDXPTSGLDSFTAHNLVKTLSRLAKGNRLVLISLHQPRSDIFRLFDLVLLMTSGTPIYLGAAQHMVQYFTAIGYPCPR

SEQ ID NO: 298 - protein, 200 aa, synthetic construct
  REGION 1..200: note = Description of sequence: hydrolysis deficient ABCG8 NBD
  VARIANT 138: note = X is any amino acid other than aspartic acid, glutamic acid or asparagine
LAIIGSSGCGRASLLDVITGRGHGGKIKSGQIWINGQPSSPQLVRKCVAHVRQHNQLLPNLTVRETLAFIAQMRLPRTFSQAQRDKRVEDVIAELRLRQCADTRVGNMYVRGLSGGERRRVSIGVQLLWNPGILILDXPTSGLDSFTAHNLVKTLSRLAKGNRLVLISLHQPRSDIFRLFDLVLLMTSGTPIYLGAAQHM

(19) United States
(12) Patent Application Publication
(10) Pub. No.: US 2025/0257148 A1
(43) Pub. Date: Aug. 14, 2025

(54) ATP-Dependent Agonists of Immune Cells Function as Anticancer Agents

(71) Applicant: Crosslink Therapeutics Inc., Boston, MA (US)

(72) Inventors: Ronald D. SEIDEL, III, Natick, MA (US); Rodolfo J. CHAPARRO, Cambridge, MA (US); John F. ROSS, Arlington, MA (US)

(21) Appl. No.: 19/195,720

(22) Filed: Apr. 30, 2025

Related U.S. Application Data
(63) Continuation of application No. PCT/US2024/045722, filed on Sep. 6, 2024.
(60) Provisional application No. 63/536,921, filed on Sep. 6, 2023.

(51) Int. Cl. C07K 16/30; C07K 14/705; C07K 16/28

(52) U.S. Cl. CPC C07K 16/30; C07K 14/70578; C07K 16/2809; C07K 16/2818; C07K 16/2878; C07K 2317/31; C07K 2317/55; C07K 2317/569; C07K 2317/622; C07K 2317/75; C07K 2317/92; C07K 2317/94; C07K 2318/00; C07K 2319/21

(57) ABSTRACT

The present disclosure provides polypeptide constructs that act as agonists of immune cell function when exposed to sufficient levels of ATP to cause their assembly into dimers or higher order complexes (e.g., trimers, tetramers, etc.). The constructs may also be conjugated to one or more therapeutic agent, chemotherapeutic agent, or labeling agent. The complexes of the constructs are capable of stimulating immune cells (e.g., cytotoxic CD8+ T cells and/or NK cells) that function to promote anti-tumor immune responses and delivering the conjugated agents into the tumor microenvironment. The constructs may be employed as anticancer agents/therapeutics for the treatment of solid tumors that have elevated levels of ATP.

This application claims the benefit of U.S. Provisional Patent Application No. 63/536,921, filed Sep. 6, 2023.

### I. INCORPORATION OF SEQUENCE LISTING

The sequence listing in ST.26 XML format entitled 2974-6 PCT_05SEP2024_ST26, created on Sep. 5, 2024, comprising 521,246 bytes, prepared according to 37 CFR 1.822 to 1.824, and submitted concurrently with the filing of this application, is incorporated herein by reference in its entirety.

### II. INTRODUCTION

Potent anti-cancer therapies remain limited due to toxic systemic effects manifesting well below an ideal therapeutic dose. Immune agonists such as cytokines and anti-T cell receptor antibodies are particularly toxic when delivered systemically, potentially leading to severe adverse events such as cytokine release syndrome and death. Strategies to increase the effective dose of immunotherapeutic agents at the tumor relative to the systemic dose/toxicity (i.e. the “therapeutic index”) include intratumoral administration and targeting of immunotherapies via linkage to antibodies against tumor-associated antigens (TAA). However, intratumor delivery is bespoke and invasive, and therefore has limited ability to reach large patient populations due to its associated surgical and logistical complexity. Newer agents that target TAAs attempt to impart a level of conditionality on the immune agonist wherein the agonist only accumulates to biologically effective concentrations at sites with sufficient density of the targeted TAA. TAA-targeted immune agonists suffer from serious limitations including, but not limited to: a small number of unique and validated TAAs expressed on the cell surface which are accessible by immunotherapeutics; low TAA density resulting in low efficacy; escape of cancers due to loss of TAA expression; TAAs that are not truly tumor specific, resulting in on-target/off-tumor toxicity; and the fact that each TAA requires a different drug of low therapeutic reach relative to systemic therapies.

Increasing the stoichiometry of immune agonism and/or TAA binding has also been explored as a means of enhancing the potency of TAA-targeted immunotherapies. For example, first generation bispecific T cell engagers such as blinatumomab comprise a single anti-TAA domain paired with a single anti-CD3 domain for a stoichiometry of 1:1. However, in the case of the bispecific T cell engager AFM11, an anti-TAA: anti-CD3 stoichiometry of 2:2 was used instead. This format was associated with more potent T cell mediated tumor cell killing in vitro, and importantly lowered the number of cytotoxic effector T cells required for tumor cell killing. However, a subsequent clinical trial of AFM11 revealed severe toxicity requiring termination of the study. A conceptually similar stoichiometric increase for the Fc domain of an antibody-like molecule showed a similar increase in in vitro potency. Such stoichiometric modifications likely act through avidity enhancements in TAA and immune receptor engagement. In addition, crosslinking of immune receptors such as Fc receptors or T cell receptors is known to amplify signal transduction through these receptors and signaling pathways, leading to more potent immune responses. Therefore, more potent tumor targeting, including reducing effector T cell and antigen density requirements, is potentially possible by engaging immune receptors with stoichiometrically enhanced immunotherapeutics. However, those enhanced immunotherapeutics have resisted clinical translation due to safety concerns.

A targeted immunotherapy could avoid the aforementioned liabilities of current immunotherapies by exploiting tumor characteristics that are generalizable to most/all tumors, critical to tumor survival, and highly expressed relative to normal tissues. The tumor microenvironment (TME) comprises the vasculature, immune and supportive cells, the extracellular matrix and local molecules around the tumor. The TME shares features across many different tumor types including a low pH, high extracellular adenosine triphosphate (ATP) content, limited oxygen levels (hypoxia), leaky vasculature and the presence of tumor proteases (e.g. MMPs). Among these features, extracellular ATP is an excellent candidate for exploitation in the generation of novel classes of immunotherapeutics as it is a product of an altered and essential tumor metabolic program that is common to most if not all tumors. Extracellular ATP has been observed at very high levels in the TME (50-1000 μM) relative to normal tissues (less than ˜0.1 μM, e.g., 0.01 μM to 0.1 μM).

### III. SUMMARY

The present disclosure includes and provides molecular constructs comprising nucleotide binding domains (NBDs) that enable the use of tumor ATP as a trigger for the conditional (ATP-dependent) assembly of potent cancer therapeutic agents at the tumor. NBD-containing constructs form dimers or higher order complexes in the presence of ATP levels found in TMEs. The NBD-containing constructs find use as, among other things, therapeutic agents for treating various cancers. Therapies employing the constructs are designed to take advantage of the fact that ATP mediated complexation (e.g., dimerization) can result in agents that recruit immune cells into the TME and/or stimulate immune cells in the TME. Stimulation may take place by, for example, multimerizing the immunomodulatory protein domains presented to immune cells as a result of ATP mediated complexation of the constructs, producing more effective stimulation of the immune cells than single immunomodulatory protein sequences that cannot cause, for example, crosslink of their receptors. Dimers or other higher order complexes of NBD-containing constructs may also act through other mechanisms including targeted ADCC (antibody-dependent cell-mediated cytotoxicity) and/or CDC (complement-dependent cytotoxicity) of tumor cells. Because the elevation in ATP levels relative to surrounding tissue is virtually universal in TMEs, the constructs provide a mechanism for treating diverse tumor types. Moreover, because the individual constructs that have not undergone assembly into dimers or higher order constructs have either limited or no ability to bring about immune stimulation, the elevated ATP levels in TMEs that can bring about dimerization or complex formation can drive strong local stimulation with a lesser effect in non-tumor tissues where ATP is at a lower concentration. Accordingly, the constructs, which can assemble immunomodulatory amino acid (aa) sequences into multivalent states in the presence of ATP levels found in TME, may be used to modulate immune cell action in TMEs and bring about selective immune-mediated anti-tumor actions.

The NBD-containing constructs also offer several other advantages including trans- and cis-targeting. Cis-targeting by the NBD-containing constructs can effect stimulation of tumor-infiltrating leukocytes (e.g., by providing immune stimulator agonists such as IL-2 or CD28) resulting in amplification of the anti-tumor immune response. When trans-targeted against TAAs, the constructs result in immune mediated cytolysis of tumor cells bearing the TAAs, which adds an additional tumor-specific action beyond that resulting from localized elevations in ATP levels. Regardless of the targeting scheme employed (e.g., cis- or trans-targeting), the constructs permit the use of diverse immunomodulatory domains derived from, for example, CD40L, CD28, IFN-g, IL-12, etc., each of which acts through receptors that require crosslink. Other advantages of the NBD-containing constructs described herein include their relatively small size, which permits penetration into tumor tissue, and their reduced diffusion from and concentration in the TME, due to their increased size when they assemble into dimers or higher order complexes on, for example, the surface of target cells.

Relative to their monomeric counterparts (constructs in the absence of ATP), dimers formed in the presence of ATP display distinctly different properties in addition to increased size and slower diffusion rates. For example, dimers of constructs (molecules) comprising an NBD, a scaffold, and a Tumor Specific Binder (TSB) with affinity for a TAA (e.g., an antigen or neoantigen expressed by a tumor cell) will dissociate from a surface bearing the target TAA more slowly than the monomeric forms of the same constructs. This is reflected in an increased avidity or half-life for the dissociation of the dimer from a surface bearing sufficient amounts of the TAA to bind both TSBs in the dimer relative to the monomeric form. Although the TSB of the monomer also provides for tumor homing and binding via the TAA, the dimer is significantly more potent and efficacious at inducing responses than the monomeric form, which has no or low activity and potency. In addition to the foregoing, ATP-driven dimerization of constructs comprising an NBD, a scaffold, and either a TSB or an immune cell binder (ICB) may cluster cell surface molecules in what is sometimes termed “capping”, “patching”, or “lattice formation”. ATP-driven dimerization can unexpectedly amplify receptor signaling beyond levels associated with more static monomeric or genetic dimer engagements. While not wishing to be bound by any theory, such results might arise from dynamic oligomerization as NBD containing constructs repeatedly homodimerize with multiple neighboring NBD containing constructs at the cell surface.

Constructs of the present disclosure may also comprise payloads including labels (e.g., radiolabels and/or sensitizers useful in radiotherapy and/or photodynamic therapy). The effect of payloads and labels in vivo may be enhanced by the ability of constructs of the present disclosure to accumulate in tumor tissues, particularly where the constructs accumulate at levels greater than expected due to kinetic and thermodynamic effects and/or the ATP mediated formation of lattices on the target cell surfaces that limit construct release or cellular degradation. Enhanced accumulation of constructs bearing payloads means that in addition to any effect the constructs may have on shaping an anti-tumor response through immune and/or apoptotic action, payloads have an increased ability to effect an anti-tumor action through increased delivery of therapeutic payloads (e.g., chemotherapeutics), radionuclides, and/or sensitizers (e.g., radiosensitizers and/or photosensitizers). Moreover, highly toxic molecules and/or molecules that produce severe side effects that are used or being considered as therapeutics (e.g., in cancer patients) can be administered as a payload attached to the NBD-containing constructs of the present disclosure. Conjugation of such molecules to NBD-containing constructs of the present disclosure permits them to be dosed at concentrations lower than necessary to achieve a therapeutic effect with the unconjugated molecules because the NBD conjugates accumulate in the ATP-rich TME and can provide a therapeutically effective level of the molecules. Conjugation therefore limits negative effects on non-tumor tissue (e.g., the molecules may be systemically safe) and/or limits the side effects experienced by the patient.

Because the NBD-containing constructs of this disclosure form dimers or higher order complexes (e.g., when the cell surface receptor aggregation results in a cap or patch), each event leading to internalization of constructs delivers at least twice the number of payload molecules to the cells as internalization of the monomeric form of the construct. Accordingly, the dimeric form of the constructs offers an advantage in the delivery of payloads such as radioisotopes or chemotherapeutics over both the monomeric form of the construct and antibodies that are specific for epitopes appearing only once on a cell surface protein and do not facilitate cell surface molecule patching or capping.

In addition to describing the constructs, the present disclosure also provides for methods of their preparation and methods of their use; including the use of the constructs to prepare a medicament and/or the use of the constructs for the purpose of treating a disease or a disorder (e.g., a cancer).

### IV. BRIEF DESCRIPTION OF THE DRAWINGS

FIG. 1 provides at A a diagram of a construct of the present disclosure comprising an NBD with an ATP binding site, a scaffold domain (scaffold aa sequence), and an immune cell activating domain (AD) joined by linker sequences. The right side of the diagram in A also shows dimerization of the construct in the presence of a sufficient ATP concentration for the binding site to be substantially occupied (e.g., a concentration in the range of the Kd of ATP binding to the NBD or a higher concentration). At B, FIG. 1 provides: (i) a first construct comprising a TSB, which is exemplified as a bivalent cell lineage or tumor-specific antibody but may be a monovalent binder such as an scFv or nanobody, and a first NBD of a heterodimerizing NBD pair joined by a linker; and (ii) a second construct comprising a second NBD of the heterodimerizing NBD pair joined by a linker. The right side of the diagram at B shows the formation of a heterodimer of the first and second constructs due to dimerization of the NBD domains in the presence of ATP. FIG. 1 at C provides on the left side a construct of the present disclosure structured around an IgFc scaffold (a KiHs-s IgFc) that comprise a NBD on one polypeptide of the construct and an ICB, AD, or TSB on the other polypeptide. The construct is shown as optionally bearing one or more payloads or labels (e.g., a chemotherapeutic that may be bound by a labile (cleavable) linker or a radiolabel). The construct substantially dimerizes in the presence of ATP as shown on the right side (e.g., at levels found in a TME but not in non-tumor peripheral tissue).

FIG. 2 provides a series of constructs (structures A to E) comprising an NBD and an AD with the constructs of structures C, D, and E also comprising scaffold sequences that may be non-dimerizing or may be interspecific. The elements of each construct are joined by independently selected optional linkers. The figure also shows the formation of dimers of the constructs in the presence of ATP. Sequences may be organized in the N- to C-terminal direction starting at either end of the constructs shown.

FIG. 3 provides a series of construct pairs (structures A to O). In A-I, the first construct in each pair comprises one or more NBDs and a TSB that binds to a TAA and the second construct of the pair comprises an NBD and an AD and/or a monovalent ICB. In J-M, the first construct in each pair comprises one or more NBDs and an ICB and the second construct of the pair comprises an NBD and an AD. Each of construct pairs E-I, L, and M has a scaffold in at least one of the constructs. In N and O, the first construct in each pair comprises a TSB and two NBDs, and the second construct of the pair comprises an NBD and an AD. The constructs in O also comprise a scaffold sequence. FIG. 3 also shows the formation of dimers of the constructs in the presence of ATP. The elements of each construct are joined by independently selected optional linkers.

FIG. 4A provides a series of constructs (see A, C, and D), pairs of constructs (B, E, F, G, and I), or a triplet of constructs (at H) that comprise two or more NBDs and can polymerize to form a complex in the presence of ATP (e.g., in a TME). In some instances, one or more of the constructs comprises a scaffold sequence and each construct may be joined by independently selected optional linkers. The figure also shows the formation of complexes in the presence of ATP comprising the constructs present in each case. Sequences may be organized in the N- to C-terminal direction starting at either end of the constructs shown.

FIG. 4B shows examples of an NBD bound to a TSB (e.g., antibody such as anti-mesothelin) that form homodimers in the presence of elevated ATP, such as is found in the TME. In (A) the construct comprises a bivalent antibody that may be monospecific or bispecific and accordingly gives rise to a tetramer or tetramer-like complex in the presence of ATP. The molecules need not comprise an Fc domain (e.g., as in VHHs or scFVs), and may comprise two antigen binding domains and remain bivalent. In (B), (C), and (D) the homodimerizing constructs comprise an NBD monovalent single chain antibody. The TSBs in (A) to (D) may be, for example, anti-HER2, anti-mesothelin, anti-EpCAM, anti-CLA-4, or anti-PSMA. When scaffold sequences are present, they may be an immunoglobulin (Ig) Fc peptide (such as an IgG1 Fc) that can bind any one or more of FcγRI, FcγRII, FcγRIII receptors and/or C1q proteins. Complexes capable of binding to one or more of those proteins can stimulate Ig-mediated (Ig-dependent) effector function responses, such as ADCC, antibody-dependent cellular phagocytosis (ADCP), and/or CDC directed against cells expressing a target (e.g., an antigen) to which the TSB component(s) may bind. Accordingly, such antibody functions may be used therapeutically to eliminate tumor cells. Alternatively, the scaffold may comprise aa sequences that do not have the ability to stimulate one or more Ig-mediated effector response (e.g., they are non-immunoglobulin aa sequences), or are Ig molecules that have the ability to stimulate Ig-mediated effector functions substantially (e.g., wholly) diminished relative to their wild-type (wt.) Ig sequences. Sequences may be organized in the N- to C-terminal direction starting at either end of the constructs shown.

FIG. 5A shows the structure of a series of polypeptide constructs bearing one or two NBDs that can homodimerize (H-NBDs) with an NBD in a second molecule of the construct in the presence of ATP. In addition, the constructs each comprise an immunoglobulin IgFc aa sequence that can stimulate one or more Ig-mediated effector functions. The constructs are oriented with the N-terminus at the top. Disulfide bonds between the Ig heavy chain aa sequences in the IgFc are not shown. The optional linker sequences may be selected independently, and may comprise rigid peptide linker sequences (e.g., repeats of Ala-Pro). The positions where H—NBDs may be located are shown by the number circles and enumerated in the table below the structures. The “---” in the table indicates the position is unfilled or is a bond or linker aa sequence. The NBDs present in the constructs may comprise a CAP and disulfide bonds for stabilization (not shown).

FIG. 5B shows the structures of a series of constructs comprising a first (1^{st}) and a second (2^{nd}) polypeptide each comprising an interspecific Ig heavy chain constant region aa sequence that together form an IgFc structure that can stimulate one or more Ig-mediated effector functions. The first and second polypeptides taken together comprise one or more (e.g., two) NBDs that homodimerize (H-NBDs) with an NBD in a second molecule of the construct in the presence of ATP. The constructs are oriented with the N-terminus at the top. The optional linker sequences may be selected independently, and may comprise rigid peptide linker sequences (e.g., repeats of Ala-Pro). For structures A-E the positions where H—NBDs may be located are shown by the number circles and enumerated in the table below the structures. The “---” in the table indicates the position is unfilled or is a bond or linker aa sequence. Structures F-L provide a series of specific constructs. Disulfide bonds between the Ig heavy chain aa sequences in the IgFc are not shown for structures A-E. Disulfide bonds linking the first and second polypeptide Ig heavy chain aa sequences in the lower hinge region are shown for structures F-L. The NBDs present in the constructs may comprise a CAP and disulfide bonds for stabilization (not shown).

FIG. 5C shows a series of constructs at A-D comprising a first (1^{st}) and a second (2^{nd}) polypeptide each comprising an interspecific Ig heavy chain constant region aa sequence that together form an IgFc structure that can stimulate one or more Ig-mediated effector functions. Structure E is a homodimer of a construct comprising homodimerizing Ig heavy chain constant region aa sequences that form an IgFc structure. The first and second polypeptides of the constructs taken together comprise one or more (e.g., two) NBDs that homodimerize (H-NBDs) with an NBD in a second molecule of the construct in the presence of ATP. The first and second polypeptides of the constructs taken together also comprise one or more (e.g., two) TSBs that may be directed to the same or different TAAs when two TSBs are present. Disulfide bonds linking the first and second polypeptide Ig heavy chain aa sequences in the lower hinge region are shown. The constructs are oriented with the N-terminus at the top. The optional linker sequences may be selected independently and may comprise rigid peptide linker sequences (e.g., repeats of Ala Pro). The NBDs present in the constructs are shown with an optional CAP aa sequence and comprise disulfide bonds for stabilization (not shown). Each construct may form a homodimer in the presence of ATP due to interactions between the NBDs and ATP molecules.

FIG. 5D shows the structures of a series of constructs depicting some possible arrangements for constructs comprising one or more NBDs that homodimerize in the presence of sufficient ATP (H-NBDs). For stabilization, the H-NBDs present in the constructs may comprise disulfide bonds and a CAP when located in an N-terminal position (not shown). The constructs further comprise as elements one or more ICBs, one or more ADs, one or more TSBs, and optionally one or more scaffold sequences. The elements are joined by one or more optional linker sequences indicated as lines between the elements. The positions where those elements may be located are shown by the number circles and enumerated in the table below the structures. Positions where an ICB(s) or AD(s) may be located are denoted as ICB/AD(s). Constructs are grouped in the table such that the first digit of the construct series indicates the position where an H-NBD is located. A “---” in the table indicates that a position is either unfilled (i.e., it is a bond or linker aa sequence) or filled by one or more AD sequences (AD(s) which may be in tandem.

FIG. 5E shows the structures of Bispecific T cell Engager-like (BiTE-like) constructs structured around a pair of interspecific scaffold aa sequences (shown as an Ig KiH sequence pair with knob and hole bearing sequences) bound by a pair of disulfide bonds. The constructs each comprise a TSB, AD, and NBD attached to the scaffold sequences. Each construct may form a homodimer in the presence of ATP due to interactions between the NBDs and ATP molecules.

FIG. 5F at A provides a protein construct 14 of the present disclosure comprising a bispecific antibody 10 having a TSB (e.g., a targeting portion) 11 with affinity for an antigen or neoantigen expressed by a tumor cell, and an ICB or AD 12 with affinity for an immune cell antigen (e.g., anti-CD3). Antibody 10 is coupled to an NBD (e.g., a homodimerizing NBD) 13 by an optional linker shown by a curved line. The linker may comprise a conjugation site permitting the preparation of the construct by joining antibody 10 to the NBD 13. In the absence of ATP (left) the protein construct 14 is in the form of monomers, whereas in the presence of ATP the protein complexes to form dimers 15 or higher order structures that more efficiently stimulate immune cell responses than the non-dimerized protein constructs. FIG. 5F at B provides a diagram of a protein construct of the present disclosure comprising a TSB 1, an AD 2, and an NBD 3 joined by independently selected optional linker sequences shown as lines between the elements. The NBD may be part of a fusion protein comprising the TSB 1 and AD 2 or may be conjugated to them at conjugation site 9. Conjugation site 9 may be within a linker as shown in the figure, or elsewhere in the molecule (e.g., the conjugation site may not be in the linker but in the TSB or AD). The figure also shows the formation of dimers of the constructs in the presence of ATP that are more efficient as stimulators of immune cell responses (right side of the figure) than the non-dimerized constructs (shown on the left).

FIG. 5G provides a diagram of a protein construct of the present disclosure comprising a TSB 1 and an AD 2 on either side (N-terminal and C-terminal) of an NBD 3 joined by independently selected optional linker sequences shown as lines between the elements. The figure also shows the formation of dimers of the constructs in the presence of ATP that are more efficient as stimulators of immune cell responses (right side of the figure) than the non-dimerized constructs (shown on the left).

FIG. 5H at A and B provides diagrams of a protein construct of the present disclosure comprising a TSB 1, an AD 2, and an NBD 3. The TSB 1 is in the form of a Fab with V_{H} 4, CH1 5, V_{L} 6, and C_{L} 7 shown along with disulfide bond 8. At A, the NBD is attached to the CH1 5 and the AD 2 is attached to C_{L} 7. In contrast, at B the NBD is attached to C_{L} 7 and the AD is attached to CH1 5. The NBD may be part of a fusion protein comprising a portion of TSB 1 or may be conjugated to it at conjugation site 9, which may be within the TSB or within a linker attached to it as shown. The figure also shows the formation of dimers of the constructs in the presence of ATP that are more efficient as stimulators of immune cell responses (right side of the figure) than the non-dimerized constructs (shown on the left).

FIG. 5H at C and D provides diagrams of a protein construct of the present disclosure comprising a TSB 1, an AD 2, and an NBD 3. AD 2 may be in the form of a Fab with V_{H} 4, CH1 5, V_{L} 6, and C_{L} 7 shown along with disulfide bond 8. At C, the NBD is attached to CH1 5 and the TSB 1 is attached to C_{L} 7. In contrast, at D the NBD is attached to C_{L} 7 and the TSB 1 is attached to CH1 5. The NBD may be part of a fusion protein comprising AD 2, or may be conjugated to it at conjugation site 9, which may be within the AD 2 or within a linker attached to it as shown. The figure also shows the formation of dimers of the constructs in the presence of ATP that are more efficient as stimulators of immune cell responses (right side of the figure) than the non-dimerized constructs (shown on the left).

Constructs depicted in any of FIGS. 1-5H may be considered to show the order of elements from the N-terminus at the top of the page to the C-terminus at the bottom of the page; however, provided the order of the elements is retained, they may also be understood to support constructs with the elements ordered with C-terminus depicted at the top of the page and N-terminus at the bottom.

FIG. 6 shows an alignment of sequences comprising NBD domain elements of: the ABCA1 gene product from UniProtKB-095477 (SEQ ID NO:1), the ABCB1 gene product from UniProtKB-P08183 (SEQ ID NO:2), the ABCC1 gene product from UniProtKB-P33527 (SEQ ID NO:3), the ABCD1 gene product from UniProtKB/Swiss-Prot: P33897 (SEQ ID NO:4), the ABCE1 gene product from UniProtKB/Swiss-Prot: P61221 (SEQ ID NO:5), the ABCF1 gene product from UniProtKB-Q8NE71 (SEQ ID NO:6), the ABCG2 gene product from UniProtKB/Swiss-Prot: P45844 (SEQ ID NO:7), and wt. TAP1 from UniProtKB-095477 (SEQ ID NO:9). A portion of the Walker A region sequence from aas 46 to 55 and the LSGGQ motif sequence from aas 152 to 156 of the TAP1 aa sequence are underlined and bolded. The hydrolytic acidic amino acid of the TAP1 NBD shown in FIG. 6 (i.e., the Asp at position 177) and the Asp (D) or Glu (E) residues are shown underlined.

FIG. 7 shows the sequence of human TAP2 from UniProtKB-Q03519 (SEQ ID NO:21).

FIG. 8 shows an SDS-polyacrylamide gel electrophoresis (PAGE) gel of a TAP1 variant bearing N676G, S677N, Q680R, E682Q, and/or Q683R substitutions. From left to right the lanes are molecular weight markers (M), reduced sample (R), empty lane, and non-reduced sample (NR).

FIG. 9 shows size exclusion chromatograms of a TAP1 NBD variant polypeptide bearing N676G, S677N, Q680R, E682Q, and/or Q683R substitutions and substitutions of cysteines present in the sequence with serines (i) in the presence of 1 mM adenosine diphosphate (ADP), resulting in a monomeric form of the polypeptide (apparent Mw 37 kDa), and (ii) in the presence of 1 mM ATP, resulting in a dimeric form of the polypeptide (apparent Mw 57 kDa).

FIG. 10 provides the results of a mass photometry assessment of the same TAP1 NBD variant subject to chromatograph in FIG. 9. The results, obtained in the presence of 1 mM ATP, indicate dimerization of the TAP1 NBD variant.

FIG. 11 provides the results of dynamic light scattering (DLS) size determination for the TAP1 in the presence of (A) ADP or (B) ATP.

FIG. 12 provides a coomassie blue stained gel of purified TAP1 NBD of SEQ ID NO: 118 modified to homodimerize and chromatographic analysis of the purified protein in the presence of ADP and ATP.

FIG. 13 shows SDS-PAGE gels from: (A) the induced expression of the TAP1 NBD polypeptides provided by SEQ ID NO: 118 and SEQ ID NO: 119; and (B) the purification of the polypeptide of SEQ ID NO:119.

FIG. 14A at A shows a ribbon diagram of a portion of a TAP1 NBD. At B, a TAP1 construct is depicted in linear form with the N-terminus at the top. The small horizontal rectangles indicate some approximate locations where substitutions may be made, including cysteine substitutions. The catalytic Asp residue (D668 in human TAP1) and the D-helix are indicated. The dashed line on the left shows a possible disulfide bond between a cysteine appearing or substituted in Coupling Helix 1 and a cysteine appearing or substituted in the NBD (see, e.g., construct 308 having a disulfide between cysteines resulting from E274C and P513C substitutions). The dashed line on the right shows a possible disulfide bond between cysteines within the NBD, which is exemplified by the disulfide bond between cysteines substituted in construct 182 (L493C: E573C). The numbers provided in A and B are construct numbers indicating the approximate locations within the protein affected by changes appearing in the indicated constructs. Constructs 175-179 and 211 relate to C-terminal deletions of the NBD. Construct 179 comprises a deletion approximately up to the N-terminus of alpha helix 9 (a9) and construct 211 a deletion up to about the N-terminus of alpha helix 8 (a8). Accordingly, construct 179 essentially deletes the a9 helix to the C-terminus of the NBD, whereas construct 211 essentially deletes the a8 helix to the C-terminus of the NBD (including the a9 helix). The human TAP1 a8 helix comprises the aa sequence THQQLME (SEQ ID NO:30) and the a9 helix comprises the aa sequence CYWAMVQ (SEQ ID NO:31). The TEV site recited in this FIG. and in FIGS. 12 and 15 is Tobacco Etch Virus site-specific protease cleavage site. The His Tag may consist of, for example, a series of eight histidine residues (HHHHHHHH, SEQ ID NO:151).

FIG. 14B provides the sequence of a series of TAP constructs. The coupling helix from TAP1 (Coupling Helix 1) comprises the aa sequence ETEFFQQNQT (SEQ ID NO: 17) and the coupling helix from TAP2 (Coupling Helix 2) comprises the aa sequence GLQTVRSFG (SEQ ID NO:18). Sequences annotated as having a “modified D-helix” comprise the rat TAP1 D-helix sequence GNQLRVQRLL (SEQ ID NO:26). The absence of a linker sequence i.e., “No Linker” is indicated by the abbreviation or “(NL)”.

FIG. 15 at A shows an SDS-PAGE analysis of an exemplary protein construct (construct 303) expression in E. coli before (lane 4) and after isopropyl β-d-1-thiogalactopyranoside (IPTG) induction (lane 5). Also shown are reduced (lane 7) and non-reduced (“NR”, lane 9) protein construct samples after purification on Talon® resin. At B, FIG. 15 shows size exclusion chromatography demonstrating the dimerization behavior of construct 303 in the presence of ADP and ATP. At C, FIG. 15 shows the melting point of construct 303 as determined by differential scanning calorimetry (DSC).

FIG. 16 shows a plot indicating the effect of C-terminal aa deletions on the melting points determined by DSC for various TAP constructs. The plot provides the melting points of several constructs with native TAP1 (filled circles) and added disulfide bonds (filled squares). Also shown as an asterisk is the melting point of construct 451, which has no N-terminal CAP (i.e., it lacks an N-terminal sequence comprising Coupling Helix 1 and Coupling Helix 2).

FIG. 17 provides a table summarizing, among other things, some structural features of various TAP1 constructs, the results of expression testing, dimerization in the presence of ATP, and construct melting points.

FIG. 18 shows constructs 156-159 having as elements an NBD, an anti-B cell maturation antigen (anti-BCMA) VHH, and an anti-CD3 aa sequence in various orders described further in Example 6.

FIG. 19 shows an SDS-PAGE gel of purified constructs 156, 157 and 159 under reducing and non-reducing (oxidized) conditions. Each construct gives a single band in both the reduced and non-reduced states.

FIG. 20 shows chromatograms from size exclusion chromatography of constructs 156, 157 and 159 in the presence of ADP or ATP.

FIG. 21 at A shows a diagram for three different constructs that demonstrate the ability to select the ATP concentration that results in dimerization of the constructs (the ATP set point). At B, the figure shows plots of the residence time (1/k_{dis}) for each of the three constructs from their target (an immobilized mesothelin polypeptide) at different ATP concentrations.

FIG. 22 shows a TAP1 NBD-scFc fusion protein construct (436) at A, and a construct comprising a TAP1 NBD-IgG knob fusion polypeptide (571) paired with an IgFc hole polypeptide (476) denoted “571:476” at B. At C the figure shows triplicate plots of dynamic light scattering (DLS) data of the constructs in the presence of ADP only (upper plot), and ADP and ATP (lower plot). The time course for serum concentrations of construct 571:476 is provided in FIG. 22 at D.

FIG. 23 shows three different constructs each comprising a pair of peptides. The numbers of the individual peptides in the constructs are shown above each peptide, and the pair of peptides is indicated by the peptide numbers separated by a colon. Construct 590:587 at A, 473:587 at B, and a genetic dimer of single chain (sc) 4-1BBL trimeric repeats at C. At D, FIG. 23 shows a plot of luminescence from Jurkat reporter cells that express luciferase in response to 4-1BB stimulation at the indicated concentrations of construct 590:587 and negative control 473:587 (left side) and in response to anti-41BB (Urelmab) and the genetic dimer of 4-1BBL (“Dimer-41BBL” see C) on the right side. Plots of triplicate measurements of luminescence from the same Jurkat cell lines stimulated with constructs 590:587 with and without Apyrase, and negative control construct 473:587 are shown at E and triplicate measures of the luminescence from the same cells in the presence of construct 590:587, negative control construct 473:587, and a modified 590:587* construct with attenuated activity are provided at F.

### V. DETAILED DESCRIPTION

### A. Definitions

As used herein amino acid (“aa” singular or “aas” plural) means the naturally occurring proteogenic amino acids incorporated into polypeptides and proteins in mammalian cell translation. Unless stated otherwise these are: L (Leu, leucine), A (Ala, alanine), G (Gly, glycine), S (Ser, serine), V (Val, valine), F (Phe, phenylalanine), Y (Tyr, tyrosine), H (His, histidine), R (Arg, arginine), N (Asn, asparagine), E (Glu, glutamic acid), D (Asp, asparagine), C (Cys, cysteine), Q (Gln, glutamine), I (Ile, isoleucine), M (Met, methionine), P (Pro, proline), T (Thr, threonine), K (Lys, lysine), and W (Trp, tryptophan). Aa also includes the aas hydroxyproline and selenocysteine, which appear in some proteins found in mammalian cells; however, unless their presence is expressly indicated, they are not understood to be included.

Substitutions of aas at specific locations in a sequence are indicated by the original aa given in single or triple letter code, the numerical position of that aa, and the aa that is substituted into the sequence in single or triple letter code. By way of example an alanine (A) at position 12 of a sequence substituted with a proline (P) would be indicated as A12P using single letter code, or Ala12Pro using triple letter code. Where more than one substitution appears in the same sequence they may be separated by a slash “/”. Accordingly, the above-mentioned A12P substitution in the same sequence as a V23A and a Q41G substitution may be indicated by the sequence A12P/V23A/Q41G.

Amino acid residues appearing in a wt. protein or reference sequence are denoted by the single letter code for the aa and the position in the sequence. For example, a cysteine appearing at position 372 is denoted as C372. Substitution of an aa within a sequence is indicated by an abbreviation starting with the single letter aa code of the original aa, followed by the numerical position of that aa, and the single letter code for the substituted aa (e.g., substitution of a cysteine at position 372 by a serine is indicated as C372S). Where the sequence of a wt. polypeptide is altered, either by addition or deletion of one or more aas, the specific residue or residue number will continue to refer to the same specific aa in the altered polypeptide (e.g., in the addition of one aa at the N-terminus of a peptide in which position 2 was Gly (G2), a reference to position G2 will still be understood to indicate the Gly that is now at position 3, unless indicated otherwise).

As used herein, a “conservative aa substitution” refers to the replacement of an aa in a protein with an aa having a similar side chain within the groups that follow. The group of aas having aliphatic side chains consists of glycine, alanine, valine, leucine, and isoleucine. The group of aas having aliphatic-hydroxyl side chains consists of serine and threonine. The group of aas having amide containing side chains consists of asparagine and glutamine. The group of aas having aromatic side chains consists of phenylalanine, tyrosine, and tryptophan. The group of aas having basic side chains consists of lysine, arginine, and histidine. The group of aas having acidic side chains consists of glutamate and aspartate. The group of aas having sulfur containing side chains consists of cysteine and methionine. Accordingly, a conservative substitution of an aa is one made with an aa within a group as defined above. In contrast, as used herein a “nonconservative aa substitution” of an aa is one made using an aa from a different group.

The terms “polypeptide” and “protein” are used interchangeably herein, and refer to a polymeric form of aas, which unless stated otherwise are the naturally occurring proteinogenic L-aas that are incorporated biosynthetically into proteins during translation in a mammalian cell.

A colon placed between positions comprising cysteines in an aa sequence as used herein indicates the location of a disulfide bond between those positions (in the non-reduced state). By way of example, C735: R721C indicates a disulfide bond formed between the Cys residue present in a sequence at position 735 and a Cys residue substituted for an Arg at position 721. The order may be reversed (e.g., R721C: C735) but has an equivalent meaning.

As used herein, the terms “antibody” or “antibodies” include, but are not limited to, antibodies from, for example, mammals, sharks, and camelids such as human IgGs (hIgG) and heavy chain only antibodies. Antibodies also include antigen binding fragments thereof including, but not limited to, Fab′ and Fv fragments. Monovalent antibody fragments include, but are not limited to, Fab, and Fv fragments. Antibodies also include monovalent single chain (sc) antibodies and antibody constructs comprising the antigen binding portion of an antibody including, but not limited to, scFv (scFv (vH-vL) or scFv (vL-vH)), nanobodies (VHH or VHH), and V_{H} heavy chain only antibody polypeptides. Throughout the disclosure the term “antibody” may be recited in conjunction with various antibody fragments or constructs. Those recitations are not intended to alter the definition of an antibody, but instead are provided to elaborate on possible types of antibody sequences contemplated at various points of the disclosure and/or for antecedent basis.

Alignments to identify corresponding residues of different sequences may be conducted using the US National Center for Biotechnology Information (NCBI) BLAST program (blastp release of BLAST+2.9.0 on the world wide web at blast.ncbi.nlm.nih.gov/Blast.cgi?PAGE=Proteins) or the European Bioinformatics Institute's Clustal Omega program (version 1.2.4 available at www.ebi.ac.uk/Tools/msa/clustalo/) using default parameters at the date of filing. Unless stated otherwise, Clustal Omega is used to identify corresponding residues in different sequences.

The percent sequence identity refers to the percentage of aas or nucleotides that are the same between two aa or nucleic acid sequences that have been aligned. Unless stated otherwise, the percentage sequence identity is determined using BLAST+release 2.9.0 employing a blastp alignment for polypeptides or a blastn alignment for nucleic acids with default parameters at the date of filing.

The term “substantially” is intended to encompass both “wholly” and “largely but not wholly” unless indicated otherwise. For example, an Ig Fc that “substantially does not induce cell lysis via ADCC or CDC” means an Ig Fc that induces no cell lysis at all or that largely but not wholly induces no cell lysis via ADCC or CDC.

As used herein, the term “about” used in connection with an amount indicates that the amount can vary by 10%. For example, “about 100” means an amount of from 90-110. Where “about” is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

The terms “patient” and “subject” are used interchangeably.

The term “payload” includes molecules or atoms that may be attached covalently or non-covalently (e.g., by chelation) to a construct of the present disclosure. Labels represent a sub-class of payloads that render the molecule or the location where the molecule is detectable.

Unless defined otherwise, all technical and scientific terms used herein have the same meaning as commonly understood by one of ordinary skill in the art to which this disclosure belongs.

It must be noted that, as used herein and in the appended claims, the singular forms “a,” “an,” and “the” include plural referents unless the context clearly dictates otherwise. Thus, for example, reference to “a T cell” includes a plurality of such T cells. This statement is intended to serve as antecedent basis for use of such exclusive terminology as “one,” “solely,” “only” and the like in connection with the recitation of claim elements, as well as use of a “negative” limitation excluding the claim limitation.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### B. Description

The microenvironment within tumors differs in a number of ways from nonmalignant tissues, including differences in any one or more of its pH, ATP content, limited oxygen levels (hypoxia), and tumor protease (e.g., MMPs) levels. Extracellular ATP levels in normal (non-tumor) tissues are typically in the range of 0.01-0.1 micromolar (μM) in normal tissues, whereas the extracellular level of ATP in solid tumors is generally in the range of 50-200 μM. The present disclosure describes and provides for the use of the ATP levels found in TMEs to bring about an immune response to cells in the tumor. More specifically, some constructs described herein may comprise an immune cell activating domain AD (i.e., an AD of an immunomodulatory molecule that binds and stimulates an immune cell) and at least one NBD that forms a substantial amount of dimers or other higher order complexes in the presence of ATP at the levels found in the TME. Dimerization or higher order complex formation of the NBD results in a complex presenting two or more immune cell ADs. The use of immune cell ADs that can stimulate immune cells such as NK cells and CD8+ T cells when presented as dimers or higher order complexes, but that do not by themselves stimulate the same immune cells to the same degree when presented singly, permits intratumoral stimulation of an immune response within the TME.

Other constructs described herein may comprise TSBs that bind to TAAs, ICBs, and at least one NBD that forms dimers or other higher order complexes in the presence of ATP at the levels found in the TME. Such constructs may optionally comprise an AD. Dimerization or higher order complex formation of the NBD results in dimers or higher order complexes that can recruit and/or stimulate immune cells in the TME. Because dimerization or other higher order complexes of such constructs do not form higher order complexes outside of the TME, they permit intratumoral stimulation of an immune response while limiting immune stimulation outside of the tumor.

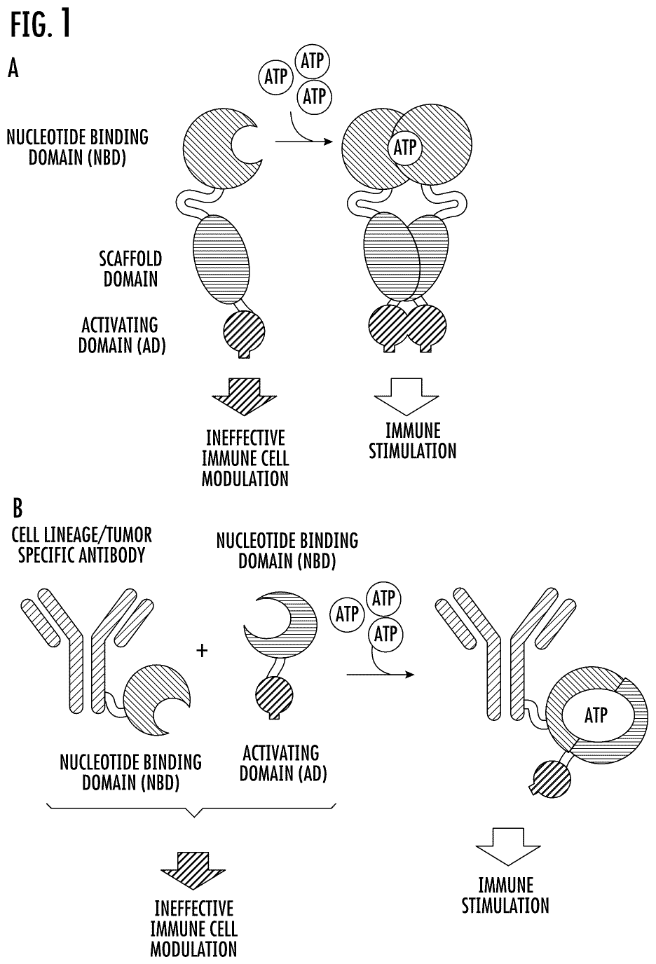

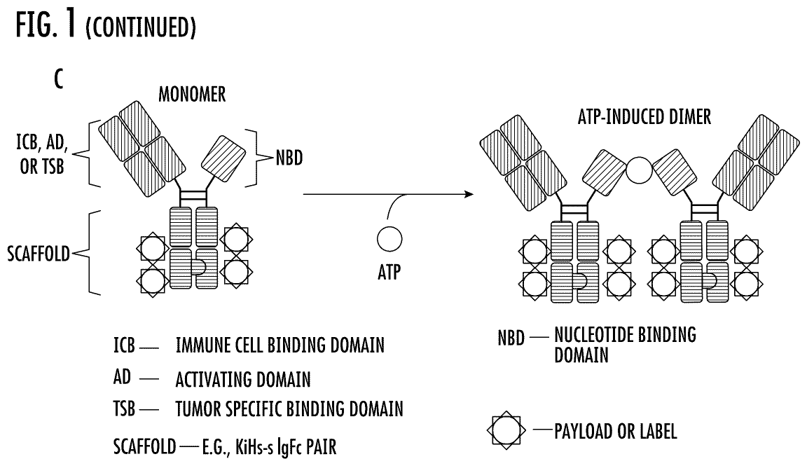

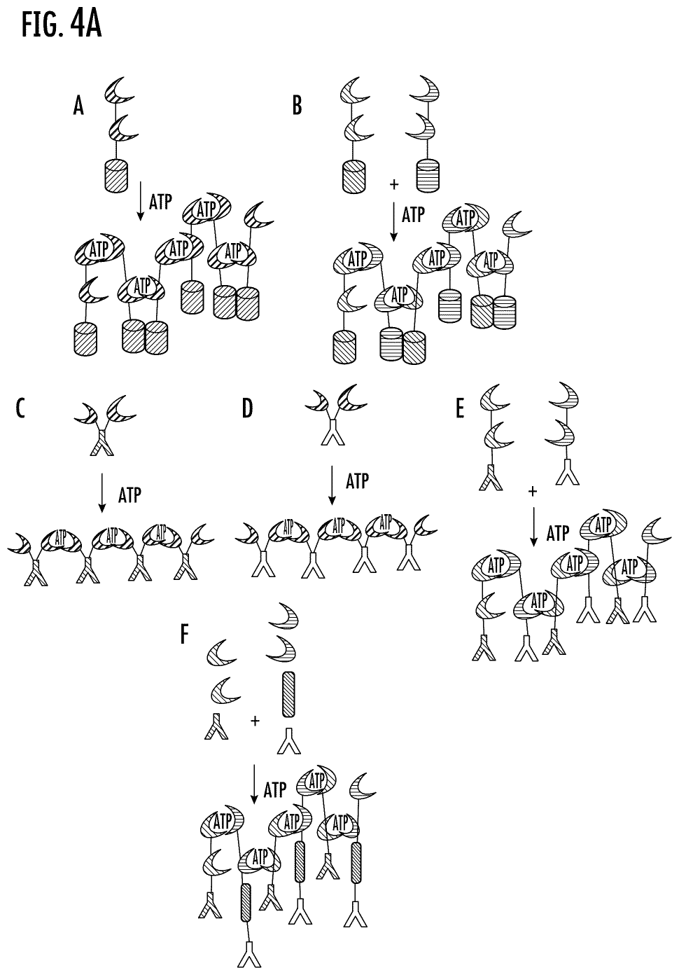

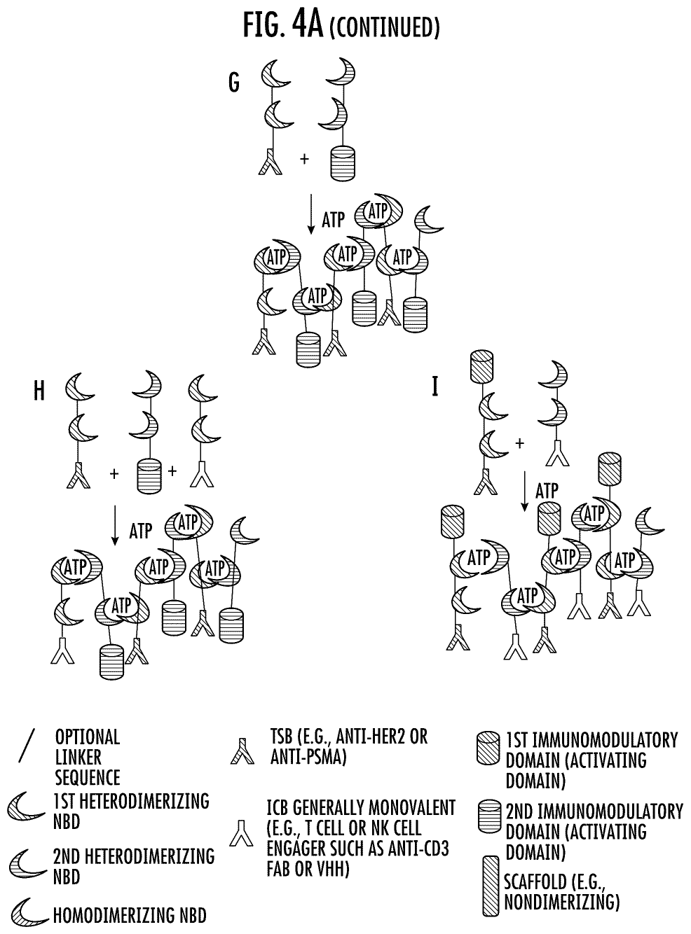

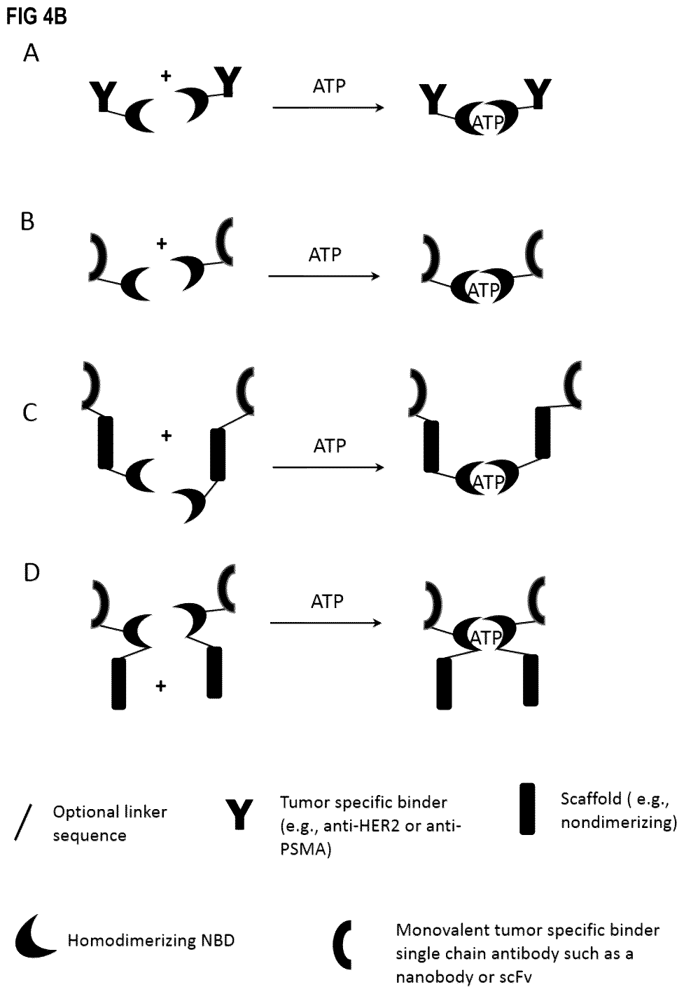

Because the ADs, tumor-specific binding domains, and ICBs of the constructs presented singly (as monomers) do not stimulate cells such as NK cells and CD8+ T effector cells, as effectively as when presented as a pair or multimer by ATP induced dimers (see, e.g., FIGS. 1-3 and 4B) or higher order complexes (see, e.g., FIG. 4A) described herein, administration of monomeric constructs limits, or substantially limits, systemic immune activation and the associated toxic effects (e.g., the molecules may be systemically safe). The constructs described herein do, however, provide immune stimulation localized to the TME where there is sufficient ATP to drive complex formation, and may thus be considered a form of ATP-dependent agonists of immune cell function whose action is limited to that environment. It is understood that dimers and higher order complexes will have higher avidity for cells in the TME as the energy of binding for two or more binding interactions is greater than the energy of binding from the singular (separate) binding interactions for individual constructs. The difference between the binding energy of the individual constructs and dimers or higher order complexes is reflected in the higher binding avidity (low Kd value) for dimers and higher order complexes than the binding affinity observed for any of the individual constructs that make up the dimers or complexes. In addition, because the complexed forms of the constructs (e.g., dimers, trimers, etc.) have an increase in size and molecular weight, their diffusion from the TME is reduced, restricting their action to the TME. Those dimers or higher order complexes that do diffuse out of the TME will dissociate into individual monomers that are ineffective or less effective at stimulating cellular responses as the ATP levels in the surrounding non-malignant tissue are too low to support their complexation. Accordingly, the immune stimulation provided by the complexes of the constructs described herein is understood to be reversible and restricted to microenvironments where ATP levels are sufficiently high to induce complexation.

The binding interactions between ATP mediated complexes of the constructs described herein may also be described in kinetic terms as opposed to the thermodynamic terms described above. Kinetically, the association rate of individual constructs with cells within a tumor is not understood to be affected by the presence or absence of bound ATP as there is little difference between the size or molecular weight of individual constructs and individual constructs with bound ATP, hence little difference in their diffusion rate and ability to access (enter) the TME. In contrast, once constructs are bound to a cell in the TME and form a dimer or higher order complex due to the high ATP levels, the effective affinity for the cells increases and the effective dissociation rate of constructs from the cell is reduced relative to the dissociation rate of individual (monomeric) constructs bound to the same cell surface antigen(s). Unexpectedly large increases in the binding affinity to surface bearing target antigens (e.g., TAAs) have been observed.

While not wishing to be bound by any particular theory, where constructs that homodimerize or heterodimerize have only a single NBD, the presence of multiple constructs bound to a cell surface may unexpectedly result in cell capping or cell surface patch formation in which the cell surface target molecules bound by the constructs are aggregated or clustered on a portion of a cell's surface. For bivalent binding molecules, like many of the dimers described herein, that do not bind to epitopes repeated on the surface of a single cell surface antigen, this phenomenon is unexpected, and may be the result of NBDs reversibly entering into alternative dimer complexes with the NBDs of more than one other molecule located within binding distance. The consequence of forming such clusters may be a local increase in the concentration of the constructs and cell surface molecules bound by the constructs, and potentially a local increase in the level of ATP relative to that found in the TME. Whether viewed from a kinetic or thermodynamic perspective, such concentration increases are understood to result in the constructs remaining bound to the cell surface with an unexpected increase in the effective binding affinity (avidity) where they may continue to affect the cell through interaction with the cell surface protein. For example, formation of cell surface clusters may substantially alter (e.g., reduce or increase the rate of, or even block) internalization of the cell surface molecules present in complexes with the construct, thereby leaving constructs present on target cell surfaces and available to induce responses by cells in the TME (e.g., tumor cells, T cell, NK cells, macrophages and the like). Capping or patch formation on cells may also be induced or amplified using constructs with more than one NBD (see, e.g., FIG. 4A)

Regardless of the mechanism and any unexpected increase in effective binding constants, the high levels of ATP present in the TME result in a biodistribution favoring accumulation of the constructs in the TME. When accessed after a single dose (e.g., given intravenously) the ratio of the maximum concentration of a construct found in the TME may be at least about 1.5 or at least 2 times higher than the level of construct circulating in a patient's plasma. Constructs of the present disclosure may accumulate to a maximum level in a TME at least 3 or at least 4 times higher than the level of construct circulating in a patient's plasma after a single dose of the construct. Constructs of the present disclosure may accumulate to a maximum level in a TME at least 5 or at least 8 times higher than the level of construct circulating in a patient's plasma after a single dose of the construct. Constructs of the present disclosure may accumulate to a maximum level in a TME at least 12 or at least 20 times higher than the level of construct circulating in a patient's plasma after a single dose of the construct. Constructs of the present disclosure may accumulate to a maximum level in a TME in a range from about 1.5 to about 2 or a range of about 2 to about 4 times higher than the level of construct circulating in a patient's plasma after a single dose of the construct. Constructs of the present disclosure may accumulate to a maximum level in a TME in a range from about 4 to about 8, or a range of about 8 to about 20 times higher than the level of construct circulating in a patient's plasma after a single dose of the construct. Constructs of the present disclosure may accumulate to a maximum level in a TME in a range of about 20 or more times higher than the level of construct circulating in a patient's plasma after a single dose of the construct. Accumulation of constructs in tumor tissue at the previously mentioned levels may also be made relative to whole blood, or serum. Similarly, accumulation of constructs in tumor tissue at the previously mentioned levels may also be made relative to peritumoral tissue or in normal (non-transformed tissue) of the organ in which the tumor appears. The above-mentioned levels of accumulation in tumor tissue may be observed whether the construct is administered intravenously or via a non-intravenous route other than intratumoral injection. Irrespective of the comparator tissue/fluid used, the constructs of the present disclosure may continue to accumulate after subsequent injections (administrations) of the construct and may persist after administration in tumor tissue longer versus an otherwise equivalent construct that does not bear an NBD capable of dimerizing (or forming higher order complexes). NBD bearing constructs administered by intratumoral injection may also persist in tumor tissue longer versus an otherwise equivalent construct that does not bear an NBD capable of dimerizing (or forming higher order complexes).

The constructs described herein may also comprise an NBD) and an Ig heavy chain aa sequences (e.g., as a scaffold) that are capable of stimulating Ig-mediated Fc responses including, but not limited to, ADCC, ADCP, and/or CDC. When complexed in the presence of ATP, those constructs become more effective stimulators of at least one Ig-mediated effector function, effectively making the scaffold an AD-like domain. Constructs bearing aa sequences capable of stimulating an effector function need not be targeted to specific cells in the TME to have an anti-tumor effect; however, they may comprise one or more TSBs that target the constructs to one or more TAAs (e.g., two independently selected TAAs) that may present on tumor cells in the TME, thereby focusing the response on the tumor cells.

### C. The Structure of ATP-Dependent Agonists of Immune Cell Function

ATP dependent agonists of immune cell function may be structured in a variety of formats that permit interaction with cells of the immune system through one or more differing mechanisms. The differing mechanisms include, but are not limited to, stimulation of ADCC, ADCP, and/or CDC mediated responses in a tumor cell environment.

One group of constructs described herein comprise one or more NBDs and one or more Ig sequences, which may also act as scaffold sequences capable of stimulating Ig-mediated effector functions. When those constructs are present in a TME with ATP at a sufficient concentration, the ability to stimulate Ig-mediated effector functions is enhanced by the formation of dimers or multimers of the construct. The resulting response by cells such as T effector cells and/or NK cells may be directly focused on tumor cells by incorporation of TSBs that target an antigen on the tumor cell.

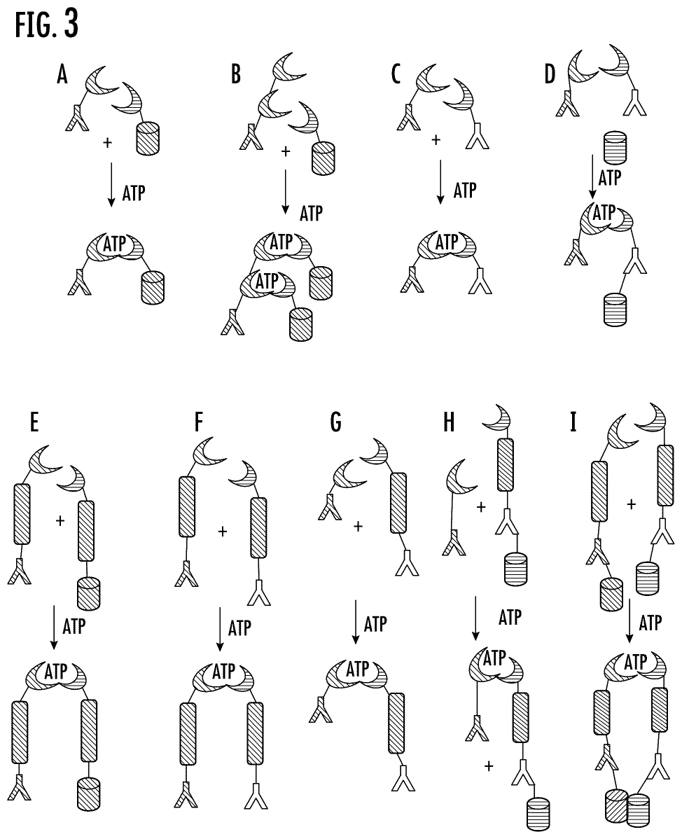

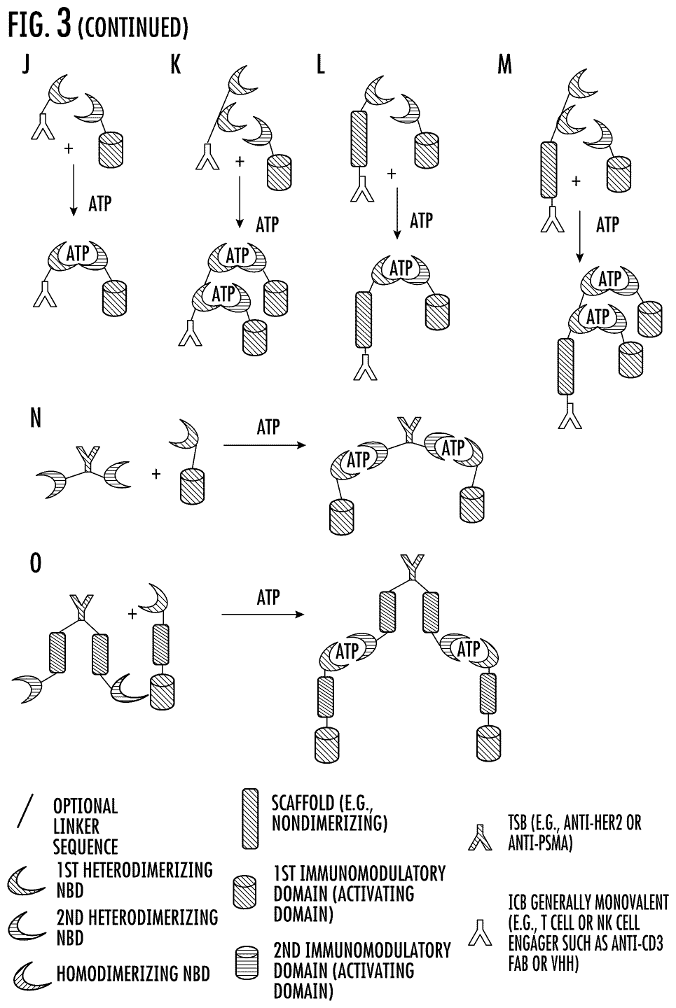

A second group of constructs described herein comprises one or more NBDs and one or more ICBs and/or one or more ADs and optionally one or more TSBs. Those constructs may further comprise a scaffold sequence (e.g., an Ig scaffold). Such constructs may comprise both (i) one or more ICBs or ADs, and (ii) one or more TSBs. Where a TSB and either an ICB or AD are located in a single construct (e.g., they are part of a single polypeptide or bound to a pair of interspecific scaffold aa sequences), the molecule may be considered BiTE-like. Where an ICB is part of a first polypeptide with a first NBD and a TSB is part of a second peptide with a second NBD, heterodimerization of the first and second polypeptides through their NBDs in the presence of ATP results in formation of a BiTE like construct (see, e.g., FIG. 3 at C, D, and F-I). Constructs comprising one or more ICBs and/or one or more TSBs may further include one or more ADs.

A third group of constructs described herein comprises one or more NBDs and one or more TSBs. Such constructs may comprise one or more scaffold sequences.

### 1. NBD-Containing Constructs Comprising Ig Sequences Capable of Stimulating Ig-Mediated Effector Functions

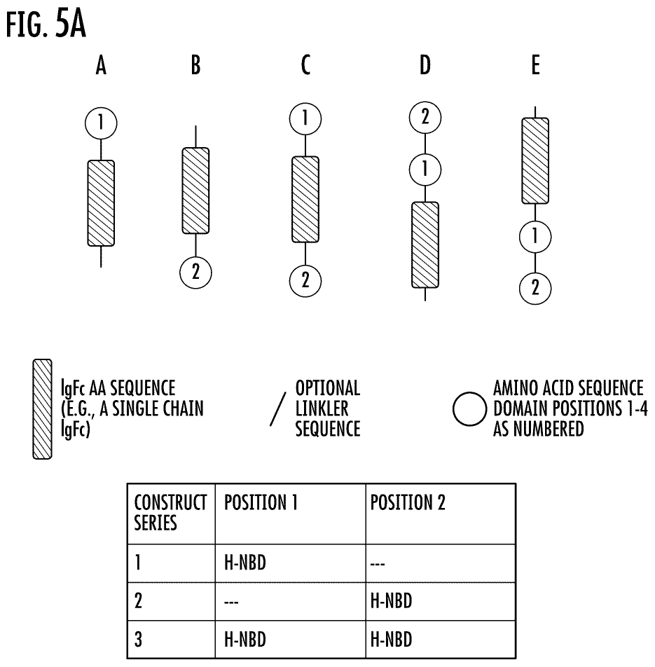

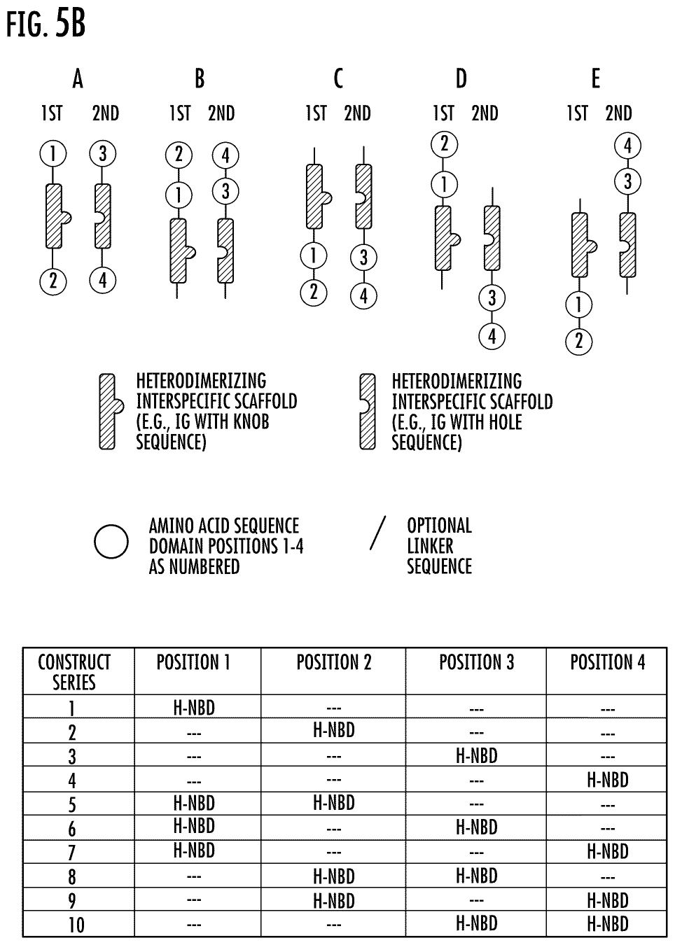

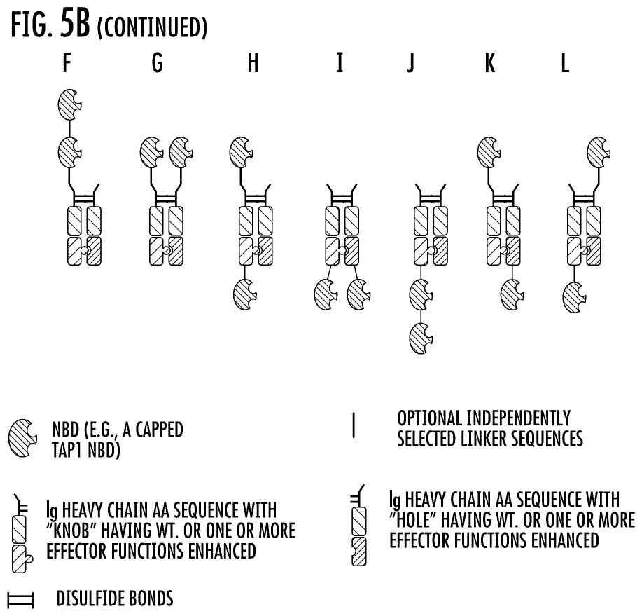

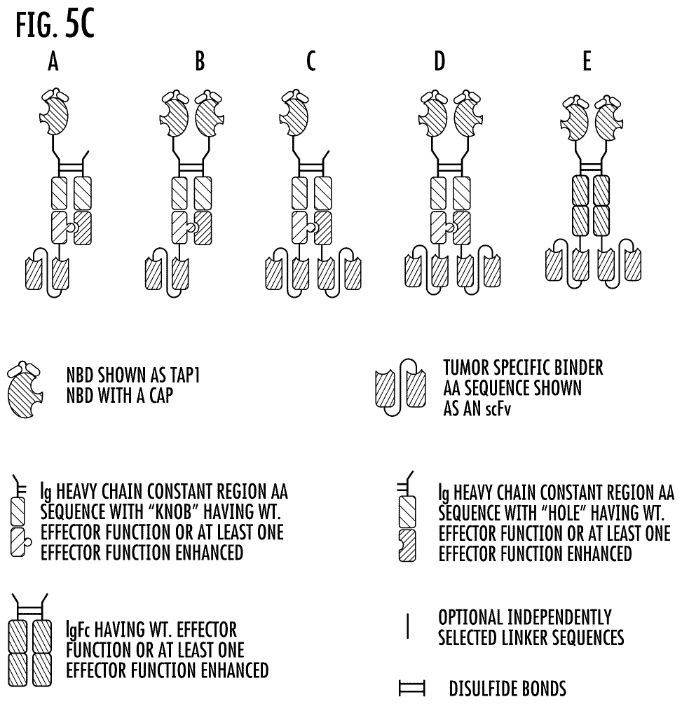

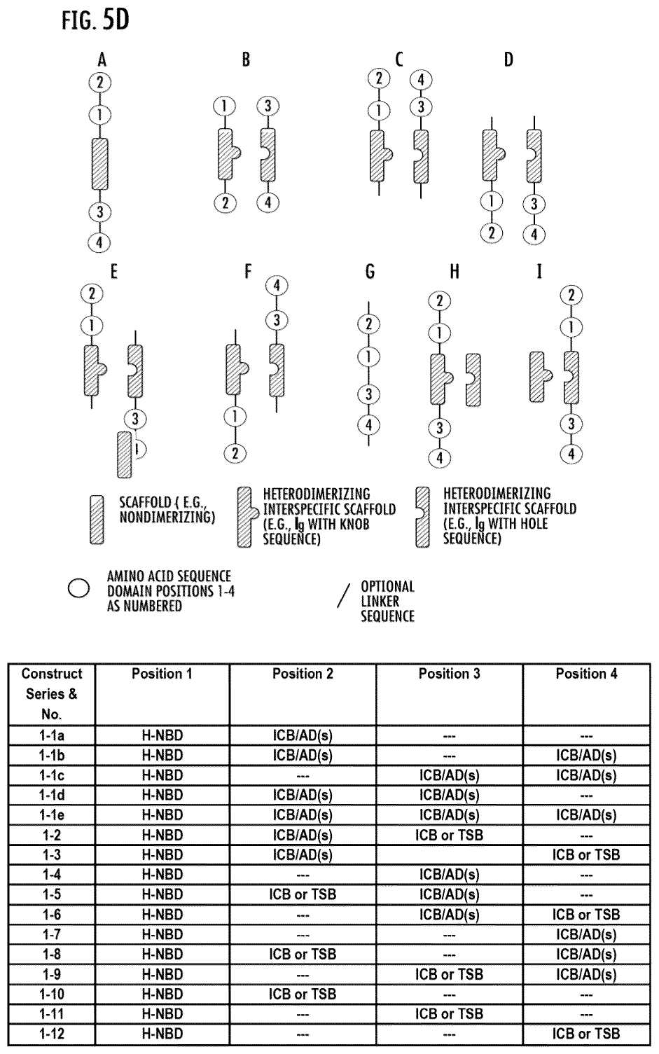

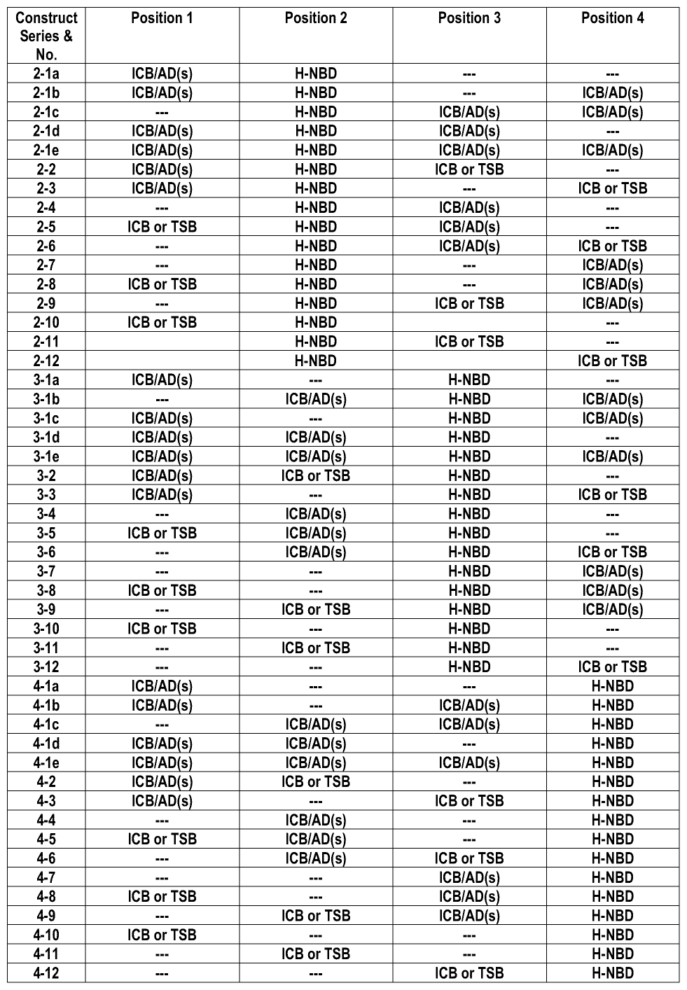

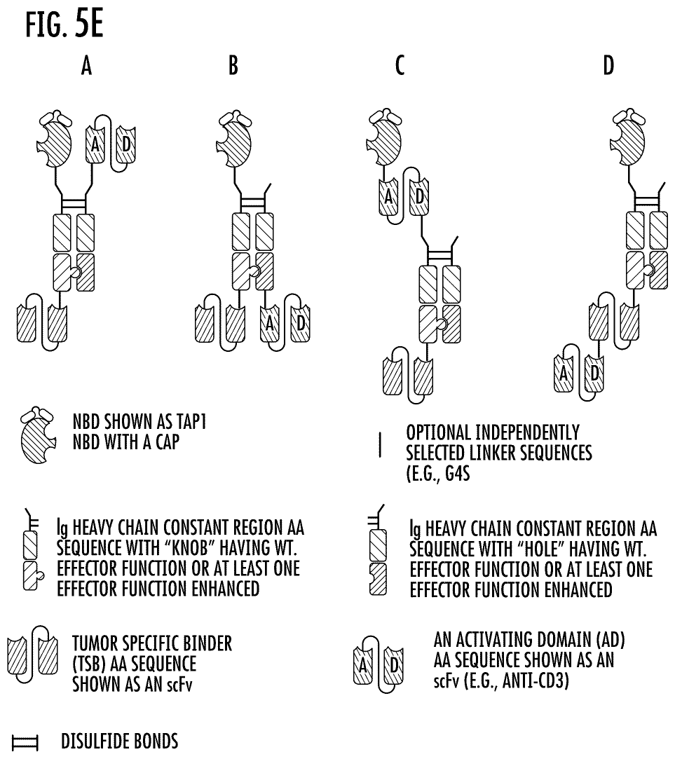

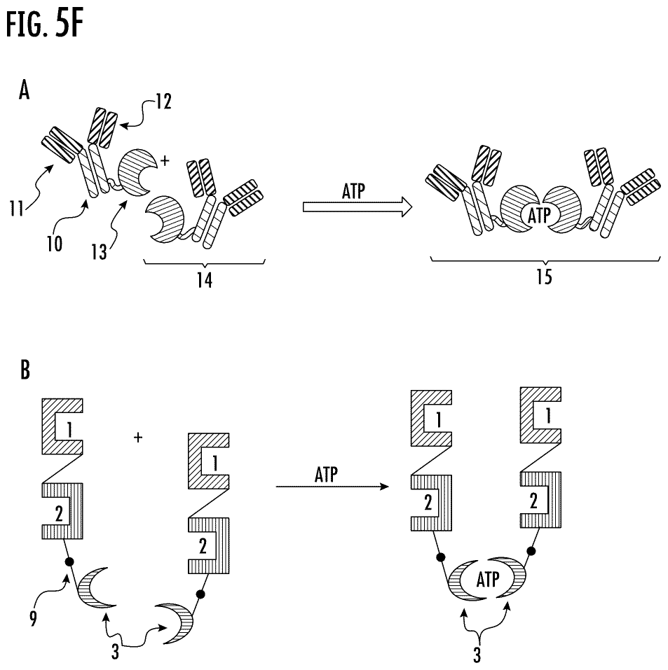

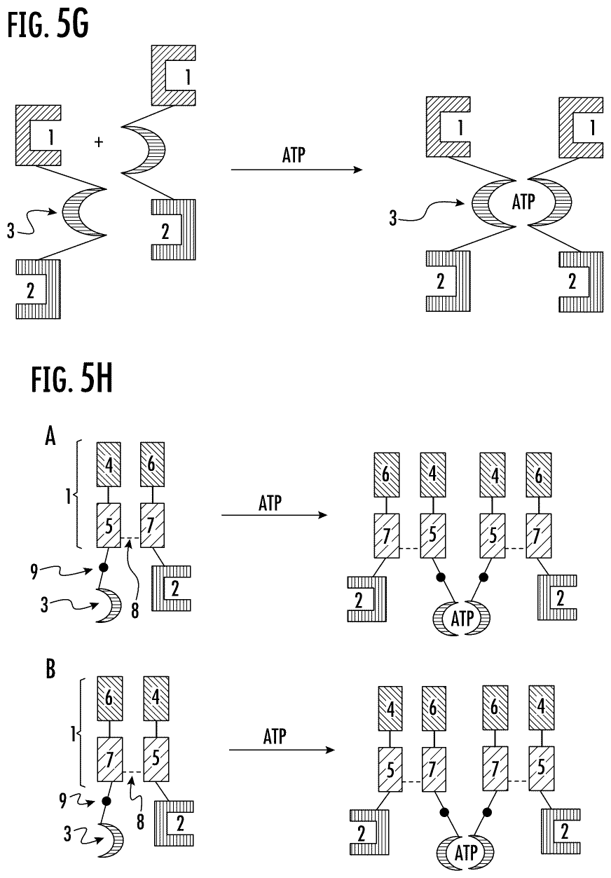

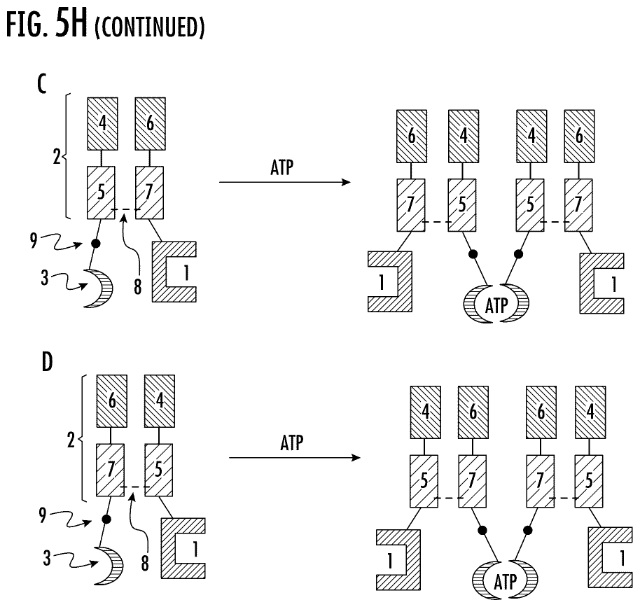

Constructs of the present disclosure that act as ATP-dependent agonists of immune cell function by stimulating any one or more antibody-related effector functions (e.g., ADCC, ADCP, and/or CDC) comprise in their simplest form at least a homodimerizing NBD aa sequence joined to an IgFc aa sequence by an optional linker; see, e.g., FIG. 5A at A and B. The constructs further may comprise a second copy of the NBD (see, e.g., FIG. 5A at C-E). Such constructs may be made asymmetrical where two polypeptides each comprising one of a pair of heterodimerizing NBDs are employed (not depicted in FIG. 5A).

Constructs capable of stimulating one or more Ig-mediated effector functions may also comprise a pair of polypeptides in which: (i) the first polypeptide is comprised of a first Ig heavy chain constant region aa sequence (e.g., comprising Ig CH2-CH3 aa sequences) and optionally an NBD aa sequence joined to the first Ig heavy chain constant region aa sequence directly or indirectly through an optional linker peptide aa sequence, and (ii) the second polypeptide is comprised of a second Ig heavy chain constant region aa sequence (e.g., comprising Ig CH2-CH3 aa sequences) and optionally an NBD aa sequence joined to the second Ig heavy chain constant region aa sequence directly or indirectly through an optional linker peptide aa sequence; wherein at least one (e.g., both) of the first and second polypeptides comprises an NBD (see, e.g., FIG. 5B). In such constructs the first and second Ig heavy chain aa sequences of the first and second polypeptides form an IgFc (e.g., a heterodimeric IgFc where they are an interspecific sequence pair) capable of stimulating an immune cell effector function. The first and second IgFc sequences may be an interspecific sequence pair such as a Knob in Hole (KiH) or KiH with disulfide (KiHs-s). Other interspecific heterodimerizing Ig heavy chain scaffold aa sequences are described herein below. The first and second polypeptides of the constructs may comprise one or more NBDs, or two or more NBDs. Structures F-L in FIG. 5B are representative of constructs having specific pairs of interspecific Ig heavy chain aa sequences that together comprise two homodimerizing NBDs. Stabilizing disulfide bonds between Ig heavy chain sequences, such as those of the lower hinge region and/or between interspecific Ig heavy chain aa sequences (e.g., KiHs-s or EW-RVTs-s), may be present in constructs of the present disclosure.

Where two or more NBD aa sequences are present as part of the same polypeptide (e.g., FIG. 5B at F), or as part of two different polypeptides of the construct (e.g., FIG. 5C at G), they are located such that they cannot form an intramolecular dimer in the presence of ATP, but rather form intermolecular associations with NBDs on a second molecule of the construct. The NBDs may be constrained in this manner by, for example, locating them a sufficient distance apart on the molecule or positioning them, e.g., using rigid linkers, in an orientation such that they cannot dimerize. It will be recognized that where two or more NBDs are present in a construct the molecule may form intermolecular dimers and/or higher order complexes such as trimers, tetramers, etc.

When constructs with an IgFc capable of stimulating one or more effector functions dimerize in the presence of ATP (e.g., in the TME), they increase in size/molecular weight, reducing their diffusion out of a tissue (e.g., out of a TME) permitting accumulation. The formation of dimers or higher order constructs may also enhance the stimulation of any one or more effector functions.

In addition to the accumulation of constructs in environments with ATP sufficiently high to drive dimerization or higher order complex formation, constructs, and their ATP mediated complexes, may further comprise a TSB as a targeting sequence (see, e.g., FIG. 4A, structure C). Alternatively, the constructs may comprise one or more (e.g., two or more) independently selected TSBs that cause not only accumulation in the TME, but also accumulation at the surface of cells comprising the specific target (the TAA) the TSB binds. A variety of TAAs may be targeted including, but not limited to, those set forth herein below. One or more (e.g., two or more) independently selected TSBs may be added to any of the constructs set forth in FIGS. 5A and 5B (e.g., at the N- and/or C-terminus of any one or more polypeptides in those constructs). Two or more TSBs may be incorporated into a construct that stimulates one or more Ig-mediated effector functions to prevent tumor cells from escaping targeted Ig-mediated effector cell function by, for example, mutating a single targeted TAA or suppressing expression of the targeted TAA. Incorporating two or more TSBs with affinity for different TAAs also permits targeting a broader array of tumor cells. FIG. 5C sets forth some specific examples of constructs comprising one or two NBDs and further comprising one or more independently selected TSB aa sequences.

Constructs described herein that stimulate effector functions may comprise IgFc scaffold aa sequences of any suitable isotype of antibody (e.g., IgG or IgM) or subtype thereof (e.g., IgG1, IgG2, etc.), provided they have the ability to stimulate the desired effector function. An IgFc portion of a construct may comprise a single chain IgFc (scFc) aa sequence having two CH2-CH3 sequences connected by a linker of sufficient length that they may pair to form a structure consistent with that in a native heavy chain antibody structure (see, e.g., SEQ ID NO:63). The IgFc portion of a construct also may be formed from two polypeptides that can homodimerize or heterodimerize as an interspecific pair.

Regardless of whether an IgFc sequence is formed as an scFc, or by a pair of homodimerizing or heterodimerizing Ig heavy chain sequences, the resultant IgFc may have wt. effector function or may comprise one or more substitutions that enhance their effector function. Accordingly, constructs may employ wt. IgFc sequences (e.g., a sequence comprising a wt. IgG sequence of SEQ ID NOs: 59 and 64-66). Alternatively, IgFc sequences may comprise one or more substitutions relative to the wt. sequence that enhance their ability to stimulate one or more effector functions (e.g., increase the potency and/or efficacy of IgFc effector function stimulus). An IgFc of a construct may comprise one or more substitutions that enhance one or more of ADCC, ADCP, and/or CDC relative to an otherwise identical construct lacking the substitution(s). Ig heavy chain constant region aa sequences that are present in an scFc that homodimerize, or that heterodimerize as an interspecific pair, may individually or as a pair comprise substitutions that enhance one or more effector functions.

Any of the NBDs of constructs that stimulate one or more effector functions may comprise an N-terminal CAP and/or a stabilizing disulfide bonded with the NBD. TAP1 and/or TAP2 NBDs present in constructs may comprise an N-terminal CAP that comprises both the coupling Helix of TAP1 and the coupling Helix of TAP2 joined by an intra-CAP linker and/or a stabilizing disulfide bond. Stabilizing disulfide bonds may be formed between cysteines appearing or substituted in a TAP1 aa sequence at L493C: E573C, C735: R721C, or C662: Q580C (or at the corresponding positions of TAP2). TAP1 NBD aa sequences may be terminated at about Q741 of SEQ ID NO:8, thereby deleting the C-terminal sequence PADAPE (SEQ ID NO: 11).

### 2. NBD-Containing Constructs Comprising ICB(s), AD(s) and Optionally TSB(s)

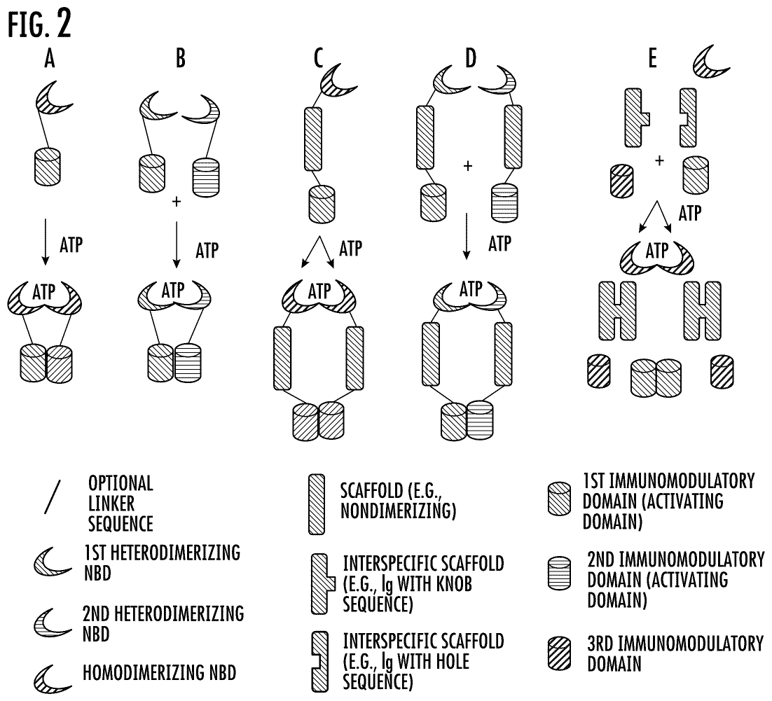

The NBD-containing constructs described herein may, in addition to one or more NBDs, comprise one or more ICBs, one or more ADs, and/or one or more TSBs, and optionally one or more scaffold sequences. As discussed in more detail below, ICBs and ADs are portions (e.g., aa sequences) of NBD-containing constructs that have affinity for cell surface molecules (e.g., antigens) of immune cells, with ADs having the ability to induce a response by the immune cell. Similarly, TSBs are portions (e.g., aa sequences) of NBD-containing constructs that have affinity for cell surface molecules of tumor cells. ICBs and ADs, which may be, for example, antibody fragments, are monovalent to avoid the potential for off-target stimulation (e.g., systemic stimulation) of the immune cells to which they bind. NBD-containing constructs comprising one or more NBDs and/or one or more ICBs and optionally one or more TSBs may appear in a variety of formats as shown, for example, in FIGS. 2-4B, and FIG. 5D.

Constructs comprising as elements one or more ICBs, one or more ADs, and/or one or more TSBs, and optionally one or more scaffold sequences may have those elements arranged in any order. FIG. 5D shows the structures of a series of constructs depicting some possible arrangements for constructs comprising one or more NBDs that homodimerize in the presence of sufficient ATP (H-NBD). For stabilization, the H-NBDs present in the constructs may comprise disulfide bonds and a CAP when located at an N-terminal position (not shown). The constructs further comprise one or more ICBs, one or more ADs, one or more TSBs, and optionally one or more scaffold sequences. Elements of the constructs may be joined by one or more independently selected optional linker sequences indicated as lines between the elements. For the purpose of the constructs in this figure, ICBs and TSBs may be monovalent (e.g., an scFv) or bivalent (e.g., as in an IgG). Each of the ICB, TSB and AD sequences are selected independently. The positions where the construct's elements may be located are shown by the number circles and enumerated in the table below the structures. Constructs are grouped in the table such that the first digit of a construct series indicates the position where an H-NBD is located. A “---” in the table indicates that a position is either unfilled (i.e., it is a bond or linker aa sequence) or filled by one or more AD sequences (AD(s)) which may be in tandem. Additional elements may be included at the N-terminus, C-terminus, or between any two elements except in the case of ADs placed in tandem where the AD sequences are directly connected or separated by an aa linker sequence. Scaffold aa sequences are optional and may be absent (structure G), and when present may be homodimerizing or non-dimerizing (structure A), or heterodimeric as in structures C-F, H and I. The constructs are oriented with the N-terminus at the top. Constructs that comprise a tumor specific antibody sequence and that do not comprise an ICB may comprise a scaffold having wt. or enhanced ADCC, CDC, and/or ADCP function(s). Constructs that have ICBs or ADs should not comprise scaffolds with wt. or enhanced ability to stimulate Ig-mediated effector functions (e.g., ADCC, CDC, or ADCP), and may have the ability to stimulate those effector functions substantially attenuated and/or wholly eliminated.

a) ATP-Dependent Agonists of Immune Cell Function Comprising NBDs and ICBs and/or ADs

NBD-containing molecular constructs described herein may comprise one or more NBDs and one or more ICBs and/or one or more ADs that bind or bind and stimulate an immune cell (e.g., CD8+ T cell, NK cell, etc.). The NBD comprises one or more ATP binding sites that when occupied by ATP result in two or more molecules of the construct forming a duplex or higher order complex (triplex, etc.) due to interactions between the construct's NBDs. Such molecular constructs optionally comprise one or more independently selected scaffold aa sequences and/or one or more independently selected linker sequences. See, e.g., FIG. 1, structure A, and FIG. 2, structures A-D. In some instances, interspecific scaffold sequences that form heterodimers may be incorporated into the constructs such that more than one type of ICB or AD may be present in the molecule as exemplified in FIG. 2 by structure E. The use of ICB(s) or AD(s) (optionally denoted as an “ICB/AD” when singular or “ICB/AD(s)” when singular or plural) that, when brought together (e.g., as a homodimer or heterodimer), can stimulate the targeted population of immune cells such as NK cells and/or T cells permits selective stimulation of an intratumoral immune response due to the high levels of ATP in the TME, while at the same time avoiding systemic activation of the targeted immune cells (e.g., NK cells or T cells) and/or an immune response in non-tumor tissues.

Constructs comprising an NBD and ICB/AD may be arranged in the N-terminal to C-terminal direction as: NBD followed by ICB/AD; or ICB/AD followed by NBD (see, e.g., FIG. 2 at A and FIG. 5D, structure G, substituted as in series No. 1-1a to 1-1d).

The constructs comprising an NBD and ICB/AD may further comprise one or more scaffold sequences that do not dimerize with other scaffold sequences (see, e.g., FIG. 2 at C with a homodimerizing NBD and the structures at D with heterodimerizing NBDs). The constructs may comprise those elements in six different orders. First, a construct may comprise from N-terminus to C terminus the NBD aa sequence, the scaffold aa sequence, and the ICB/AD. Second, a construct may comprise from N-terminus to C-terminus the NBD aa sequence, the ICB/AD and the scaffold aa sequence. Third, a construct may comprise from N-terminus to C-terminus the ICB/AD, the scaffold aa sequence, and the NBD aa sequence. Fourth, a construct may comprise from N-terminus to C-terminus the ICB/AD, the NBD aa sequence, and the scaffold aa sequence. Fifth, a construct may comprise from N-terminus to C-terminus the scaffold aa sequence, the ICB/AD, and the NBD aa sequence. Lastly, a construct may comprise from N-terminus to C-terminus the scaffold aa sequence, the NBD aa sequence, and the ICB/AD. Where two of such constructs that comprise a pair of NBDs capable of heterodimerization are combined, they may form a complex in the presence of ATP (see, e.g., FIG. 2 at D).

The constructs comprising an NBD, an ICB/AD and one or more scaffold aa sequences may comprise
(i) a first polypeptide comprised of a first scaffold aa sequence (e.g., comprising an Ig heavy chain constant CH2-CH3 aa sequence) and optionally a first NBD aa sequence joined to the first scaffold aa sequence directly or by a linker aa sequence, and
(ii) a second polypeptide comprised of a second scaffold aa sequence (e.g., comprising an Ig heavy chain constant CH2-CH3 aa sequence) and optionally a second NBD aa sequence joined to the second scaffold aa sequence directly or by a linker aa sequence;
wherein
(i) at least one of the first and second polypeptides comprises an NBD aa sequence,
(ii) at least one of the first and second polypeptides comprises an ICB or AD aa sequence,
(iii) each NBD aa sequence comprises one or more ATP binding sites and can, in the presence of ATP, homodimerize or heterodimerize with a cognate non-identical second NBD,
(iv) the first and second scaffold sequences form a dimer via interactions between the first and second scaffold sequences (e.g., an IgFc), and
(v) each linker sequence present is selected independently. (See, e.g., FIG. 5B at A-E.)

In such constructs, the first and second polypeptide sequences form a dimer via interactions between the first and second scaffold sequences, which may be Ig or non-Ig aa sequences. The scaffold sequences of the first and second peptides may be, for example, a pair of heavy chain constant region aa sequences (CH1-CH2) that form an IgFc structure when they dimerize. The scaffold sequences that form a dimer may also be a pair of interspecific Ig sequences, such as KiH or KiHs-s variants of IgG. The scaffold sequences may also be a light chain constant region (Cλ or Cκ) aa sequence paired with a heavy chain CH1 region aa sequence as a dimer.

b) ATP-Dependent Agonists of Immune Cell Function Comprising NBDs and ICBs and/or ADs and Further Comprising a TSB

Constructs comprising NBDs and ICBs and/or ADs may further comprise one or more sequences (e.g., TSBs) that permit targeting of the constructs to cells or tissues expressing the cognate binding partner of the targeting aa sequence. Where the target is a TAA, the TSB permits direct targeting of tumor cells. NBD-containing constructs comprising a TSB associated with one or more ICB and/or one or more ADs are depicted, for example, by the constructs of FIG. 5D. See, e.g., FIG. 5D, structures B-F, H and I, when substituted as in Construct Series No. 1-2, 1-5, 2-8, 2-2, 2-5, 3-8, 3-2, 3-5, 3-8, 4-2, 4-5, and 4-8 where the “ICB or TSB” is selected to be a TSB. Specific examples of such constructs are also provided, for example, in FIG. 5E and FIG. 5F.

3. NBD-Containing Constructs Comprising TSBs and Optionally Scaffolds with Effector Function Substantially Diminished

The NBD-containing constructs described herein may comprise one or more NBD aa sequences, one or more TSB aa sequences, and optionally one or more scaffold aa sequences with one or more Ig-mediated effector functions substantially diminished (e.g., wholly eliminated). The size and affinity of such constructs for the TAA to which the TSB binds increases upon formation of a dimer (see, e.g., FIG. 4B at A-D) or higher order complexes (see, e.g., FIG. 4A at C) in the presence of ATP at the levels found in the TME. The change in size and affinity may lead to accumulation in the TME. Such constructs may act by blocking checkpoint proteins such as CTLA-4 (e.g., where the TSB is based on the variable region of ipilimumab, which acts in an antagonist CTLA-4 function). Alternatively, the construct may block the binding of ligands for receptors or prevent receptors on tumor cells from functional cell signaling (e.g., as Trastuzumab prevents EGF dependent tumor cells from responding to EGF by binding to the HER2). See Zhao et al. Antibodies (Basel) 10 (1): 7 (2021).

Constructs comprising as elements one or more NBDs, one or more TSBs, and optionally one or more scaffold aa sequences may have those elements arranged in any order. Structures A-I of FIG. 5D depict a series of possible arrangements for such constructs when substituted as in Construct Series Nos. 1-10 to 1-12, 2-10 to 2-12, 3-10 to 3-12, or 4-10 to 4-12 when the “ICB or TSB” is a TSB. Some positions where the NBDs and TSBs may be located are shown by the number circles and enumerated in the table below the structures. Constructs are grouped in the table such that the first digit of the construct series indicates the position where an H-NBD is located. The constructs may comprise additional NBDs or TSBs, resulting in constructs with two or more NBDs and/or two or more TSBs. Each of the TSB sequences in any construct is selected independently. For stabilization, the H-NBDs present in the constructs may comprise disulfide bonds and a CAP when located at an N-terminal position (not shown). Elements of the construct may be joined by one or more independently selected optional linker sequences indicated as lines between the elements in the accompanying figures. For the purpose of the constructs in this figure, TSBs may be monovalent (e.g., an scFv), bivalent (e.g., as in an IgG), or bispecific (directed to two distinct TAAs).

For the purpose of NBD-containing constructs comprising TSBs, an “---” in the table of FIG. 5D generally indicates that a position is unfilled (i.e., it is a bond or linker aa sequence). It is noted, however, that positions marked as “---” may be filled by one or more ICB or AD sequences (AD(s), which may be in tandem. Additional elements may be included at the N-terminus, C-terminus, or between any two elements except in the case of ICB/ADs placed in tandem where the AD sequences are directly connected or separated by an aa linker sequence. Scaffold aa sequences are optional and may be absent (structure G), and when present may be homodimerizing or non-dimerizing (structure A), or heterodimeric as in structures C-F, H and I.

4. NBD-Containing Constructs that Heterodimerize Through their NBDs in the Presence of ATP

TSBs and ICB/AD(s) may be incorporated into NBD-containing constructs that form heterodimers or other higher order complexes (e.g., trimers) through a pair of NBDs that are cognate binding partners (e.g., TAP1 and TAP2) in the presence of ATP at the concentration found in, for example, the TME environment. Heterodimer formation may be driven by the incorporation of a first (1^{st}) heterodimerizing NBD in the first member of the pair (first subconstruct or first polypeptide) of constructs and a second (2^{nd}) heterodimerizing NBD in the second member of the pair (second subconstruct or second polypeptide) of constructs. The 1^{st} and 2^{nd} heterodimerizing NBDs act as cognate binding partners. The pairs of members of the constructs may be divided into six groups, wherein the individual polypeptides or subconstructs (e.g., made up of peptides dimerized through their scaffolds) comprise the elements set forth in Table 1.

TABLE 1
Organization of some heterodimeric constructs

| Heterodimeric | Polypeptide or | | First functional | Second functional | |
|---|---|---|---|---|---|
| Construct | Subconstruct | NBD | aa sequence | aa sequence | Scaffold |
| 1 | first | First NBD† | One or more ICB | optional AD | optionally present § |
| | second | Second NBD† | One or more ICB | optional AD | optionally present § |
| 2 | first | First NBD | One or more ICB | optional AD | optionally present § |
| | second | Second NBD | One or more AD | optional AD | optionally present § |
| 3 | first | First NBD | One or more AD | optional AD | optionally present § |
| | second | Second NBD | One or more AD | optional AD | optionally present § |
| 4 | first | First NBD | One or more TSB | optional AD | optionally present ‡ |
| | second | Second NBD | One or more ICB | optional AD | optionally present ‡ |
| 5 | first | First NBD | One or more TSB | optional AD | optionally present ‡ |
| | second | Second NBD | One or more AD | optional AD | optionally present ‡ |
| 6 | first | First NBD | One or more TSB | optional AD | optionally present ‡ |
| | second | Second NBD | One or more TSB | optional AD | optionally present ‡ |

†The First and Second NBDs form a heterodimerizing pair (e.g., the first NBD is from TAP1 and the second NBD is from TAP2).
§ Scaffolds present in constructs comprising an ICB and/or AD typically are incapable of stimulating Ig-mediated immune cell effector functions such as ADCC, ADCP, and/or CDC.
‡ Scaffolds present in constructs comprising a TSB are optionally capable of stimulating Ig-mediated immune cell effector functions such as ADCC, ADCP, and/or CDC, and may bear one or more substitutions that enhance any one or more of those functions.

Where more than one ICB, AD and/or TSB are present in the heterodimeric constructs, they are each selected independently. In the case of constructs with two or more ADs, it is possible to reconstitute an active heterodimeric AD (e.g., from the IL-12 superfamily-IL-12, IL-23, IL-27 and IL-35) by locating one subunit of the heterodimer in the first polypeptide or subconstruct and the second subunit of the heterodimer in the second polypeptide or subconstruct.

In order to exemplify the type of such NBD-containing construct pairs encompassed by the present disclosure, some of which are summarized in Table 1, a series of non-limiting examples of such NBD-containing construct pairs is set forth in FIG. 3. In vivo the dimerized constructs may direct immune cells bound by the ICB/AD to interact with target tumor cells recognized by a TSB, bringing about an immune response to the target tumor cells. Such constructs permit the selective targeting of tumor cells in the TME because they require the elevated ATP of the TME for complex formation, and thereby limit off-target binding and stimulation of immune cells. The paired constructs comprising TSBs and ICBs may be augmented by the incorporation of one or more ADs (that may be the same or different), which may require dimerization brought about by interaction of the first and second NBD-containing constructs to stimulate the target immune cells (e.g., as in the case of IL-12 superfamily members).

Entries A and B of FIG. 3 each provide a pair of constructs in which the first NBD-containing construct comprises (e.g., from N-terminus to C-terminus or from C-terminus to N-terminus) one or more 1^{st} heterodimerizing NBDs linked to a TSB and the second NBD-containing construct comprises (e.g., from N-terminus to C terminus) a cognate 2^{nd} heterodimerizing NBD and an AD. The elements of the constructs are joined by optional linker sequences that are independently selected. Such constructs permit the targeting of ADs to tumor cells in the TME as they require the elevated ATP of the TME to heterodimerize, and thereby limit off-target binding and avoid systemic stimulation of immune cells by the AD. In addition, a mix of more than one construct comprising a 2^{nd} heterodimerizing NBD and non-identical ADs may be employed to create the constructs set forth in FIG. 3 at B, which upon ATP mediated complex formation may function to present more than one AD to the immune cells. The use of more than one 2^{nd} heterodimerizing NBD and the subunits of heterodimeric ADs (e.g., in the IL-12 superfamily) in constructs such as those in FIG. 3 at D may also result in reconstituted AD activity (e.g., IL-12 activity). Although not shown in FIG. 3 at A or B, either or both of the heterodimerizing constructs in A or B may also comprise a scaffold aa sequence (see, e.g., entry E in FIG. 3).

An exemplary pair of constructs that illustrates heterodimerization of the type shown in entries A or B of FIG. 3 may comprise, for example: (i) a first NBD-containing construct comprising one or more TAP1 NBDs linked to a TSB that has affinity for a TAA (e.g., mesothelial (MSLN), EpCAM, and CTLA-4); and (ii) a second NBD-containing construct comprising a TAP2 NBD that acts as the cognate binding partner of the TAP1 NBD in the presence of ATP and an IL-2 sequence. When exposed to ATP at the levels present in the TME, such constructs heterodimerize and can act to stimulate T cells present in the TME.

Entries C and D of FIG. 3 each provide a heterodimerizing pair of constructs in which the first NBD-containing construct comprises (e.g., from N-terminus to C-terminus) one or more 1^{st} heterodimerizing NBDs linked to a TSB and the second NBD-containing construct comprises (e.g., from N-terminus to C terminus) a cognate 2^{nd} heterodimerizing NBD and an ICB. In the presence of ATP the constructs form a BiTE-like construct. The elements of the constructs are joined by optional linker sequences that are independently selected. Either or both of the first and second constructs of the heterodimerizing pair may comprise a scaffold sequence (see, e.g., FIG. 3 at F to I). Constructs comprising a TSB and/or an ICB may further comprise an AD as in entries D, E, H, and I. Where an AD is present in a construct that also comprises an ICB, the AD may be selected from those that require presentation to the T-cell as a dimer or higher order complex (e.g., TNF family members such as 4-1BBL) for immune cell stimulation to prevent off-target stimulation.

A pair of constructs that exemplify entry C of FIG. 3 comprises, for example: (i) a first NBD-containing construct comprising (a) a TAP1 NBD linked to (b) a TSB; and (ii) a second NBD-containing construct comprising (a) a TAP2 NBD that acts as the cognate binding partner of the TAP1 NBD in the presence of ATP and (b) a monovalent ICB that binds T cells (e.g., an anti-CD3 antibody fragment or single chain antibody construct such as a nanobody or scFv). The TSB may be an antibody, antibody fragment, or single chain antibody construct such as an scFv or nanobody) that has affinity for a TAA expressed on a tumor cell surface (e.g., mesothelial (MSLN), EpCAM, and CTLA-4).

Entries A to I of FIG. 3 appear as combinations of two molecular constructs. Because the first construct comprising the TSB in those combinations is separate from the second construct comprising an AD and/or an ICB, a population (mixture) comprising more than one type of first or second construct may be formed. For example, a mixture of first and second constructs depicted in any one of entries A to I of FIG. 3 may be formed wherein the mixture comprises two or more first constructs each having TSBs targeting different TAAs. A mixture of first and second constructs depicted in any one of entries A or B of FIG. 3 may be formed wherein the mixture comprises two or more second constructs each having one or more ADs directed to different immune cell receptors. Similarly, a mixture of first and second constructs depicted in any one of entries C to I of FIG. 3 may be formed wherein the mixture comprises two or more second constructs each having different ICBs directed to different immune cell surface antigens. In addition to populations where only one of the ICB, TSB, or AD is varied, the mixtures of first and second constructs may comprise two or more first and two or more second constructs that vary in two or more elements selected from the ICB, TSB, and AD.

Entries J through M of FIG. 3 each provide a pair of constructs in which the first NBD-containing construct comprises (e.g., from N-terminus to C terminus) one or more 1^{st} heterodimerizing NBDs linked to an ICB and the second NBD-containing construct comprises (e.g., from N-terminus to C terminus) a cognate 2^{nd} heterodimerizing NBD and an AD. The elements of the constructs are joined by optional linker sequences that are independently selected. Such constructs permit the activation of immune cells in the TME while avoiding any substantial activation outside of the TME as they require elevated ATP to heterodimerize, and thereby limit off-target binding and avoid systemic stimulation of immune cells by the AD. In addition, the ADs of the constructs set forth in either K or M may be the same or different and may require dimerization to function on the target immune cell.

Exemplary pairs of constructs that illustrate heterodimerizing constructs of the type found in FIG. 3 at J and K comprise, for example: (i) a first NBD-containing construct comprising one or more TAP1 NBDs linked to an ICB (e.g., an antibody, antibody fragment, or single chain antibody construct such as a VHH or nanobody) that has affinity for CD28 expressed on a T cell surface; and (ii) a second NBD-containing construct comprising a TAP2 NBD that acts as the cognate binding partner of the TAP1 NBD in the presence of ATP and an IL-2 sequence. Such constructs when complexed into a heterodimer by ATP at the levels present in the TME can act to stimulate T cells present in that environment.

Constructs and compositions comprising a mixture of constructs each comprising one or more TSBs (e.g., one or two) or one or more constructs comprising two or more TSBs directed against different TAAs find use in therapeutic applications where the use of more than one TSB directed against different TAAs (or different epitopes of a TAA) can avoid tumors escaping therapy targeted at a single TAA. Similarly, the use of more than one ICB and/or AD can recruit more than one type of immune cell and provide a more vigorous immune response to the tumor cells, thereby effectuating a more robust immune response to the tumor than would occur using monotherapy with a single ICB and/or AD.

### 5. NBD-Containing Constructs Comprising Two or More NBDs and the Formation of Complexes of NBD-Containing Molecular Constructs

The NBD-containing constructs described herein may comprise two or more NBDs that permit the constructs to effectively polymerize into complexes in the presence of ATP. As explained elsewhere, constructs may be arranged such that two NBDs within any given molecule do not interact to form an intramolecular dimer (they do not bind in cis), but instead interact with NBD(s) on other molecules (bind in trans). The two or more NBDs may be arranged as a tandem pair. When elements like NBDs are placed in tandem they are adjacent to each other in the construct and are not separated by any element of the constructs other than an intervening linker aa sequence (e.g., in the case of NBDs there are no intervening ADs, TSBs, or ICBs present between the tandem NBDs). NBDs may also be located in the construct so that they are not placed in tandem. NBD-containing constructs comprising two or more NBDs (e.g., in tandem) may also be formed from constructs comprising ICB/AD(s) or TSBs by the addition of one or more NBDs.

Complexes of NBD-containing molecular constructs comprising at least two NBDs can be formed by placing the constructs in an environment, such as a TME, where ATP levels are sufficiently high for ATP to occupy the binding sites. Such complexes are exemplified in FIG. 3 at N and O, and in FIG. 4A. Where, as in FIG. 4A, more than one NBD aa sequence is incorporated into constructs of the present disclosure, the constructs may, in effect, reversibly polymerize in the presence of ATP to produce complexes. The complexes formed in the presence of ATP may be dimers, trimers, tetramers or even higher order structures. See, e.g., FIG. 4A. Incorporating more than one NBD in the constructs may permit maximization of potency and efficacy and may affect other construct properties. For example, constructs that form complexes in the presence of ATP that are polyvalent for an element expressed on the surface of target cells (e.g., polyvalent TSBs or ICB/AD(s) may display an increased effective affinity for the target cells and form larger complexes that may remain in the TME longer, thereby effectively extending the in vivo half-life, etc.

Complexes may comprise at least two NBDs arranged in tandem, with at most a linker aa sequence (e.g., a rigid linker) separating the NBD aa sequences (see FIG. 4A, structures A, B, and E), and an AD, ICB and/or TSB. Alternatively, the NBDs may be separately attached to the TSB or ICB (see FIG. 4A, structures C and D). NBD-containing constructs used to form complexes may comprise homodimerizing or heterodimerizing NBDs. Although not shown in FIG. 4A, homodimerizing NBDs in the same construct molecule may be prevented from self-associating by constructing the molecule such that a productive ATP binding interface between the domains cannot be formed. This may be accomplished by, for example, placement of the NBDs away from each other on the protein (e.g., the ATP binding sites are on opposite faces of the construct), limiting the distance on a construct between the NBDs, and/or by the use of a rigid peptide linker between the NBDs.

NBD-containing constructs comprising at least two NBDs and an AD are exemplified in FIG. 4A by structures A and B. Structure A employs an NBD that homodimerizes in the presence of ATP and may form dimers and/or a larger construct as shown in FIG. 4A, structure A. In contrast, structure B comprises a structure that forms a heterodimer (not shown) or polymer in the presence of ATP. When exposed to ATP at the levels present in the TME, the constructs may form large complexes presenting ADs (e.g., as AD homodimers or heterodimers). Complexes of such NBD-containing constructs may stimulate the targeted immune cells, such as NK cells and/or T cells, in the TME. The ADs present in the complex may be the same (e.g., FIG. 4A, structure A) or different (e.g., FIG. 4A, structure B). For example, the aa sequence of the 1^{st} AD and the 2^{nd} AD present in a complex may be identical and lead to activation of an immune cell response when presented in a dimer or higher order complex. Alternatively, the 1^{st} AD and the 2^{nd} AD present in a complex may be from different molecules (e.g., different interleukins) that when presented together cause immune cell activation. It is also possible that the aa sequence of the 1^{st} AD and the 2^{nd} AD present in a complex are different (nonidentical) and when combined form an active fragment of a heterodimeric immunomodulator such as IL-12, IL-23, IL-27, or IL-35, each of which comprises two subunits. NBD-containing constructs, each comprising a single NBD, may be assembled into a complex comprising two different (non-identical) ADs in the presence of ATP. Alternatively, active AD comprised of two subunits can assemble into an active AD using two constructs, each comprising a single NBD, in the presence of ATP (see, e.g., FIG. 2, structures A and B).

In addition to the NBD-containing constructs described above, this disclosure provides for NBD-containing constructs that comprise at least two NBDs and a TSB exemplified in FIG. 4A, structure C, and/or an ICB aa sequence, exemplified by FIG. 4A, structure D. Such constructs optionally comprise one or more independently selected scaffold aa sequences and/or one or more independently selected linker sequences. TSBs include, but are not limited to, antibodies (e.g., scFv and/or nanobodies) that bind to TAAs (e.g., HER-2, mesothelial (MSLN), EpCAM, CTLA-4, and the like). ICBs include molecules including, but not limited to, antibodies that bind to immune cell surface antigens (e.g., CD3, CD8, or CD4 on αβ T cells, TRGV9 on δγ T cells, CD16 on NK cells, etc.). Unless stated otherwise, ICBs are monovalent to avoid stimulation of immune cells outside of the TME and have sequences that can lead to ADCC or CDC modified to substantially limit or prevent such responses.

Once present in the TME where ATP levels permit the NBD domains to form higher order complexes (e.g., dimers), constructs that comprise at least two NBDs and a TSB and/or an ICB form large complexes that can lead to immune stimulation. Constructs comprising at least two NBDs, a scaffold comprising an IgFc, and a TSB can form a complex in the TME (see, e.g., FIG. 4A, structure C) that can, for example, lead to ADCC or CDC. Constructs comprising at least two NBDs and an ICB can form a complex in the TME (see, e.g., FIG. 4A, structure D) that can, for example, lead to stimulation of the targeted immune cells due to crosslinking of the target antigen (e.g., CD3 on CD8+ T cells or CD16 on NK cells) on their surface.

Combining an NBD-containing construct comprising at least two NBDs and a TSB (e.g., FIG. 4A, structure C) and an NBD-containing construct comprising at least two NBDs and an ICB (e.g., FIG. 4A, structure D) may result in complexes that comprise both TSBs and ICBs. The use of 1^{st} heterodimerizing NBDs joined to the TSB and 2^{nd} heterodimerizing NBDs joined to the ICB ensures the complex formed in the presence of ATP contains both ICBs and TSBs and can stimulate immune cell responses to the target tumor cell. This is exemplified with mixtures of constructs comprising at least two NBDs and a TSB and constructs that comprise at least two NBDs and an ICB forming large complexes in the TME (see, e.g., the structures in FIG. 4A at E and F where the NBDs are shown in tandem). Complexes resulting from such structures in the presence of sufficient ATP can bind immune cells to tumor targets via the TSB and at the same time bring about activation of the immune cell by crosslinking of the receptors on the immune cell's surface (e.g., via the monomeric ICB that is now in the polyvalent complex).

The TSB and/or ICB aa sequences present in each construct used to form a complex need not be the same. Accordingly, the complexes of FIG. 3, structures A to I, and FIG. 4A, structures C to I, may comprise constructs with more than one TSB directed to different TAAs (e.g., two or more TSBs) and, when present, more than one ICB. Using two or more different TSBs directed against different TAAs in a single complex not only permits the complex formed from constructs comprising two or more NBDs to be active against tumors expressing different tumor antigenic targets, but also may limit tumor escape from therapy by loss of tumor antigen expression. Using different ICBs in a single complex may also permit the stimulation of an immune response from more than one type of immune cell (e.g., both CD8+ T cells and NK cells). It is also possible to incorporate ADs into complexes that form in the TME, including incorporation of ADs into complexes containing a construct that includes a TSB. This can be done, for example, by combining one or more constructs that comprise an AD and an NBD (e.g., two or more NBDs) in the complex such as is exemplified in FIG. 4A at H, or by combining one or more constructs that comprise an AD, an NBD (e.g., two or more NBDs), and a TSB in the complex such as is exemplified in FIG. 4A at I.

Because the TSBs, ICBs, and ADs are provided by different constructs that enter into the complex formed in the TME, the composition of the complex can be controlled by mixing different constructs (e.g., prior to or during administration to a patient). Thus, a single construct may find use in multiple different therapeutic combinations.

### D. Elements of NBD-Containing Constructs

1. NBDs and Interactions with ATP

NBDs for incorporation into the constructs described herein may be selected based on several criteria. Where the constructs are intended for human therapeutic use, the NBDs are preferentially derived from human ATP binding proteins to limit their immunogenicity and are preferably able to undergo solution phase dimerization or higher complex formation when exposed to sufficient ATP. Where non-human proteins that are immunogenic are employed, the immunogenic portions may be humanized by altering the immunogenic regions to more closely match, or completely match, their human homologs. The solution phase formation of dimers or higher order complexes used in the constructs described herein occurs at ATP concentrations found in the target TME, but not in normal tissues. Accordingly, an NBD for use in the constructs provided herein may have, for example, a dissociation constant for ATP from about an order of magnitude above (10 times above) the upper level found in the extracellular space of normal tissues (i.e., about 1 μM or higher) up to about the level of ATP found in the TME. In some cases, the affinity of an NBD will be from about 1 μM to about 200 μM. For example, the ATP affinity of an NBD may be in a range from about 1 μM to about 5 μM or from about 5 μM to about 25 μM. The ATP affinity of an NBD may be in a range from about 10 μM to about 25 μM or from about 25 μM to about 200 μM. The ATP affinity of an NBD may be in a range from about 25 μM to about 50 μM. In other examples, the affinity of an NBD may be in a range from about 50 μM to about 100 μM or from about 100 μM to about 200 μM. An NBD for use in the constructs provided herein may have, for example, a dissociation constant for ATP greater than about 5 μM or greater than about 10 μM.

NBDs of some proteins form homodimers in the presence of sufficient levels of ATP, while others form heterodimers in the presence of sufficient levels of ATP. Both homodimerizing NBDs and heterodimerizing NBDs may be employed in various aspects of the polypeptide constructs described herein. Where it is desirable to have the NBDs form homodimers in solution when sufficient levels of ATP are present, aa residues at the interface between the dimers may be modified to either remove or substitute aa residues that interfere with interface formation between two NBD molecules. Alternatively, it is possible to substitute aas not contributing to the interactions between the two NBD molecules with aas that will contribute to interactions resulting in homodimer formation. Sequences from TAP1 and TAP2 are known to form heterodimers and may be employed in the constructs described herein for heterodimer formation. Sequences from ABCG5 and ABCG8 are also known to form heterodimers and may be employed in the constructs described herein for heterodimer formation. See, e.g., Alam et al., Annu. Rev. Biophys. 2023. 52:275-300.

### a) ATP-Binding Cassettes as NBDs

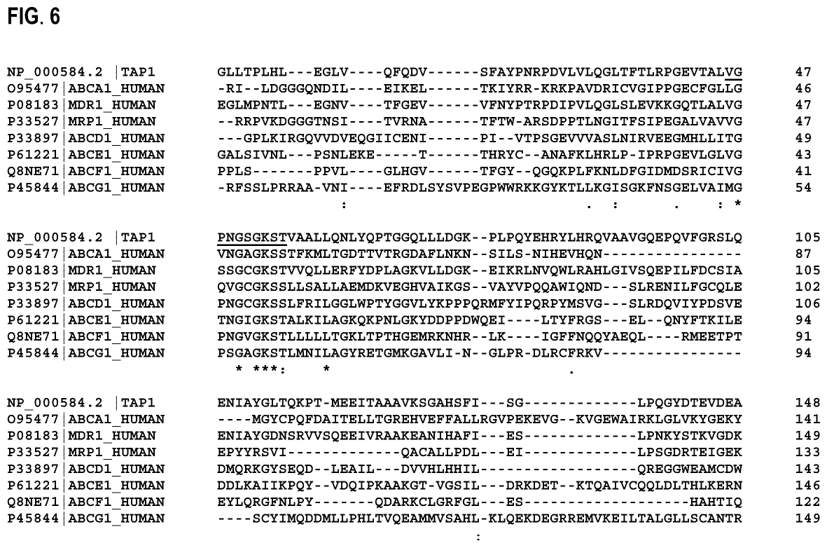

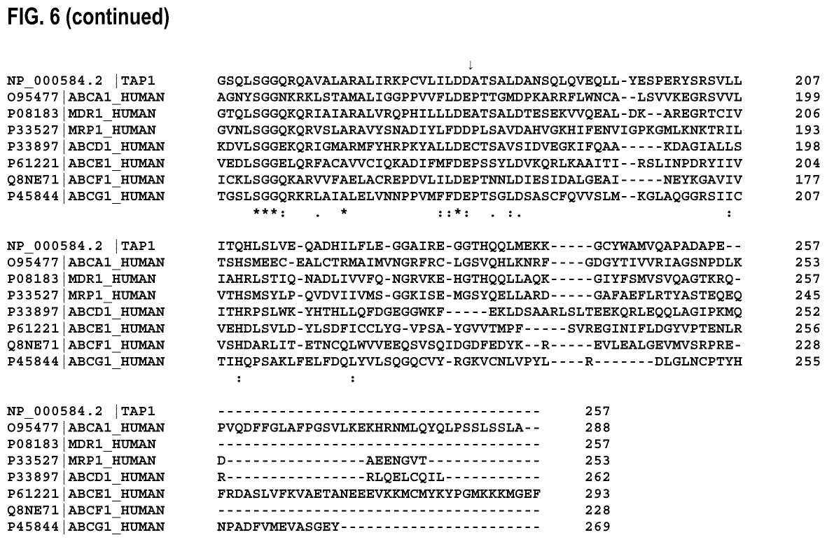

A number of proteins comprise NBDs that are suitable for use as NBDs of the constructs described herein. Among those proteins, ATP-binding cassette (ABC) transporters represent suitable candidates in part because they are an abundant transporter family that is highly conserved and as such unlikely to be immunogenic. ABC transporters are a large superfamily of membrane proteins with diverse functions that utilize the energy from ATP hydrolysis to facilitate transport of substrates either into or out of the cytoplasm. See, e.g., Locher, Philos Trans R Soc Lond B Biol Sci., 364 (1514): 239-245 (2009), and references cited therein. The human genome is thought to code for 48 or 49 distinct ABC transporters. Id. The ABC transporters are divided into seven distinct family members, ABC1, MDR/TAP, MRP, ALD, OABP, GCN20, and White. A representative member of each family including TAP1 are aligned in FIG. 6. The NBD of the ATP-binding cassette family is substantially conserved and comprises two sub-domains. Id. The first resembles RecA protein and contains P-loops (Walker-A motifs). The second subdomain, referred to as the “helical sub-domain,” comprises an LSGGQ motif (SEQ ID NO:10). Id. The NBDs of the transporters assemble as dimers in head-to-tail orientation forming two ATP binding and hydrolysis sites sandwiched at their interface (see FIG. 5). In that orientation the LSGGQ motif (SEQ ID NO: 10) in each of the two domains is adjacent to the opposing Walker-A sequence at the ATP-sandwich interface. Such interfaces are described, for example, in Loo et al., (2002) J Biol Chem., 277 (44): 41303-41306, and Smith et al., (2002) Mol Cell. 10 (1): 139-149, at FIG. 3A. When a nucleotide (ATP) is not present in the binding sites there is a gap at the domain interface and dimers do not readily form. When ATP is bound, the NBD dimerizes and hydrolyzes the bound ATP, after which the dimer can then dissociate (see, e.g., Locher 2009 supra). Stabilized dimers can be formed by including aa substitutions that make the NBD domain sequences ATP hydrolysis deficient or substantially ATP hydrolysis deficient (e.g., by reducing the ATP hydrolysis rate by one or more orders of magnitude (see, e.g., Vakkasoglu et al., “D-helix influences dimerization of the ATP-binding cassette (ABC) transporter associated with antigen processing 1 (TAP1) nucleotide-binding domain,” (2107) PloS ONE, 12 (5): e0178238, available at doi. Org/10.1371/journal. Pone.0178238)). For example, the hydrolytic Asp or Glu residue found in NBD proteins may be substituted by an Asn or Gin residue (see, e.g., FIG. 3).

Where it is desirable to have the NBDs of ABC proteins form homodimers in solution when sufficient ATP is present, residues at the interface between the dimers may be modified to either remove or substitute aa residues that interfere with interface formation, or to substitute aas not contributing to the interface interactions with aas that can contribute to the interactions. By way of example, it is known that the NBD of rat TAP1 homodimerizes in solution and can be used as a model for the formation of the ATP-sandwich interface to bring about solution phase homodimerization in the presence of ATP (see, e.g., Vakkasoglu et al. 2017 supra). Alignment of an NBD aa sequence with a sequence known to undergo homodimerization, e.g., the rat TAP1 NBD, can be used to guide alterations in a target sequence. Alignments may be carried out using the US National Center for Biotechnology Information (NCBI) BLAST program or the European Bioinformatics Institute's Clustal Omega program as previously indicated. As indicated above, substitutions in the D-helix of human TAP1 that reflect the sequence of the corresponding D-helix in rat TAP1 enhance the homodimerization of human TAP1 sequences.

An NBD aa sequence of an ABC containing protein may comprise all or part of the NBD domain of a Phospholipid-Transporting ATPase (ABC or ABCA1 transporter) encoded by the ABCA1 gene, UniProtKB-095477 (SEQ ID NO:1). An NBD may comprise a sequence having at least about 90% or at least about 95% sequence identity to SEQ ID NO:1. Alternatively, an NBD may have at least about 96% or at least about 98% sequence identity to SEQ ID NO:1.

An NBD aa sequence may comprise all or part of the NBD domain of Multidrug Resistance Protein 1 (MDR/TAP) encoded by the ABCB1 gene, UniProtKB-P08183 (SEQ ID NO:2). An NBD may comprise a sequence having at least about 90% or at least about 95% sequence identity to SEQ ID NO:2. An NBD may comprise a sequence having at least about 96% or at least about 98% sequence identity to SEQ ID NO:2.

An NBD aa sequence may comprise all or part of the NBD domain of a Multidrug resistance-associated protein (MRP, Multidrug resistance-associated protein 1) encoded by the ABCC1 gene, UniProtKB-P33527 (SEQ ID NO: 3). An NBD may comprise a sequence having at least about 90% or at least about 95% sequence identity to SEQ ID NO:3. Alternatively, an NBD may have at least about 96% or at least about 98% sequence identity to SEQ ID NO: 3.

An NBD aa sequence may comprise all or part of the NBD domain of an ATP-binding cassette subfamily D member 1 protein (ALD, Adrenoleukodystrophy Protein) encoded by the ABCD1 gene, UniProtKB/Swiss-Prot: P33897 (SEQ ID NO:4). An NBD may comprise a sequence having at least about 90% or at least about 95% sequence identity to SEQ ID NO:4. Alternatively, an NBD may have at least about 96% or at least about 98% sequence identity to SEQ ID NO:4.

An NBD aa sequence may comprise all or part of the NBD domain of an ATP-binding cassette subfamily E member 1 protein (OABP, 2′-5′-Oligoadenylate-Binding Protein) encoded by the ABCE1 gene, UniProtKB/Swiss-Prot: P61221 (SEQ ID NO:5). An NBD may comprise a sequence having at least about 90% or at least about 95% sequence identity to SEQ ID NO:5. Alternatively, an NBD may have at least about 96% or at least about 98% sequence identity to SEQ ID NO:5.

An NBD aa sequence may comprise all or part of the NBD domain of an ATP-binding cassette subfamily F member 1 protein (GCN20, TNF alpha-Inducible ATP-Binding Protein) encoded by the ABCF1 gene, UniProtKB-Q8NE71 (SEQ ID NO:6). An NBD may comprise a sequence having at least about 90% or at least about 95% sequence identity to SEQ ID NO:6. Alternatively, an NBD may have at least about 96% or at least about 98% sequence identity to SEQ ID NO:6.

An NBD aa sequence may comprise all or part of the NBD domain of an ATP-binding cassette subfamily G member 1 protein (White, Homolog Of Drosophila White) encoded by the ABCG2 gene, UniProtKB/Swiss-Prot: P45844 (SEQ ID NO:7). An NBD may comprise a sequence having at least about 90% or at least about 95% sequence identity to SEQ ID NO:7. Alternatively, an NBD may have at least about 96% or at least about 98% sequence identity to SEQ ID NO:7.

### (1) TAP1 and TAP2 ATP Binding Sequences as NBDs

TAP1 and TAP2 are cognate binding partners that couple ATP binding and their heterodimerization with peptide translocation across inner cell membranes and are members of the ABC transporter superfamily that represents a source of NBDs for use in the constructs provided herein. Rat TAP1 NBD is known to undergo homodimerization in the presence of ATP. The NBD of human TAP1 does not undergo homodimerization in the presence of ATP, but can be induced to undergo homodimerization using previously defined substitutions. See, e.g., Vakkasoglu et al. (2017) supra. Substitutions leading to human TAP1 homodimerization in the presence of ATP include those at one or more of (e.g., each of) N676, S677, Q680, E682, and Q683, which are bolded and italicized in SEQ ID NO:8 (shown below). For example, the TAP1 substitutions leading to homodimerization may include one or more (e.g., all) of N676G, S677N, Q680R, E682Q, and Q683R substitutions. The portions (e.g., domains) of TAP1 and TAP2 proteins employed as NBDs may also include substitutions of one or more cysteines present in the sequence (e.g., with serine residues) to prevent undesirable disulfide bond formation leading to non-functional folding or undesirable intermolecular disulfide bond formation. TAP1 and TAP2 NBDs may also include a His tag, for example at the C-terminus of the domain, for purification of constructs comprising those NBDs.

A human TAP1 may, for example, comprise the aa sequence:

(Reference Sequence: NP_000584.2,)
SEQ ID NO: 8

| | |
|---|---|
| 1 | MASSRCPAPR GCRCLPGASL AWLGTVLLLL ADWVLLRTAL |
| | PRIFSLLVPT ALPLLRVWAV |
| 61 | GLSRWAVLWL GACGVLRATV GSKSENAGAQ GWLAALKPLA |
| | AALGLALPGL ALFRELISWG |
| 121 | APGSADSTRL LHWGSHPTAF VVSYAAALPA AALWHKLGSL |
| | WVPGGQGGSG NPVRRLLGCL |
| 181 | GSETRRLSLF LVLVVLSSLG EMAIPFFTGR LTDWILQDGS |
| | ADTFTRNLTL MSILTIASAV |
| 241 | LEFVGDGIYN NTMGHVHSHL QGEVEGAVLR QETEFFQQNQ |
| | TGNIMSRVTE DTSTLSDSLS |
| 301 | ENLSLFLWYL VRGLCLLGIM LWGSVSLIMV TLITLPLLEL |
| | LPKKVGKWYQ LLEVQVRESL |
| 361 | AKSSQVAIEA LSAMPTVRSF ANEEGEAQKF REKLQEIKTL |
| | NQKEAVAYAV NSWTTSISGM |
| 421 | LLKVGILYIG GQLVTSGAVS SGNLVTEVLY QMQFTQAVEV |
| | LLSIYPRVQK AVGSSEKIFE |
| 481 | YLDRTPRCPP SGLLTPLHLE GLVQFQDVSF AYPNRPDVLV |
| | LQGLTFTLRP GEVTALVGPN |
| 541 | GSGKSTVAAL LQNLYQPTGG QLLLDGKPLP QYEHRYLHRQ |
| | VAAVGQEPQV FGRSLQENIA |
| 601 | YGLTQKPTME EITAAAVKSG AHSFISGLPQ GYDTEVDEAG |
| | SQLSGGQRQA VALARALIRK |
| 661 | PCVLILDDAT SALDANSQLQ VEQLLYESPE RYSRSVLLIT |
| | QHLSLVEQAD HILFLEGGAI |
| 721 | REGGTHQQLM EKKGCYWAMV QAPADAPE; |

where a nucleotide binding sequence comprises aas 492-748; see, e.g., NCBI Reference Sequence NP_000584.3 or UniProtKB-Q03518 (version 3, i.e., Q03518.3). The catalytic Asp residue at position 668 is bolded and italicized, as are residues N676, S677, Q680, E682, and Q683, which are associated with homodimerization.

Accordingly, an NBD aa sequence from human TAP1 for incorporation into the constructs described herein may comprise, for example, an aa sequence having greater than about 90% or greater than about 95% sequence identity to the NBD aa sequence of NCBI Reference Sequence: NP_000584.3 (aas 492-748) or UniProtKB-Q03518.3 (aas 492-748):

(SEQ ID NO: 9)

| | |
|---|---|
| 1 | GLLTPLHLEG LVQFQDVSFA YPNRPDVLVL QGLTFTLRPG |
| | EVTALVGPNG SGKSTVAALL |
| 61 | QNLYQPTGGQ LLLDGKPLPQ YEHRYLHRQV AAVGQEPQVF |
| | GRSLQENIAY GLTQKPTMEE |
| 121 | ITAAAVKSGA HSFISGLPQG YDTEVDEAGS QLSGGQRQAV |
| | ALARALIRKP CVLILDDATS |
| 181 | ALDANSQLQV EQLLYESPER YSRSVLLITQ HLSLVEQADH |
| | ILFLEGGAIR EGGTHQQLME |
| 241 | KKGCYWAMVQ APADAPE. |

Alternatively, an NBD may have at least about 96% or at least about 98% sequence identity to SEQ ID NO:9. Any TAP1 NBD sequence employed in the constructs of the present disclosure may be Cys-less, having C662 and C735 (aas C171 and C242 as renumbered in SEQ ID NO:9) substituted by aas other than Cys (e.g., C662S and/or C735S substitutions). Residues N676, S677, Q680, E682, and Q683 of SEQ ID NO:8, associated with homodimerization, appear as N185, S186, Q189, E191, and Q192 in SEQ ID NO:9 and are bolded and italicized. D177 is also bolded and italicized. An NBD aa sequence from human TAP1 may, for example, comprise an aa sequence having greater than about 97% or greater than about 98% sequence identity to at least 230 contiguous aas of the NBD of the aa sequence set forth in NCBI Reference Sequence: NP_000584.2 (aas 492-748) provided as SEQ ID NO:9.

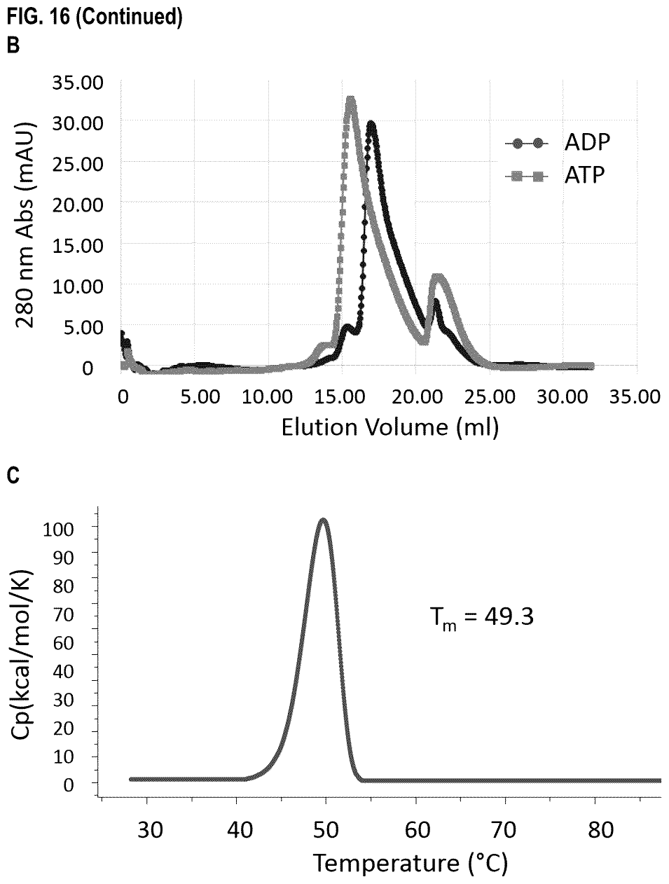

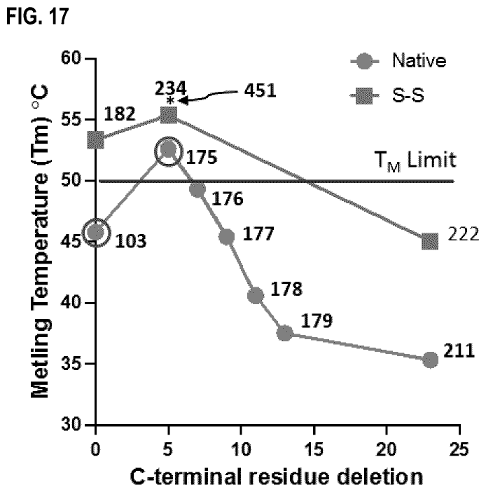

The C-terminal sequence of TAP1 (PADAPE (SEQ ID NO:11) may be deleted to increase the melting point (thermal stability, see FIG. 17) of TAP1 NBDs. A homodimerizing TAP1 polypeptide suitable for use in constructs of the present disclosure may comprise the sequence of SEQ ID NO: 12. A homodimerizing TAP1 polypeptide suitable for use in constructs of the present disclosure may comprise an aa sequence having greater than about 90% or greater than about 95% sequence identity to the NBD aa sequence of NCBI Reference Sequence: NP_000584.3 (aas 492-748) or UniProtKB-Q03518.3 (aas 492-748):

(SEQ ID NO: 12)

| | |
|---|---|
| 1 | GLLTPLHLEG LVQFQDVSFA YPNRPDVLVL QGLTFTLRPG |
| | EVTALVGPNG SGKSTVAALL |
| 61 | QNLYQPTGGQ LLLDGKPLPQ YEHRYLHRQV AAVGQEPQVF |
| | GRSLQENIAY GLTQKPTMEE |
| 121 | ITAAAVKSGA HSFISGLPQG YDTEVDEAGS QLSGGQRQAV |
| | ALARALIRKP CVLILDDATS |
| 181 | ALDANSQLQV EQLLYESPER YSRSVLLITQ HLSLVEQADH |
| | ILFLEGGAIR EGGTHQQLME |
| 241 | KKGCYWAMVQ A. |

Alternatively, an NBD may have at least about 96% or at least about 98% sequence identity to SEQ ID NO: 12. Residues N676, S677, Q680, E682, and Q683 (see SEQ ID NO:8), associated with homodimerization, appear as N185, S186, Q189, E191, and Q192 in SEQ ID NO: 12 and are bolded and italicized. An NBD aa sequence from human TAP1 may, for example, comprise an aa sequence having greater than 97% or greater than 98% sequence identity to at least 230 contiguous aas of the NBD of the aa sequence set forth in NCBI Reference Sequence: NP_000584.2 (aas 492-748) provided as SEQ ID NO:12.

A homodimerizing TAP1 polypeptide suitable for use in constructs of the present disclosure may comprise the sequence

(SEQ ID NO: 291)

| | |
|---|---|
| 1 | GLLTPLHLEG LVQFQDVSFA YPNRPDVLVL QGLTFTLRPG |
| | EVTALVGPNG SGKSTVAALL |
| 61 | QNLYQPTGGQ LLLDGKPLPQ YEHRYLHRQV AAVGQEPQVF |
| | GRSLQENIAY GLTQKPTMEE |
| 121 | ITAAAVKSGA HSFISGLPQG YDTEVDEAGS QLSGGQRQAV |
| | ALARALIRKP CVLILDDATS |
| 181 | ALDANSQLQV EQLLYESPER YSRSVLLITQ HLSLVEQADH |
| | ILFLEGGAIR EGGTHQQLME |
| 241 | KKGCYWAMVQ. |

Alternatively, an NBD may have at least about 96% or at least about 98% sequence identity to SEQ ID NO: 291. Residues N676, S677, Q680, E682, and Q683 (see SEQ ID NO:8), associated with homodimerization, appear as N185, S186, Q189, E191, and Q192 in SEQ ID NO:291 and are bolded and italicized. An NBD aa sequence from human TAP1 may, for example, comprise an aa sequence having greater than 97% or greater than 98% sequence identity to at least 230 contiguous aas of the NBD of the aa sequence set forth in NCBI Reference Sequence: NP_000584.2 (aas 492-748) provided as SEQ ID NO:291.

An NBD aa sequence from human TAP1 for incorporation into the constructs described herein may comprise substitutions at aa residues corresponding to C662 (e.g., C662S) and C735 (e.g., C735S) of SEQ ID NO:8 that remove cysteine residues, and substitutions at aas corresponding to one or more of N676, S677, Q680, E682, and Q683 (e.g., N676G, S677N, Q680R, E682Q, and/or Q683R) of SEQ ID NO:8, converting the D-helix of the human protein into the D-helix of rat TAP1. The NBD aa sequence from human TAP1 may also comprise a substitution at the position corresponding to D668 of SEQ ID NO:8 (position D177), which renders the NBD ATP hydrolysis-deficient (e.g., a D668N, D668A, or D668Q substitution). A D668N substitution is shown in SEQ ID NO: 13, but for mammalian cell expression a D668A substitution may also be employed. Those substitutions are bolded and italicized in SEQ ID NO: 13 below. Accordingly, a homodimerizing TAP1 polypeptide that may be employed as an NBD in a construct of the present disclosure may, for example, comprise the following aa sequence:

(SEQ ID NO: 13)

| | |
|---|---|
| 1 | GLLTPLHLEG LVQFQDVSFA YPNRPDVLVL QGLTFTLRPG |
| | EVTALVGPNG SGKSTVAALL |
| 61 | QNLYQPTGGQ LLLDGKPLPQ YEHRYLHRQV AAVGQEPQVF |
| | GRSLQENIAY GLTQKPTMEE |
| 121 | ITAAAVKSGA HSFISGLPQG YDTEVDEAGS QLSGGORQAV |
| | ALARALIRKP SVLILDNATS |
| 181 | ALDAGNQLRV QRLLYESPER YSRSVLLITQ HLSLVEQADH |
| | ILFLEGGAIR EGGTHQQLME |
| 241 | KKGSYWAMVQ APADAPE. |

A homodimerizing TAP1 polypeptide that may be employed as an NBD in a construct of the present disclosure may comprise an aa sequence having greater than about 90% or greater than about 95% sequence identity to SEQ ID NO: 13, while optionally retaining substitutions at C662, C735, D668, N676, S677, Q680, E682, and Q683. Alternatively, the NBD aa sequence may have greater than 97% or greater than 98% sequence identity to at least about 230 or at least about 240 contiguous aas of the sequence provided in SEQ ID NO:13, while optionally retaining substitutions at C662, C735, D668, N676, S677, Q680, E682, and/or Q683.

A homodimerizing TAP1 polypeptide, with a substitution at the position corresponding to D668 of SEQ ID NO: 8 (appearing as X177 below) to render it ATP hydrolysis-deficient, that is suitable as an NBD in a construct of the present disclosure may, for example, comprise the aa sequence of SEQ ID NO: 13 with the PADAPE deleted:

(SEQ ID NO: 14)

| | |
|---|---|
| 1 | GLLTPLHLEG LVQFQDVSFA YPNRPDVLVL QGLTFTLRPG |
| | EVTALVGPNG SGKSTVAALL |
| 61 | QNLYQPTGGQ LLLDGKPLPQ YEHRYLHRQV AAVGQEPQVF |
| | GRSLQENIAY GLTQKPTMEE |
| 121 | ITAAAVKSGA HSFISGLPQG YDTEVDEAGS QLSGGORQAV |
| | ALARALIRKP SVLILDXATS |
| 181 | ALDAGNQLRV QRLLYESPER YSRSVLLITQ HLSLVEQADH |
| | ILFLEGGAIR EGGTHQQLME |
| 241 | KKGSYWAMVQ A, |

X may be any aa other than E, or D, to render the sequence ATP hydrolysis deficient and may also exclude N to avoid the introduction of an Asn (N) linked glycosylation sit. For example, X may be N, Q, or A, and for expression in mammalian cells X may be, for example, Q or A. In an embodiment, X is A.

A homodimerizing TAP1 polypeptide that may be employed as an NBD in a construct of the present disclosure may comprise an aa sequence having greater than about 90% or greater than about 95% sequence identity to SEQ ID NO: 14, while optionally retaining substitutions at C662, C735, D668, N676, S677, Q680, E682, and Q683. Alternatively, the NBD aa sequence may have greater than 97% or greater than 98% sequence identity to at least about 230 or at least about 240 contiguous aas of the sequence provided in SEQ ID NO:14, while optionally retaining substitutions at C662, C735, D668, N676, S677, Q680, E682, and/or Q683.

A homodimerizing TAP1 polypeptide, with a substitution at the position corresponding to D668 of SEQ ID NO: 8 (appearing as X177 below) to render it ATP hydrolysis-deficient, that is suitable as an NBD in a construct of the present disclosure may, for example, comprise the aa sequence:

(SEQ ID NO: 292)

| | |
|---|---|
| 1 | GLLTPLHLEG LVQFQDVSFA YPNRPDVLVL QGLTFTLRPG |
| | EVTALVGPNG SGKSTVAALL |
| 61 | QNLYQPTGGQ LLLDGKPLPQ YEHRYLHRQV AAVGQEPQVF |
| | GRSLQENIAY GLTQKPTMEE |
| 121 | ITAAAVKSGA HSFISGLPQG YDTEVDEAGS QLSGGQRQAV |
| | ALARALIRKP SVLILDXATS |
| 181 | ALDAGNQLRV QRLLYESPER YSRSVLLITQ HLSLVEQADH |
| | ILFLEGGAIR EGGTHQQLME |
| 241 | KKGSYWAMVQ, |

X may be any aa other than E, or D, to render the sequence ATP hydrolysis deficient and may also exclude N to avoid the introduction of an Asn (N) linked glycosylation site. For example, X may be N, Q, or A, and for expression in mammalian cells X may be, for example, Q or A. In an embodiment, X is A.

A homodimerizing TAP1 polypeptide that may be employed as an NBD in a construct of the present disclosure may comprise an aa sequence having greater than about 90% or greater than about 95% sequence identity to SEQ ID NO:292, while optionally retaining substitutions at C662, C735, D668, N676, S677, Q680, E682, and Q683. Alternatively, the NBD aa sequence may have greater than 97% or greater than 98% sequence identity to at least about 230 or at least about 240 contiguous aas of the sequence provided in SEQ ID NO:292, while optionally retaining substitutions at C662, C735, D668, N676, S677, Q680, E682, and/or Q683.

An NBD aa sequence from human TAP1 may, for example, comprise substitutions corresponding to those at C662 (e.g., C662S) and C735 (e.g., C735S) of SEQ ID NO:8, appearing as s175 and S247. The NBD aa sequence from human TAP1 may also, for example, comprise substitutions corresponding to those at one or more of N676, S677, Q680, E682, and Q683 (e.g., N676G, S677N, Q680R, E682Q, and/or Q683R) in SEQ ID NO:8, which are bolded and italicized in the below sequence:

(SEQ ID NO: 15)

| | |
|---|---|
| 1 | PPSGLLTPLH LEGLVQFQDV SFAYPNRPDV LVLQGLTFTL |
| | RPGEVTALVG PNGSGKSTVA |
| 61 | ALLQNLYQPT GGQLLLDGKP LPQYEHRYLH RQVAAVGQEP |
| | QVEGRSLQEN IAYGLTQKPT |
| 121 | MEEITAAAVK SGAHSFISGL PQGYDTEVDE AGSQLSGGQR |
| | QAVALARALI RKPSVLILDN |
| 181 | ATSALDAGNQ LRVQRLLYES PERYSRSVLL ITQHLSLVEQ |
| | ADHILFLEGG AIREGGTHQQ |
| 241 | LMEKKGSYWA MVQAPADAPE, |

while optionally retaining substitutions at C662, C735, D668, N676, S677, Q680, E682, and Q683. Alternatively, the NBD aa sequence may have greater than 97% or greater than 98% sequence identity to at least about 250 or at least about 260 contiguous aas of the sequence provided in SEQ ID NO: 15 while optionally retaining substitutions at C662, C735, D668, N676, S677, Q680, E682, and/or Q683.

In addition, the NBD aa sequence from human TAP1 may comprise a substitution at the Asp corresponding to D668 (e.g., a D668N, D668A, or D668Q substitution) of SEQ ID NO:8, which renders the NBD ATP hydrolysis-deficient. Accordingly, an NBD polypeptide aa sequence of TAP1 may, for example, comprise the aa sequence:

(SEQ ID NO: 16)

| | |
|---|---|
| 1 | PPSGLLTPLH LEGLVQFQDV SFAYPNRPDV LVLQGLTFTL |
| | RPGEVTALVG PNGSGKSTVA |
| 61 | ALLQNLYQPT GGQLLLDGKP LPQYEHRYLH RQVAAVGQEP |
| | QVFGRSLQEN IAYGLTQKPT |
| 121 | MEEITAAAVK SGAHSFISGL PQGYDTEVDE AGSQLSGGQR |
| | QAVALARALI RKPSVLILDX |
| 181 | ATSALDAGNQ LRVQRLLYES PERYSRSVLL ITQHLSLVEQ |
| | ADHILFLEGG AIREGGTHQQL |
| 240 | MEKKGSYWAM VQAPADAPE, |

X may be any aa other than E, or D, to render the sequence ATP hydrolysis deficient and may also exclude N to avoid the introduction of an Asn (N) linked glycosylation site. For example, X may be N, Q, or A, and for expression in mammalian cells X may be, for example, Q or A.

A homodimerizing TAP1 polypeptide that may be employed as an NBD in a construct of the present disclosure may comprise an aa sequence having greater than about 90% or greater than about 95% sequence identity to SEQ ID NO: 16, while optionally retaining substitutions at C662, C735, D668, N676, S677, Q680, E682, and/or Q683. Alternatively, the NBD aa sequence may have greater than 97% or greater than 98% sequence identity to at least about 240 or at least about 250 contiguous aas of the sequence provided in SEQ ID NO:16, while optionally retaining substitutions at C662, C735, D668, N676, S677, Q680, E682, and/or Q683. The C-terminal sequence PADAPE (SEQ ID NO:11) may be deleted from SEQ ID NO:16.

In order to stabilize TAP NBD sequences, the coupling helix aa sequence of TAP1, ETEFFQQNQT (aas 2-11, SEQ ID NO:17), and the coupling helix aa sequence of TAP2, GLQTVRSF (SEQ ID NO: 18), separated by linkers comprising a G4S aa sequence (SEQ ID NO:40) may be incorporated as an N-terminal “CAP” on a TAP aa sequence (e.g., a TAP1 sequence) employed as an NBD in a construct disclosed herein. Accordingly, a capped homodimerizing human TAP1 NBD aa sequence may comprise the aa sequence:

(SEQ ID NO: 19)

| | |
|---|---|
| 1 | ETEFFQQNQT GGGGSGLQTV RSFGGGGGSG GSGLLTPLHL |
| | EGLVQFQDVS FAYPNRPDVL |
| 61 | VLQGLTFTLR PGEVTALVGP NGSGKSTVAA LLQNLYQPTG |
| | GQLLLDGKPL PQYEHRYLHR |
| 121 | QVAAVGQEPQ VFGRSLQENI AYGLTQKPTM EEITAAAVKS |
| | GAHSFISGLP QGYDTEVDEA |
| 181 | GSQLSGGQRQ AVALARALIR KPSVLILDDA TSALDAGNQL |
| | RVQRLLYESP ERYSRSVLLI |
| 241 | TQHLSLVEQA DHILFLEGGA IREGGTHQQL MEKKGSYWAM |
| | VQAPADAPE. |

That sequence comprises substitutions at positions C662 (e.g., C662S), C735 (e.g., C735S), N676, S677, Q680, E682, and Q683 (e.g., N676G, S677N, Q680R, E682Q, and/or Q683R) in SEQ ID NO:8, which are bolded and italicized. In addition, the NBD aa sequence from human TAP1 may comprise a substitution at the aa corresponding to D668 of SEQ ID NO:8 (D209), which renders the NBD ATP hydrolysis-deficient:

(SEQ ID NO: 20)

| | |
|---|---|
| 1 | ETEFFQQNQT GGGGSGLQTV RSFGGGGGSG GSGLLTPLHL |
| | EGLVQFQDVS FAYPNRPDVL |
| 61 | VLQGLTFTLR PGEVTALVGP NGSGKSTVAA LLQNLYQPTG |
| | GQLLLDGKPL PQYEHRYLHR |
| 121 | QVAAVGQEPQ VFGRSLQENI AYGLTQKPTM EEITAAAVKS |
| | GAHSFISGLP QGYDTEVDEA |
| 181 | GSQLSGGQRQ AVALARALIR KPSVLILDXA TSALDAGNQL |
| | RVQRLLYESP ERYSRSVLLI |
| 241 | TQHLSLVEQA DHILFLEGGA IREGGTHQQL MEKKGSYWAM |
| | VQAPADAPE; |

X may be any aa other than E, or D, to render the sequence ATP hydrolysis deficient and may also exclude N to avoid the introduction of an Asn (N) linked glycosylation site. For example, X may be N, Q, or A, and for expression in mammalian cells X may be, for example, Q or A. A capped homodimerizing TAP1 polypeptide suitable as an NBD in a construct of the present disclosure may comprise an aa sequence having greater than about 90% or greater than about 95% sequence identity to SEQ ID NO: 19 or SEQ ID NO:20, while optionally retaining the CAP sequence and/or the substitutions at C662, C735, D668, N676, S677, Q680, E682, and/or Q683. Alternatively, the NBD aa sequence may have greater than 97% or greater than 98% sequence identity to at least about 230 or at least about 240 contiguous aas of the sequence provided in SEQ ID NO: 19 or SEQ ID NO:20, while optionally retaining the CAP sequence and/or the substitutions at C662, C735, D668, N676, S677, Q680, E682, and/or Q683. Alternatively, the NBD aa sequence may have greater than 97% or greater than 98% sequence identity to at least about 270 or at least about 280 contiguous aas of the sequence provided in SEQ ID NO: 19 or SEQ ID NO:20.

A capped homodimerizing human TAP1 NBD aa sequence may comprise the aa sequence:

(SEQ ID NO: 293)

| | |
|---|---|
| 1 | ETEFFQQNQT GGGGSGLQTV RSFGGGGGSG GSGLLTPLHL |
| | EGLVQFQDVS FAYPNRPDVL |
| 61 | VLQGLTFTLR PGEVTALVGP NGSGKSTVAA LLQNLYQPTG |
| | GQLLLDGKPL PQYEHRYLHR |
| 121 | QVAAVGQEPQ VFGRSLQENI AYGLTQKPTM EEITAAAVKS |
| | GAHSFISGLP QGYDTEVDEA |
| 181 | GSQLSGGQRQ AVALARALIR KPSVLILDDA TSALDAGNQL |
| | RVQRLLYESP ERYSRSVLLI |
| 241 | TQHLSLVEQA DHILFLEGGA IREGGTHQQL MEKKGSYWAM |
| | VQ. |

That sequence comprises substitutions at positions C662 (e.g., C662S), C735 (e.g., C735S), N676, S677, Q680, E682, and Q683 (e.g., N676G, S677N, Q680R, E682Q, and/or Q683R) in SEQ ID NO:8, which are bolded and italicized. In addition, the NBD aa sequence from human TAP1 may comprise a substitution at the aa corresponding to D668 of SEQ ID NO:8 (D209), which renders the NBD ATP hydrolysis-deficient:

(SEQ ID NO: 294)

| | |
|---|---|
| 1 | ETEFFQQNQT GGGGSGLQTV RSFGGGGGSG GSGLLTPLHL |
| | EGLVQFQDVS FAYPNRPDVL |
| 61 | VLQGLTFTLR PGEVTALVGP NGSGKSTVAA LLQNLYQPTG |
| | GQLLLDGKPL PQYEHRYLHR |
| 121 | QVAAVGQEPQ VEGRSLQENI AYGLTQKPTM EEITAAAVKS |
| | GAHSFISGLP QGYDTEVDEA |
| 181 | GSQLSGGQRQ AVALARALIR KPSVLILDXA TSALDAGNQL |
| | RVQRLLYESP ERYSRSVLLI |
| 241 | TQHLSLVEQA DHILFLEGGA IREGGTHQQL MEKKGSYWAM |
| | VQ; |

X may be any aa other than E, or D, to render the sequence ATP hydrolysis deficient and may also exclude N to avoid the introduction of an Asn (N) linked glycosylation site. For example, X may be N, Q, or A, and for expression in mammalian cells X may be, for example, Q or A.

A capped homodimerizing TAP1 polypeptide suitable as an NBD in a construct of the present disclosure may comprise an aa sequence having greater than about 90% or greater than about 95% sequence identity to SEQ ID NO: 293 or SEQ ID NO:294, while optionally retaining the CAP sequence and/or the substitutions at C662, C735, D668, N676, S677, Q680, E682, and/or Q683. Alternatively, the NBD aa sequence may have greater than 97% or greater than 98% sequence identity to at least about 240 contiguous aas of the sequence provided in SEQ ID NO: 293 or SEQ ID NO:294, while optionally retaining the CAP sequence and/or the substitutions at C662, C735, D668, N676, S677, Q680, E682, and/or Q683. Alternatively, the NBD aa sequence may have greater than 97% or greater than 98% sequence identity to at least about 270 or at least about 280 contiguous aas of the sequence provided in SEQ ID NO:293 or SEQ ID NO:294.

In any construct encompassed by the present disclosure where a TAP1 NBD (e.g., an aa sequence comprising any of SEQ ID NOs: 9, 12-16, 19, or 20) is present, the Asp (D) residue corresponding to D668 may be substituted to render the TAP1 polypeptide hydrolysis deficient. The position corresponding to D668 may be any aa other than D to render the sequence ATP hydrolysis deficient. The position corresponding to D668 may be any aa other than D or N to render the sequence ATP hydrolysis deficient and to avoid the introduction of an Asn (N) linked glycosylation site. The position corresponding to D668 may be N, Q, or A, and for expression in mammalian cells may be, for example, Q or A. In an embodiment, the position corresponding to D668 may be A (Ala). In addition, to facilitate expression in mammalian cells, S542 (see SEQ ID NO:8) or the corresponding positions in any other TAP1 aa sequences (e.g., S51 in SEQ ID NOs: 9 and 12) may be substituted with an aa other than serine or threonine, removing a site linked to degradation without the introduction of a glycosylation site. For example, S54 may be substituted with an alanine to give an S54A substitution.

Additional substitutions at the interface between TAP monomers (i.e. the dimer interface) that either increase ATP binding or dimer stability may be incorporated into the NBDs of protein constructs described herein, including constructs comprising an NBD of SEQ ID NOs: 9, 12-16, 19, 20, and 291-294. Such substitutions in TAP1 include, but are not limited to, Q701H substitutions described by Procko et al. in Molecular Cell 24:51-62 (2006) and/or E564R substitutions described by Procko and Gaudet in Biochemistry 47 (21): 5699-708 (2008). Accordingly, an NBD present in a construct of the present disclosure may comprise a TAP1 aa sequence bearing C662S, C735S, N676G, S677N, Q680R, E682Q, Q683R, and D668N substitutions and optionally a Q701H substitution.

As discussed below, intrachain disulfide bonds may be incorporated into TAP1 NBD sequences to increase the stability (e.g., thermal stability as assessed by melting point). Stabilizing disulfide bonds include those from Cys residues substituted at positions L493 and E573 of the TAP1 sequence, resulting in formation of an L493C: E573C disulfide in SEQ ID NO:8, or the corresponding positions in constructs comprising an NBD of SEQ ID NOs: 9, 12-16, 19, and 20. Other stabilizing disulfide bonds that may be incorporated into any of those NBD sequences include, but are not limited to, a disulfide formed between: i) the cysteine at C735 and a cysteine substituted for E722 (a C735: E722C disulfide); and ii) the cysteine at C662 and a cysteine substituted for R694 (a C662: R694C disulfide). Substitutions at the interface between TAP monomers and stabilizing disulfides may be incorporated into the NBDs used in the constructs disclosed herein.

Some NBDs from TAP proteins that do not homodimerize, but rather heterodimerize with a cognate NBD, are used to prepare pairs of constructs (see, e.g., FIG. 2 at B and D and FIG. 3) or complexes of constructs (see, e.g., FIG. 4A) of the present disclosure. For example, a pair of constructs bearing human TAP1 NBDs and human TAP2 NBDs can be prepared. The pair of constructs comprising TAP1 and TAP2 NBDs will form heterodimers through interaction of TAP1 and TAP2 in the presence of sufficient ATP. Using NBDs such as TAP1 and TAP2 that can heterodimerize permits different specific combinations of activating sequences to be combined for immune cell stimulation. By way of example, a construct comprising IL-12A (p35), an optional linker aa sequence, and TAP1 can heterodimerize in the presence of sufficient ATP with a construct comprising IL-12B (p40), an optional linker aa sequence, and TAP2 to form a pair of constructs presenting an active IL-12AB heterodimer. Such constructs may also comprise a scaffold aa sequence such as an Ig Fc aa sequence if desired.

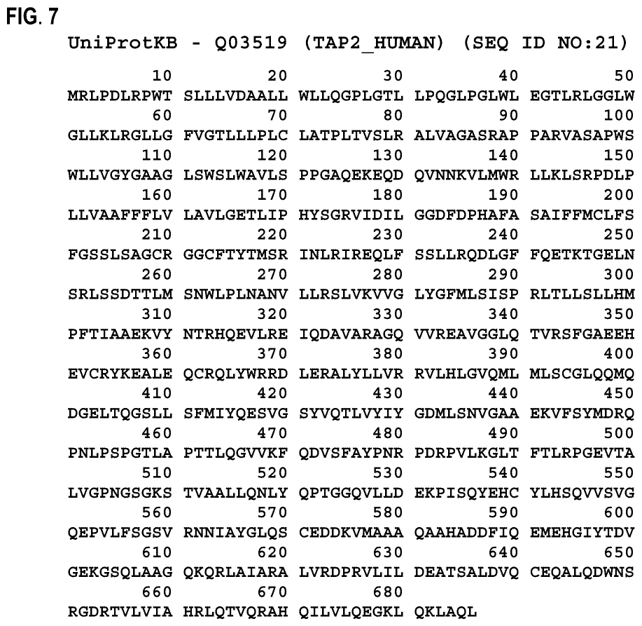

FIG. 7 provides the sequence of human TAP2. In some cases, an NBD of a construct of the present disclosure may comprise an aa sequence from the NBD from human TAP2. For example, the NBD may comprise an aa sequence having greater than about 90% or greater than about 95% sequence identity to the NBD aa sequence of UniProtKB-Q03519 (TAP2_HUMAN):

(SEQ ID NO: 21)

| | |
|---|---|
| 1 | MRLPDLRPWT SLLLVDAALL WLLQGPLGTL LPQGLPGLWL |
| | EGTLRLGGLW GLLKLRGLLG |
| 61 | FVGTLLLPLC LATPLTVSLR ALVAGASRAP PARVASAPWS |
| | WLLVGYGAAG LSWSLWAVLS |
| 121 | PPGAQEKEQD QVNNKVLMWR LLKLSRPDLP LLVAAFFFLV |
| | LAVLGETLIP HYSGRVIDIL |
| 181 | GGDFDPHAFA SAIFFMCLFS FGSSLSAGCR GGCFTYTMSR |
| | INLRIREQLF SSLLRQDLGF |
| 241 | FQETKTGELN SRLSSDTTLM SNWLPLNANV LLRSLVKVVG |
| | LYGFMLSISP RLTLLSLLHM |
| 301 | PFTIAAEKVY NTRHQEVLRE IQDAVARAGQ VVREAVGGLQ |
| | TVRSFGAEEH EVCRYKEALE |
| 361 | QCRQLYWRRD LERALYLLVR RVLHLGVQML MLSCGLQQMQ |
| | DGELTQGSLL SFMIYQESVG |
| 421 | SYVQTLVYIY GDMLSNVGAA EKVFSYMDRQ PNLPSPGTLA |
| | PTTLQGVVKF QDVSFAYPNR |
| 481 | PDRPVLKGLT FTLRPGEVTA LVGPNGSGKS TVAALLQNLY |
| | QPTGGQVLLD EKPISQYEHC |
| 541 | YLHSQVVSVG QEPVLFSGSV RNNIAYGLQS CEDDKVMAAA |
| | QAAHADDFIQ EMEHGIYTDV |
| 601 | GEKGSQLAAG QKQRLAIARA LVRDPRVLIL DEATSALDVQ |
| | CEQALQDWNS RGDRTVLVIA |
| 661 | HRLQTVQRAH QILVLQEGKL QKLAQL. |

For example, an NBD of a construct of the present disclosure may comprise an aa sequence having greater than about 90% or greater than about 95% sequence identity to the NBD of that sequence.

An NBD in a construct described herein may, for example, comprise an aa sequence having greater than about 90% or greater than about 95% sequence identity to the human TAP2 aa sequence:

(SEQ ID NO: 22)

| | |
|---|---|
| 1 | GTLAPTTLQG VVKFQDVSFA YPNRPDRPVL KGLTFTLRPG |
| | EVTALVGPNG SGKSTVAALL |
| 61 | QNLYQPTGGQ VLLDEKPISQ YEHCYLHSQV VSVGQEPVLF |
| | SGSVRNNIAY GLQSCEDDKV |
| 121 | MAAAQAAHAD DFIQEMEHGI YTDVGEKGSQ LAAGQKQRLA |
| | IARALVRDPR VLILDEATSA |
| 181 | LDVQCEQALQ DWNSRGDRTV LVIAHRLQTV QRAHQILVLQ |
| | EGKLQK. |

The NBD may also comprise an aa sequence having greater than 96% or greater than 98% sequence identity to SEQ ID NO:22.

A substitution removing the catalytic acidic aa residue corresponding to the D668N substitution of TAP1 rendering TAP2 NBDs ATP hydrolysis deficient may be incorporated into a TAP2 NBD aa sequence (e.g., an E176N, E176A, or E176Q substitution).

### (2) Modified TAP1 and TAP2 Sequences as NBDs

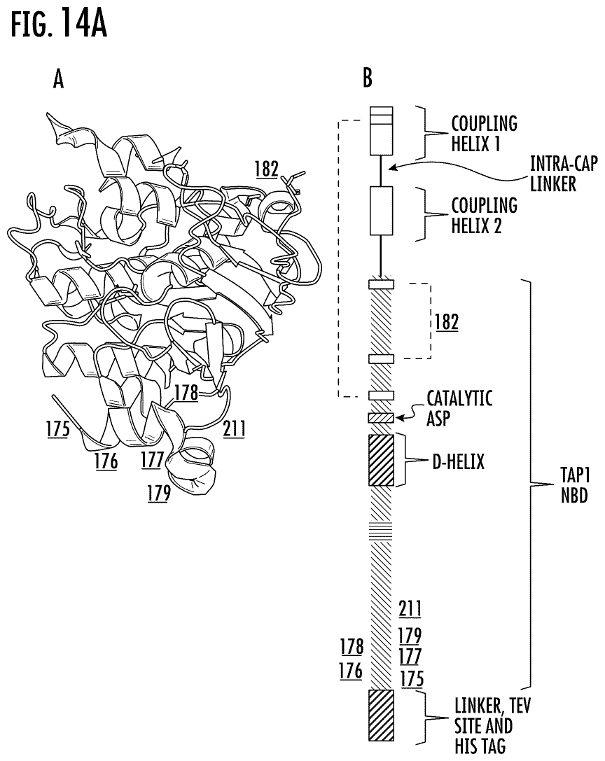

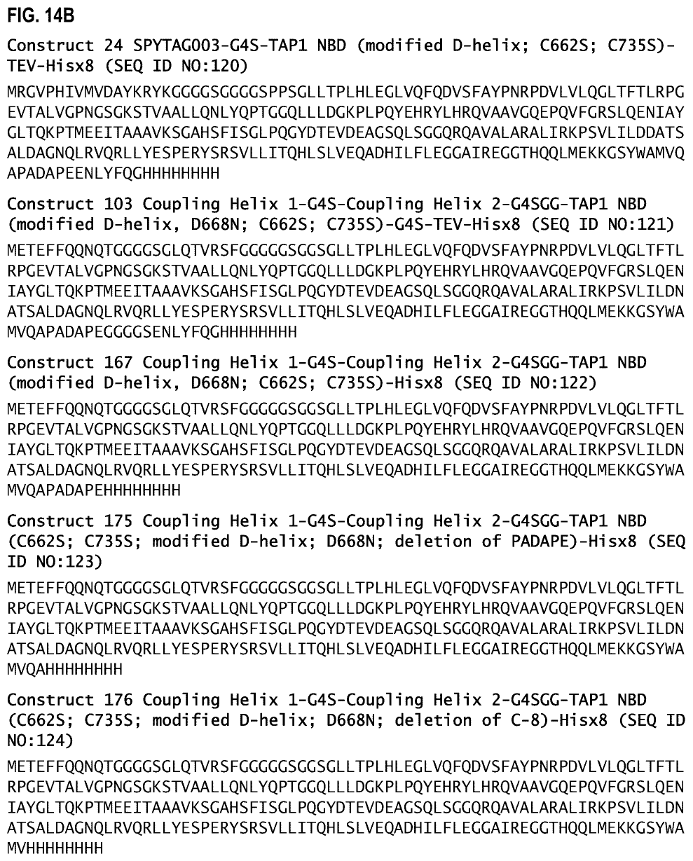

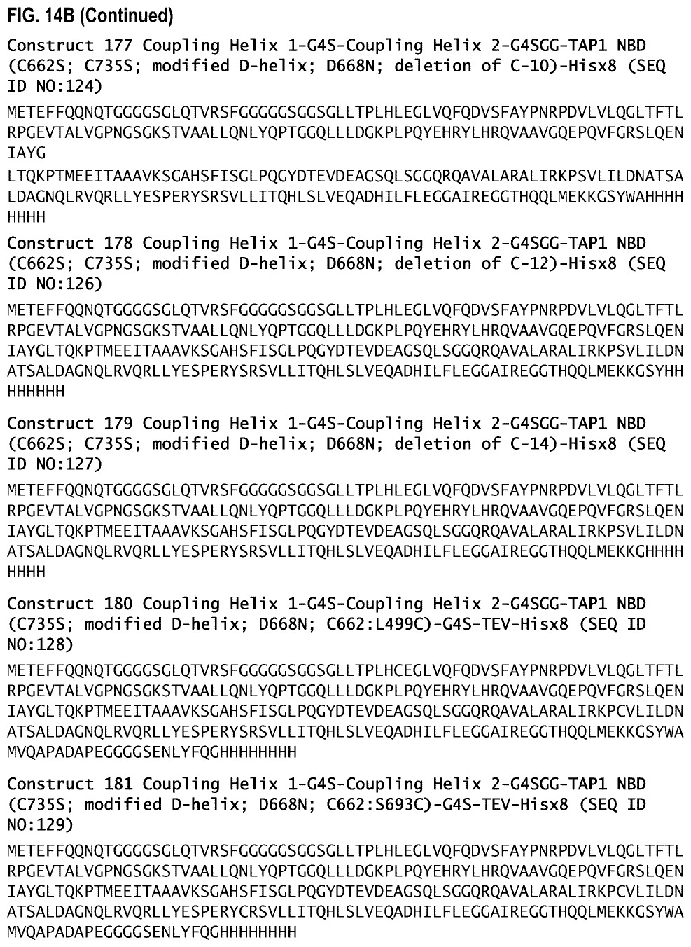

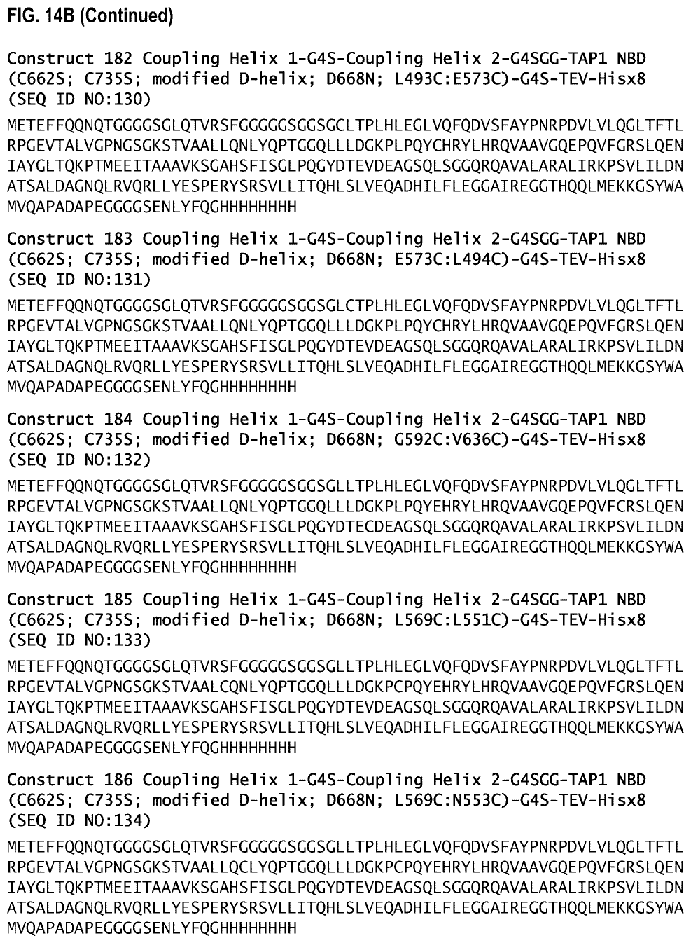

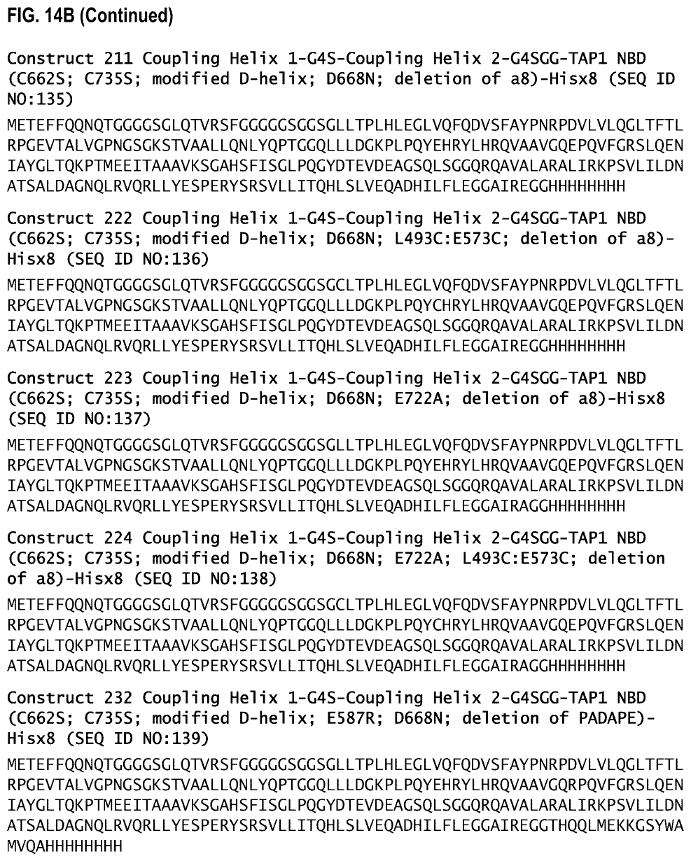

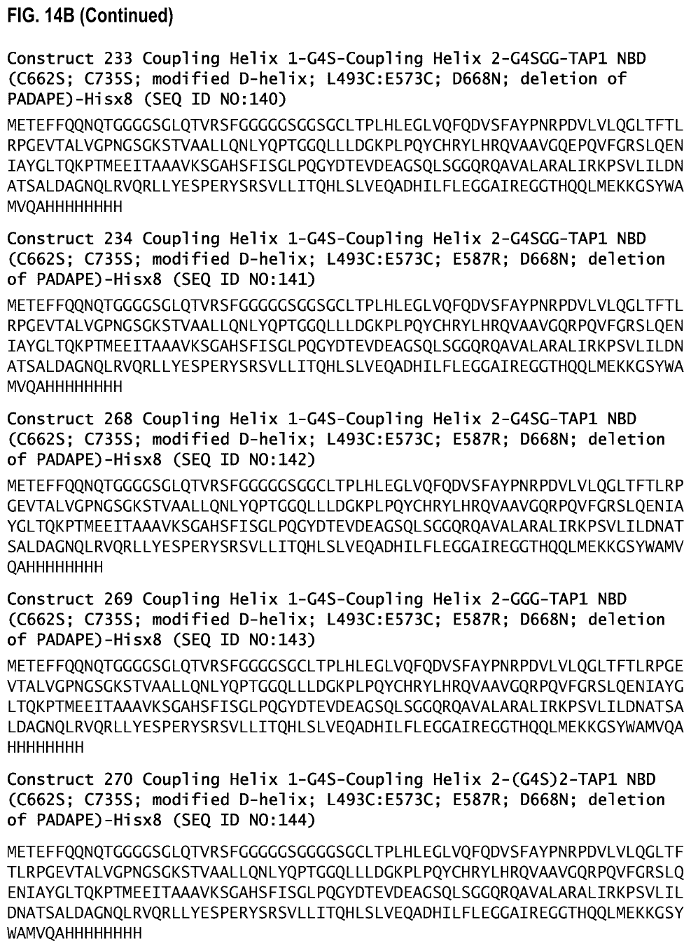

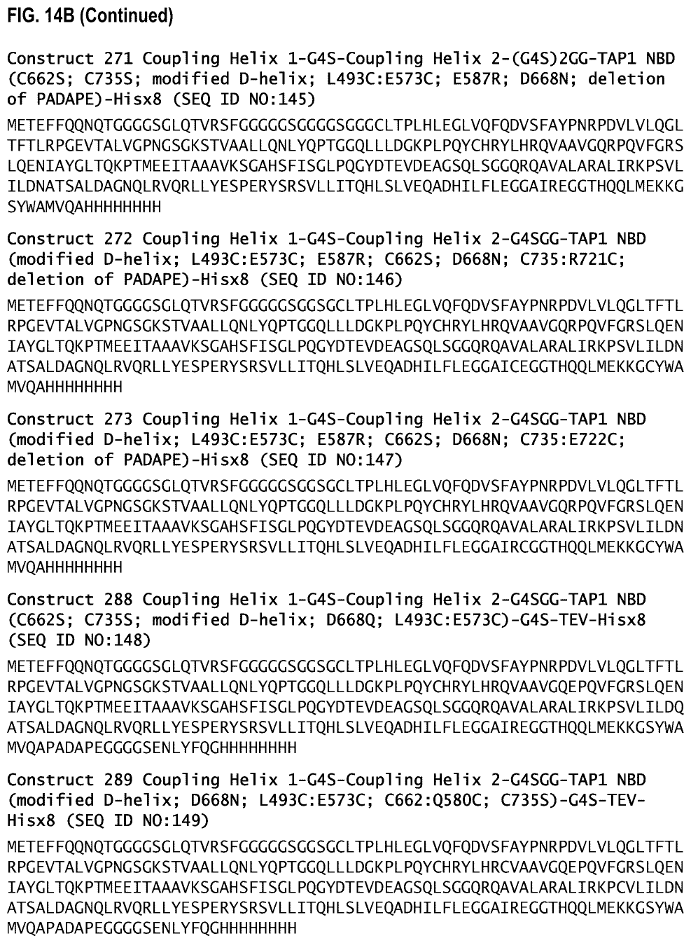

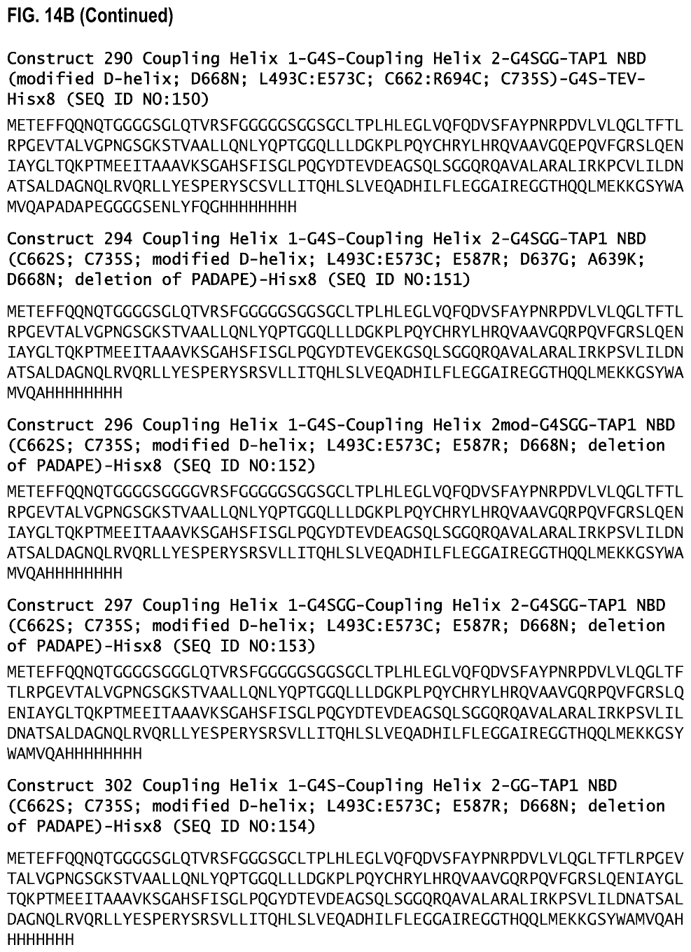

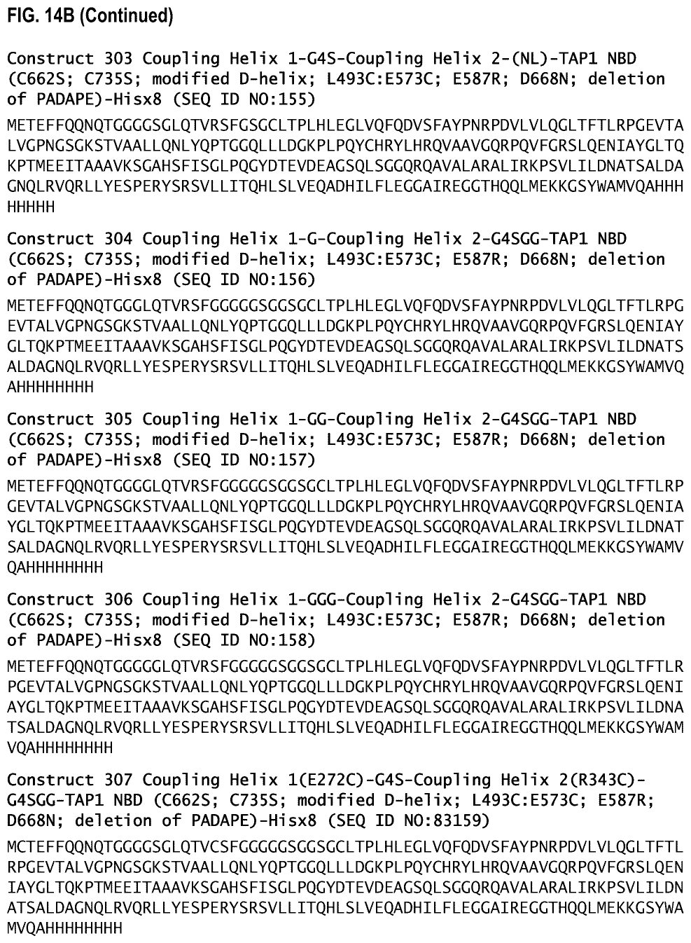

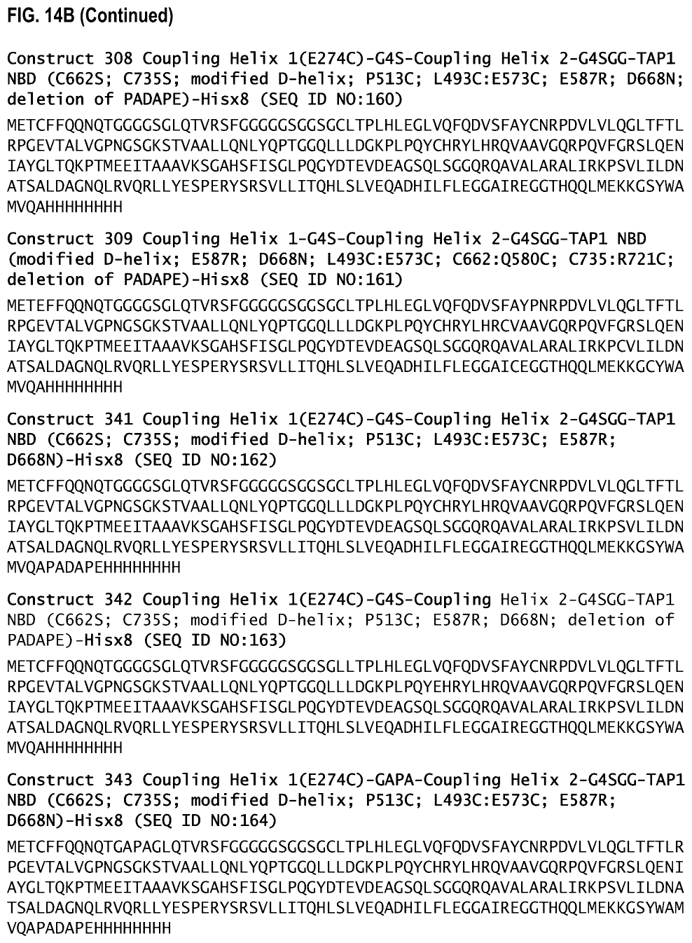

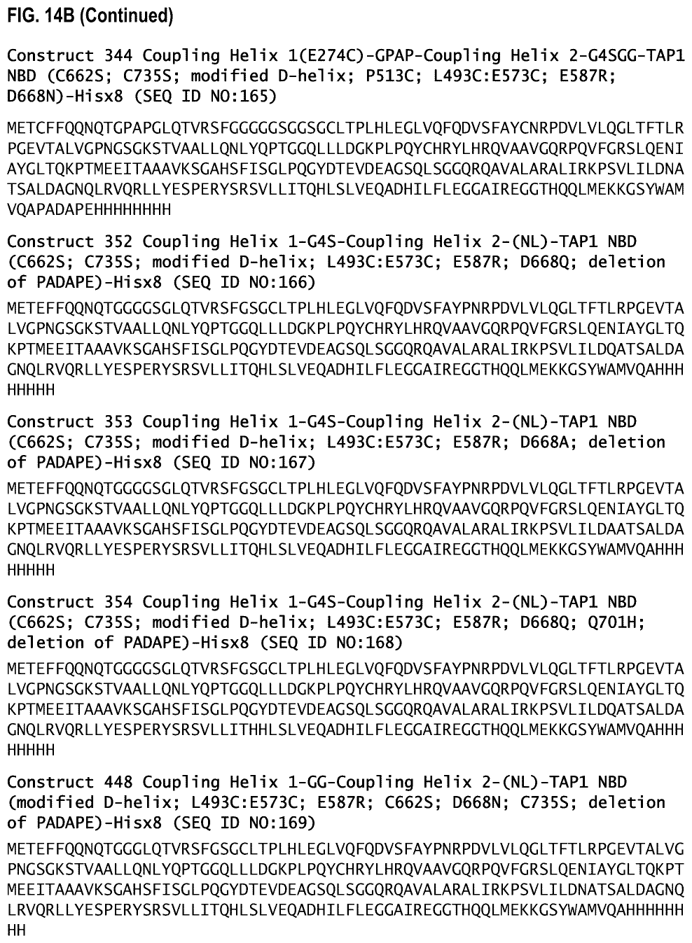

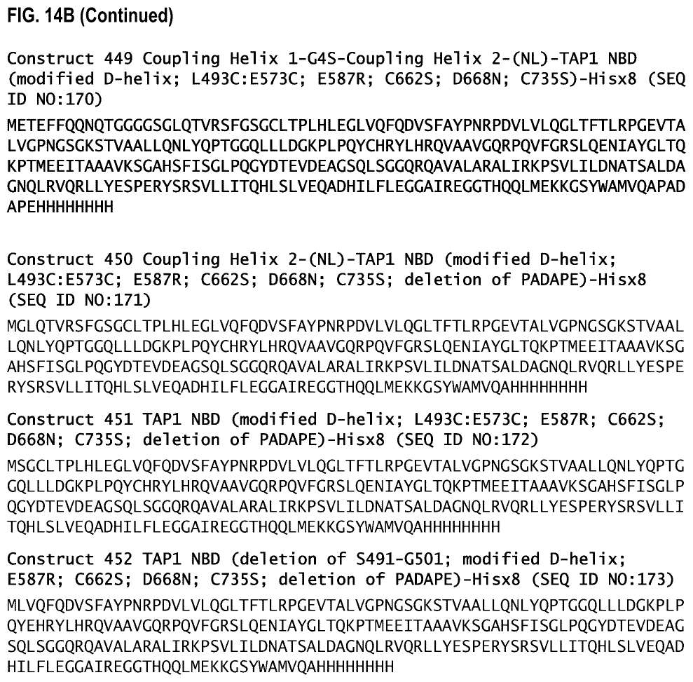

The NBDs of TAP1 and/or TAP2 undergo dimerization at ATP levels found in TMEs. Development of therapeutics that advantageously employ that dimerization may be benefitted by enhancement of dimerization specifically in the presence of ATP (as opposed to ADP and/or other nucleotides) and/or modifications that result in stable proteins (e.g., modifications that enhance thermal stability of each NBD and/or dimers of the NBD). Such modifications may include, for example, aa deletions, insertions, and/or substitutions, and additionally the formation of disulfide bonds. To assess the effect of various modifications, a construct (construct 103) was prepared comprising in the N-terminal to C-terminal direction: a CAP (specifically the coupling helix from TAP1, the coupling helix from TAP2, see, e.g., SEQ ID NOs: 17 and 18), the TAP1 NBD rendered ATP hydrolysis deficient by a substitution of an Asn for the catalytic Asp at position 668 (D668N), and a histidine tag for purification. The overall structure of construct 103 Coupling Helix 1-Coupling Helix 2-TAP1 NBD (Cysteine-less-D-loop modified, D668N)-HIS can be understood by reference to FIG. 14A at B. The sequence of construct 103 is provide in FIG. 14B.

C-terminal deletions from the NBD of construct 103 show that deletion of up to 6 aas (up to position 742 of SEQ ID NO:8) increases the melting point above 50° C., but further deletions result in a decrease in the melting point. This can be seen in FIG. 17 when comparing the melting temperature I of construct 103 with that of constructs 175-179 and 211. Those constructs comprise C-terminal deletions of the NBD. Construct 179 comprises a deletion approximately up to the N-terminus of alpha helix 9 (a9, CYWAMVQ (SEQ ID NO:23) and construct 211 a deletion up to about the N-terminus of alpha helix 8 (a8, THQQLME (SEQ ID NO:24). Accordingly, construct 179 essentially deletes the a9 helix to the C-terminus of the NBD, whereas construct 211 essentially deletes the a8 helix to the C-terminus of the NBD (including the a9 helix).

Formation of a disulfide bond by introduction of Cys residues at positions L493 and E573 (L493C: E573C disulfide) results in an increase in the Tm as can be seen by comparison of the Tm measurements of, for example, constructs 103 and 182 in FIG. 17. Disulfide bonds formed at other locations may also stabilize the NBD of TAP1. Disulfide bonds may, for example, be formed between C735: E722C or C662: R694C of TAP1. Although those disulfide bonds do not result in a substantial change in Tm, they may stabilize or alter the protein's properties (e.g., reduce susceptibility to proteolysis or resistance against thermal denaturation such as irreversible denaturation leading to precipitation or aggregation). Like the L493C: E573C disulfide bond discussed above, disulfide bonds formed between cysteines present in the TAP1 sequence (C662 or C735) and a cysteine substituted into the TAP1 sequence at, for example, R721C or Q580C may result in substantial increases in Tm (e.g., relative to construct 103). For example, the C735: R721C disulfide bond of construct 272 and the C662: Q580C disulfide bond of construct 289 substantively increase the Tm of the NBD above 50° C.

Substitutions that enhance the ability of TAP1 sequences to homodimerize include substitutions in the D-helix of the TAP1 NBD, including substitutions at positions N676, S677, Q680, E682, and/or Q683 (see SEQ ID NO: 8). Substitutions of N676G, S677N, Q680R, E682Q, and Q683R in the human TAP1 D-helix (NSQLQVEQLL, SEQ ID NO:25) convert the human D-helix sequence to that found in rat TAP1 (GNQLRVQRLL, SEQ ID NO:26) that can homodimerize in the presence of sufficient ATP.

The introduction of an E587R of human TAP1 (see SEQ ID NO:8) has been found to decrease non-specific dimerization in the presence of ADP. Constructs with TAP1 E587R substitutions (or the corresponding substitution in, for example, TAP2 at E552R) display more ATP specific dimerization relative to constructs comprising the NBD of TAP1 lacking that substitution.

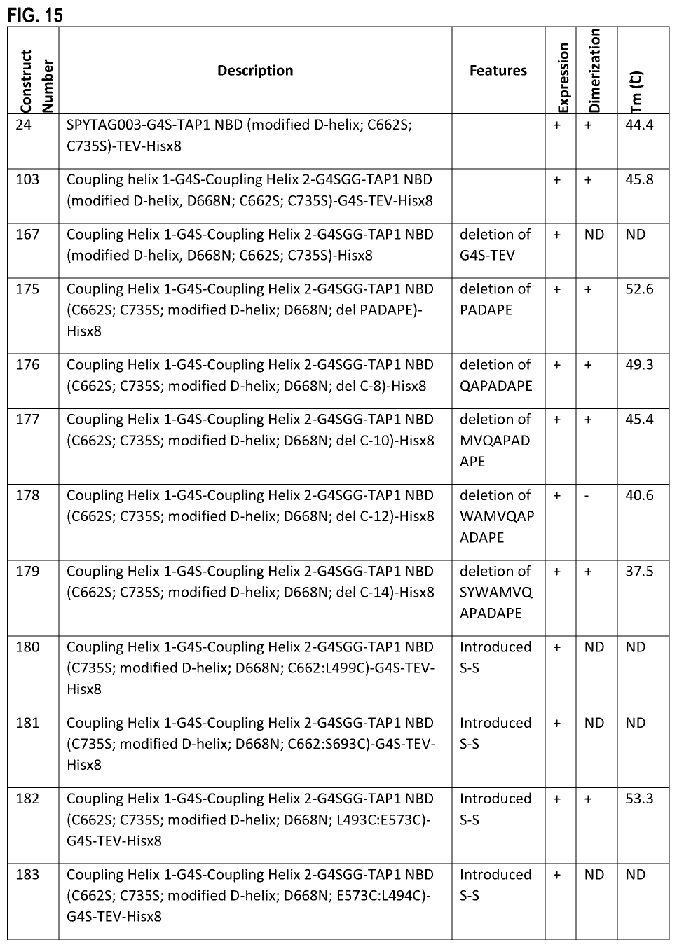

Separately, or in addition to the above-mentioned modifications, an N-terminal CAP aa sequence or the presence of an N-terminal AD or N-terminal targeting sequence may be added to the NBD for a variety of purposes. While N-terminal CAP additions are not necessary for thermal stability of the NBD, they can enhance its thermal stability relative to constructs having “uncapped” native TAP1 NBD sequences. It is noted that construct 451, which parallels construct 182 and includes several stabilizing substitutions but lacks a CAP, has the highest single Tm (56.3° C.) of any NBD construct identified herein. FIGS. 14B and 15 provide sequences and structures for the constructs.

CAP sequences, or other sequences (an AD and/or targeting sequence) placed N-terminal to the NBD sequence, can produce, for example, increased expression levels relative to the constructs having an “uncapped” N-terminal TAP1 NBD. CAP structures may take a variety of forms including, but not limited to, aa sequences comprising the coupling helices of TAP1 and TAP2 and Ig Fc sequences which may also function as scaffold sequences.

As the coupling helix of TAP1 and the coupling helix of TAP2 are known to interact, their incorporation into the bispecific ATP-dependent agonist disclosed herein as part of a CAP structure provides a self-assembling structure stabilizing the adjacent portions of the molecule (e.g., the NBD of TAP1). In an embodiment, a CAP structure present N-terminal to a TAP NBD (e.g., TAP1 NBD) comprises in the N-terminal to C-terminal direction: (i) a TAP1 coupling helix, (ii) an optional independently selected intra-CAP linker, (iii) a TAP2 coupling helix, and (iv) an optional independently selected linker between the TAP2 coupling helix and the TAP NBD aa sequence (CAP-NBD linker). More specifically, the CAP may comprise: (i) a TAP1 coupling helix comprising SEQ ID NO:17, an optional independently selected intra-CAP linker, a TAP2 coupling helix comprising SEQ ID NO:18, and an optional independently selected CAP-NBD linker.

The intra-CAP linker between Coupling Helix 1 and Coupling Helix 2 is optional. When the intra-CAP linker is present it may be, for example, from 1 aa to about 10 aas or from 1 aa to 5 aas in length. Alternatively, the intra-CAP linker may be from 6 aas to about 10 aas in length. The intra-CAP linker may comprise, for example, a single aa, such as a G or an A residue. An intra-CAP linker may comprise a GG, GGG, or GGGS (SEQ ID NO:30) aa sequence. The intra-CAP linker may comprise one or two G4S (SEQ ID NOs: 40-41) aa sequences.

The CAP-NBD linker between Coupling Helix 2 and the NBD is optional. When there is no CAP-NBD linker present, Coupling Helix 2 is fused directly to the N-terminus of the NBD aa sequence. Constructs with no CAP-NBD linker may produce the products that express in E. coli with fewer degradation (proteolytic) products relative to constructs having a CAP-NBD linker. When the CAP-NBD linker is present it may be, for example, from 1 aa to about 10 aas, or from 1 aa to 5 aas. The CAP-NBD linker may comprise, for example, a single aa, such as a G or an A residue. A CAP-NBD linker may comprise a GG, GGG, or GGGS (SEQ ID NO:30) aa sequence. The CAP-NBD linker may comprise one or two G4S (SEQ ID NOs: 40-41) aa sequences.

Either or both of the intra-CAP or CAP-NBD linkers may be rigid linkers, e.g., a polymer comprising Ala and Pro residues, such as a polymer comprising repeats of the sequence Ala-Pro.

### (3) CFTR ATP Binding Cassettes as NBDs

In some cases an NBD of a construct of the present disclosure may comprise an aa sequence from the NBD from human cystic fibrosis transmembrane conductance regulator (CFTR) or an aa sequence having greater than about 90% or greater than about 95% sequence identity to a human CFTR. For example, the NBD may comprise an aa sequence having greater than about 90% or greater than about 95% sequence identity to the NBD aa sequence, GenPep Accession 6MSM_A.

(SEQ ID NO: 27)

| | |
|---|---|
| 1 | MQRSPLEKAS VVSKLFFSWT RPILRKGYRQ RLELSDIYQI |
| | PSVDSADNLS EKLEREWDRE |
| 61 | LASKKNPKLI NALRRCFFWR FMFYGIFLYL GEVTKAVQPL |
| | LLGRIIASYD PDNKEERSIA |
| 121 | IYLGIGLCLL FIVRTLLLHP AIFGLHHIGM QMRIAMFSLI |
| | YKKTLKLSSR VLDKISIGQL |
| 181 | VSLLSNNLNK FDEGLALAHF VWIAPLQVAL LMGLIWELLQ |
| | ASAFCGLGFL IVLALFQAGL |
| 241 | GRMMMKYRDQ RAGKISERLV ITSEMIENIQ SVKAYCWEEA |
| | MEKMIENLRQ TELKLTRKAA |
| 301 | YVRYFNSSAF FFSGFFVVFL SVLPYALIKG IILRKIFTTI |
| | SFCIVLRMAV TRQFPWAVQT |
| 361 | WYDSLGAINK IQDFLQKQEY KTLEYNLTTT EVVMENVTAF |
| | WEEGFGELFE KAKQNNNNRK |
| 421 | TSNGDDSLFF SNFSLLGTPV LKDINFKIER GQLLAVAGST |
| | GAGKTSLLMV IMGELEPSEG |
| 481 | KIKHSGRISF CSQFSWIMPG TIKENIIFGV SYDEYRYRSV |
| | IKACQLEEDI SKFAEKDNIV |
| 541 | LGEGGITLSG GQRARISLAR AVYKDADLYL LDSPFGYLDV |
| | LTEKEIFESC VCKLMANKTR |
| 601 | ILVTSKMEHL KKADKILILH EGSSYFYGTF SELQNLQPDF |
| | SSKLMGCDSF DQFSAERRNS |
| 661 | ILTETLHRFS LEGDAPVSWT ETKKQSFKQT GEFGEKRKNS |
| | ILNPINSIRK FSIVQKTPLQ |
| 721 | MNGIEEDSDE PLERRLSLVP DSEQGEAILP RISVISTGPT |
| | LQARRRQSVL NLMTHSVNQG |
| 781 | QNIHRKTTAS TRKVSLAPQA NLTELDIYSR RLSQETGLEI |
| | SEEINEEDLK ECFFDDMESI |
| 841 | PAVTTWNTYL RYITVHKSLI FVLIWCLVIF LAEVAASLVV |
| | LWLLGNTPLQ DKGNSTHSRN |
| 901 | NSYAVIITST SSYYVFYIYV GVADTLLAMG FFRGLPLVHT |
| | LITVSKILHH KMLHSVLQAP |
| 961 | MSTLNTLKAG GILNRFSKDI AILDDLLPLT IFDFIQLLLI |
| | VIGAIAVVAV LQPYIFVATV |
| 1021 | PVIVAFIMLR AYFLQTSQQL KQLESEGRSP IFTHLVTSLK |
| | GLWTLRAFGR QPYFETLFHK |
| 1081 | ALNLHTANWF LYLSTLRWFQ MRIEMIFVIF FIAVTFISIL |
| | TTGEGEGRVG IILTLAMNIM |
| 1141 | STLQWAVNSS IDVDSLMRSV SRVFKFIDMP TEGKPTKSTK |
| | PYKNGQLSKV MIIENSHVKK |
| 1201 | DDIWPSGGQM TVKDLTAKYT EGGNAILENI SFSISPGQRV |
| | GLLGRTGSGK STLLSAFLRL |
| 1261 | LNTEGEIQID GVSWDSITLQ QWRKAFGVIP QKVFIFSGTF |
| | RKNLDPYEQW SDQEIWKVAD |
| 1321 | EVGLRSVIEQ FPGKLDFVLV DGGCVLSHGH KQLMCLARSV |
| | LSKAKILLLD QPSAHLDPVT |
| 1381 | YQIIRRTLKQ AFADCTVILC EHRIEAMLEC QQFLVIEENK |
| | VRQYDSIQKL LNERSLFRQA |
| 1441 | ISPSDRVKLF PHRNSSKCKS KPQIAALKEE TEEEVQDTRL |
| | SNSLEVLFQ. |

An NBD may, for example, comprise the aa sequence of a human CFTR aa sequence

(SEQ ID NO: 28)

| | |
|---|---|
| 1 | VLKDINFKIE RGQLLAVAGS TGAGKTSLLM VIMGELEPSE |
| | GKIKHSGRIS FCSQFSWIMP |
| 60 | GTIKENIIFG VSYDEYRYRS VIKACQLEED ISKFAEKDNI |
| | VLGEGGITLS GGQRARISLA |
| 121 | RAVYKDADLY LLDSPFGYLD VLTEKEIFES CVCKLMANKT |
| | RILVTSKMEH LKKADKILIL |
| 181 | HEGSSYFYGT FSELQNLQPD F. |

An NBD may comprise an aa sequence having greater than about 90% or greater than about 95% sequence identity to SEQ ID NO:28. Alternatively, the NBD may, for example, comprise an aa sequence having greater than 97% or greater than 99% sequence identity to SEQ ID NO: 28. The NBD may also comprise an aa sequence having greater than 96% or greater than 98% sequence identity to SEQ ID NO:28. Any of those CFTR NBD sequence embodiments may comprise a substitution rendering it hydrolysis deficient.

An NBD may, for example, comprise an aa sequence having greater than 80% or greater than 85% sequence identity to the human CFTR aa sequence, Ser-NBD1 [387-646 (D405-436)] described in Atwell et al., (2010), Protein Engineering, Design & Selection, 23 (5) 375-384.

(SEQ ID NO: 29)

| | |
|---|---|
| 1 | SXTTTEVVME NVTAFWEEGG TPVLKDINFK IERGQLLAVA |
| | GSTGAGKTSL LMVIMGELEP |
| 61 | SEGKIKHSGR ISFCSQFSWI MPGTIKENII FGVSYDEYRY |
| | RSVIKACQLE EDISKFAEKD |
| 121 | NIVLGEGGIT LSGGQRARIS LARAVYKDAD LYLLDSPFGY |
| | LDVLTEKEIF ESCVCKLMAN |
| 181 | KTRILVTSKM EHLKKADKIL ILHEGSSYFY GTFSELQNLQ |
| | PDFSSKLMX, |

where X at position 2 may be absent or Leu; and X at position 229 is absent or G. Alternatively, the NBD may, for example, comprise an aa sequence having greater than about 90% or greater than about 95% sequence identity to SEQ ID NO:29. The NBD may also comprise an aa sequence having greater than 96% or greater than 98% sequence identity to SEQ ID NO:29. An NBD may comprise an aa sequence having greater than about 90% or greater than about 95% sequence identity to aas 1-216 of SEQ ID NO:29, where X at position 2 may be absent or Leu.

### (4) ABCG5 and ABCG8 ATP Binding Sequences as NBDs

ABCG5 and ABCG8 are cognate binding partners that couple ATP binding and their heterodimerization with sterol translocation across cell membranes and represent a source of NBDs for use in the constructs provided herein. Because ABCG5 and ABCG8 are capable of heterodimerization, their NBDs may be used to prepare constructs that, like TAP1 and TAP2 can form heterodimeric constructs.

ABCG5 has the aa sequence:

SEQ ID NO: 285
MGDLSSLTPGGSMGLQVNRGSQSSLEGAPATAPEPHSLGILHASYSVSHR
VRPWWDITSCRQQWTRQILKDVSLYVESGQIMCILGSSGSGKTTLLDAMS
GRLGRAGTFLGEVYVNGRALRREQFQDCFSYVLQSDTLLSSLTVRETLHY
TALLAIRRGNPGSFQKKVEAVMAELSLSHVADRLIGNYSLGGISTGERRR
VSIAAQLLQDPKVMLFDEPTTGLDCMTANQIVVLLVELARRNRIVVLTIH
QPRSELFQLFDKIAILSFGELIFCGTPAEMLDFFNDCGYPCPEHSNPFDF
YMDLTSVDTQSKEREIETSKRVQMIESAYKKSAICHKTLKNIERMKHLKT
LPMVPFKTKDSPGVFSKLGVLLRRVTRNLVRNKLAVITRLLQNLIMGLFL
LFFVLRVRSNVLKGAIQDRVGLLYQFVGATPYTGMLNAVNLFPVLRAVSD
QESQDGLYQKWQMMLAYALHVLPFSVVATMIFSSVCYWTLGLHPEVARFG
YFSAALLAPHLIGEFLTLVLLGIVQNPNIVNSVVALLSIAGVLVGSGFLR
NIQEMPIPFKIISYFTFQKYCSEILVVNEFYGLNFTCGSSNVSVTTNPMC
AFTQGIQFIEKTCPGATSRFTMNFLILYSFIPALVILGIVVFKIRDHLIS
R; Uniprot entry Q9H222-1 (canonical sequence).

An NBD aa sequence from ABCG5 that can heterodimerize with ABCG8 NBD sequences and can be used to prepare constructs that heterodimerize in the presence of ATP may comprise the sequence: RPWWDITSCR QQWTRQILKD VSLYVESGQI MCILGSSGSG KTTLLDAMSG RLGRAGTELG EVYVNGRALR REQFQDCESY VLOSDTLLSS LTVRETLHYT ALLAIRRGNP GSFQKKVEAV MAELSLSHVA DRLIGNYSLG GISTGERRRV SIAAQLLQDP KVMLEDEPTT GLDCMTANQI VVLLVELARR NRIVVLTIHQ PRSELFQLED KIAILSFGEL IFCGTPAEML DEENDCGYPC PE (SEQ ID NO:286). Alternatively, an ABCG5 NBD aa sequence that may be employed in the constructs of the present disclosure may have at least about 96% or at least about 98% sequence identity to SEQ ID NO:286. An NBD aa sequence from human ABCG5 may, for example, comprise an aa sequence having greater than 97% or greater than 98% sequence identity to at least 230 contiguous aas of the NBD aa sequence set forth in SEQ ID NO:286.

A hydrolysis deficient ABCG5 NBD corresponding to SEQ ID NO:286 may comprise the sequence that can be used to prepare constructs of the present disclosure: RPWWDITSCR QQWTRQILKD VSLYVESGQI MCILGSSGSG KTTLLDAMSG RLGRAGTELG EVYVNGRALR REQFQDCFSY VLQSDTLLSS LTVRETLHYT ALLAIRRGNP GSFQKKVEAV MAELSLSHVA DRLIGNYSLG GISTGERRRV SIAAQLLQDP KVMLEDXPTT GLDCMTANQI VVLLVELARR NRIVVLTIHQ PRSELFQLED KIAILSFGEL IFCGTPAEML DFFNDCGYPC PE (SEQ ID NO:295) where X may be any aa other than E, or D, to render the sequence ATP hydrolysis deficient and may also exclude N to avoid the introduction of an Asn (N) linked glycosylation site.

Alternatively, an ABCG5 NBD aa sequence that may be employed in the constructs of the present disclosure may have at least about 96% or at least about 98% sequence identity to SEQ ID NO:295. An NBD aa sequence from human ABCG5 may, for example, comprise an aa sequence having greater than 97% or greater than 98% sequence identity to at least 230 contiguous aas of the NBD aa sequence set forth in SEQ ID NO:295.

An NBD aa sequence from ABCG5 that can heterodimerize with ABCG8 NBD sequences and can be used to prepare constructs that heterodimerize in the presence of ATP may also comprise the sequence:

(SEQ ID NO: 287)
GSSGS GKTTLLDAMS GRLGRAGTFL GEVYVNGRALRREQFQDCFS
YVLQSDTLLS SLTVRETLHY TALLAIRRGN PGSFQKKVEA
VMAELSLSHV ADRLIGNYSL GGISTGERRR VSIAAQLLQD
PKVMLFDEPT TGLDCMTANQ IVVLLVELARRNRIVVLTIH.

Alternatively, an ABCG5 NBD aa sequence that may be employed in the constructs of the present disclosure may have at least about 96% or at least about 98% sequence identity to SEQ ID NO:287. An NBD aa sequence from human ABCG5 may, for example, comprise an aa sequence having greater than 97% or greater than 98% sequence identity to at least 150 contiguous aas of the NBD aa sequence set forth in SEQ ID NO:287.

A hydrolysis deficient ABCG5 NBD corresponding to SEQ ID NO:287 may comprise the sequence: GSSGS GKTTLLDAMS GRLGRAGTEL GEVYVNGRAL RREQFQDCES YVLQSDTLLS SLTVRETLHY TALLAIRRGN PGSFQKKVEA VMAELSLSHV ADRLIGNYSL GGISTGERRR VSIAAQLLQD PKVMLFDXPT TGLDCMTANQ IVVLLVELAR RNRIVVLTIH (SEQ ID NO:296) where X may be any aa other than E, or D, to render the sequence ATP hydrolysis deficient and may also exclude N to avoid the introduction of an Asn (N) linked glycosylation site.

Alternatively, an ABCG5 NBD aa sequence that may be employed in the constructs of the present disclosure may have at least about 96% or at least about 98% sequence identity to SEQ ID NO:296. An NBD aa sequence from human ABCG5 may, for example, comprise an aa sequence having greater than 97% or greater than 98% sequence identity to at least 150 contiguous aas of the NBD aa sequence set forth in SEQ ID NO:296.

ABCG8 has the aa sequence:

(SEQ ID NO: 288)
MAGKAAEERGLPKGATPQDTSGLQDRLFSSESDNSLYFTYSGQPNTLEVR
DLNYQVDLASQVPWFEQLAQFKMPWTSPSCQNSCELGIQNLSFKVRSGQM
LAIIGSSGCGRASLLDVITGRGHGGKIKSGQIWINGQPSSPQLVRKCVAH
VRQHNQLLPNLTVRETLAFIAQMRLPRTFSQAQRDKRVEDVIAELRLRQC
ADTRVGNMYVRGLSGGERRRVSIGVQLLWNPGILILDEPTSGLDSFTAHN
LVKTLSRLAKGNRLVLISLHQPRSDIFRLFDLVLLMTSGTPIYLGAAQHM
VQYFTAIGYPCPRYSNPADFYVDLTSIDRRSREQELATREKAQSLAALFL
EKVRDLDDFLWKAETKDLDEDTCVESSVTPLDTNCLPSPTKMPGAVQQFT
TLIRRQISNDFRDLPTLLIHGAEACLMSMTIGFLYFGHGSIQLSFMDTAA
LLFMIGALIPFNVILDVISKCYSERAMLYYELEDGLYTTGPYFFAKILGE
LPEHCAYIIIYGMPTYWLANLRPGLQPFLLHFLLVWLVVFCCRIMALAAA
ALLPTFHMASFFSNALYNSFYLAGGFMINLSSLWTVPAWISKVSFLRWCF
EGLMKIQFSRRTYKMPLGNLTIAVSGDKILSVMELDSYPLYAIYLIVIGL
SGGFMVLYYVSLRFIKQKPSQDW; Uniprot entry Q9H221-1
(canonical sequence).

An NBD aa sequence from ABCG8 that can heterodimerize with ABCG5 NBD sequences and can be used to prepare constructs that heterodimerize in the presence of ATP may comprise the sequence: LEVRDLNYQV DLASQVPWFE QLAQFKMPWT SPSCONSCEL GIQNLSFKVR SGQMLAIIGS SGCGRASLLD VITGRGHGGK IKSGOIWING QPSSPOLVRK CVAHVROHNQ LLPNLTVRET LAFIAQMRLP RTFSQAQRDK RVEDVIAELR LRQCADTRVG NMYVRGLSGG ERRRVSIGVQ LLWNPGILIL DEPTSGLDSF TAHNLVKTLS RLAKGNRLVL ISLHQPRSDI FRLEDLVLLM TSGTPIYLGA AQHMVQYFTA IGYPCPR (SEQ ID NO:289).

Alternatively, an ABCG8 NBD aa sequence that may be employed in the constructs of the present disclosure may have at least about 96% or at least about 98% sequence identity to SEQ ID NO:289. An NBD aa sequence from human ABCG8 may, for example, comprise an aa sequence having greater than 97% or greater than 98% sequence identity to at least 230 contiguous aas of the NBD aa sequence set forth in SEQ ID NO:289.

A hydrolysis deficient ABCG8 NBD corresponding to SEQ ID NO:289 may comprise the sequence: LEVRDLNYQV DLASQVPWFE QLAQFKMPWT SPSCONSCEL GIQNLSFKVR SGQMLAIIGS SGCGRASLLD VITGRGHGGK IKSGQIWING QPSSPOLVRK CVAHVROHNQ LLPNLTVRET LAFIAQMRLP RTESQAQRDK RVEDVIAELR LRQCADTRVG NMYVRGLSGG ERRRVSIGVQ LLWNPGILIL DXPTSGLDSF TAHNLVKTLS RLAKGNRLVL ISLHQPRSDI FRLEDLVLLM TSGTPIYLGA AQHMVQYFTA IGYPCPR (SEQ ID NO:297); where X may be any aa other than E, or D, to render the sequence ATP hydrolysis deficient and also exclude N to avoid the introduction of an Asn (N) linked glycosylation site.

Alternatively, an ABCG8 NBD aa sequence that may be employed in the constructs of the present disclosure may have at least about 96% or at least about 98% sequence identity to SEQ ID NO:297. An NBD aa sequence from human ABCG8 may, for example, comprise an aa sequence having greater than 97% or greater than 98% sequence identity to at least 230 contiguous aas of the NBD aa sequence set forth in SEQ ID NO:297.

An NBD aa sequence from ABCG8 that can heterodimerize with ABCG5 NBD sequences and can be used to prepare constructs that heterodimerize in the presence of ATP may also comprise the sequence:

(SEQ ID NO: 290)
LAIIGSSGCG RASLLDVITG RGHGGKIKSG QIWINGQPSS
PQLVRKCVAH VRQHNQLLPN LTVRETLAFI AQMRLPRTFS
QAQRDKRVED VIAELRLRQC ADTRVGNMYV RGLSGGERRR
VSIGVQLLWN PGILILDEPT SGLDSFTAHN LVKTLSRLAK
GNRLVLISLH QPRSDIFRLF DLVLLMTSGT PIYLGAAQHM.

Alternatively, an ABCG8 NBD aa sequence that may be employed in the constructs of the present disclosure may have at least about 96% or at least about 98% sequence identity to SEQ ID NO:290. An NBD aa sequence from human ABCG8 may, for example, comprise an aa sequence having greater than 97% or greater than 98% sequence identity to at least 190 contiguous aas of the NBD aa sequence set forth in SEQ ID NO:290.

A hydrolysis deficient ABCG8 NBD corresponding to SEQ ID NO:290 may comprise the sequence: LAIIGSSGCG RASLLDVITG RGHGGKIKSG QIWINGQPSS PQLVRKCVAH VRQHNQLLPN LTVRETLAFI AQMRLPRTES QAQRDKRVED VIAELRLRQC ADTRVGNMYV RGLSGGERRR VSIGVQLLWN PGILILDXPT SGLDSFTAHN LVKTLSRLAK GNRLVLISLH QPRSDIFRLE DLVLLMTSGT PIYLGAAQHM (SEQ ID NO:298); where X may be any aa other than E, or D, to render the sequence ATP hydrolysis deficient and also exclude N to avoid the introduction of an Asn (N) linked glycosylation site.

Alternatively, an ABCG8 NBD aa sequence that may be employed in the constructs of the present disclosure may have at least about 96% or at least about 98% sequence identity to SEQ ID NO:298. An NBD aa sequence from human ABCG8 may, for example, comprise an aa sequence having greater than 97% or greater than 98% sequence identity to at least 190 contiguous aas of the NBD aa sequence set forth in SEQ ID NO:298.

### (5) Substitutions in NBD Sequences and ATP Mediated Dimer Formation

Substitutions that affect the ATP mediated dimerization or higher order complex formation may alter the concentration at which homodimers or heterodimers of polypeptides comprising NBDs form as reflected in the EC50 for dimerization. Substitutions affecting the EC50 for ATP mediated dimerization may be incorporated into the NBD domains described herein.

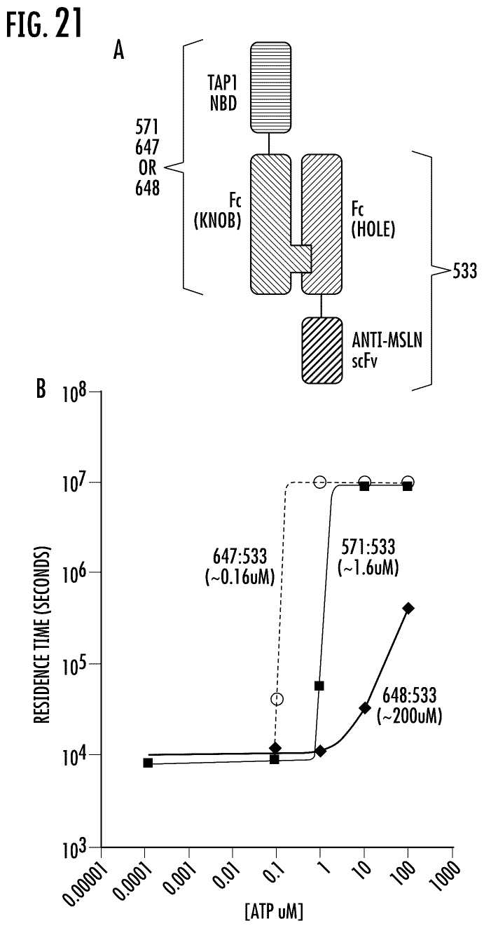

Substitutions at positions S542 (e.g., S542A); Q586 (e.g., Q586A), C662 (e.g., C662S), D667 (e.g., D667A); and C735 (e.g., C735S) have been shown to alter the EC50 for ATP induced dimerization or higher order complex formation of TAP1 NBD containing constructs. In addition, a disulfide bond formed between cysteines substituted at L493 and E573 (L493C: E573C as in construct 571) also affects the alteration in EC50. See, for reference, SEQ ID NO:8, Example 7 including constructs 571, 647 and 648, and FIG. 21. Although the amino acid positions provided are with reference to the TAP1 sequence provided in SEQ ID NO:8, the corresponding positions in other TAP1 sequences (e.g., SEQ ID NOs: 9, 12-14, 291, and 292), TAP2 sequences, and other ATP-binding cassette (ABC) transporters may be obtained by alignment with SEQ ID NO:8 (see, e.g., FIG. 6). The corresponding positions in TAP2 are S507, Q551, R626, and D631. There is no aa directly corresponding to C735 of TAP1 in TAP2. The disulfide bonded cysteine substituted aas in TAP1 (L493C: E573C) correspond to T458 and E538 that may be substituted with cysteines and disulfide bonded in TAP2.

Based on the foregoing, the NBDs of TAP1 and/or TAP2 may comprise substitutions at any one or more of the above-mentioned positions resulting in modified EC50 values for ATP mediated dimerization or higher order complex formation in addition to any one or more other substitutions (e.g., substitutions that render the NBDs ATP hydrolysis deficient). The NBDs of other ABC transporters may similarly comprise conservative substitutions or non-conservative substitutions at the corresponding positions in their sequences in addition to any one or more other substitutions. The NBD of TAP1 may comprise a substitution at D667 (e.g., D667A); and the NBDs of TAP2 and other ABC transporters may comprise a substitution (e.g., an alanine) at the corresponding aa position (see, e.g., construct 647). The NBD of TAP1 may comprise a substitution at Q586 (e.g., Q586A); and the NBDs of TAP2 and other ABC transporters may comprise a substitution (e.g., an alanine) at the corresponding aa position (see, e.g., construct 648). The NBD of TAP1 may comprise substitutions at L493C and E573C resulting in disulfide bond formation; and substitutions at S542 (e.g., S542A), C662 (e.g., C662S), and C735 (e.g., C735S); and the NBDs of TAP2 and other ABC transporters may comprise substitutions at the corresponding aa positions (see, e.g., construct 571).

TAP1, TAP2 and other ABC transporters may comprise substitutions that can affect the EC50 for ATP mediated dimerization or higher order complex formation of ABC transporters in the Q loop amino acids. For example, TAP1 may comprise substitutions at Q586 and/or E587 and TAP2 may comprise substitutions at Q551 and/or E552. TAP1 and TAP2 NBD aa sequences may comprise nonconservative substitutions of the magnesium binding aa Q586 in TAP1 or Q551 in TAP2, for example with alanine or another non-charged aa (e.g., Gly, Leu, or Ile). Corresponding substitutions may be made in other ABC transporter NBD aa sequences. Any of the substitutions may be conservative or nonconservative and are not exclusive of other substitutions in the NBD aa sequences.

TAP1, TAP2 and other ABC transporters may comprise substitutions that can affect the EC50 for ATP mediated dimerization or higher order complex formation of ABC transporters in the Walker region aas. For example, TAP1 may comprise substitutions of any one or more of N540, G541, S542, G543, K544, S545, and/or T546 and TAP2 may comprise substitutions at any one or more of N505, G506, S507, G508, K509, S510 (Mg), and/or T511. TAP1 and/or TAP2 NBD aa sequences may comprise substitutions of the magnesium binding aas S545 in TAP1 or S510 in TAP2, for example with alanine or another non-charged aa (e.g., Gly, Leu, or Ile). Corresponding substitutions may be made in other ABC transporter NBD aa sequences. Any of the substitutions may be conservative or nonconservative and are not exclusive of other substitutions in the NBD aa sequences.

TAP1, TAP2 and other ABC transporters may comprise substitutions that can affect the EC50 for ATP mediated dimerization or higher order complex formation of ABC transporters outside of the Walker region aas. For example, TAP1 may comprise substitutions of any one or more of Q586 and D668 and TAP2 may comprise substitutions at any one or more of Q551 and E632, each of which interact with water and/or magnesium in the nucleotide binding pocket. TAP1 and/or TAP2 NBD aa sequences may comprise substitutions of those aas, for example with alanine or another non-charged aa (e.g., Gly, Leu, or Ile). Corresponding substitutions may be made in other ABC transporter NBD aa sequences. Any of the substitutions may be conservative or nonconservative and are not exclusive of other substitutions in the NBD aa sequences.

TAP1, TAP2 and other ABC transporters may comprise substitutions of aas binding to the adenine or ribose of ATP that can affect the EC50 for ATP mediated dimerization or higher order complex formation of ABC transporters. For example, TAP1 may comprise substitutions at any one or more of Y412 (binding to adenine) and V520 (binding to ribose) and TAP2 may comprise substitutions at any one or more of Y477 (binding to adenine) and V585 (binding to ribose). TAP1 and/or TAP2 NBD aa sequences may comprise substitutions of those aas, for example with alanine or another non-charged aa (e.g., Gly, Leu, or Ile). Corresponding substitutions may be made in other ABC transporter NBD aa sequences. Any of the substitutions may be conservative or nonconservative and are not exclusive of other substitutions in the NBD aa sequences.

### 2. Linkers

The constructs of the present disclosure optionally have independently selected linkers located between any two elements. Independently selected linker sequences may also be placed at the end of aa sequences of the constructs of the present disclosure for stability and protection from proteolysis. The linkers can be located between any two of the NBD aa sequence, the scaffold aa sequence, and the AD. Linkers are typically comprised of aa sequences from 4 to 50 aas in length (e.g., from 4 to 25 aas or from 25 to 50 aas) and may be chosen for, among other things, their rigidity. Linkers are each selected independently.

In some instances, linkers are flexible aa sequences that are comprised of glycine, serine, and/or alanine residues. In other instances, linkers are aa sequences comprised of glycine and serine. For example, the sequence GGGS (SEQ ID NO:30) or the sequence GGGGS (also denoted G₄S or G4S, SEQ ID NO:40) may appear or be repeated from 1-10 times in the linkers (GGGS)₁₋₁₀ (SEQ ID NOs: 30-39, respectively) or (GGGGS)₁₋₁₀ (SEQ ID NOs: 40-49, respectively). In some cases where linkers comprise a GGGS or GGGGS sequence, those sequences may appear from 1-5 or from 5-10 times.

Rigid linkers may be employed where it is desirable to maintain a substantially fixed distance or spatial separation between the domains to reduce or substantially eliminate unfavorable interactions between domains of the constructs (e.g., between NBDs in the same construct). For example, a pair of NBDs present in a single molecule may be kept from self-associating through the use of rigid linkers. Exemplary rigid peptide or polypeptide linkers include linkers comprising the sequence EAAAK (SEQ ID NOs: 50, 198-216) which may appear in the linker from 1-20 times (i.e., 1, 2, 3, 4, 5, 6, 7, 8, 9, 10, 11, 12, 13, 14, 15, 16, 17, 18, 19, or 20 times). In some cases, the EAAAK sequence may be repeated from 1-5 times or from 5-10 times. In some cases, the EAAAK sequence may be repeated from 11-15 times or from 16-20 times. Some rigid linkers may comprise greater than 30% or greater than 40% proline residues on a molar basis. Other rigid linkers may comprise from 30% to 40%, or from 40% to 60% proline residues on a molar basis. Rigid peptide linkers may include, but are not limited to, linkers having a length of 4 to 40 (e.g., 4-20 or 20-40) aas comprised of repeats comprising (Lys-Pro) (SEQ ID NO:51 in general and specifically according to length, SEQ ID NOs: 217-235), (Glu-Pro) (SEQ ID NO:52 in general and specifically according to length, SEQ ID NOs: 236-254), and (Thr-Pro-Arg) (SEQ ID NO:53 in general and specifically according to length, SEQ ID NOs: 255-274). Rigid peptide linkers may comprise repeats of Ala-Pro from 4 to 40 aas in length (SEQ ID NO:54 in general and specifically according to length, SEQ ID NOs: 55, 56, 57, 58 and 275-284), including but not limited to 4 aas (AP2) (SEQ ID NO:55), 6 aas (AP3) (SEQ ID NO:56), 8 aas (AP4) (SEQ ID NO:57), or 10 aas (AP5) (SEQ ID NO:58) in length. Other rigid linkers include linkers comprising repeats of the dipeptide sequence KP or EP from 2 to 10 or 10 to 20 times (SEQ ID NOs: 51 in general and specifically according to repetition, SEQ ID NOs: 217-235 and SEQ ID NO:52 in general and specifically according to length, SEQ ID NOs: 236-254, respectively). Rigid peptide linkers placed at the C-terminus of a TAP1 NBD of any of the sequences of SEQ ID NOs: 9, 12-16, 19, and 20 may take the place of the PADAPE (SEQ ID NO:11) sequence.

Where constructs of the present disclosure comprise a payload (e.g., a chemotherapeutic agent or label), any linker connecting the payload to the construct may be cleavable or non-cleavable (stable) in vivo and particularly in the TME. A stable linker ensures that less of the payload (e.g., cytotoxic payload) separates from the construct before reaching a tumor cell, which improves the safety (e.g., limits patient side effects due to non-tumor target action) and limits the dosage necessary to achieve a therapeutic effect.

Where it is desirable for payloads to separate from the construct(s) they may be joined by a cleavable (labile) linker based on, for example chemical motifs including disulfides, hydrazones, peptides, or acid-sensitive cleavable linkers such as carbonate linkers (see, e.g., the polyethylene glycol-acid-sensitive cleavable carbonate linker of Sacituzumab govitecan). Linkers comprising an amino acid sequence susceptible to cleavage by one or more (e.g., two or more) proteases found in the TME may be utilized as cleavable linkers. Linker aa sequences may be selected such that they are susceptible to cleavage by proteases expressed at higher levels in the TME than in one or more non-tumor tissues, or such that the protease is expressed at a level higher than in peritumor tissue). For example, amino acid sequences, including, but not limited to, those cleavable by legumain, matriptase-1, matriptase-2, Cathepsin-S, MMP1, MMP2, MMP3, MMP7, MMP8, MMP12, MMP13, MMP 14, or furin may be used as cleavable linkers in light of the proteases expression in one or more tumors and sequence specific cleavage. Some protease sensitive sequences that may be incorporated into linkers cleavable by those proteases follow.

Legumain has a strict specificity for cleavage after Asp and Asn residues and is expressed at significantly lower levels in normal cells than in tumors or tumor-associated macrophages (TAMs). Accordingly, sequences comprising one or more Asn and/or Asp are suitable for use as legumain cleavable linkers.

Matriptase-1 and matriptase-2 substrates comprise an Arg or Lys at the P1 position and preferentially substrates having an aa with a small side chain, such as Ala and Gly, at the P2 position.

Cathepsin S cleaves substrates with a-Val-Val-Arg-sequence, particularly when the sequence is imbedded in a polypeptide with at least two aa residues on each side of the cleavage site.

Matrix metalloproteinases (MMPs) are a family of calcium-dependent zinc-containing endopeptidases that cleave at tetra peptide sequences with a Leu residue on their carboxy end. Table 2, which follows, provides tripeptide sequences preceding the bond to the cleaved leucine for several MMPs. The bond to the Leu that is specifically proteolyzed is indicated by a “˜”. The peptides are listed in rank order of their cleavage rates for each of the proteases.

TABLE 2

| MMP1 | MMP2 | MMP3 | MMP7 | MMP8 | MMP9 | MMP12 | MMP13 | MMP14 |
|---|---|---|---|---|---|---|---|---|
| PLN~ | PAG~ | PAG~ | PLG~ | PLG~ | PLG~ | PLG~ | PAG~ | PLG~ |
| PLG~ | VAN~ | PAN~ | VLG~ | PLN~ | PAG~ | PLN~ | PLG~ | PAG~ |
| | PLG~ | VAN~ | PLN~ | VAN~ | PLN~ | VAN~ | | VAN~ |

Based on work by Eckhard et al. Matrix Biol. (2016) 49, 37-60.
Furin is expressed in tumors at levels higher than normal tissue adjacent to the tumor. See e.g., Azevedo et al., Cancers (Basel) 2023 Aug. 1; 15 (15): 3909, doi: 10.3390/cancers15153909. Furin displays sequence selectivity for the motif Arg-Xaa-Lys-Arg or Arg-Xaa-Arg-Arg, wherein Xaa is any amino acid. In some cases, Xaa may be selected from the group consisting of D, E, F, G, H, K, L, P, Q, R, S, T, and V, or the group consisting of H, K, and R. See, e.g., Hosaka et al., J. Bio. Chem., 266 (19) 12127-12130 (1991).

Sequences sensitive to numerous other proteases may be utilized in cleavable linkers including, but not limited to, cysteine-aspartic acid protease (caspase) family members such as Caspase 7; Enterokinase, Granzyme Tobacco etch virus protease (TEV protease), and ADAM proteases (A Disintegrin And Metalloproteinase) family members.

### 3. Scaffolds

Scaffold aa sequences may be incorporated into the constructs described herein as a means of structural organization, to increase the half-life of the molecules in vivo, and, where capable of inducing ADCC, ADCP, and/or CDC, to act as a basis for those effector functions. The scaffolds may be capable of forming dimers and higher order complexes (e.g., trimers). In certain instances, scaffolds may be formed from an aa sequence and its non-identical cognate aa sequence binding partner (interspecific sequences) that selectively interact to selectively form a specific heterodimer (interspecific heterodimer). Alternatively, scaffolds may be non-dimerizable (non-dimerizing) in which case they do not form dimers or higher order complexes. Scaffolds incorporated into the constructs described herein may, for example, comprise non-Ig aa sequences (e.g., an XTEN polypeptide, leucine zipper, or albumin polypeptides), or Ig aa sequences that can dimerize, heterodimerize (i.e., form interspecific pairs), or remain monomeric (e.g., monomeric Fc (mFc) and monomeric CH3 (mCH3). See, e.g., Liu et al., Front Immunol. 2017; 8:38. Scaffolds comprising Ig CH2-CH3 region sequences (e.g., scFc sequences) may also function as monomeric scaffolds even they comprise separate polypeptides (e.g., a pair of interspecific Fc sequences such as a KiH sequence pair), particularly if the separate polypeptides are joined by disulfide bonds (e.g., as in an KiHs-s sequence pair).

Scaffolds, and particularly IgFc scaffolds, may provide a variety of functions including stabilizing the molecule, extending its circulating half-life in blood, and providing other immune system related functions associated with phagocytosis and cytotoxicity (e.g., ADCC, ADCP, and/or CDC). Scaffold sequences (e.g., Ig scaffold sequences) placed at the N-terminal to an NBD may act as a CAP structure.

Scaffold polypeptide sequences may comprise Ig heavy chain constant region polypeptide sequences that function as a dimerization or multimerization sequence. Immunoglobulin scaffolds, such as heavy chain constant regions may include cysteines that form disulfide bonds between scaffold sequences that spontaneously formed during cellular expression of constructs. Where the scaffold comprises an Ig aa sequence, it may comprise an Ig heavy chain constant region (CH2-CH3) polypeptide aa sequence (e.g., an IgFc aa sequence) that may dimerize with a second Ig CH2-CH3 aa sequence. Any one or more cysteines involved in interchain disulfide bonds that stabilize Ig heavy chain dimers may be substituted (e.g., with an alanine or serine) so that the Ig heavy chain sequence cannot homodimerize or heterodimerize and form interchain disulfide bonds.

A scaffold polypeptide may comprise the wt. Homo sapiens IgG1 Fc polypeptide sequence: 1 DKTHTCPPCP APELLGGPSV FLFPPKPKDT LMISRTPEVT CVVVDVSHED PEVKENWYVD 61 GVEVHNAKTK PREEQYNSTY RVVSVLTVLH QDWLNGKEYK CKVSNKALPA PIEKTISKAK 121 GQPREPQVYT LPPSREEMTK NQVSLTCLVK GFYPSDIAVE WESNGQPENN YKTTPPVLDS 181 DGSFFLYSKL TVDKSRWQQG NVFSCSVMHE ALHNHYTOKS LSLSPGK (SEQ ID NO:59) (see NCBI GenPept ACCESSION 7LBL_A), which may form homodimers stabilized by interchain disulfide bonds during expression. A scaffold may comprise an aa sequence having greater than about 90% or greater than about 95% sequence identity to SEQ ID NO:59. A scaffold may also comprise an aa sequence having greater than 96% or greater than 98% sequence identity to SEQ ID NO:59.

Ig Fc aa sequences may comprise CH2 and/or CH3 domains modified to prevent dimerization, with the resulting sequences existing in monomeric form. One set of substitutions that may be included in IgG1 heavy chain constant region aa sequences that substantially or completely blocks dimerization with another IgG1 aa sequence includes L351S, T366R, L368H and P395K (corresponding to L131S, T146R, L348H, and P175K in SEQ ID NO:59). See Ying et al. (2014) mAbs 6 (5): 1201-1210. IgG1 Fc sequences bearing those substitutions remain in soluble monomeric form capable of high affinity binding to FcγRI (Kd on the order of 10 nM) and bind to the neonatal Fc receptor (FcRn) that prevents lysosomal degradation of antibodies thereby extending their in vivo half-life. Id. At the same time, monomeric IgG1 sequences bearing those substitutions fail to provide effector functions including Fc-mediated CDC and ADCC by natural killer cells consistent with their failure to bind to the FcγRIIIa receptor. Id.

An IgG1 sequence comprising those sequence substitutions suitable as a monomeric (non-dimerizing) Fc sequence (mFc) scaffold in the molecules described herein may comprise the aa sequence

(SEQ ID NO: 60)

| | |
|---|---|
| 1 | APELLGGPSV FLFPPKPKDT LMISRTPEVT CVVVDVSHED |
| | PEVKFNWYVD GVEVHNAKTK |
| 61 | PREEQYNSTY RVVSVLTVLH QDWLNGKEYK CKVSNKALPA |
| | PIEKTISKAK GQPREPQVYT |
| 121 | SPPSRDELTK NQVSLRCHVK GFYPSDIAVE WESNGQPENN |
| | YKTTKPVLDS DGSFFLYSKL |
| 181 | TVDKSRWQQG NVFSCSVMHE ALHNHYTQKS LSLSPGK. |

A scaffold may comprise an aa sequence having greater than about 90% or greater than about 95% sequence identity to SEQ ID NO:60, while retaining the substitutions that cause it to remain monomeric. A scaffold may also comprise an aa sequence having greater than 96% or greater than 98% sequence identity to SEQ ID NO:60, while retaining the substitutions that cause it to remain monomeric.

Stabilizing disulfide bonds may be introduced into the sequence of SEQ ID NO:60 between cysteines substituted for L242 and K334 (indicated as C* in SEQ ID NO:61 and SEQ ID NO:62), and/or between cysteines substituted for P343 and A431 (indicated as C** in SEQ ID NO:62). Accordingly, a monomeric IgG1 Fc scaffold sequence may comprise the aa sequence

```
  (SEQ ID NO: 61)
    1 APELLGGPSV FC*FPPKPKDT LMISRTPEVT CVVVDVSHED PEVKENWYVD GVEVHNAKTK
  61 PREEQYNSTY RVVSVLTVLH QDWLNGKEYK CKVSNKALPA PIEC*TISKAK GQPREPQVYT
121 SPPSRDELTK NQVSLRCHVK GFYPSDIAVE WESNGQPENN YKTTKPVLDS DGSFFLYSKL
181 TVDKSRWQQG NVFSCSVMHE ALHNHYTQKS LSLSPGK,
or the aa sequence:
(SEQ ID NO: 62)
    1 APELLGGPSV FC*FPPKPKDT LMISRTPEVT CVVVDVSHED PEVKENWYVD GVEVHNAKTK
  61 PREEQYNSTY RVVSVLTVLH QDWLNGKEYK CKVSNKALPA PIEC*TISKAK GQC**REPQVYT
121 SPPSRDELTK NQVSLRCHVK GFYPSDIAVE WESNGQPENN YKTTKPVLDS DGSFFLYSKL
181 TVDKSRWQQG NVFSCSVMHE C**LHNHYTQKS LSLSPGK.
```

A scaffold may comprise an aa sequence having greater than about 90% or greater than about 95% sequence identity to SEQ ID NO: 61 or SEQ ID NO:62, while retaining the substitutions that cause it to remain monomeric. A scaffold may also comprise an aa sequence having greater than 96% or greater than 98% sequence identity to SEQ ID NO:61 or SEQ ID NO: 62, while retaining the substitutions that cause it to remain monomeric.

Non-dimerizing scaffolds may also be prepared as human single chain Fc (scFc) dimers. See, e.g., Zhou et al., Biomaterials 117:24-31 (2017). Such scFc dimers may be based on IgG1 and have the sequence

```
(SEQ ID NO: 63)
    1 DKTHTCPPCP APELLGGPSV FLFPPKPKDT LMISRTPEVT CVVVDVSHED PEVKENWYVD
  61 GVEVHNAKTK PREEQYNSTY RVVSVLTVLH QDWLNGKEYK CKVSNKALPA PIEKTISKAK
121 GQPREPQVYT LPPSRDELTK NQVSLTCLVK GFYPSDIAVE WESNGQPENN YKTTPPVLDS
181 DGSFFLYSKL TVDKSRWQQG NVFSCSVMHE ALHNHYTQKS LSLSPGGGGG SGGGGSGGGG
241 SGGGGSGGGG SGGGGSDKTH TCPPCPAPEL LGGPSVELFP PKPKDTLMIS RTPEVTCVVV
301 DVSHEDPEVK FNWYVDGVEV HNAKTKPREE QYNSTYRVVS VLTVLHQDWL NGKEYKCKVS
361 NKALPAPIEK TISKAKGQPR EPQVYTLPPS RDELTKNQVS LTCLVKGFYP SDIAVEWESN
421 GQPENNYKTT PPVLDSDGSF FLYSKLTVDK SRWQQGNVFS CSVMHEALHN HYTQKSLSLS
481 PG,
```

in which a G4S linker sequence that is bolded and italicized joins the two IgFc sequences. A scaffold may, for example, comprise an aa sequence having greater than about 90% or greater than about 95% sequence identity to SEQ ID NO:63. The scaffold may also comprise an aa sequence having greater than 96% or greater than 98% sequence identity to SEQ ID NO:63. The scaffold construct of SEQ ID NO:63 is competent for the induction of ADCC, ADCP, and CDC, but substitutions including “LALA” and/or other substitutions which are discussed below may be introduced to selectively alter the ability of the scFc to suppress or induce effector functions.

A scaffold polypeptide may comprise the wt. Homo sapiens IgG2 Fc polypeptide of SEQ ID NO:64 (see GenBank AAN76044, aas 99-32: 1 STKGPSVEPL APCSRSTSES TAALGCLVKD YFPEPVTVSW NSGALTSGVH TFPAVLOSSG 61 LYSLSSVVTV PSSNFGTQTY TCNVDHKPSN TKVDKTVERK CCVECPPCPA PPVAGPSVFL 121 FPPKPKDTLM ISRTPEVTCV VVDVSHEDPE VQFNWYVDGV EVHNAKTKPR EEQENSTERV 181 VSVLTVVHQD WINGKEYKCK VSNKGLPAPI EKTISKTKGQ PREPQVYTLP PSREEMTKNQ 241 VSLTCLVKGF YPSDIAVEWE SNGQPENNYK TTPPMLDSDG SFFLYSKLTV DKSRWQQGNV 301 FSCSVMHEAL HNHYTOKSLS LSPGK, optionally modified to prevent dimerization. Alternatively, the scaffold may, for example, comprise an aa sequence having greater than about 90% or greater than about 95% sequence identity to SEQ ID NO:64. The scaffold may also comprise an aa sequence having greater than 96% or greater than 98% sequence identity to SEQ ID NO:64.

A scaffold polypeptide may comprise the wt. Homo sapiens IgG3 Fc polypeptide of SEQ ID NO:65 (see, e.g., GenBank AAW65947, aas 19-24: 1 HKPSNTKVDK RVELKTPLGD TTHTCPPCPA PELLGGPSVF LFPPKPKDTL MISRTPEVTC 61 VVVDVSHEDP EVKENWYVDG VEVHNAKTKP REEQYNSTYR VVSVLTVLHQ DWLNGKEYKC 121 KVSNKALPAP IEKTISKAKG QPREPQVYTL PPSRDELTKN QVSLTCLVKG FYPSDIAVEW 181 ESNGQPENNY KTTPPVLDSD GSFFLYSKLT VDKSRWQQGN VFSCSVMHEA LHNHYTQKSL 241 SLSPGK, optionally modified to prevent dimerization. Alternatively, the scaffold may, for example, comprise an aa sequence having greater than about 90% or greater than about 95% sequence identity to SEQ ID NO:65. The scaffold may also comprise an aa sequence having greater than 96% or greater than 98% sequence identity to SEQ ID NO: 65.

A scaffold polypeptide may comprise the wt. Homo sapiens IgG4 Fc polypeptide of SEQ ID NO:66 1 PPCPSCPAPE FLGGPSVFLF PPKPKDTLMI SRTPEVTCVV VDVSQEDPEV QFNWYVDGVE 61 VHNAKTKPRE EQFNSTYRVV SVLTVLHQDW LNGKEYKCKV SNKGLPSSIE KTISKAKGQP 121 REPQVYTLPP SQEEMTKNQV SLTCLVKGFY PSDIAVEWES NGQPENNYKT TPPVLDSDGS 181 FFLYSRLTVD KSRWQEGNVF SCSVMHEALH NHYTOKSLSL SPG, optionally modified to prevent dimerization. Alternatively, the scaffold may comprise an aa sequence having greater than about 90% or greater than about 95% sequence identity to SEQ ID NO:66. The scaffold may also comprise an aa sequence having greater than 96% or greater than 98% sequence identity to SEQ ID NO:66.

Alternative non-dimerizing (non-multimerizing) scaffold domains include, but are not limited to, Human Serum Albumin (I) fusions (see, e.g., Mandrup et al., nature.com/articles/s42003-021-01790-2. on the World Wide Web) anti-HSA binding domains, including but not limited to anti-HSA peptides (see e.g., helix-loop-helix peptides in pubs.acs.org/doi/10.1021/acs.molpharmaceut.2c00106 on the www), antibody and antibody fragments (e.g. scFvs, FABs, etc.) and VHH domains (see semanticscholar.org/paper/Serum-albumin % E2%80%90binding-VHHs-with-variable-pH-enable-Faassen-Ryan/d34256a0d39a0ab92db9195210fa0fc7430758b6 on the www). Ig κ or Ig λ light chain constant regions or Ig heavy chain CH1 sequences may also be employed as scaffolds. Any of the foregoing may be subject to PEGylation or Lipidation to extend their half-lives. (see pubs.acs.org/doi/10.1021/acsmedchemlett.8b00226 on the World Wide Web).

Any one or more cysteines in Ig heavy chain aa sequences used as a scaffold may be substituted (e.g., with an alanine or serine) so that the Ig heavy chain sequence cannot dimerize (e.g., homodimerize) and form interchain disulfide bonds. Alternatively, where it is desirable to form stable disulfide bonds between Ig heavy chain constant region aa sequences, cysteines may be introduced into the sequences or one or more of the hinge region disulfide sequences may be utilized.

### a) Interspecific Immunoglobulin Fc Scaffold Polypeptides

Where an asymmetric pairing between two scaffold sequences is present in a construct of the present disclosure, aa sequences that selectively form heterodimers with a specific cognate counterpart sequence can be employed. Such cognate sequence pairs or “interspecific” sequences may be Ig Fc polypeptide sequence variants. A number of such interspecific polypeptide sequences have been described including, but not limited to, knob-in-hole without (KiH) or with (KiHs-s) a stabilizing disulfide bond, HA-TF, ZW-1, 7.8.60, DD-KK, EW-RVT, EW-RVTs-s, and A107 sequences.

One interspecific binding pair based on IgG1 Fc sequences comprises a T366Y substitution in the first sequence and a Y407T substitution in the second sequence (or the corresponding residues of other IgGs), which affect the CH3 domain interface. See Ridgway et al., Protein Engineering 9:7, 617-621 (1996).

A knob-in-hole or KiH interspecific binding pair involves the formation of a knob by a T366W substitution in a first aa sequence, and a hole in the complementary Ig Fc sequence formed by the triple substitutions T366S, L368A and Y407V. See Xu et al. mAbs 7:1, 231-242 (2015). A related KiHs-s interspecific binding pair that includes Cys residues to form a stabilizing interchain disulfide bond comprises a first Ig Fc polypeptide with Y349C, T366S, L368A, and Y407V substitutions and a second Ig Fc polypeptide with S354C and T366W substitutions. The stabilizing disulfide bond may form between the Y349C and the S354C when the sequences are co-expressed. See, e.g., Brinkmann and Konthermann, mAbs 9:2, 182-212 (2015). Ig Fc polypeptide sequences, either with or without knob-in-hole modifications, can be stabilized by the formation of disulfide bonds between the Ig Fc polypeptides (e.g., the hinge region disulfide bonds).

KiH, KiHs-s, and other interspecific binding sequence pairs based upon Ig sequences are summarized in Table 3. The table includes cross references (provided in braces “{ }”) to the numbering of the wt. IgG1 Fc sequence set forth in SEQ ID NO:59.

TABLE 3
Interspecific immunoglobulin sequences and their
cognate counterpart interspecific sequences

| | Substitutions in | Substitutions in the second interspecific |
|---|---|---|
| Interspecific | the first interspecific | polypeptide (counterpart sequence or |
| Pair Name | polypeptide sequence | cognate binding partner) sequence) |
| KiH | T366W | T366S/L368A/Y407V |
| | {T146W} | {T146S/L148A/Y187V} |
| KiHs-s | T366W/S354C* | T366S/L368A/Y407V/Y349C* |
| | {T146W/S134C*} | {T146S/L148A/Y187V/Y129C*} |
| HA-TF | S364H/F405A | Y349T/T394F |
| | {S144H/F185A} | {Y129T/T174F} |
| ZW1 | T350V/L351Y/F405A/Y407V | T350V/T366L/K392L/T394W |
| | {T130V/L131Y/F185A/Y187V} | {T130V/T146L/K172L/T174W} |
| 7.8.60 | K360D/D399M/Y407A | E345R/Q347R/T366V/K409V |
| | {K140D/D179M/Y187A} | {E125R/Q127R/T146V/K189V} |
| DD-KK | K409D/K392D | D399K/E356K |
| | {K189D/K172D} | [D179K/E136K} |
| EW-RVT | K360E/K409W | Q347R/D399V/F405T |
| | {K140E/K189W} | {Q127R/D179V/F185T} |
| EW-RVTs-s | K360E/K409W/Y349C* | Q347R/D399V/F405T/S354C* |
| | {K140E/K189W/Y129C*} | {Q127R/D179V/F185T/S134C*} |
| A107 | K370E/K409W | E357N/D399V/F405T |
| | {K150E/K189W} | {E137N/D179V/F185T} |

Table 3 is modified from Ha et al., Frontiers in Immunol. Vol. 7 Article 394, pages 1-16 (2016), doi: 10.3389/fimmu.2016.00394
*aa forms a stabilizing disulfide bond.

Suitable scaffold polypeptides also include interspecific “SEED” sequences having 45 residues derived from IgA in an IgG1 CH3 domain of the interspecific sequence and 57 residues derived from IgG1 in the IgA CH3 in its counterpart interspecific sequence. See, e.g., Ha et al., Frontiers in Immunol. Vol. 7, Article 394, pages 1-16 (2016), doi: 10.3389/fimmu.2016.00394 and citations therein.

A scaffold polypeptide suitable for use in constructs described herein may comprise the aa sequence of an interspecific binding sequence and/or its counterpart interspecific binding sequence selected from the group consisting of: KiH; KiHs-s; HA-TF; ZW-1; 7.8.60; DD-KK; EW-RVT; EW-RVTs-s; A107; or SEED sequences. IgFc based scaffolds may comprise substitutions that suppress or enhance effector functions (e.g., ADCC, ADCP, and/or CDC) relative to the effect observed with the wt. sequence under otherwise identical conditions. The effector function affected may be ADCC or ADCP. The effector function affected may be CDC.

A scaffold polypeptide suitable for use in constructs described herein may comprise an IgG1 Fc sequence with a T146W KiH sequence substitution, and its counterpart interspecific binding partner polypeptide comprises an IgG1 sequence having T146S, L148A, and Y187V KiH sequence substitutions, where the scaffold polypeptide comprises a sequence having at least 90% or at least 95% sequence identity to at least 210 (e.g., at least 220, or all 227) contiguous aas of the wt. IgG1 of SEQ ID NO:59. The scaffold polypeptide may comprise a KiH substitution and have at least 96% or at least 97% sequence identity to at least 210 (e.g., at least 220, or all 227) contiguous aas of the wt. IgG1 of SEQ ID NO:59. Such scaffold polypeptides optionally comprise substitutions at one or more of: L234 and L235 (e.g., L234A/L235A “LALA” or L234F/L235E); N297 (e.g., N297A); P331 (e.g. P331S); L351 (e.g., L351K); T366 (e.g., T366S); P395 (e.g., P395V); F405 (e.g., F405R); Y407 (e.g., Y407A); and K409 (e.g., K409Y), that among other things may suppress effector function. Those substitutions appear at: L14 and L15 (e.g., L14A/L15A “LALA” or L14F/L15E); N77 (e.g., N77A); P111 (e.g. P111S) L131 (e.g., L131K); T146 (e.g., T146S); P175 (e.g., P175V); F185 (e.g., F185R); Y187 (e.g., Y187A); and K189 (e.g., K189Y) in the wt. IgG1 sequence of SEQ ID NO:59. Alternatively, IgFc based scaffolds may comprise substitutions that enhance effector functions.

A scaffold polypeptide suitable for use in constructs described herein may comprise an IgG1 Fc sequence with a T146W KiH sequence substitution, and its counterpart interspecific binding partner polypeptide comprises an IgG1 sequence having T146S, L148A, and Y187V KiH sequence substitutions, where the scaffold polypeptide comprises a sequence having at least 90% or at least 95% sequence identity to at least 210 (e.g., at least 220, or all 227) contiguous aas of the wt. IgG1 of SEQ ID NO:59. The scaffold polypeptide may comprise a KiH substitution and have at least 96% or at least 97% sequence identity to at least 210 (e.g., at least 220, or all 227) contiguous aas of the wt. IgG1 of SEQ ID NO:59. Such scaffold polypeptide sequence(s) may comprise additional substitutions such as L14 and/or L15 substitutions (e.g., “LALA” substitutions L234A and L235A) and/or an N77 substitution (N297 e.g., N297A or N297G).

A scaffold polypeptide suitable for use in constructs described herein may comprise an IgG1 Fc sequence with T146W and S134C KiHs-s substitutions, and its counterpart interspecific binding partner polypeptide comprises an IgG1 sequence having T146S, L148A, Y187V and Y129C KiHs-s substitutions, where the scaffold polypeptide comprises a sequence having at least 90% or at least 95% sequence identity to at least 210 (e.g., at least 220, or all 227) contiguous aas of the wt. IgG1 of SEQ ID NO:59. The scaffold polypeptide may comprise a KiHs-s substitution and have at least 96% or at least 97% sequence identity to at least 210 (e.g., at least 220, or all 227) contiguous aas of the wt. IgG1 of SEQ ID NO:59. Such scaffold polypeptide sequence(s) may comprise additional substitutions such as L14 and/or L15 substitutions (e.g., “LALA” substitutions L234A and L235A) and/or an N77 substitution (N297 e.g., N297A or N297G) or substitutions that enhance one or more effector functions.

A scaffold polypeptide suitable for use in constructs described herein may comprise an IgG1 Fc sequence with S144H and F185A HA-TF substitutions, and its counterpart interspecific binding partner polypeptide comprises an IgG1 sequence having Y129T and T174F HA-TF substitutions, where the scaffold polypeptide comprises a sequence having at least 90% or at least 95% sequence identity to at least 210 (e.g., at least 220, or all 227) contiguous aas of the wt. IgG1 of SEQ ID NO:59. The scaffold polypeptide may comprise an HA-TF substitution and have at least 96% or at least 97% sequence identity to at least 210 (e.g., at least 220, or all 227) contiguous aas of the wt. IgG1 of SEQ ID NO:59. Such scaffold polypeptide sequence(s) may comprise additional substitutions such as L14 and/or L15 substitutions (e.g., “LALA” substitutions L234A and L235A) and/or an N77 substitution (N297 e.g., N297A or N297G) or substitutions that enhance one or more effector functions.

A scaffold polypeptide suitable for use in constructs described herein may comprise an IgG1 Fc sequence with T130V, L131Y, F185A, and Y187V ZW1 substitutions, and its counterpart interspecific binding partner polypeptide comprises an IgG1 sequence having T130V, T146L, K172L, and T174W ZW1 substitutions, where the scaffold polypeptide comprises a sequence having at least 90% or at least 95% sequence identity to at least 210 (e.g., at least 220, or all 227) contiguous aas of the wt. IgG1 of SEQ ID NO:59. The scaffold polypeptide may comprise a ZW1 substitution and have at least 96% or at least 97% sequence identity to at least 210 (e.g., at least 220, or all 227) contiguous aas of the wt. IgG1 of SEQ ID NO:59. Such scaffold polypeptide sequence(s) may comprise additional substitutions such as L14 and/or L15 substitutions (e.g., “LALA” substitutions L234A and L235A) and/or an N77 substitution (N297 e.g., N297A or N297G) or substitutions that enhance one or more effector functions.

A scaffold polypeptide suitable for use in constructs described herein may comprise an IgG1 Fc sequence with K140D, D179M, and Y187A 7.8.60 substitutions, and its counterpart interspecific binding partner polypeptide comprises an IgG1 sequence having T130V, E125R, Q127R, T146V, and K189V 7.8.60 substitutions, where the scaffold polypeptide comprises a sequence having at least 90% or at least 95% sequence identity to at least 210 (e.g., at least 220, or all 227) contiguous aas of the wt. IgG1 of SEQ ID NO:59. The scaffold polypeptide may comprise a 7.8.60 substitution and have at least 96% or at least 97% sequence identity to at least 210 (e.g., at least 220, or all 227) contiguous aas of the wt. IgG1 of SEQ ID NO:59. Such scaffold polypeptide sequence(s) may comprise additional substitutions such as L14 and/or L15 substitutions (e.g., “LALA” substitutions L234A and L235A) and/or an N77 substitution (N297 e.g., N297A or N297G) or substitutions that enhance one or more effector functions.

A scaffold polypeptide suitable for use in constructs described herein may comprise an IgG1 Fc sequence with K189D and K172D DD-KK substitutions, and its counterpart interspecific binding partner polypeptide comprises an IgG1 sequence having T130V, D179K and E136K DD-KK substitutions, where the scaffold polypeptide comprises a sequence having at least 90% or at least 95% sequence identity to at least 210 (e.g., at least 220, or all 227) contiguous aas of the wt. IgG1 of SEQ ID NO:59. The scaffold polypeptide may comprise a DD-KK substitution and have at least 96% or at least 97% sequence identity to at least 210 (e.g., at least 220, or all 227) contiguous aas of the wt. IgG1 of SEQ ID NO:59. Such scaffold polypeptide sequence(s) may comprise additional substitutions such as L14 and/or L15 substitutions (e.g., “LALA” substitutions L234A and L235A) and/or an N77 substitution (N297 e.g., N297A or N297G) or substitutions that enhance one or more effector functions.

A scaffold polypeptide suitable for use in constructs described herein may comprise an IgG1 Fc sequence with K140E and K189W EW-RVT substitutions, and its counterpart interspecific binding partner polypeptide comprises an IgG1 sequence having T130V, Q127R, D179V, and F185T EW-RVT substitutions, where the scaffold polypeptide comprises a sequence having at least 90% or at least 95% sequence identity to at least 210 (e.g., at least 220, or all 227) contiguous aas of the wt. IgG1 of SEQ ID NO:59. The scaffold polypeptide may comprise an EW-RTV substitution and have at least 96% or at least 97% sequence identity to at least 210 (e.g., at least 220, or all 227) contiguous aas of the wt. IgG1 of SEQ ID NO:59. Such scaffold polypeptide sequence(s) may comprise additional substitutions such as L14 and/or L15 substitutions (e.g., “LALA” substitutions L234A and L235A) and/or an N77 substitution (N297 e.g., N297A or N297G) or substitutions that enhance one or more effector functions.

A scaffold polypeptide suitable for use in constructs described herein may comprise an IgG1 Fc sequence with K140E, K189W, and Y129C EW-RVTs-s substitutions, and its counterpart interspecific binding partner polypeptide comprises an IgG1 sequence having T130V, Q127R, D179V, F185T, and S134C EW-RVTs-s substitutions, where the scaffold polypeptide comprises a sequence having at least 90% or at least 95% sequence identity to at least 210 (e.g., at least 220, or all 227) contiguous aas of the wt. IgG1 of SEQ ID NO:59. The scaffold polypeptide may comprise an EW-RTVs-s substitution and have at least 96% or at least 97% sequence identity to at least 210 (e.g., at least 220, or all 227) contiguous aas of the wt. IgG1 of SEQ ID NO:59. Such scaffold polypeptide sequence(s) may comprise additional substitutions such as L14 and/or L15 substitutions (e.g., “LALA” substitutions L234A and L235A) and/or an N77 substitution (N297 e.g., N297A or N297G) or substitutions that enhance one or more effector functions.

A scaffold polypeptide suitable for use in constructs described herein may comprise an IgG1 Fc sequence with K150E and K189W A107 substitutions, and its counterpart interspecific binding partner polypeptide comprises an IgG1 sequence having T130V, E137N, D179V, and F185T A107 substitutions, where the scaffold polypeptide comprises a sequence having at least 90% or at least 95% sequence identity to at least 210 (e.g., at least 220, or all 227) contiguous aas of the wt. IgG1 of SEQ ID NO:59. The scaffold polypeptide may comprise an A10-7 substitution and have at least 96% or at least 97% sequence identity to at least 210 (e.g., at least 220, or all 227) contiguous aas of the wt. IgG1 of SEQ ID NO:59. Such scaffold polypeptide sequence(s) may comprise additional substitutions such as L14 and/or L15 substitutions (e.g., “LALA” substitutions L234A and L235A) and/or an N77 substitution (N297 e.g., N297A or N297G) or substitutions that enhance one or more effector functions.

As an alternative to the use of Ig CH2 and CH3 heavy chain constant regions as interspecific scaffold sequences, immunoglobulin Ig κ or Ig λ light chain constant regions can be utilized with Ig CH1 sequences as an interspecific sequence pair. A scaffold sequence may comprise an Ig κ chain constant region of SEQ ID NO:67 or an Ig λ chain constant region of SEQ ID NO:68

```
(SEQ ID NO: 67)
  1 TVAAPSVFIF PPSDEQLKSG TASVVCLLNN
      FYPREAKVQW KVDNALQSGN SQESVTEQDS
61 KDSTYSLSST LTLSKADYEK HKVYACEVTH
      QGLSSPVTKS ENRGEC;
Ig κ chain constant region
or
(SEQ ID NO: 68)
  1 GQPKANPTVT LEPPSSEELQ ANKATLVCLI
      SDFYPGAVTV AWKADGSPVK AGVETTKPSK
61 QSNNKYAASS YLSLTPEQWK SHRSYSCQVT
      HEGSTVEKTV APTECS.
Ig λ chain constant region
```

A light chain constant region scaffold sequence may also comprise a sequence having at least 90% or at least 95% sequence identity to SEQ ID NOs: 67 or 68. The CH1 sequence paired with a light chain constant region may comprise the aa sequence:

```
(SEQ ID NO: 69)
  1 FTVRETASTK GPSVFPLAPS SKSTSGGTAA
      LGCLVKDYFP EPVTVSWNSG ALTSGVHTEP
61 AVLQSSGLYS LSSVVTVPSS SLGTQTYICN
      VNHKPSNTKV DKKVEPKSCD KT.
```

A CH1 sequence used as a scaffold may also comprise a sequence having at least 90% or at least 95% sequence identity to SEQ ID NO:69.

Ig CH1 and Ig κ sequences may be modified to increase their affinity for each other, and accordingly the stability of any heterodimer formed utilizing them. Substitutions that increase the affinity and stability of CH1-Ig κ heterodimers include those identified as the MD13 combination in Chen et al., MAbs, 8 (4): 761-774 (2016). In MD13 variants each of the CH1 and Ig κ aa sequences comprises two substitutions. The Ig CH1 sequence is modified to contain S64E and S66V substitutions (S70E and S72V of SEQ ID NO:69). The Ig κ sequence is modified to contain S69L and T71S substitutions (S68L and T70S of SEQ ID NO:69).

### b) Modification of Effector Function in Ig Fc Scaffolds

Interactions of the IgG class of antibodies that lead to effector functions, including ADCC and ADCP, occur through Fc region engaging members of the Fcγ family of receptors (FcγRs). The human protein family is comprised of FcγRI (CD64), FcγRII (CD32, which includes isoforms FcγRIIa, FcγRIIb, and FcγRIIc), and FcγRIII (CD16, which includes isoforms FcγRIIIa and FcγRIIIb). See, e.g., Lazar et al. (2006) PNAS: 103 (11), 4005-4010. Interactions that lead to CDC may arise through IgFc interactions with complement C1q protein.

Substitutions that can alter effector functions of Ig sequences may be incorporated into Ig Fc sequences used as scaffolds in the constructs described herein. Both non-interspecific homodimerizing sequences such as wt. IgGs (e.g., SEQ ID NOs: 59 and 64-66) and interspecific Ig sequences my include substitutions that alter binding to either or both of an FcγR or C1q, and alter ADCC, ADCP, and/or CDC effector function.

### (1) Diminishing or Complete Suppression of IgFc Effector Function

Ig heavy chain constant region aa sequences used as scaffolds may also comprise one or more substitutions that can substantially diminish the ability to stimulate one or more Ig-mediated effector functions (e.g., ADCP, CDC, and/or ADCC) relative to the wt. sequences. For example, the scaffold may comprise an IgG1 sequence bearing substitutions at one or more of L234, L235, G236, G237, P238, S239 and/or P331 (appearing as L14, L15, G16, G17, P18, S19 and/or P111 in SEQ ID NO:59 or the corresponding positions in any of SEQ ID NOs: 60-66).

A scaffold polypeptide may comprise a Homo sapiens IgG1 Fc aa sequence of SEQ ID NO:70, which comprises a LALA substitution (L234A, L235A substitutions). IgG1 heavy chain constant regions with LALA substitutions do not effectively bind either FcγR or C1q, and have substantially diminished or completely suppressed ADCC, ADCP, and CDC stimulus functions. The positions of substitutions in wt. IgG1 sequences may be converted to positions provided in SEQ ID NOs: 59 or 70 by deducting 220 aas from the indicated position. Accordingly, the L234A, L235A substitutions appear as bolded and underlined Ala residues at positions 14 and 15, respectively, of S ID NO: 70: 1 DKTHTCPPCP APEAAGGPSV FLFPPKPKDT LMISRTPEVT CVVVDVSHED PEVKENWYVD 61 GVEVHNAKTK PREEQYNSTY RVVSVLTVLH QDWLNGKEYK CKVSNKALPA PIEKTISKAK 121 GQPREPQVYT LPPSRDELTK NQVSLTCLVK GFYPSDIAVE WESNGQPENN YKTTPPVLDS 181 DGSFFLYSKL TVDKSRWQQG NVESCSVMHE ALHNHYTOKS LSLSPG. The sequence set forth in SEQ ID NO:70 may be optionally modified to prevent dimerization by the addition of L351S, T366R, L368H and P395K substitutions (corresponding to L131S, T146R, L348H, and P175K in SEQ ID NO:59). A scaffold may comprise an aa sequence having greater than about 90% or greater than about 95% sequence identity to SEQ ID NO: 70. The scaffold may also comprise an aa sequence having greater than 96% or greater than 98% sequence identity to SEQ ID NO:70. Any one or more cysteines in SEQ ID NO:70 may be substituted (e.g., with an alanine or serine) so that the Ig heavy chain sequence cannot homodimerize and form interchain disulfide bonds.

Substitutions at positions D270, K322, P329 and/or P331 (corresponding to D50, K102, P109 and P111 in SEQ ID NO:59) lead to reduced binding to C1q relative to the wt. IgG1 protein, and hence a reduction in CDC. Any one or more of those substitutions may be included in an IgG1 sequence of SEQ ID NOs: 16 or 17. In an embodiment, an IgG1 heavy chain constant region aa sequence used as a scaffold comprises a LALA substitution (L234A, L235A) and a P331S substitution:

```
(SEQ ID NO: 71)
    1 DKTHTCPPCP APEAAGGPSV FLFPPKPKDT
        LMISRTPEVT CVVVDVSHED PEVKENWYVD
  61 GVEVHNAKTK PREEQYNSTY RVVSVLTVLH
        QDWLNGKEYK CKVSNKALPA SIEKTISKAK
121 GQPREPQVYT LPPSREEMTK NQVSLTCLVK
        GFYPSDIAVE WESNGQPENN YKTTPPVLDS
181 DGSFFLYSKL TVDKSRWQQG NVESCSVMHE
        ALHNHYTQKS LSLSPGK
```

or a sequence having at least 90% sequence identity to that sequence. Alternatively, the scaffold comprises an aa sequence having at least 95% or at least 98% sequence identity to SEQ ID NO:71.

Other substitutions that can substantially diminish one or more antibody related effector functions of IgG1 antibodies (e.g., ADCC, ADCP, and/or CDC responses) include, but are not limited to: Leu235Glu; Ser228Pro/Leu235Glu; Leu234Ala/Leu235Ala/Pro329Gly; Pro331Ser/Leu234Glu/Leu235Phe; Asp265Ala; Gly237Ala; Glu318Ala; Glu233Pro; and Gly236Arg/Leu328Arg. As indicated above, the corresponding location of those substitutions in the IgG1 Fc sequence provided as SEQ ID NO:59 can be obtained by subtracting 220 from the indicated positions. In an embodiment, at least ADCC is substantially diminished by the substitutions. In an embodiment at least ADCP is substantially diminished by the substitutions. In an embodiment, at least CDC is substantially diminished by the substitutions. In an embodiment, at least complement fixation is substantially diminished by the substitutions. See, e.g., Saunders 2019 and citations therein.

Substitutions in IgG2 and IgG4 antibody sequences that can substantially diminish one or more antibody related effector functions include, but are not limited to: H268N/V309L/A330S/P331S (IgG2m4); V234A/G237A/P238Sr/H268A/V309L/A330S/P331S (IgG2g); L234A/L235/G237A/P238S/H268A/A330S/P331S (IgG10); and S228P/F234A/L235A (IgG4PAA). See, e.g., Saunders 2019 and citations therein.

### (2) Enhancement of Ig Fc Effector Function

Among the substitutions that can enhance one or more antibody related effector functions (e.g., ADCC, ADCP, and/or CDC responses) of scaffold sequences comprising an IgG1 aa sequence relative to the corresponding wt. sequence are the individual substitutions S239D and 1332E, the double substitutions S239D/1332E, and the triple substitutions S239D/1332E/A330L in human IgG1. See Lazar et al., 2006. The substitutions corresponding to S239D, 1332E, and A330L in the human IgG1 Fc sequence (SEQ ID NO:59) are S19D, 1112E, and A110L. In an embodiment, the substitutions are the double and triple mutants S239D/1332E or S239D/1332E/A330L. In an embodiment, the IgG1 Fc substitutions may be the triple substitution S239D/1332E/A330L, which permits enhancement of ADCC without substantial alteration of CDC function (see Lazar et al., 2006).

Other substitutions that enhance one or more antibody related effector functions in IgG1 antibodies include, but are not limited to: S298A/E333A/K334A; S239D/A330L/1332E; S239D/1332E; G236A/S239d/A330L/1332E; G236A; S239D/1332E/G236A; L234Y/G236W/S298A; F243L/R292P/Y300L/V305l/P396L; K326W/E333S; K326A/E333S; K326M/E333S; C221D/D222C; S267E/H268F/S324T; H268F/S324T; and E345R. See Saunders (2019) Front. Immunol. 10:1296.doi: 10.3389/fimmu.2019.01296. The corresponding location in the IgG1 Fc sequence of SEQ ID NO:59 can be obtained by subtracting 220 from the indicated positions. In an embodiment at least ADCC is enhanced by the substitutions. In an embodiment at least ADCP is enhanced by the substitutions. In an embodiment at least CDC is enhanced by the substitutions. In an embodiment at least complement fixation is enhanced by the substitutions.

### 4. Immune Cell Binding Domains and Activating Domains

The protein constructs of the present disclosure comprise aa sequences that bind to and may interact with immune cells (e.g., T cells and NK cells). Those aa sequences may generally be described as immune cell binding domains (also referred to as an “immune cell binder”, ICB or ICBs when plural), and as a subset thereof that binds immune cells and elicits a response (e.g., induce granule dependent or granule independent cytotoxic responses) termed “immune cell activating domains” (AD or ADs when plural).

When ATP dependent complexation (e.g., dimerization) of constructs brings together two or more ICBs and/or ADs directed to the same immune cell or the same immune cell surface (target) molecule, the effective affinity of the ATP mediated complex is increased relative to a monomeric ICB/AD due to the bivalent or multivalent interaction of the complex with the cells.

ICBs that bind and do not act as agonists of immune cell function are typically antibodies or antibody-related molecules/aa sequences (e.g., an antigen binding fragment of an antibody, Fab, Fab′, single chain antibody, scFv, peptide aptamer, or nanobody). ICBs may function as cell lineage specific binders when they display affinity for a molecule selectively expressed on cells of one or more specific lineages. Unless stated otherwise, ICBs are generally understood to be monovalent to prevent individual molecules from crosslinking their cellular target molecule (e.g., an immune cell surface antigen) and off target stimulation of those target immune cells. Being monovalent, individual ICBs are generally incapable of acting as an agonist and stimulating the immune cells to which they bind, but they may act as an antagonist of their target molecule's natural ligand (e.g., monovalent antibody aa sequences based on Ipilimumab can bind CTLA-4 and antagonize its interactions with CD28). When two or more (e.g., multiple) constructs bearing ICB aa sequences directed to the same target molecule are complexed through ATP induced dimerization or multimerization (see, e.g., FIG. 4A at D or E), or are bound on the surface of a tumor cell (e.g., using a construct such as in FIG. 3 at C), the ICB aa sequence(s) may function as ADs. Accordingly, while ADs have the ability to stimulate various immune cell responses upon binding to immune cells (e.g., in a bivalent or polyvalent state), ICBs lacking that ability function as immune cell targeting sequences that bind to cells expressing their cognate binding partner (cellular target molecule).

ADs of the constructs of the present disclosure include a variety of molecules or fragments of molecules (e.g., fragments of cytokines) that are capable of binding to and stimulating immune cell response (e.g., anti-tumor cell immune responses), particularly when two or more ADs are presented (e.g., as dimers or higher order complexes). ADs include immunomodulatory protein aa sequences (e.g., aa sequences of various interleukins) and other molecules (e.g., antibodies such as Fab, Fab′, single chain antibodies, scFvs, peptide aptamers, or nanobodies) that can bind and functionally engage receptors on the surface of immune cells to produce a response (e.g., induce granule dependent or granule independent cytotoxic responses). ADs are typically ineffective (have limited potency and/or efficacy) in the induction of immune cell responses when presented in monomeric form in, for example, the TME. When two or more (e.g., multiple) constructs bearing ADs are localized in a complex through ATP induced dimerization or multimerization (e.g., bound on the surface of an immune cell as a dimer using a construct such as in FIG. 2 at A-E or FIG. 4 at A or B) or are bound on the surface of a tumor cell (e.g., using a construct such as in FIG. 3 at A, B, or D or FIG. 4A at D or B), the ADs become more effective (e.g., have a higher potency and/or efficacy) in the induction of immune cell responses than constructs with the ADs in monomeric form. The presence of two or more ADs in an ATP induced complex (e.g., dimer, heterodimer, or multimer) facilitates crosslinking of the AD's cellular receptors and stimulation of a receptor mediated response. Some ADs that require crosslinking of their cellular receptors for maximal cellular activation include, but are not limited to, CD40L, IFN-γ, IL-12, and CD28 ligands such as CD80 or CD86.

Immune cell responses to the ATP-dependent dimerization or multimerization of the constructs of the present disclosure may include, but are not limited to, granule dependent and/or granule independent responses, for example from stimulation of NK cells and/or T cells (e.g., CD8+ T cells). Some responses, including cytotoxic responses from T cells and NK cells, may be due to the direct binding of constructs of the present disclosure to those immune cells. Other responses may be the indirect result of activation of other immune cells. By way of example, recruitment and/or activation of macrophages by constructs of the present disclosure may result from activation of NK cells that release chemoattractant IFNγ or CCL5 or by the direct binding of a construct of the present disclosure (e.g., bearing a chemokine as an AD) to the macrophage.

Table 4 sets forth examples of immune cells and ICBs, a number of which may also function as ADs (e.g., CD3). It should be noted that portions of the Ig scaffolds that are capable of stimulating ADCC, ADCP, and/or CDC are considered separately. Sequences stimulating ADCC, ADCP, and/or CDC may be expressly excluded from ICBs and/or ADs, and still may be present, optionally present, or absent from immunoglobulin scaffold aa sequences.

### a) Immune Cell Binding Domains (ICBs)

ICBs may be directed to a number of different immune cells that appear or may be recruited into the TME. Such immune cells and the targets of their corresponding ICBs include those set forth in Table 4 below.

TABLE 4
Some Immune cells and their corresponding ICB targets and ICBs

| | |
|---|---|
| | ICB target protein/ICB (e.g., antibody or antibody- |
| Immune cell | related molecules‡ directed against target) |

Lymphoid cell types

| | |
|---|---|
| CD4+ T cell | CD3/anti-CD3γ or anti-CD3ε; αβTCR/anti-TCRα |
| | chain or anti-TCRβ chain; CD4/anti-CD4 |
| CD4+ T regs | CD25/anti-CD25 |
| CD8+ T cell | CD3/anti-CD3γ or anti-CD3ε; αβTCR/anti-TCRα |
| | chain or anti-TCRβ chain CD8/anti-CD8 |
| δγ T cell | δγ TCR/anti-TCRδ chain or anti-TCRγ |
| | chain (e.g., TRGV9/anti-TRGV9 |
| NK Cell | CD16/anti-CD16 |

Myeloid cell type

| | |
|---|---|
| Neutrophils | CD16/anti-cD16; CD66b/anti-CD66b; CD15/anti-CD15 |
| Mast Cells | FcεR1α/anti-FcεR1α; CD117/anti-CD117 |
| Eosinophils | CD193/anti- CD193 |
| Basophils | FcεR1α/anti- FcεR1α |
| Macrophages | CD68/anti-CD68 |
| | M1-like: CD68/anti-CD68 |
| | M2-like: CD68/anti-CD68; CD163/anti-CD163; CD206/anti-CD206 |
| MDSC (myeloid derived | CD84/anti-CD84; CXCR1/anti-CXCR1; DR5/anti-DR5; CD13/anti-CD13; |
| suppressor cells) | CD33/anti-CD33; CD34/anti-CD34; CD16/anti-CD16 |

‡Antibody related molecules include, but are not limited to, antigen binding fragments of an antibody, Fabs, Fab's, and polypeptides comprising single chain antibody, scFv, peptide aptamer, or nanobody aa sequences.

ICBs such as those listed in Table 4 may be incorporated into a first NBD-containing construct that can undergo ATP mediated formation of homodimers, heterodimers, and/or higher order complexes. NBD-containing constructs that comprise an ICB can, for example, undergo ATP mediated complexation with a second NBD-containing construct comprising a TSB (TSB) directed against a TAA. The complex, which may be described as “BiTE-like,” produces trans-targeting of tumor cells leading to their cytolysis (see, e.g., FIG. 3 at C, D, and F-I, and also complexes in FIG. 4A at E, F, H, and I). The incorporation of antibody-related molecules/aa sequences including, but not limited to, anti-CD3, anti-TCR, and/or anti-CD16 as ICBs/ADs in such trans-targeting complexes may produce potent cytolytic action. Cytolytic activity may be enhanced when ICBs that interact with more than one type of immune cell are employed. Some exemplary ICBs are provide below.

An ICB polypeptide may comprise an scFv, anti-human CD3 based upon the UCHT-1 monoclonal antibody having the sequence: AIQMTQSPSS LSASVGDRVT ITCRASQDIR NYLNWYQQKP GKAPKLLIYY TSRLESGVPS RESGSGSGTD YTLTISSLQP EDFATYYCQQ GNTLPWTFGQ GTKVEIKGGG GSGGGGSGGG GSGGGGSGGG SEVOLVESGG GLVQPGGSLR LSCAASGYSF TGYTMNWVRQ APGKGLEWVA LINPYKGVTT YADSVKGRFT ISVDKSKNTA YLQMNSLRAE DTAVYYCARS GYYGDSDWYF DVWGQGTLVT VSS (SEQ ID NO:72). The ICB may also comprise an aa sequence having greater than about 90% or greater than about 95% sequence identity to SEQ ID NO:72. In addition the ICB may comprise an aa sequence having greater than 96% or greater than 98% sequence identity to SEQ ID NO: 72.

An ICB polypeptide may comprise an scFv, anti-human CD3 based upon the UCHT-1 monoclonal antibody having the sequence: AIQMTQSPSSLSASVGDRVTITCRASQDIRNYLNWYQQKPGKAPKLLIYYT SRLESGVPSRFSGSGSGTDYTLTISSLQPEDFATYYCQQGNTLPWTFGQGTKVEIKGGGGGGGGSGGGG SGGGGSGGGSEVOLVESGGGLVQPGGSLRLSCAASGYSFTGYTMNWVRQAPGKGLEWVALINPYKGVSTY NQKFKDRFTISVDKSKNTAYLQMNSLRAEDTAVYYCARSGYYGDSDWYFDVWGQGTLVTVSS (SEQ ID NO: 73). The ICB may also comprise an aa sequence having greater than about 90% or greater than about 95% sequence identity to SEQ ID NO:73. In addition, the ICB may comprise an aa sequence having greater than 96% or greater than 98% sequence identity to SEQ ID NO:73.

An ICB polypeptide may comprise an anti-human CD3 VHH having the sequence: QVQLVESGGG LVQPGGSLRL SCAASGSIFS ANTMGWYRQA PGKORELVAG MNTSGSTVYG DSVKGRFTIS RDNAKNIAYL QMNSLIPEDT AVYYCTLVQR GPNYWGOGTO VTVSS (SEQ ID NO:74). The ICB may also comprise an aa sequence having greater than about 90% or greater than about 95% sequence identity to SEQ ID NO: 74. In addition the ICB may comprise an aa sequence having greater than 96% or greater than 98% sequence identity to SEQ ID NO:74.

An ICB polypeptide may comprise an anti-human CD3 scFv (vH-vL) based upon the L2K antibody (see, e.g., Dreier et al., Int. J. Cancer. 2002; 100:690-697. doi: 10.1002/ijc.10557) having the sequence: DVQLVQSGAE VKKPGASVKV SCKASGYTFT RYTMHWVRQA PGOGLEWIGY INPSRGYTNY ADSVKGRETI TTDKSTSTAY MELSSLRSED TATYYCARYY DDHYCLDYWG QGTTVTVSSG GGGSGGGGSG GGGSDIVLTQ SPATLSLSPG ERATLSCRAS QSVSYMNWYQ QKPGKAPKRW IYDTSKVASG VPARESGSGS GTDYSLTINS LEAEDAATYY CQQWSSNPLT FGGGTKVEIK (SEQ ID NO:75). The ICB may also comprise an aa sequence having greater than about 90% or greater than about 95% sequence identity to SEQ ID NO:75. In addition the ICB may comprise an aa sequence having greater than 96% or greater than 98% sequence identity to SEQ ID NO:75.

An ICB polypeptide may comprise an anti-human CD3 scFv (vH-vL) based upon the L2K antibody having the sequence set forth in SEQ ID NO:76, which includes a disulfide bond between the cysteine substitutions at Q43C and G99C (G233C as shown): DVQLVQSGAE VKKPGASVKV SCKASGYTFT RYTMHWVRQA PGCGLEWIGY INPSRGYTNY ADSVKGRFTI TTDKSTSTAY MELSSLRSED TATYYCARYY DDHYCLDYWG QGTTVTVSSG GGGSGGGGSG GGGSDIVLTQ SPATLSLSPG ERATLSCRAS QSVSYMNWYQ QKPGKAPKRW IYDTSKVASG VPARESGSGS GTDYSLTINS LEAEDAATYY CQQWSSNPLT FGCGTKVEIK (SEQ ID NO:76). The ICB may also comprise an aa sequence having greater than about 90% or greater than about 95% sequence identity to SEQ ID NO:76. In addition the ICB may comprise an aa sequence having greater than 96% or greater than 98% sequence identity to SEQ ID NO:76.

An ICB polypeptide may comprise an anti-human CD3 scFv (vH-vL) based upon the L2K antibody having the sequence set forth in SEQ ID NO:77, which includes a disulfide bond between the cysteine substitutions at Q43C and G100C (G234C as shown): DVQLVQSGAE VKKPGASVKV SCKASGYTFT RYTMHWVRQA PGCGLEWIGY INPSRGYTNY ADSVKGRFTI TTDKSTSTAY MELSSLRSED TATYYCARYY DDHYCLDYWG QGTTVTVSSG GGGSGGGGSG GGGSDIVLTQ SPATLSLSPG ERATLSCRAS QSVSYMNWYQ QKPGKAPKRW IYDTSKVASG VPARFSGSGS GTDYSLTINS LEAEDAATYY CQQWSSNPLT FGGCTKVEIK (SEQ ID NO:77). The ICB may also comprise an aa sequence having greater than about 90% or greater than about 95% sequence identity to SEQ ID NO:77. In addition the ICB may comprise an aa sequence having greater than 96% or greater than 98% sequence identity to SEQ ID NO:77.

An ICB polypeptide may comprise an anti-human CD3 scFv (vH-vL) based upon the L2K antibody having the sequence set forth in SEQ ID NO:78, which includes a disulfide bond between the cysteine substitutions at G44C and G99C (G233C as shown): DVQLVQSGAE VKKPGASVKV SCKASGYTFT RYTMHWVRQA PGQCLEWIGY INPSRGYTNY ADSVKGRFTI TTDKSTSTAY MELSSLRSED TATYYCARYY DDHYCLDYWG QGTTVTVSSG GGGSGGGGSG GGGSDIVLTQ SPATLSLSPG ERATLSCRAS QSVSYMNWYQ QKPGKAPKRW IYDTSKVASG VPARFSGSGS GTDYSLTINS LEAEDAATYY CQQWSSNPLT FGCGTKVEIK (SEQ ID NO:78). The ICB may also comprise an aa sequence having greater than about 90% or greater than about 95% sequence identity to SEQ ID NO:78. In addition the ICB may comprise an aa sequence having greater than 96% or greater than 98% sequence identity to SEQ ID NO:78.

An ICB polypeptide may comprise an anti-human CD3 scFv (vH-vL) based upon the L2K antibody having the sequence set forth in SEQ ID NO:79, which includes a disulfide bond between the cysteine substitutions at G44C and G100C (G234C as shown): 1 DVQLVQSGAE VKKPGASVKV SCKASGYTFT RYTMHWVRQA PGQCLEWIGY INPSRGYTNY 61 ADSVKGRETI TTDKSTSTAY MELSSLRSED TATYYCARYY DDHYCLDYWG QGTTVTVSSG 121 GGGSGGGGSG GGGSDIVLTQ SPATLSLSPG ERATLSCRAS QSVSYMNWYQ QKPGKAPKRW 181 IYDTSKVASG VPARFSGSGS GTDYSLTINS LEAEDAATYY COQWSSNPLT FGGCTKVEIK (SEQ ID NO:79). The ICB may also comprise an aa sequence having greater than about 90% or greater than about 95% sequence identity to SEQ ID NO:79. In addition, the ICB may comprise an aa sequence having greater than 96% or greater than 98% sequence identity to SEQ ID NO:79.

The GGGS (SEQ ID NO:30) and/or GGGGS (SEQ ID NO:40) containing repeats in any of SEQ ID NOs: 72-73 or 75-79 are linker sequences between domains.

An ICB may comprise, for example, an aa sequence that binds to CD16 (e.g., an anti-CD16 scFv or nanobody aa sequence). Anti-CD16 VHH polypeptides that may be employed as an ICB may comprise one of: 1 EVOLVESGGG LVOPGESLTL SCVVAGSIFS FAMSWYRQAP GKERELVARI GSDDRVTYAD 61 SVKGRFTISR DNIKRTAGLQ MNSLKPEDTA VYYCNAQTDL RDWTVREYWG QGTQVTVSS (SEQ IDO:80); or 1 EVOLVESGGE LVQPGGSLRL SCAASGLTES SYNMGWERRA PGKEREFVAS ITWSGRDTFY 61 ADSVKGRFTI SRDNAKNTVY LQMSSLKPED TAVYYCAANP WPVAAPRSGT YWGQGTQVTV 121 SS (SEQ ID NO:81). Alternatively, an ICB may comprise a sequence having at least 90% or at least 95% sequence identity with SEQ ID NOs: 80 or 81. An ICB may also comprise a sequence having at least 96% or at least 98% sequence identity to at least 100 or at least 110 contiguous aas of SEQ ID NOs: 80 or 81.

### b) Immune Cell Activating Domains (ADs)

AD sequences may be derived from a variety of sources including interleukins and antibodies that bind to interleukin receptors or other cell proteins. ADs include, but are not limited to, antibodies (e.g., scFVs or nanobodies) and antigen binding fragments of antibodies such as: anti-TCRα or anti-TCRβ chain, anti-CXCR1, anti-CD3γ, anti-CD3ε, anti-TCRδ chain or anti-TCRγ chain (e.g., TRGV9/anti-TRGV9, anti-CD4, anti-CD13, anti-CD15, anti-CD16 (FcγRIII), anti-CD257 (anti-BAFF), anti-CD28, anti-CD152 (anti-CTLA-4), anti-CD33, anti-CD40, anti-CD40L, anti-CD34, anti-CD66b, anti-CD68, anti-CD84, anti-CD117, anti-CD137 (anti-41BB), anti-CD163, anti-CD193, anti-CD206, and anti-FcεR1a. ADs include, but are not limited to, CD3, CD80, CD86, CD137L, 4-1BBL, IL-2, IL-7, IL-12, IL-15, IL23, IL27, IL-35, PD1, PDL1, TNF, lymphotoxin α, lymphotoxin αβ, interferon-γ (IFN-γ), and TNF polypeptides that functionally engage receptors on immune cells effecting their activity.

ADs may comprise, for example, aa sequences of TNF superfamily members such as TNF, lymphotoxin α, lymphotoxin αβ, and BAFF (CD257). ADs may also comprise, for example, aa sequences of B7 superfamily members, such as CD80 and CD86. An AD may also comprise, for example, the aa sequence of all or part of IL-15, IL-12 (e.g., an scIL12), IL-2, or IL-7 (e.g., all or part of the extracellular domain(s). An AD may comprise all or part of the extracellular portions of the p35 and p40 aa sequences of IL-12 (e.g., one or both subunits of human IL-12). An AD may comprise aa sequences having greater than 95% or greater than 98% sequence identity to the p35 and p40 extracellular domains of IL-12.

By way of example, some ADs that may be employed in constructs that homodimerize (see, e.g., FIG. 2 at A and C) or form homomeric complexes (see, e.g., FIG. 4A at A) in the presence of ATP levels found in the TME include, but are not limited to, immunomodulatory aa sequences of: IL-15, IL-12, or IL-7. Antibody related sequences (e.g., single chain scFv or nanobody sequences) that bind to PD1 (e.g., an anti-PD1 scFv or nanobody aa sequence) or an aa sequence that binds to PDL1 (e.g., an anti-PDL1 scFv or nanobody aa sequence) may be employed as ADs. Similarly, an AD may comprise an aa sequence that binds to CD40 (e.g., an anti-CD40 scFv or nanobody aa sequence or a CD40L aa sequence) or an aa sequence that binds to CD40L (e.g., an anti-CD40L scFv or nanobody aa sequence, or a CD40 aa sequence). An AD may comprise an aa sequence that binds to CD137/4-1BB (e.g., an anti-CD137 scFv or nanobody aa sequence, or a CD137L/4-1BBL aa sequence). An AD may comprise an aa sequence that binds to IL-10 (e.g., an anti-IL-10 scFv or nanobody aa sequence) or an aa sequence that binds to IL-10R (e.g., an anti-IL-10R scFv or nanobody aa sequence).

An AD may comprise an aa sequence that binds to CD3 (e.g., an anti-CD3 scFv or nanobody aa sequence), which may also function as an ICB. An AD may comprise an aa sequence that binds to CTLA-4 (e.g., an anti-CTLA-4 scFv or nanobody aa sequence). An AD may comprise an aa sequence that binds to CD28 (e.g., an anti-CD28 scFv or nanobody aa sequence). An AD may comprise an interferon-γ (IFN-γ) aa sequence, or an aa sequence that binds to the IFN-γ receptor (e.g., an anti-IFN-γ receptor scFv or nanobody aa sequence). An AD may comprise an aa sequence that binds to CD16 (e.g., an anti-CD16 scFv or nanobody aa sequence).

By way of example an AD that may be employed in constructs that homodimerize (see, e.g., FIG. 2 at A and C) or form homomeric complexes (see, e.g., FIG. 4 at A) in the presence of ATP levels found in the TME include, but are not limited to, immunomodulatory aa sequences of: IL-15, IL-12, or IL-7. Antibody related sequences (e.g., single chain scFv or nanobody sequences) having anti-PD1, anti-PDL1, anti-CD40, anti-CD40L, anti-CD137/4-1BB, anti-IL-10, or anti-IL10R binding activity may also be employed.

In addition to the foregoing, nucleic acids, including nucleic acids with CpG repeats or one or more IMT504 sequences, may be used as ADs and may be incorporated into homodimerizing or heterodimerizing NBD-containing constructs. Nucleic acids, however, require coupling to other portions of the protein constructs prepared, for example, by cellular expression.

### (1) IL-2

An AD may comprise an IL-2 aa sequence capable of binding and stimulating signaling from its cognate IL-2 receptor (IL-2R). Wt. IL-2 binds to a heterotrimeric IL-2R comprising IL-2Rα (CD25), IL-2Rβ (CD122), and IL-2Rγ (CD132) subunits. The sequences of human IL-2 and all three of the human IL-2R subunits are known. See, e.g., published PCT application WO 2020/132138 A1.

IL-2 ADs may comprise one or more insertions, deletions, or substitutions resulting in decreased binding to IL-2Rα, which minimizes or substantially reduces the activation of Tregs. Alternatively, or in addition to alterations in IL-2 sequences that lead to modified IL-2Rα interactions, IL-2 MOD aa sequences may include insertions, deletions, or substitutions that result in decreased binding to IL-2Rβ and/or IL-2Rγ subunits that reduce affinity for the IL-2R. IL-2 sequences that bind to the IL-2R, and that have altered affinities for IL-2Rα, IL-2Rβ and/or IL-2Rγ, are disclosed in WO 2020/132138 A1, WO 2019/051091, and WO 2020/132297.

Suitable IL-2 ADs for incorporation into a protein construct described herein may comprise an aa sequence having at least 90% or at least 95% aa sequence identity to the wt. IL-2 aa sequence: APTSSSTKKT QLQLEHLLLD LQMILNGINN YKNPKLTRML TFKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO: 105). Alternatively, the IL-2 MOD may comprise an aa sequence having at least 96% or at least 98% aa sequence identity to the IL-2 aa sequence of SEQ ID NO: 105. A suitable IL-2 AD sequence may comprise an aa sequence having 100% identity to SEQ ID NO: 105, or an aa sequence with at least one aa insertion, deletion, or substitution in the aa sequence of SEQ ID NO: 105. IL-2 ADs may comprise substitutions with limited binding to IL-2Rα and/or IL-2Rβ and as a result decreased binding to the IL-2R. Such substitutions in IL-2 are described in, for example, WO 2020/132138 A1, WO 2019/051091, and WO 2020/132297. Examples of such substitutions include those at H16 and F42, which are highlighted and italicized in SEQ ID NO: 105. Substitutions at those positions include replacement of H16 and F42 with aas other than His and Phe, respectively. H16 and F42 may also be replaced by independently selected Ala or Thr residues to produce, for example the H16A F42A variant of an IL-2 sequence (e.g., SEQ ID NO:105). Other IL-2 variants include the substitutions: H16A F42T; H16T F42A, or H16T F42T.

### (2) CD80

An AD may comprise the CD80 polypeptide aa sequence: 1 VIHVTKEVKE VATLSCGHNV SVEELAQTRI YWOKEKKMVL TMMSGDMNIW PEYKNRTIED 61 ITNNLSIVIL ALRPSDEGTY ECVVLKYEKD AFKREHLAEV TLSVKADEPT PSISDFEIPT 121 SNIRRIICST SGGFPEPHLS WLENGEELNA INTTVSQDPE TELYAVSSKL DENMTTNHSE 181 MCLIKYGHLR VNQTENWNTT K (SEQ ID NO: 106). Alternatively, an AD may comprise a sequence having at least 90% or at least 95% sequence identity with SEQ ID NO:106. An AD may also comprise a sequence having at least 96% or at least 98% sequence identity to at least 170 or 180 contiguous aas of SEQ ID NO: 106.

### (3) CD86

An AD may comprise the CD86 polypeptide aa sequence comprising the CD86 (IgV domain): 1 LKIQAYFNET ADLPCQFANS QNQSLSELVV FWQDQENLVL NEVYLGKEKF DSVHSKYMNR 61 TSFDSDSWTL RLHNLQIKDK GLYQCIIHHK KPTGMIRIHQ MNSELSVLAN FSQPEIVPIS 121 NITENVYINL TCSSIHGYPE PKKMSVLLRT KNSTIEYDGI MQKSQDNVTE LYDVSISLSV 181 SFPDVTSNMT IFCILETDKT RLLSSPESIE LEDPQPPPDH IP (SEQ ID NO:107); or the CD86 IgV domain: 1 LKIQAYFNET ADLPCQFANS QNQSLSELVV FWQDQENLVL NEVYLGKEKF DSVHSKYMGR 61 TSFDSDSWTL RLHNLQIKDK GLYQCIIHHK KPTGMIRIHQ MNSELSVLA (SEQ ID NO: 108).

Alternatively, an AD may comprise a sequence having at least 90% or at least 95% sequence identity with SEQ ID NOs: 107 or 108. An AD may also comprise a sequence having at least 96% or at least 98% sequence identity to at least 100 or at least 110 contiguous aas of SEQ ID NOs: 107 or 108.

### (4) IL-12

An AD may comprise an aa sequence that binds the IL-12 receptor as an agonist (e.g., IL-12 or an anti-CD28 scFv or nanobody aa sequence). IL-12 and members of the IL-12 superfamily are heterodimeric. An AD may comprise a single chain IL-12 (scIL-12) that comprises the sequence: 1 IWELKKDVYV VELDWYPDAP GEMVVLTCDT PEEDGITWTL DQSSEVLGSG KTLTIQVKEF 61 GDAGQYTCHK GGEVLSHSLL LLHKKEDGIW STDILKDQKE PKNKTFLRCE AKNYSGRFTC 121 WWLTTISTDL TFSVKSSRGS SDPQGVTCGA ATLSAERVRG DNKEYEYSVE CQEDSACPAA 181 EESLPIEVMV DAVHKLKYEN YTSSFFIRDI IKPDPPKNLQ LKPLKNSRQV EVSWEYPDTW 241 STPHSYFSLT FCVQVQGKSK REKKDRVFTD KTSATVICRK NASISVRAQD RYYSSSWSEW 301 ASVPCSGGGG SGGGGSGGGG SRNLPVATPD PGMFPCLHHS QNLLRAVSNM LOKARQTLEF 361 YPCTSEEIDH EDITKDKTST VEACLPLELT KNESCLNSRE TSFITNGSCL ASRKTSEMMA 421 LCLSSIYEDL KMYQVEFKTM NAKLLMDPKR QIFLDQNMLA VIDELMQALN ENSETVPQKS 481 SLEEPDEYKT KIKLCILLHA FRIRAVTIDR VMSYLNAS (SEQ ID NO:109), which comprises a fragment of the p40 subunit, a linker repeat of GGGGS, and a fragment of the p35 subunit. Alternatively, an AD may comprise a sequence having at least 90% or at least 95% sequence identity with SEQ ID NO: 109. An AD may also comprise a sequence having at least 96% or at least 98% sequence identity to at least 480 or at least 500 contiguous aas of SEQ ID NO: 109.

As discussed above, the constructs of the present disclosure may comprise NBDs that heterodimerize in the presence of ATP. Accordingly, a complex between a first construct and a second construct that comprises heterodimerizing NBDs can be used to assemble an active IL-12 super family member as an AD (see, e.g., FIG. 2 at B and D where the first and second ADs are subunits of a member of that superfamily). For example, an active IL-12 AD heterodimer may be formed in the presence of ATP by employing:
(i) a first construct comprising a p40 subunit (as a 1^{st} immunomodulatory domain) comprising the sequence: 1 IWELKKDVYV VELDWYPDAP GEMVVLTCDT PEEDGITWTL DQSSEVLGSG KTLTIQVKEF 61 GDAGQYTCHK GGEVLSHSLL LLHKKEDGIW STDILKDQKE PKNKTFLRCE AKNYSGRETC 121 WWLTTISTDL TESVKSSRGS SDPQGVTCGA ATLSAERVRG DNKEYEYSVE CQEDSACPAA 181 EESLPIEVMV DAVHKLKYEN YTSSFFIRDI IKPDPPKNLQ LKPLKNSRQV EVSWEYPDTW 241 STPHSYFSLT FCVQVQGKSK REKKDRVFTD KTSATVICRK NASISVRAQD RYYSSSWSEW 301 ASVPCS (SEQ ID NO:110), or a sequence having at least 90% or at least 95% sequence identity with SEQ ID NO:110; and
(ii) a second construct comprising a p35 subunit (as a 2^{nd} immunomodulatory domain) comprising t sequence: 1 RNLPVATPDP GMFPCLHHSQ NLLRAVSNML QKARQTLEFY PCTSEEIDHE DITKDKTSTV 061 EACLPLELTK NESCLNSRET SFITNGSCLA SRKTSEMMAL CLSSIYEDLK MYQVEFKTMN 121 AKLLMDPKRQ IFLDQNMLAV IDELMQALNF NSETVPQKSS LEEPDFYKTK IKLCILLHAF 181 RIRAVTIDRV MSYLNAS (SEQ ID NO:111), or a sequence having at least 90% or at least 95% sequence identity with SEQ ID NO:111.

### (5) PD1

An AD may comprise all or part of an aa sequence that binds to PD1 (e.g., an anti-PD1 scFv or nanobody aa sequence) or an aa sequence that binds to PDL1 (e.g., an anti-PDL1 scFv or nanobody aa sequence).

### (6) CD40

An AD may comprise an aa sequence that binds to CD40 (e.g., an anti-CD40 scFv or nanobody aa sequence or a CD40L aa sequence) or an aa sequence that binds to CD40L (e.g., an anti-CD40L scFv or nanobody aa sequence, or a CD40 aa sequence). An AD may comprise the CD40L the sequence: 1 GDQNPQIAAH VISEASSKTT SVLQWAEKGY YTMSNNLVTL ENGKOLTVKR QGLYYIYAQV 61 TFCSNREASS QAPFIASLCL KSPGRFERIL LRAANTHSSA KPCGQQSIHL GGVFELQPGA 121 SVFVNVTDPS QVSHGTGFTS FGLLKL (SEQ ID NO:112), or an aa sequence having at least 90% or at least 95% sequence identity to SEQ ID NO:112. Alternatively, a polypeptide that may be employed as an AD comprises a trimer of CD40L aa sequences separated by linker sequences (e.g., linker sequences comprising GGGGS repeats SEQ ID NO:40): 1 GDQNPQIAAH VISEASSKTT SVLQWAEKGY YTMSNNLVTL ENGKOLTVKR QGLYYIYAQV 61 TFCSNREASS QAPFIASLCL KSPGRFERIL LRAANTHSSA KPCGQQSIHL GGVFELQPGA 121 SVFVNVTDPS QVSHGTGFTS FGLLKLGGGG SGGGGSGGGG SGDONPQIAA HVISEASSKT 181 TSVLQWAEKG YYTMSNNLVT LENGKOLTVK RQGLYYIYAQ VTFCSNREAS SQAPFIASLC 241 LKSPGRFERI LLRAANTHSS AKPCGQQSIH LGGVFELQPG ASVFVNVTDP SQVSHGTGET 301 SFGLLKLGGG GSGGGGSGGG GSGDQNPQIA AHVISEASSK TTSVLQWAEK GYYTMSNNLV 361 TLENGKQLTV KRQGLYYIYA QVTFCSNREA SSQAPFIASL CLKSPGRFER ILLRAANTHS 421 SAKPCGQQSI HLGGVFELQP GASVFVNVTD PSQVSHGTGF TSFGLLKL (SEQ ID NO: 113), or an aa sequence having at least 90% or at least 95% sequence identity to SEQ ID NO:113.

### (7) 4-1BBL

An AD may comprise an aa sequence that binds to CD137/4-1BB (e.g., an anti-CD137 scFv or nanobody aa sequence, or a CD137L/4-1BBL aa sequence). A 4-1BBL polypeptide that may be employed as an AD may comprise the 4-1BBL the sequence: 1 ACPWAVSGAR ASPGSAASPR LREGPELSPD DPAGLLDLRQ GMFAQLVAQN VLLIDGPLSW 61 YSDPGLAGVS LTGGLSYKED TKELVVAKAG VYYVFFQLEL RRVVAGEGSG SVSLALHLQP 121 LRSAAGAAAL ALTVDLPPAS SEARNSAFGF QGRLLHLSAG QRLGVHLHTE ARARHAWQLT 181 QGATVLGLER VTPEIPAGLP SPRSE (SEQ ID NO:114), or the 4-1BBL sequence: 1 PAGLLDLRQG MFAQLVAQNV LLIDGPLSWY SDPGLAGVSL TGGLSYKEDT KELVVAKAGV 61 YYVFFQLELR RVVAGEGSGS VSLALHLQPL RSAAGAAALA LTVDLPPASS EARNSAFGEQ 121 GRLLHLSAGQ RLGVHLHTEA RARHAWQLTQ GATVLGLFRV TPEIPA (SEQ ID NO:115). A 4-1BBL AD may comprise an aa sequence having at least 90% or at least 95% sequence identity to SEQ ID NO:114 or SEQ ID NO:115. Alternatively, a polypeptide that may be employed as an AD may comprise a trimer of 4-1BBL aa sequences separated by linker sequences (e.g., linker sequences comprising GGGGS (SEQ ID NO:40) repeats): 1 ACPWAVSGAR ASPGSAASPR LREGPELSPD DPAGLLDLRQ GMFAQLVAQN VLLIDGPLSW 61 YSDPGLAGVS LTGGLSYKED TKELVVAKAG VYYVFFQLEL RRVVAGEGSG SVSLALHLQP 121 LRSAAGAAAL ALTVDLPPAS SEARNSAFGF QGRLLHLSAG QRLGVHLHTE ARARHAWQLT 181 QGATVLGLER VTPEIPAGLP SPRSEGGGGS GGGGSGGGGS ACPWAVSGAR ASPGSAASPR 241 LREGPELSPD DPAGLLDLRQ GMFAQLVAQN VLLIDGPLSW YSDPGLAGVS LTGGLSYKED 301 TKELVVAKAG VYYVFFQLEL RRVVAGEGSG SVSLALHLQP LRSAAGAAAL ALTVDLPPAS 361 SEARNSAFGF QGRLLHLSAG QRLGVHLHTE ARARHAWQLT QGATVLGLFR VTPEIPAGLP 421 SPRSEGGGGS GGGGSGGGGS ACPWAVSGAR ASPGSAASPR LREGPELSPD DPAGLLDLRQ 481 GMFAQLVAQN VLLIDGPLSW YSDPGLAGVS LTGGLSYKED TKELVVAKAG VYYVFFQLEL 541 RRVVAGEGSG SVSLALHLOP LRSAAGAAAL ALTVDLPPAS SEARNSAFGF QGRLLHLSAG 601 QRLGVHLHTE ARARHAWOLT QGATVLGLER VTPEIPAGL PSPRSE (SEQ ID NO: 116). A 4-1BBL AD may comprise an aa sequence having at least 90% or at least 95% sequence identity to SEQ ID NO: 116.

### (8) CD3

An AD may comprise an aa sequence that binds to CD3 (e.g., an anti-CD3 scFv or nanobody aa sequence). As discussed above, such ADs may function as ICBs.

An AD may comprise an anti-human CD3 scFv aa sequence based upon the UCHT-1 monoclonal antibody, which comprises the aa sequence of SEQ ID NOs: 72 or 73. An AD may comprise an aa sequence having greater than about 90% or greater than about 95% sequence identity to SEQ ID NOs: 72 or 73. In addition, an AD may also comprise an aa sequence having greater than 96% or greater than 98% sequence identity to SEQ ID NOs: 72 or 73.

An AD may comprise an anti-human CD3 VHH aa sequence comprising the sequence of SEQ ID NO:74. An AD may comprise an aa sequence having greater than about 90% or greater than about 95% sequence identity to SEQ ID NO:74. In addition, an AD may also comprise an aa sequence having greater than 96% or greater than 98% sequence identity to SEQ ID NO:74

An AD may comprise an anti-human CD3 aa sequence based upon the anti-human CD3 L2K antibody. An L2K based AD may comprise an anti-human CD3 scFv (vH-vL) having the aa sequence of SEQ ID NOs: 75 or 76. An AD may comprise an aa sequence having greater than about 90% or greater than about 95% sequence identity to SEQ ID NOs: 75 or 76. In addition, an AD may also comprise an aa sequence having greater than 96% or greater than 98% sequence identity to SEQ ID NOs: 75 or 76.

An L2K based AD may comprise an anti-human CD3 scFv (vH-vL) having the aa sequence of SEQ ID NOs: 77 or 78. An AD may comprise an aa sequence having greater than about 90% or greater than about 95% sequence identity to SEQ ID NOs: 75 or 76. In addition, an AD may also comprise an aa sequence having greater than 96% or greater than 98% sequence identity to SEQ ID NOs: 75 or 76.

An L2K based AD may comprise an anti-human CD3 scFv (vH-vL) having the aa sequence of SEQ ID NO: 79. An AD may comprise an aa sequence having greater than about 90% or greater than about 95% sequence identity to SEQ ID NO:79. In addition, an AD may also comprise an aa sequence having greater than 96% or greater than 98% sequence identity to SEQ ID NO:79.

### (9) CD28

An AD may comprise an aa sequence that binds to CD28 (e.g., an anti-CD28 scFv or nanobody aa sequence). An anti-CD28 scFv that may be used as an AD may comprise the sequence: 1 DIELTQSPAS LAVSLGORAT ISCRASESVE YYVTSLMOWY QQKPGOPPKL LIFAASNVES 61 GVPARESGSG SGTNESLNIH PVDEDDVAMY FCQQSRKVPY TFGGGTKLEI KRGGGGSGGG 121 GSGGGGSQVK LQQSGPGLVT PSQSLSITCT VSGFSLSDYG VHWVROSPGQ GLEWLGVIWA 181 GGGTNYNSAL MSRKSISKDN SKSQVELKMN SLQADDTAVY YCARDKGYSY YYSMDYWGQG 241 TTVTVSS (SEQ ID NO:117). Alternatively, an AD may comprise a sequence having at least 90% or at least 95% sequence identity with SEQ ID NO: 117. An AD may also comprise a sequence having at least 96% or at least 98% sequence identity to at least 220 or at least 230 contiguous aas of SEQ ID NO:117.

### (10) CD16

An AD may comprise an aa sequence that binds to CD16 (e.g., an anti-CD16 scFv or nanobody aa sequence). Anti-CD16 VHH polypeptides that may be employed as an AD may comprise the aa sequence of SEQ ID NO: 80 or SEQ ID NO:81. Alternatively, an AD may comprise a sequence having at least 90% or at least 95% sequence identity with SEQ ID NOs: 80 or 81. An AD may also comprise a sequence having at least 96% or at least 98% sequence identity to at least 100 or at least 110 contiguous aas of SEQ ID NOs: 80 or 81.

### (11) IFN-γ

An AD may comprise an interferon-γ (IFN-γ) polypeptide sequence or an aa sequence that binds to the IFN-γ receptor (e.g., an anti-IFN-γ receptor scFv or nanobody aa sequence). An scIFN-γ polypeptide that may be employed as an IFN-γ AD may comprise the sequence: 1 MQDPYVKEAE NLKKYFNAGH SDVADNGTLF LGILKNWKEE SDRKIMQSQI VSFYFKLFKN 61 FKDDQSIQKS VETIKEDMNV KFFNSNKKKR DDFEKLTNYS VTDLNVORKA IDELIQVMAE 121 FSTEEQQEGP YVKEAENLKK YFNAGHSDVA DNGTLFLGIL KNWKEESDRK IMQSQIVSFY 181 FKLFKNFKDD QSIQKSVETI KEDMNVKFEN SNKKKRDDFE KLTNYSVTDL NVQRKAIHEL 241 IQVMAELSPA AKTGKRKRSQ MLFRG (SEQ ID NO:82). Alternatively, an AD may comprise a sequence having at least 90% or at least 95% sequence identity with SEQ ID NO:82. An AD may also comprise a sequence having at least 96% or at least 98% sequence identity to at least 220 or at least 230 contiguous aas of SEQ ID NO:82.

### (12) TNF

An AD may comprise a Tumor necrosis factor alpha (TNFα) polypeptide sequence or an aa sequence that binds to a TNF receptor (e.g., TNFR2, which is expressed on cells of the immune system). ADs may comprise an anti-TNFR scFv or nanobody aa sequence and, in particular, an anti-TNFR2 scFv or nanobody aa sequence.

An AD may comprise a single chain TNF (scTNF) that comprises the sequence: 1 SSRTPSDKPV AHVVANPQAE GQLOWLNRRA NALLANGVEL RDNOLVVPSE GLYLIYSQVL 61 FKGQGCPSTH VLLTHTISRI AVSYQTKVNL LSAIKSPCQR ETPEGAEAKP WYEPIYLGGV 121 FQLEKGDRLS AEINRPDYLD FAESGQVYFG IIALGGGSGG GSGGGSSSRT PSDKPVAHVV 181 ANPQAEGQLQ WLNRRANALL ANGVELRDNQ LVVPSEGLYL IYSQVLFKGQ GCPSTHVLLT 241 HTISRIAVSY QTKVNLLSAI KSPCQRETPE GAEAKPWYEP IYLGGVFQLE KGDRLSAEIN 301 RPDYLDFAES GQVYFGIIAL GGGSGGGSGG GSSSRTPSDK PVAHVVANPQ AEGQLQWLNR 361 RANALLANGV ELRDNOLVVP SEGLYLIYSQ VLFKGOGCPS THVLLTHTIS RIAVSYQTKV 421 NLLSAIKSPCQRETPEGAEAKPWYEPIYLGGVFQLEKGDRLSAEINRPDYLDFAESGQVYFGIIAL (SEQ ID NO:83), which comprises three repeats of a fragment of TNF separated by a linker comprised of GGGS repeats. Alternatively, an AD may comprise a sequence having at least 90% or at least 95% sequence identity with SEQ ID NO:83. An AD may also comprise a sequence having at least 96% or at least 98% sequence identity to at least 480 or at least 500 contiguous aas of SEQ ID NO:83.

### (13) Nucleic Acids

ADs do not have to be comprised of an aa sequence and may, for example, be comprised of nucleic acid sequences. Non-limiting examples of nucleic acid ADs include nucleic acid sequences with CpG repeats (e.g., a CpG oligodeoxynucleotide sequence) or IMT504 (TCATCATTTTGTCATTTTGTCATT (SEQ ID NO:87; see, e.g., Insula et al., 2007 Stem Cells, 25:1047-1054) optionally having 1, 2 or 3 nucleotide substitutions, deletions, or insertions.

### 5. Tumor-Specific Binding Domains (TSBs)

A tumor-specific binding domain of the constructs described herein (also referred to as tumor-specific binder, TSB singular and TSBs plural) is a polypeptide aa sequence or sequences that functions as a targeting sequence directing the construct to tumor cells by binding to target molecules expressed on or associated with the tumor cell surface (e.g., a tumor associated antigen or neoantigen). TSBs include, for example, polypeptides and other molecules such as antibodies, which include antigen binding fragments of antibodies, Fabs, Fab's, single chain antibodies, scFvs, peptide aptamers, and nanobodies. In some instances, TSBs are selected from nanobodies, scFabs, and scFvs. Bispecific antibodies that target two different TAAs or two different epitopes on a TAA may be employed as TSBs. The TSBs may be expressed as part of a peptide of a construct described herein. Alternatively, TSBs may be joined to the polypeptide of a construct via enzymatic or non-enzymatic (e.g., via a crosslinking agent) means (see, e.g., FIG. 5F where a bispecific antibody is joined to an NBD by a linker; however, the antibody could be joined to the linker by a crosslinking agent).

The molecules targeted by TSBs may be tumor-associated antigens (TAAs) whose cell surface expression is restricted, or substantially restricted, to one or more tumor cell types. TAAs are a subset of cancer associated antigens (CAA), whose cell surface expression is restricted, or substantially restricted, to one or more types of cancer cells found principally in tumors. Although the constructs described herein are principally designed to act in the TME, because target TAAs may also be present on the surface cells of non-solid cancers (e.g., leukemias), constructs targeting TAAs/CAAs also found on the surface cells of non-solid cancers may be utilized in the treatment of non-solid cancers in a patient separately or in addition to the treatment of solid tumors in that patient. In addition to molecules whose expression is restricted or substantially restricted to tumor cells, other molecules present on the surface of a tumor cell may be targeted including, but not limited to, cell proteins expressed on normal (non-transformed) cells whose expression is upregulated on the tumor cell's surface. Checkpoint proteins (e.g., V-domain Ig suppressor of T cell activation or “VISTA”, T-cell immunoglobulin and mucin domain 3 or “Tim-3”, and Programmed Death Ligand 1 or “PD-L1”), which are expressed on normal cells and on many tumor cells (e.g., transformed tumor cells), may also serve as target molecules for TSBs. Targeting checkpoint proteins, which are often upregulated by tumor cells as a means of immune evading or escaping therapeutics, both offer a target that may be abundantly expressed on the tumor cell's surface and provide checkpoint inhibition by the TSB. Proteins not substantially restricted to expression on transformed tumor cells may be considered as targets of TSBs, particularly when they are upregulated and expressed at higher levels on tumor cells, because immune responses directed by an NBD-containing construct comprising a TSB is limited to the TME where ATP levels are sufficiently high to permit pairing or complexation of the TSB with, for example, an NBD-containing construct comprising an AD, ICB, or scaffold capable of inducing ADCC, ADCP, and/or CDC (see, e.g., FIGS. 3, 4A, and 4B).

Some TAAs associated with a solid tumor that may be targeted by TSBs include, but are not limited to: carcinoembryonic antigen (CEA), mesothelin (MSLN), Erb-B2 Receptor Tyrosine Kinase 2 (HER2 or ERBB2), Epithelial Cell Adhesion Molecule (EPCAM), Vascular Endothelial Growth Factor Receptor (VEGF), Six transmembrane epithelial antigen of the prostate (STEAP), Epidermal Growth Factor Receptor (EGFR), Glypican-3 (GPC3), mucin 17 (MUC17), Prostate-Specific Membrane Antigen (PSMA), mucin 1 (MUC1), mucin 16 (MUC16), trophoblast cell surface antigen (TROP2), fibroblast growth factor receptor 2 (FGFR2b), claudin 6 (CLDN6), CD276 (B7-H3), carbonic anhydrase (CA9), podoplanin (PDPN), alkaline phosphatase, placental-like (ALPP, e.g., ALPPL2), Anthrax toxin receptor 1 (ANTXR1), claudin 18 (CLDN18), folate hydrolase-1 (FOLH1), guanylyl cyclase C (GUCY2C), interleukin-13 receptor 13 subunit alpha-2 (IL13RA2), podocalyxin (PODXL), prostate stem cell antigen (PSCA), Protein Tyrosine Kinase 7 (PTK7), Folate receptor 1 (FOLR1), V-domain Ig suppressor of T cell activation (VISTA), T-cell Ig and mucin-domain containing-3 (TIM-3), PD-L1, CTLA-4, tissue factor (human), c-Met tyrosine kinase, CD22, CD79b, CD19, CD30, folate receptor alpha (FRα), Nectin-4, B7H3, cMET, and lymphocyte-activation gene 3 (LAG-3).

Some TAAs associated with a solid tumor that may be targeted by TSBs include, but are not limited to: Six transmembrane epithelial antigen of the prostate (STEAP), prostate stem cell antigen (PSCA), and Prostate-Specific Membrane Antigen (PSMA).

Some TAAs associated with a solid tumor that may be targeted by TSBs include, but are not limited to: Mesothelial (MSLN), EpCAM, and CTLA-4.

Some TAAs associated with a solid tumor that may be targeted by TSBs include, but are not limited to: claudin 6 (CLDN6), claudin 18 (CLDN18), Podocalyxin (PODXL), and placental-like (ALPP, e.g., ALPPL2).

Some TAAs associated with a solid tumor that may be targeted by TSBs include, but are not limited to: MUC1, MUC16, or MUC 17.

Some checkpoint proteins that may serve as targets of TSBs include, but are not limited to, VISTA, TIM-3, PD-L1, CTLA-4, or LAG-3.

A TSB polypeptide may be an anti-human mesothelin scFv polypeptide related to the murine-derived SS1 antibody, also employed in Amatuximab having the sequence: QVQLQQSGPE LEKPGASVKI SCKASGYSFT GYTMNWVKQS HGKSLEWIGL ITPYNGASSY NQKERGKATL TVDKSSSTAY MDLLSLTSED SAVYFCARGG YDGRGFDYWG SGTPVTVSSG XGGSGGGGSG GGGSDIELTQ SPAIMSASPG EKVTMTCSAS SSVSYMHWYQ QKSGTSPKRW IYDTSKLASG VPGRESGSGS GNSYSLTISS VEAEDDATYY COQWSKHPLT FGSGTKVEIK, where X is V or G (SEQ ID NO:88). The TSB polypeptide may also comprise an aa sequence having greater than about 90% or greater than about 95% sequence identity to that sequence. Alternatively, the TSB polypeptide may comprise an aa sequence having greater than 96% or greater than 98% sequence identity to that sequence.

A TSB polypeptide may comprise an anti-human mesothelin scFv (vH-vL) polypeptide related to the monoclonal antibody YP218, having the sequence: QEQLVESGGG LVQPGASLTL TCTASGIDES RYYMCWVRQA PGKGLEGIAC IYIGGSGSTY YASWAKGRFT ISKASSTTVT LQMTSLTAAD TATYFCARGT NLNYIFRLWG PGTLVTVSSG XGGSGGGGSG GGGSDVVMTQ TPASVSEPVG GTVTIKCQAS QRISSYLSWY QQKPGORPKL LIFGASTLAS GVPSREKGSG SGTEYTLTIS DLECADAATY YCQSYAYFDS NNWHAFGGGT EVVV, where X is V or G (SEQ ID NO:89). The TSB polypeptide may also comprise an aa sequence having greater than about 90% or greater than about 95% sequence identity to that sequence. Alternatively, the TSB polypeptide may comprise an aa sequence having greater than 96% or greater than 98% sequence identity to that sequence.

A TSB polypeptide may comprise an anti-human mesothelin scFv (vL-vH) polypeptide related to the monoclonal antibody YP218, having the sequence: DVVMTQTPAS VSEPVGGTVT IKCQASQRIS SYLSWYQQKP GORPKLLIFG ASTLASGVPS RFKGSGSGTE YTLTISDLEC ADAATYYCOS YAYFDSNNWH AFGGGTEVVV GXGGSGGGGS GGGGSQEQLV ESGGGLVQPG ASLTLTCTAS GIDESRYYMC WVRQAPGKGL EGIACIYIGG SGSTYYASWA KGRFTISKAS STTVTLQMTS LTAADTATYF CARGTNLNYI FRLWGPGTLV TVSS, where X is V or G (SEQ ID NO:90). The TSB polypeptide may also comprise an aa sequence having greater than about 90% or greater than about 95% sequence identity to that sequence. Alternatively, the TSB polypeptide may comprise an aa sequence having greater than 96% or greater than 98% sequence identity to that sequence.

A TSB polypeptide may comprise an anti-human mesothelin scFv (vH-vL) polypeptide related to the monoclonal antibody 15B6, having the sequence: EVQLQQSGPV LVKPGASVKI SCKASGYSFT GYYMHWVRQS NGKSLEWIGR INPYTGVPSY KHNEKDKASL TVDKSSSTAY MELHSLTSED SAVYYCAREL GGYWGQGTTL TVSSGXGGSG GGGSGGGGSQ AVVTQESALT TSPGETVTLT CRSSTGAVTT GNYPNWVQEK PDHLFTGLIA GTNNRAPGVP ARESGSLIGD KAALTITGAQ TEDEAIYFCA LWFSSHWVFG GGTKLTVLG, where X is V or G (SEQ ID NO:91). The TSB polypeptide may also comprise an aa sequence having greater than about 90% or greater than about 95% sequence identity to that sequence. Alternatively, the TSB polypeptide may comprise an aa sequence having greater than 96% or greater than 98% sequence identity to that sequence.

A TSB polypeptide may comprise an anti-human mesothelin scFv (vL-vH) polypeptide related to the monoclonal antibody 15B6, having the sequence: PDHLFTGLIA GTNNRAPGVP ARESGSLIGD KAALTITGAQ TEDEAIYFCA LWFSSHWVFG GGTKLTVLGG XGGSGGGGSG GGGSEVQLQQ SGPVLVKPGA SVKISCKASG YSFTGYYMHW VRQSNGKSLE WIGRINPYTG VPSYKHNEKD KASLTVDKSS STAYMELHSL TSEDSAVYYC ARELGGYWGQ GTTLTVSS, where X is V or G (SEQ ID NO: 92). The TSB polypeptide may also comprise an aa sequence having greater than about 90% or greater than about 95% sequence identity to that sequence. Alternatively, the TSB polypeptide may comprise an aa sequence having greater than 96% or greater than 98% sequence identity to that sequence.

A TSB polypeptide may comprise an anti-human mesothelin scFv (vL-vH) polypeptide related to the monoclonal antibody 15B6 with a disulfide linkage between the vL and vH positions G102C and K43C (K168C as shown), having the sequence: QAVVTQESAL TTSPGETVTL TCRSSTGAVT TGNYPNWVQE KPDHLFTGLI AGTNNRAPGV PARESGSLIG DKAALTITGA QTEDEAIYFC ALWESSHWVE GCGTKLTVLG GGGGSGGGGS GGGGSEVQLQ QSGPVLVKPG ASVKISCKAS GYSFTGYYMH WVRQSNGCSL EWIGRINPYT GVPSYKHNEK DKASLTVDKS SSTAYMELHS LTSEDSAVYY CARELGGYWG QGTTLTVSS (SEQ ID NO:93). The TSB polypeptide may also comprise an aa sequence having greater than about 90% or greater than about 95% sequence identity to that sequence. Alternatively, the TSB polypeptide may comprise an aa sequence having greater than 96% or greater than 98% sequence identity to that sequence.

A TSB polypeptide may comprise an anti-human mesothelin scFv (vL-vH) polypeptide related to the monoclonal antibody 15B6 with a disulfide linkage between the vL and vH positions G102C and S44C (S169C as shown), having the sequence: QAVVTQESAL TTSPGETVT LTCRSSTGA VTTGNYPNWV QEKPDHLFTG LIAGTNNRAP GVPARESGSL IGDKAALTIT GAQTEDEAIY FCALWESSHW VFGCGTKLTV LGGGGGSGGG GSGGGGSEVQ LOQSGPVLVK PGASVKISCK ASGYSFTGYY MHWVRQSNGK CLEWIGRINP YTGVPSYKHN FKDKASLTVD KSSSTAYMEL HSLTSEDSAV YYCARELGGY WGOGTTLTVSS (SEQ ID NO:94). The TSB polypeptide may also comprise an aa sequence having greater than about 90% or greater than about 95% sequence identity to that sequence. Alternatively, the TSB polypeptide may comprise an aa sequence having greater than 96% or greater than 98% sequence identity to that sequence.

A TSB polypeptide may comprise an anti-human mesothelin scFv (vL-vH) polypeptide related to the monoclonal antibody 15B6 with a disulfide linkage between the vL and vH positions G103C and K43C (K168C as shown), having the sequence: QAVVTQESAL TTSPGETVTL TCRSSTGAVT TGNYPNWVQE KPDHLFTGLI AGTNNRAPGV PARFSGSLIG DKAALTITGA QTEDEAIYFC ALWESSHWVE GGCTKLTVLG GGGGSGGGGS GGGGSEVOLQ QSGPVLVKPG ASVKISCKAS GYSFTGYYMH WVRQSNGCSL EWIGRINPYT GVPSYKHNEK DKASLTVDKS SSTAYMELHS LTSEDSAVYY CARELGGYWG QGTTLTVSS (SEQ ID NO:95). The TSB polypeptide may also comprise an aa sequence having greater than about 90% or greater than about 95% sequence identity to that sequence. Alternatively, the TSB polypeptide may comprise an aa sequence having greater than 96% or greater than 98% sequence identity to that sequence.

A TSB polypeptide may comprise an anti-human mesothelin scFv (vL-vH) polypeptide related to the monoclonal antibody 15B6 with a disulfide linkage between the vL and vH positions G103C and S44C (S169C as shown), having the sequence: QAVVTQESAL TTSPGETVTL TCRSSTGAVT TGNYPNWVQE KPDHLFTGLI AGTNNRAPGV PARFSGSLIG DKAALTITGA QTEDEAIYFC ALWESSHWVE GGCTKLTVLG GGGGSGGGGS GGGGSEVQLQ QSGPVLVKPG ASVKISCKAS GYSFTGYYMH WVRQSNGKCL EWIGRINPYT GVPSYKHNEK DKASLTVDKS SSTAYMELHS LTSEDSAVYY CARELGGYWG QGTTLTVSS (SEQ ID NO:96). The TSB polypeptide may also comprise an aa sequence having greater than about 90% or greater than about 95% sequence identity to that sequence. Alternatively, the TSB polypeptide may comprise an aa sequence having greater than 96% or greater than 98% sequence identity to that sequence.

A TSB binder polypeptide may comprise an anti-human mesothelin (VHH) polypeptide related to the monoclonal antibody SD1, having the sequence: QVOLVOSGGG LVQPGGSLRL SCAASDEDFA AYEMSWVRQA PGQGLEWVAI ISHDGIDKYY TDSVKGRFTI SRDNSKNTLY LQMNTLRAED TATYYCLRLG AVGQGTLVTV SSS (SEQ ID NO:97). The TSB polypeptide may also comprise an aa sequence having greater than about 90% or greater than about 95% sequence identity to that sequence. Alternatively, the TSB polypeptide may comprise an aa sequence having greater than 96% or greater than 98% sequence identity to that sequence.

A TSB polypeptide may comprise an anti-human EpCAM scFv (vH-vL) polypeptide related to the monoclonal antibody MT201 and Adecatumumab, having the sequence: EVQLLESGGG VVQPGRSLRL SCAASGFTES SYGMHWVRQA PGKGLEWVAV ISYDGSNKYY ADSVKGRFTI SRDNSKNTLY LQMNSLRAED TAVYYCAKDM GWGSGWRPYY YYGMDVWGQG TTVTVSSGXG GSGGGGSGGG GSELQMTQSP SSLSASVGDR VTITCRTSQS ISSYLNWYQQ KPGQPPKLLI YWASTRESGV PDRFSGSGSG TDFTLTISSL QPEDSATYYC QQSYDIPYTF GQGTKLEIKR TV, where X is V or G (SEQ ID NO:98). The TSB polypeptide may also comprise an aa sequence having greater than about 90% or greater than about 95% sequence identity to that sequence. Alternatively, the TSB polypeptide may comprise an aa sequence having greater than 96% or greater than 98% sequence identity to that sequence.

A TSB polypeptide may comprise an anti-human EpCAM scFv (vL-vH) polypeptide related to the monoclonal antibody MT201 and Adecatumumab, having the sequence: ELQMTQSPSS LSASVGDRVT ITCRTSQSIS SYLNWYQQKP GQPPKLLIYW ASTRESGVPD RESGSGSGTD FTLTISSLQP EDSATYYCQQ SYDIPYTFGQ GTKLEIKRTV GXGGSGGGGS GGGGSEVOLL ESGGGVVQPG RSLRLSCAAS GFTFSSYGMH WVRQAPGKGL EWVAVISYDG SNKYYADSVK GRETISRDNS KNTLYLQMNS LRAEDTAVYY CAKDMGWGS GWRPYYYYGMD VWGQGTTVTV SS, where X is V or G (SEQ ID NO:99). The TSB polypeptide may also comprise an aa sequence having greater than about 90% or greater than about 95% sequence identity to that sequence. Alternatively, the TSB polypeptide may comprise an aa sequence having greater than 96% or greater than 98% sequence identity to that sequence.

A TSB polypeptide may comprise an anti-human CTLA-4 scFv (vL-vH) polypeptide related to Ipilimumab, having the sequence: EIVLTQSPGT LSLSPGERAT LSCRASQSVG SSYLAWYQQK PGQAPRLLIY GAFSRATGIP DRESGSGSGT DETLTISRLE PEDFAVYYCQ QYGSSPWTFG QGTKVEIKRG GGGSGGGGSG GGGSQVQLVE SGGGVVQPGR SLRLSCAASG FTFSSYTMHW VRQAPGKGLE WVTFISYDGN NKYYADSVKG RFTISRDNSK NTLYLQMNSL RAEDTAIYYC ARTGWLGPED YWGQGTLVTV SS (SEQ ID NO:100). The TSB polypeptide may also comprise an aa sequence having greater than about 90% or greater than about 95% sequence identity to that sequence. Alternatively, the TSB polypeptide may comprise an aa sequence having greater than 96% or greater than 98% sequence identity to that sequence.

A TSB polypeptide may comprise an anti-human CTLA-4 scFv (vL-vH) polypeptide related to Ipilimumab with a disulfide linkage between the vL and vH positions Q101C and K43C (K167C as shown), having the sequence: EIVLTQSPGT LSLSPGERAT LSCRASQSVG SSYLAWYQQK PGQAPRLLIY GAFSRATGIP DRESGSGSGT DETLTISRLE PEDFAVYYCQ QYGSSPWTFG CGTKVEIKRG GGGSGGGGSG GGGSQVOLVE SGGGVVQPGR SLRLSCAASG FTFSSYTMHW VRQAPGCGLE WVTFISYDGN NKYYADSVKG RFTISRDNSK NTLYLQMNSL RAEDTAIYYC ARTGWLGPED YWGQGTLVTVS S (SEQ ID NO:101). The TSB polypeptide may also comprise an aa sequence having greater than about 90% or greater than about 95% sequence identity to that sequence. Alternatively, the TSB polypeptide may comprise an aa sequence having greater than 96% or greater than 98% sequence identity to that sequence.

A TSB polypeptide may comprise an anti-human CTLA-4 scFv (vL-vH) polypeptide related to Ipilimumab with a disulfide linkage between the vL and vH positions Q101C and G44C (G167C as shown), having the sequence: IVLTQSPGTL SLSPGERATL SCRASQSVGS SYLAWYQQKP GQAPRLLIYG AFSRATGIEP DRESGSGSGT DETLTISRLE PEDFAVYYCQ QYGSSPWTFG CGTKVEIKRG GGGSGGGGSG GGGSQVQLVE SGGGVVQPGR SLRLSCAASG FTESSYTMHW VRQAPGKCLE WVTFISYDGN NKYYADSVKG RFTISRDNSK NTLYLQMNSL RAEDTAIYYC ARTGWLGPED YWGQGTLVTV SS (SEQ ID NO:102). The TSB polypeptide may also comprise an aa sequence having greater than about 90% or greater than about 95% sequence identity to that sequence. Alternatively, the TSB polypeptide may comprise an aa sequence having greater than 96% or greater than 98% sequence identity to that sequence.

A TSB polypeptide may comprise an anti-human CTLA-4 scFv (vL-vH) polypeptide related to Ipilimumab with a disulfide linkage between the vL and vH positions G102C and K43C (K167C as shown), having the sequence: EIVLTQSPGT LSLSPGERAT LSCRASQSVG SSYLAWYQQK PGQAPRLLIY GAFSRATGIP DRESGSGSGT DETLTISRLE PEDFAVYYCQ QYGSSPWTFG QCTKVEIKRG GGGSGGGGSG GGGSQVOLVE SGGGVVQPGR SLRLSCAASG FTESSYTMHW VRQAPGCGLE WVTFISYDGN NKYYADSVKG RFTISRDNSK NTLYLQMNSL RAEDTAIYYCA RTGWLGPED YWGQGTLVTVS S (SEQ ID NO:103). The TSB polypeptide may also comprise an aa sequence having greater than about 90% or greater than about 95% sequence identity to that sequence. Alternatively, the TSB polypeptide may comprise an aa sequence having greater than 96% or greater than 98% sequence identity to that sequence.

A TSB polypeptide may comprise an anti-human CTLA-4 scFv (vL-vH) polypeptide related to Ipilimumab with a disulfide linkage between the vL and vH positions G102C and G44C (G168C as shown), having the sequence: EIVLTQSPGT LSLSPGERAT LSCRASQSVG SSYLAWYQQK PGQAPRLLIY GAFSRATGIP DRESGSGSGT DETLTISRLE PEDFAVYYCQ QYGSSPWTFG QCTKVEIKRG GGGSGGGGSG GGGSQVOLVE SGGGVVQPGR SLRLSCAASG FTFSSYTMHW VRQAPGKCLE WVTFISYDGN NKYYADSVKG RFTISRDNSK NTLYLQMNSL RAEDTAIYYC ARTGWLGPED YWGQGTLVTVS S (SEQ ID NO:104). The TSB polypeptide may also comprise an aa sequence having greater than about 90% or greater than about 95% sequence identity to that sequence. Alternatively, the TSB polypeptide may comprise an aa sequence having greater than 96% or greater than 98% sequence identity to that sequence.

The linker sequences in SEQ ID NOs: 88-104 are bolded and italicized.

### 6. Payloads/Labels

Payloads and labels (e.g., drug molecules) may be attached to or incorporated into the constructs to facilitate their use as therapeutics and/or diagnostics. Among the types of payloads/labels that may be incorporated into the constructs of the present disclosure are those that permit detection of the labeled constructs location, for use in medical imaging and the like, and those that function as therapeutics: Some suitable payloads/labels include (i) radiolabels; (ii) radio-opaque labels and other contrast agents, (iii) optical labels (e.g., fluorescent, UV/Visible, near infrared labels such as IRdye700DX (IR700)), (iv) photoacoustic labels (e.g., near-infrared photoacoustic imaging (NIR-PAI), (v) MRI/NMR labels or contrast agents, (vi) SPECT labels (e.g., ¹²³I, ^{99m}Tc, ²⁰¹Tl, or ¹¹¹In), (vii) positron emission tomography (PET) labels, (ix) paramagnetic labels, (x) chemotherapeutic or cytotoxic agents, and the like. Some labels, like radionuclides that may be used for diagnostic and therapeutic purposes, may fall in more than one of the above-mentioned categories.

On an average basis the constructs disclosed herein may comprise from about 0.1 to about 0.5 mole or from about 0.5 to 1.0 moles of independently selected payloads (e.g., independently selected radionuclide labels or chemotherapeutics) per mole of construct. On an average basis the constructs disclosed herein may comprise from about 1.0 to about 2.0 moles or from about 2.0 to 4.0 moles of independently selected payloads per mole of construct. On an average basis the constructs disclosed herein may comprise from about 4.0 to about 6.0 moles or from about 6.0 to 8.0 moles of independently selected payloads per mole of construct. On an average basis the constructs disclosed herein may comprise from about 8.0 to about 10.0 moles or more than 10 moles of independently selected payloads per mole of construct.

As discussed above, the ability of the constructs described herein to accumulate in tumor tissues permits dosing at levels that are below therapeutic and/or toxic levels in non-tumor tissues (e.g., plasma, blood, serum, and/or peritumoral tissues) while the level accumulating in tumor tissues is at or above therapeutic and/or diagnostic levels. Accordingly, constructs of the present disclosure, including those bearing a payload and/or label, may be administered at levels that are sub-therapeutic in non-tumor tissues, while accumulating intratumorally to levels that are at or above the minimum therapeutic/diagnostic levels necessary for procedures including, but not limited to, diagnostic (e.g., imaging) and/or therapeutic action (e.g., by delivery of a nuclide).

Radioactive payloads (radiolabels) may take several forms, including, but not limited to, radiolabels that are chelated to a construct of the present disclosure via a chelating group that is covalently or non-covalently attached to the constructs, sometimes referred to as indirect labeling via complexation or chelation, typically used with ions of metallic radionuclides. Radiolabels may also be covalently attached to a construct of the disclosure directly by addition of the nuclide to the construct (e.g., tyrosine labeling with lodgen and NaI, or formation of phosphotyrosine or phosphoserine), or indirectly by or addition of a radiolabeled moiety. Lastly, radiolabels may be incorporated into a construct of the disclosure during the translation of all or part of the construct in a cell or cell-free translation system.

Constructs of the present disclosure may be modified to bear a chelating functionality that ultimately binds to a radionuclide that is typically in the form of a divalent or trivalent cation of a radioactive metal. Numerous chelators may be conjugated to molecules of the constructs disclosed herein including, but not limited to: diethylenetriaminepentaacetic acid (DTPA); 2,2′,2″,2″-(1,4,7,10-tetraazacyclododecane-1,4,7,10-tetrayl)tetraacetic acid (DOTA); 1,4,7-triazacyclononane-1,4,7-triacetic acid (NOTA); and 1,4,8,11-tetraazacyclotetradecane-1,4,8,11-tetraacetic acid (TETA). For example, DOTA forms complexes with ⁶⁷Ga, ⁹⁰Y and ¹¹¹In and may be used to radiolabel constructs with those nuclides. Similarly, NOTA can be employed to radiolabel molecules with ⁶⁴Cu. Deferoxamine (DFO) is a Zr chelator suitable for forming DFO ⁸⁹Zr labeled constructs. The chelator 4-(1,4,8,11-tetraazacyclotetradec-1-yl)-methyl benzole acid tetrahydrochloride (CPTA) may be used for copper (e.g., 64Cu) chelation. DFO-MAL-Cys-MZHER2 may be used for the chelation of 89Zr (see e.g., Xu et al. EJNMMI Res. 2020; 10:58). Deferasirox may be used to form iron chelating conjugates of the constructs, see e.g., Piolatto et al., Sci Rep 11, 12581 (2021). https://doi.org/10.1038/s41598-021-91983-w. (iron binding) may be used to form iron chelating conjugates of the constructs, see e.g., Piolatto et al., Sci Rep 11, 12581 (2021). https://doi.org/10.1038/s41598-021-91983-w. Other molecules comprising chelating groups such as porphyrins, polyamines, crown ethers, bis-thiosemicarbazones, polyoximes, and the like may also be utilized. Chelating groups may be coupled to the constructs using standard chemistries such as N-hydroxysuccinimide to link to amines (e.g., lysine side chains) or maleimides to link to sulfhydryl's of cysteines.

Indirect radiolabeling of the constructs described herein may be accomplished by addition of a radiolabeled moiety. For example, moieties comprising a radiolabel may be linked to the constructs using reagents containing N-succinimidyl or N-hydroxysuccinimide groups reactive with amines, or reagent comprising maleimide groups reactive with sulfhydryl groups of cysteines. By way of example, an N-succinimidyl-3-[halo]benzoate labeled with any halogen nuclide (e.g., a nuclide of fluorine, iodine, or bromine) may be utilized to label a construct at exposed lysine residues. Other reagents used for labeling of the constructs include, but are not limited to halogen radionuclide labeled: N-succinimidyl-4-iodobenzoate (PIB); N-succinimidyl-3-iodobenzoate (SIB); N-succinimidyl-5-iodo-3-pyridine carboxylate (SIPC); tetrafluorophenyl 4-fluoro-3-iodobenzoate (TFIB); Bolton-Hunter reagent; iodo-N-(2-aminoethyl) maleimide (IBM); (4-isothiocyanatobenzylammonio) undecahydroclosododecaborate (DABI); 2,3,5,6-tetrafluorophenyl-3-(nidocarboranyl) propionate (TCP); N-succinimidyl 5-guanidinomethyl-3-iodobenzoate (iso-SGMIB); N-succinimidyl 4-guanidinomethyl-3-iodobenzoate (SGMIB); and 1-(3-[125I]iodophenyl) maleimide (IPM).

Labeling of the constructs during translation either in culture or in cell free systems will typically comprise the incorporation of nitrogen, oxygen, sulfur, and/or hydrogen radioisotopes. Phosphorous labels are potentially added as a post translational modification. Incorporation of nuclides of any of those elements may be accomplished by adding labeled amino acids to the translation systems employed, or in the case of phosphorous a suitably labeled phosphorus compound (labeled ATP). The same process may be used to incorporate stable nuclides that may be used in imaging (e.g., such as 13C MRI imaging).

Radionuclides that may be incorporated into constructs include, but are not limited to: ¹¹C, ¹⁴C, ¹³N, ¹⁵O, ³²P, ³³P, ⁴⁷Sc, ⁵¹Cr, ⁵⁷Co, ⁵⁸Co, ¹⁸F, ⁵⁹Fe, ⁶²Cu, ⁶⁷Cu, ⁶⁷Ga, ⁶⁷Ga, ⁷⁵Br, ⁷⁵Se, ⁷⁵Se, ⁷⁶Br, ⁷⁷As, ⁷⁷Br, ^{80m}Br, ⁸⁹Sr, ⁹⁰Y, ⁹⁵Ru, ⁹⁷Ru, ⁹⁹Mo, ^{94m}Tc, ^{99m}Tc, ^{103m}Rh, ¹⁰³Ru, ¹⁰⁵Rh, ¹⁰⁵Ru, ¹⁰⁷Hg, ¹⁰⁹Pd, ¹⁰⁹Pt, ¹¹¹Ag, ¹¹¹|n, ^{113m}In, ¹¹⁹Sb, ^{121m}Te, ^{122m}Te, ^{125m}Te, ¹¹¹In, ¹²³I, ¹²⁴I, ¹²⁵I, ¹²⁶I, ¹³¹I, ¹³³I, ¹⁴²Pr, ¹⁴³Pr, ¹⁴⁹Pm, ¹⁵²Dy, ¹⁵³Sm, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Po, ²¹⁷At, ²¹⁹Rn, ²²¹Fr, ²²³Ra, ²²⁴Ac, ²²⁵Ac, ²⁵⁵Fm or Th²²⁷. Nuclides that may be incorporated into constructs may include: ¹¹C, ¹⁴C, ⁶²Cu, ⁶⁴Cu, ¹³N, ¹⁸F, ³⁵S. ⁶⁷Ga, ⁶⁸Ga, ¹⁵O, ^{94m}Tc, ^{99m}Tc, ¹¹¹In, ¹²³I, ¹²⁴I, ¹²⁵I, ¹³¹I, and ⁹⁰Y. Tritium (³H) may also be incorporated into the constructs and may be introduced during translation using labeled amino acids, by indirect labeling where a tritiated group or amino acid is added, or even by an exchange reaction. Tritium, although a weak beta radiation emitter offers the advantages of having a half-life of about 12 years, and being able to be introduced at multiple sites even in a single amino acid, thereby being able to increase the amount of radiolabel present in a molecule of construct.

As discussed above, the stoichiometry of a payload (e.g., a label and/or conjugated chemotherapeutic agent) to construct may be constrained (e.g., within a range). For example, on a molar average basis, one or more atoms of radionuclide or other payload (e.g., a chemotherapeutic or non-radioactive label) may be added per molecule of construct (e.g., two or more atoms of a radionuclide or another payload per molecule of construct). In addition, more than one type of radionuclide, and/or other payload may be added to a construct such that all payloads on a construct are not identical. For example, a construct may bear a TFIB containing a radionuclide of fluorine (e.g., ¹⁸F) and/or iodine (e.g., ¹²⁵I, ¹²⁶I, ¹³¹I, or ¹³³I), and in addition, either a radiosensitizer or photosensitizer. See, e.g., Petrov et al. Int. J. Mol. Sci. 2022, 23, 13789. It is also possible to incorporate a stable label such as a ¹³C and a ¹²⁵I radiolabel into the same molecule, or even a stable label and two radionuclides.

One or more independently selected radiosensitizers (also referred to as a radiation sensitizer) may be incorporated into the constructs or administered in conjunction with the constructs of the present disclosure, particularly where the constructs are radiolabeled. Small-molecule radiation sensitizing agents that may be administered/utilized with the constructs described herein include cisplatin, apaziquone, AQ4N, curcumin, dihydroartemisinin, docetaxel, doxorubicin, genistein, gemcitabine, 5-fluorouracil, misonidazole, mitomycin C, nelfinavir, papaverine, paclitaxel (arresting cells in the G2/M phase most sensitive to radiation damage), papaverine (papaverine hydrochloride), resveratrol, RRX-001, TH-302, and/or tirapazamine. See, e.g., Gong et al., International Journal of Nanomedicine 2021:16 1083-1102.

As with radiosensitizers, constructs of the present invention may be labeled with one or more independently selected photosensitizers and the molecules employed in photodynamic therapy. A wide variety of molecules are suitable as photosensitizers including, but not limited to, various phthalocyanines and porphyrins (e.g., verteporfin).

Active therapeutic or chemotherapeutic agents may be incorporated into the constructs as payloads to form an NBD-containing construct-drug conjugate. Therapeutic agent payloads include, but are not limited to: the microtubule inhibitors monomethyl auristatin E (MMAE), monomethyl auristatin F (MMAF), mertansine (also called DM1), and forms emtansine; the DNA binder calicheamicin; the topoisomerase inhibitors deruxtecan, SN-38 (active form of irinotecan) and exatecan. Other therapeutic agent payloads include, but are not limited to, siRNAs and cytotoxic agents (e.g., ravtansine (DM4) and pyrrolobenzodiazepine (PBD).

The constructs of the present disclosure may include one or more molecules of a fluorescent, ultraviolet/visible (UV/Vis), and/or near IR payload or label attached to each construct. Fluorescent and UV/Vis labels included, but are not limited to, small molecules (e.g., fluorescein and rhodamine) that may be covalently attached directly or indirectly through a linker to molecules of a construct disclosed herein. As an alternative to small molecule fluorescent labels, fluorescent protein sequence(s), e.g., green fluorescent protein sequences, may serve as the label. The fluorescent sequence may be covalently attached by a bifunctional crosslinker or may be translated as a fusion protein with a peptide of the construct.

Near infrared payloads/labels include, but are not limited to, the NIR fluorophore IRDye800CW (800CW from LI-COR), Alexa Fluor 680 (available as its NHS Ester from ThermoFisher Scientific). See, e.g., Chillers et al., Mol. Pharmaceutics 2017, 14, 1623-1633. Other infrared labels include IRdye700DX, which is a is a phthalocyanine dye that acts as a photosensitizer and has been employed in photoimmunotherapy. See e.g., Mitsunaga et al. 2012, Nat Med.; 17 (12): 1685-1691. doi: 10.1038/nm.2554 and Akalux® IV.

Where payloads and labels are not translated as part of the construct, they may be covalently joined to the construct directly by a bond to an amino acid of the construct, or indirectly through a linker. Payloads may be conjugated to the constructs using the same types of chemistries described for forming antibody drug conjugates. For example, N-hydroxysuccinimide, N-succinimide, and/or maleimide containing reagents can be used to link payloads to the constructs. Isocyanates and isothiocyanates may also be used to form conjugates of the constructs. Acid anhydrides permit the formation of, for example ester, amide, or thioester linkages to the constructs. See, e.g., Pelted et al. J Nanobiotechnology, 2019; 17:90. Enzyme-mediated conjugation also provides site-specific binding of payloads to constructs. Enzyme mediated approaches for preparing payload conjugates of constructs involves the use of enzymes such as a sortase or a transglutaminase that recognize two complementary motifs, more specifically a first motif on the construct and a second motif on a peptide/polypeptide attached to the payload. The enzymes can catalyze enzyme mediated conjugation by joining those motifs.

Linkers used to attach payloads may be cleavable so that the payload/label stays attached to the conjugate and is released in vivo (e.g., at an intratumoral location). Linkers suitable for joining payloads, including therapeutic agents and labels, are discussed above. Whether attached directly or indirectly through a linker, payloads/labels may be attached at specific locations on the constructs of the present disclosure (e.g., at a specific amino acid), or in specific portions of the construct (e.g., attached to the scaffold). An amino acid in the NBD or in a scaffold sequence may serve as the point of modification or attachment of a linker or a payload/label. Where constructs comprise an immunoglobulin scaffold (e.g., a heavy chain constant region such as IgFc) the scaffold may be used as a location for incorporating one or more payload and/or one or more label molecules. For example, the side chains of amino acids, including lysines and/or cysteines, located in a solvent accessible portion of the construct (e.g., a scaffold aa sequence) may be used as the point of attachment. Where a suitable amino acid (such as a solvent accessible lysine or cysteine, e.g., for NHS or maleimide coupling) is not present, the amino acid may be added to the construct of the present disclosure using the tools of molecular biology. Genetic expansion to incorporate amino acids not found among the naturally occurring proteinogenic L-amino acids may also be used to achieve site specific introduction of one or more payload molecules. For example, the cyclopropene derivative of lysine, CypK, may be introduced to couple payloads using an inverse-electron-demand Diels-Alder reaction (see, e.g., Oller-Salvia Angew Chem Int Ed Engl. 2018 Mar. 5; 57 (11): 2831-2834).

### E. Methods of Preparation

The present disclosure includes and provides for the preparation of the constructs disclosed herein by transcription and translation of nucleic acids encoding the constructs in mammalian cells (CHO cells), yeast cells (e.g., pica pastoris), Spodoptera cells, or prokaryotic cells including bacterial cells (e.g., E. coli cells). The constructs are purified from the media used to culture the cells expressing the protein constructs. While the constructs may be expressed in both eukaryotic and prokaryotic cells, they constructs may be advantageously modified to avoid secondary modifications that reduce expression levels or result in proteins subject to proteolytic degradation. One modification that may lead to suboptimal expression in mammalian cells relates to the substitutions employed to create hydrolysis deficient NBDs. While substitution of D668 of the TAP1 NBD with an aa other than Asp may render the NBD hydrolysis deficient or substantially hydrolysis deficient, substitution with Asn may also result in the N-linked glycosylation of protein in mammalian cells leading to suboptimal expression, proteolytic degradation, and/or misfolding. As such, D668 may be substituted by any amino acid other than Asp (e.g., D668N, D668A, or D668Q substitutions) for expression in prokaryotic cells such as E. coli. TAP-1 NBD-containing constructs more suitable for expression in mammalian cells may comprise a D668 substitution with an aa other than Asp or Asn (e.g., D668A or D668Q). Amino acids corresponding to catalytic Asp (D668) of TAP1 in TAP2 and other ATP-binding cassette NBDs employed in the constructs of the present disclosure may be substituted in the same manner as TAP1's NBD to avoid the introduction of post translational modification sequence. Constructs may be obtained by translation that is accomplished using the steps of contacting a vector comprising a nucleic acid encoding a construct of the present disclosure (e.g., a nucleic acid such as a DNA expression plasmid) with cells capable of expressing the encoded construct. Following expression of the construct by the cell (which may be controlled by the use of an inducible promoter), the construct is harvested from the culture (generally the culture medium and the construct purified. Example 8 illustrates one such method for obtaining constructs using cellular expression. Cell that transiently express a construct or cell lines that have stably integrated one or more copy of a nucleic acid sequence encoding a construct may also be employed in methods of construct preparation. Cell free translation or coupled transcription translation may also be used to prepare the constructs or portions of the constructs in place of cell-based expression.

Portions of the constructs disclosed herein may also be separately prepared, such as by cellular expression as discussed above, cell-free expression, chemical synthesis, etc., and then conjugated together using one or more chemical or enzymatic processes. Indeed, enzymatic means, self-catalyzed protein splicing, or chemical crosslinking (e.g., with a heterobifunctional crosslinker) can be used to couple (e.g. covalently attach), for example, ADs to NBDs, TSBs to NBDs, and/or NBDs to ICBs. Where the components to be coupled are both comprised of aa sequences, enzymatic means and self-catalyzed protein splicing represent coupling options that permit both the stoichiometry and structure of the coupling reaction, while substantially avoiding potential secondary coupling reactions. For example, sortases and split inteins may be utilized to append a sequence comprising an NBD to an AD, a TSB binder, or an ICB. See, e.g., Bhagawati et al., Proc Natl Acad Sci USA, 116 (44): 22164-22172 (2019). Alternatively, the enzyme catalyzed SpyTag/SpyCatcher system, which may employ the CnaB2 domain of the FbaB protein from Streptococcus pyogenes, can be utilized for conjugating an AD to a polypeptide comprising an NBD. See, e.g., Reddington and Howarth, Curr. Opin. in Chem. Biol., 29:94-99 (2015). Skilled artisans will recognize that individual portions of the constructs may be chemically modified in such a manner that when contacted they can form a complete construct, such as by providing a solvent accessible cysteine on one portion of the construct and attaching a reactive maleimide group to the second portion. The maleimide and cysteine can undergo a Michael reaction in aqueous solution resulting in a covalent linkage.

By way of example, heterodimerizing NBDs (e.g., from TAP1 and TAP2) can be expressed in cells and purified (e.g., by affinity and/or size exclusion chromatography). Separate aliquots of the NBDs can then be joined to an antibody that acts as a TSB and an AD to produce a pair of molecules that can heterodimerize in the presence of ATP to form a molecule capable of immune stimulation that targets tumor cells (see e.g., FIG. 1 at B). This is exemplified in FIG. 1 at B and FIG. 3 at N/O. In another example, aliquots of heterodimerizing NBDs can be separately joined to a TSB and an ICB that can heterodimerize in the presence of ATP to form a BiTE-like molecule capable of immune stimulation (see e.g., FIG. 3 at C). More complex structures including, but not limited to, those set forth in FIGS. 1-5, can be formed by expressing or synthesizing portions of the constructs separately and joining them chemically (e.g., with heterobifunctional crosslinkers) or enzymatically using, for example, the enzyme catalyzed reactions set forth above. The linkages resulting from joining separate portions of constructs may be covalent or noncovalent (e.g., biotin avidin linkages).

In addition to pure enzymatic and chemical conjugation methods for coupling components of the constructs described herein, affinity-guided methods that combine the specificity of protein-protein interactions with chemical conjugation may be employed. Affinity guided chemical conjugations offer efficient, facile, and specific chemical conjugation reactions. One such method, PEptide-Directed Photo-cross-linking (PEDIP) has been described by Park et al. (2018), Bioconjug. Chem., 29:3240-3244, and demonstrated for coupling polypeptides to antibody heavy chain constant regions. See also Kishomoto et al., (2019), Bioconjug Chem., 30 (3): 697-702.

Where the AD is not an aa sequence that can be translated in a biological system (e.g., it is a nucleic acid such as a CpG oligodeoxynucleotide), it may be coupled to the other portions of the molecule using, for example, bifunctional crosslinkers (e.g., homobifunctional or heterobifunctional crosslinkers).

The constructs, or portions thereof, may be purified using a variety of methods known in the art. For example, where the construct comprises an IgFc region as a scaffold, it may be purified by protein A or protein G chromatography. Constructs bearing affinity tags such as His tags may be purified by immobilized metal affinity chromatography (IMAC) on metal chelate affinity columns such as nickel or cobalt affinity columns. In addition, constructs may be purified by nucleotide affinity chromatography or dye-ligand affinity chromatography using Cibacron Blue F3GA, Procion Blue HB, or Reactive blue 2 as affinity ligands. Size exclusion chromatography may be employed either alone or in combination with affinity chromatography or IMAC to purify the products.

### F. Formulations and Methods of Treatment

### 1. Formulations

The NBD-containing constructs and complexes disclosed herein (see, e.g., FIGS. 1-5H) may be formulated into pharmaceutical compositions comprising one or more pharmaceutically acceptable ingredients (e.g., excipients). Pharmaceutical compositions may comprise: (i) one or more constructs of the present disclosure and (ii) one or more pharmaceutically acceptable excipients, e.g., nonionic surfactants, stabilizers, buffering agents, etc., which are known in the art and accordingly not discussed in detail herein. Pharmaceutically acceptable excipients are described in a variety of publications including but certainly not limited to “Remington: The Science and Practice of Pharmacy”, 19th Ed. (1995), or latest edition, Mack Publishing Co; A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer. Pharmaceutical Assoc., and updated editions of the foregoing.

The present disclosure also provides pharmaceutical compositions comprising a nucleic acid or a recombinant expression vector that encodes all or part of one or more constructs of the present disclosure.

Pharmaceutical compositions of the present disclosure will generally be in the form of aqueous or other solutions. The disclosure also contemplates pharmaceutical compositions in the form of powders, granules, tablets, pills, suppositories, capsules, sprays, and the like. The composition may be formulated according to the routes of administration described below.

The present disclosure provides a composition comprising at least one construct of the present disclosure and saline (e.g., 0.9% or about 0.9% NaCl), optionally buffered to a suitable pH (e.g., with a phosphate buffer) that is sterile and is free of detectable pyrogens and/or other toxins, or the detectable pyrogens and/or other toxins are below permissible limits.

Accordingly, the present disclosure includes and provides for the use of the NBD-containing constructs of the present disclosure in the preparation of a medicament for use in therapeutic applications, including but not limited to treating cancers in patients (e.g., mammalian patients including humans). The disclosure also includes and provides for the use of the NBD-containing constructs of the present disclosure in the treatment of mammalian patients (e.g., humans), including their use for the treatment of cancers in those patients.

The compositions may be prepared for parenteral and/or other forms of administration. The constructs or compositions comprising the constructs may be administered by any suitable route, such as intravenously, intramuscularly, subcutaneously, intratumorally, or intralymphatically. The constructs may also be administered in a treatment regimen in conjunction with one or more additional agents that act synergistically or non-synergistically with the constructs. The additional agents may be administered prior, simultaneously (separately or in admixture), or subsequent to the constructs. For example, constructs of the present disclosure (e.g., those with IL-12 activating domains) may be administered with immune checkpoint inhibitors such as Pembrolizumab (Keytruda®, targeting PD-1), Avelumab (Bavencio®, targeting PD-L1), or Ipilimumab (Yervoy®, targeting CTLA-4).

Because cancers may escape from therapy employing the constructs of the present disclosure by expression of CD39, which hydrolyzes ATP to ADP, or by expression of CD38, which begins a cascade of reactions cleaving NAD to adenosine that can inhibit T cell action, inhibitors of one or both of those molecules may be administered in conjunction with any therapeutic use of the constructs. The inhibitors may be administered prior to, concurrently with (e.g., in admixture or separately), and/or subsequent to the CD38 and/or CD39 inhibitors. Inhibitors of CD38 include, but are not limited to, Daratumumab (Darzalex®), Isatuximab (Sarclisa®), and combinations thereof. Inhibitors of CD39 include, but are not limited to, ceritinib (Zykadia®) (see J Immunother Cancer 2022 August; 10 (8): e004660. doi: 10.1136/jitc-2022-004660).

### 2. Methods of Treatment

Any solid tumor that contains elevated levels of ATP relative to normal tissue may be treated utilizing the constructs of the present disclosure. Although cancers such as lymphomas may not form solid tumors, they may collect or aggregate (e.g., in a tissue) to form “liquid tumors.” Provided the aggregated cells produce localized elevated extracellular ATP levels, the cancerous cells giving rise to those aggregates may be subject to treatment by the constructs described herein. Indeed, even small aggregates or individual cells that produce elevated extracellular ATP in their immediate vicinity may be treatable with the constructs described herein. Treatable tumors include, but are not limited to, mesotheliomas (e.g., pleural, peritoneal, pericardial or testicular), melanomas, sarcomas (e.g., synovial sarcoma, soft-tissue sarcoma, osteosarcoma and liposarcoma), carcinomas (e.g., adenocarcinoma, basal cell carcinoma, squamous cell carcinoma, and transitional cell carcinoma), carcinosarcomas, lymphomas, and germ cell tumors. Representative carcinomas include, but are not limited to, colorectal carcinoma, breast carcinoma, neuroendocrine tumors, lung carcinoma, and gastric carcinoma. For example, the tumors may be lung, liver, skin, gastrointestinal, pancreatic, brain, or reproductive tissue (e.g., ovarian sarcoma) tumors.

The method of treatment may comprise, for example, administering one or more constructs of the present disclosure, or one or more molecules comprising one or more nucleic acid sequences encoding one or more constructs of the present disclosure, to a patient or subject. NBD-containing constructs may be administered at levels that are systemically safe (e.g., sub-therapeutic and/or showing at most limited side effects in systemic circulation), but that accumulate at therapeutically effective amounts and/or levels in the TME as dimers due to ATP-driven dimerization and/or due to targeting by TSBs when present. Depending on the specific elements present in the constructs, they may act through a variety of mechanisms including, but not limited to, the induction of Ig-mediated effector functions, activation of immune cells (e.g., NK cells, T cell (such as effector T cells), neutrophils, and/or macrophages), suppression or depletion of myeloid suppressor and/or T reg cells in the TME, and/or removal of check point protein blockade of immune cell function. In addition to the foregoing, NBDs alone, or as part of a construct comprising other elements, may act to lower ATP in the TME by sequestration, or where the NBDs are hydrolytically active by degradation. The reduction in ATP can lead to reduced purinergic signaling which has been shown to mediate a variety of cancer-related processes, including cell migration, resistance to cytotoxic therapy, and immune regulation, consistent with the abundance of ATP-regulated purinergic receptors on cancer and stromal and various immune cell types. See, e.g., Draganov & Lee, Adv Exp Med. Biol. 2021; 1270:73-87 (doi: 10.1007/978-3-030-47189-7_5.

While the constructs of the present disclosure permit therapeutic treatments resulting from immune responses directed against tumor cells, their use with various payloads (e.g., radiolabels (radionuclides), radiosensitizer, photosensitizer, chemotherapeutic agents etc.) permits additional treatment options and combination therapies. NBD-containing constructs comprising one or more payload(s), optionally comprising one or more scaffolds, and only one NBD may accumulate in the TME as dimers due to ATP-driven dimerization. Constructs of the present disclosure comprising (i) one or more payloads, and an NBD, or (ii) one or more payloads, an NBD, and a scaffold (e.g., an immunoglobulin scaffold or non-immunoglobulin scaffold) but lacking a TSB and ICB may be used to effect treatment by acting as a tissue (e.g., tumor) specific/elective payload delivery mechanism due to dimerization in a high ATP environment such as that found in the TME. While the constructs themselves may be used for therapeutic purposes (e.g., induction of ADCC, ADCP, or CDC) in the tumor, presence of one or more payloads permits one or more additional therapies to be conducted. The additional therapies include, but are not limited to, chemotherapy when at least one payload is a chemotherapeutic agent, radiotherapy when at least one payload is a radionuclide or radiosensitizer, and photo dynamic therapy when at least one of the payloads is a photosensitizer. Addition of an ICB or AD to form constructs comprising (i) an ICB or AD, one or more payloads, and an NBD, or (ii) an ICB or AD, one or more payloads, an NBD, and a scaffold permits a combination of ICB or AD-mediated immunotherapy and delivery of the payload(s) to effect one or more second additional therapies (e.g., chemotherapy, radio therapy, photodynamic therapy, etc.). Addition of a TSB to form constructs comprising (i) a TSB, one or more payloads, and an NBD, or (ii) a TSB, one or more payloads, an NBD, and a scaffold not only adds to tumor selective targeting that can enhance payload selective delivery, but also permits a combination of TSB-mediated immunotherapy and delivery of the payload(s) to effect one or more additional therapies (e.g., chemotherapy, radiotherapy, photodynamic therapy, etc.). Formation of constructs by addition of a TSB and an ICB or AD to form constructs comprising (i) a TSB, an ICB or AD, one or more payloads, and an NBD, or (ii) a TSB, an ICB or AD, one or more payloads, an NBD, and a scaffold permits one or more immunotherapies to be combined with delivery of the payload(s) to effect one or more additional therapies. Where any of the constructs comprises a detectable label (e.g., a radionuclide or radio-opaque label), they may also be used for diagnostic purposes. Such constructs may be administered at levels that are systemically safe in one or more non-tumor tissues (e.g., in plasma or peritumor tissues) and even potentially at subtherapeutic levels in non-target tissue such as the TME, but accumulate in the TME to therapeutic levels.

The one or more payloads present may be used for the diagnosis or therapy of a patient. For example, chemotherapeutic payloads that may be attached to the construct by a cleavable linker may be released (e.g., substantially, or wholly) in the TME. Where the one or more payloads comprise one or more labels that permit detection of a construct (e.g. labels permitting MRI or CT detection), the construct can be used to determine the location and size of tumors, which assists in evaluating the course of therapy and/or surgical intervention. Where the one or more payload(s) comprise a radiolabel and/or radiosensitizer, the constructs can be used in radioimmunotherapy. Similarly, where the constructs comprise a photosensitizer, the constructs may be used in photodynamic therapy. Constructs comprising a radiolabel, and additionally a radiosensitizer and/or photosensitizer, may be provided at doses that are systemically safe (even potentially at subtherapeutic levels) in one or more non-tumor tissues (e.g., in plasma or peritumoral tissue) but accumulate to therapeutic levels in the TME providing (i) a radiation source that may be supplemented by an external radiation source, and (ii) a sensitizing agent, that may be cleavably linked to the construct. Where radiolabeled constructs comprise a photosensitizer, radiotherapy and/or photodynamic therapy is possible. Delivery of one or more radiolabels and a sensitizer can be accomplished by providing both the radionuclide(s) and sensitizers on the same molecule which can homodimerize. Alternatively, a pair of heterodimerizing NBD-constructs wherein the first construct comprises the radiolabel (radionuclide(s)) and the second construct comprises the sensitizer may be employed. The formation and accumulation of the heterodimer in the tumor provides both the radionuclide and sensitizer in the TME, thereby facilitating the therapeutic treatment by permitting the use of photodynamic therapy and/or radiotherapy potentially supplemented by application of radiation (e.g., X-Ray radiation) from an external source.

NBD-containing constructs comprising a TSB and one or more payload(s) accumulate in the TME as dimers or higher order complexes and target tumor cells. Such constructs may be administered at levels that are systemically safe (even potentially at subtherapeutic levels) in one or more non-tumor tissues (e.g., in plasma or peritumor tissues) but which accumulate in the TME to therapeutic levels. In addition to any effect that engaging the tumor cell causes by engagement by an ATP-driven dimer or higher order complex of the construct(s) (e.g., induction of apoptosis), the payload(s) may deliver additional effects that contribute to the patient's diagnosis or therapy. For example, chemotherapeutic payloads may act on the tumor and labels that permit detection of the tumor assist in determining the course of therapy and/or surgical intervention. Where the payload(s) comprise a radiolabel or radiosensitizer, the TSB-containing constructs can be used in radioimmunotherapy. Similarly, where the constructs comprise a photosensitizer, the constructs may be used in photodynamic therapy. Constructs comprising a TSB, a radiolabel, and additionally a radiosensitizer and/or photosensitizer may be provided at doses that are systemically safe (even potentially at subtherapeutic levels) in one or more non-tumor tissues (e.g., in plasma or peritumoral tissue) but accumulate to therapeutic levels in the TME providing (i) a radiation source that may be supplemented by an external radiation source, and (ii) a sensitizing agent that may be cleavably linked to the construct. Where such radiolabeled constructs comprise a photosensitizer, radiotherapy and/or photodynamic therapy is possible. Delivery of one or more radiolabels and a sensitizer can be accomplished by providing both the radionuclide(s) and sensitizers on the same construct molecule which can homodimerize. Alternatively, a pair of heterodimerizing NBD-containing constructs wherein the first construct comprises the radionuclide(s) (radiolabel(s) and the second construct comprises the sensitizer may be employed. The formation and accumulation of the heterodimer in the tumor provides both the radionuclide and sensitizer in the TME, thereby facilitating the therapeutic treatment by permitting the use of photodynamic therapy and/or radiotherapy potentially supplemented by application of radiation from an external source.

NBD-containing constructs comprising one or more ICB and/or one or more AD aa sequences and one or more payload(s) accumulate in the TME as dimers or higher order complexes and induce one or more immune responses that impact tumor cells directly or indirectly as bystanders. Such constructs may be administered at levels that are systemically safe (even potentially at subtherapeutic levels) in one or more non-tumor tissues (e.g., in plasma or peritumor tissues), but which accumulate in the TME to therapeutic levels due to ATP-driven dimerization and/or higher order complex formation. In addition to any immune response(s) elicited by the ICB(s) and/or AD(s) in the ATP-driven dimers or higher order complexes of the construct(s) (e.g., cytotoxic T cell activation), any payload present may deliver additional effects that contribute to the patient's diagnosis or therapy. For example, chemotherapeutic payloads may act on the tumor and labels that permit detection of the tumor assist in determining the course of therapy and/or surgical intervention. Where the payload(s) comprise a radiolabel or radiosensitizer, the construct comprising an ICB and/or AD can be used in radioimmunotherapy. Similarly, where the constructs comprise a photosensitizer, the constructs may be used in photodynamic therapy. Constructs comprising an ICB and/or AD, a radiolabel, and additionally a radiosensitizer and/or photosensitizer may be provided at doses that are systemically safe (even potentially at subtherapeutic levels) in one or more non-tumor tissues (e.g., in plasma or peritumoral tissue) but accumulate to therapeutic levels in the TME providing (i) a radiation source that may be supplemented by an external radiation source, and (ii) a sensitizing agent, that may be cleavably linked to the construct. Where such radiolabeled constructs comprise a photosensitizer, radiotherapy and/or photodynamic therapy is possible in addition to any immunotherapeutic effect of the construct (e.g., cytotoxic T cell activation). Delivery of one or more radiolabels and a sensitizer can be accomplished by providing both the radionuclide(s) and sensitizers on the same molecule which can homodimerize. Alternatively, a pair of heterodimerizing NBD-constructs wherein the first construct comprises the radionuclide(s) (radiolabel(s) and the second construct comprises the sensitizer may be employed. The formation and accumulation of the heterodimer in the tumor provides both the radionuclide and sensitizer in the TME, thereby facilitating the therapeutic treatment by permitting the use of photodynamic therapy and/or radiotherapy potentially supplemented by application of radiation from an external source.

Efficacy of treatments employing the NBD-containing constructs described herein, measured for example by reduction in tumor size, tumor number, circulating tumor nucleic acids etc., may be enhanced by the use of more than one type of therapeutic regimen. As discussed above, the NBD-containing constructs may induce, for example, one or more type of immune response that will result in targeted killing of tumor cells. Additional therapeutic regimens to enhance therapeutic efficacy may comprise the use of one or more chemotherapeutic agents as a payload or by coadministration. Additional therapeutic regimens to enhance therapeutic efficacy also include the use of a radiolabel, a radiosensitizer, and/or a photosensitizer as payload on the NBD-containing constructs of the present disclosure. In addition to increased efficacy, the use of such payloads increases the safety (reduced side effects) of the therapy. This is particularly true where radiosensitizers and photosensitizers are employed as it is possible to irradiate the area(s) in which tumors are located with radiation and/or light of a suitable frequency (e.g., near IR), and to limit the dose of radiation and light.

It is also possible to improve the selectivity and safety of treatments employing the NBD-containing constructs described herein by the use of two or more TSBs that target different TAAs expressed on the same tumor cells. Alternatively, the two or more TSBs may target at least two distinct epitopes (non-overlapping epitopes) expressed on a TAA.

To obtain selective action by the constructs described herein in target tissue(s) (e.g., in a TME), the constructs should dimerize or form higher order complexes (dimerization/complexation) at ATP concentrations found in those target tissue(s) but not at the ATP concentrations found in non-target tissue. To determine if a patient or subject may benefit from therapy using the constructs described herein, and/or select constructs that will undergo dimerization/complexation at the ATP concentrations in their target tissue(s), the methods and uses of constructs described herein, including methods of capping or lattice formation described below, may include measurement of the ATP concentration in a target tissue, a non-target tissue, or both a target and a non-target tissue. Measurements in the tissues surrounding a target tissue (e.g., peritumor tissue) may also be used in place of, or in addition to, measurements of ATP concentrations in a non-target tissue. Depending on how much ATP mediated dimerization/complexation in tissues surrounding the target is acceptable and/or desirable, particularly in the therapeutic setting, the measurements in the surrounding non-target tissue may be more or less informative in the selection of constructs that dimerize/complex at suitable ATP concentrations. For example, where potential damage to one or more surrounding tissues during therapy is a critical concern, it may be more suitable to select constructs with an EC50 for dimerization/complexation at an ATP concentration slightly above the concentration in the target tissue so that a significant portion of the construct may dimerize/complex in that tissue (even if less than half of the construct(s) in that tissue dimerize) because even less will dimerize/complex in the surrounding tissue(s) where ATP is at a lower concentration.

Increasing the fraction of constructs that are in ATP mediated dimers/complexes increases their localization to target and their residence time in that target tissue particularly where they comprise TSB elements in part because of the increase in the effective binding energy, reflected as increased avidity (affinity) for the corresponding TAA. Increasing the fraction of constructs that are in ATP mediated dimers/complexes is particularly desirable for constructs bearing payloads (e.g., radiolabels, and chemotherapeutics) that do not require external stimulation (radiation or light in the case of photodynamic therapy) to effect treatment. It is also beneficial for payloads that require external stimulation to effect treatment (e.g., photosensitizers or radiosensitizers) that when combined with other treatments can substantially limit damage to non-target tissues, particularly in those instances where the external stimulus can be precisely applied to the target tissue (e.g., light or radiation focused on target issue from one or more external sources).

As discussed above, those ATP measurements may be made to assist in selecting constructs that dimerize at a suitable ATP concentration to produce the therapeutic effect associated with formation of dimers/complexes of the constructs, while limiting side effects due to action on non-target tissues. Measurements of ATP levels in non-target tissues and/or target tissues may be made prior to or during treatment with an NBD-containing construct described herein. ATP concentration measurements in target and non-target tissues may also be made subsequent to any one or more administrations of the construct. The concentration in target tissue such as in a solid tumor's TME, in the vicinity of non-solid cancers where the cells localize (accumulate in a given location such as an organ or tissue), or in non-target tissue(s) may be made by methods known in the art (see, e.g., Rajendran et al., Biol Bull. 2016; 231 (1): 73-84). Methods for assessing ATP concentration in vivo include but are not limited to: Magnetic Resonance Imaging (MRI)/Magnetic Resonance Spectroscopy (MRS), including phosphorous MRI (see, e.g., Gams et al., Curr. Oncol. 2021, 28, 5041-5053 and Malik Galijašević et al. Cancers 2021, 13, 3569). Additionally, the use of liquid chromatography-mass spectrometry, microelectrode measurement, and molecular probes (e.g., fluorescent or luminescent probes) may be utilized (e.g., of biopsy samples).

As the typical ATP concentration in non-target tissue is typically 0.01 μM to 0.1 μM (100 nM) the EC50 ATP induced dimerization/complexation may be selected to be higher than in the non-target tissue ATP concentration range. The concentration in target tissues such as the TME may be from about 50 to about 1,000 μM or even higher and the EC50 for ATP induced dimerization/complexation may be selected to be below, or at about the lower end of the target tissue concentration range to slightly above the concentration found in a target tissue. Accordingly, some EC50 value ranges for ATP induced dimerization/complexation consistent with target tissue selective use of the constructs or compositions described herein include the following ranges. The construct or composition may have an EC50 for ATP induced dimerization/complexation from about 0.1 μM to about 0.5 μM of ATP, or from about 0.5 μM to about 5.0 μM of ATP. The construct or composition may also have an EC50 for ATP induced dimerization/complexation from about 5.0 μM to about 50 μM of ATP, or from about 50 μM to about 250 μM of ATP. Additionally, the constructs may also have an EC50 for ATP induced dimerization/complexation from about 250 μM ATP to about 1 mM ATP or more. Generally, the highest target tissue specific action and lowest off target action may be obtained by selecting constructs having an EC50 from about 0.2 times to about the ATP concentration in the target tissue (e.g., up to about twice the concentration of ATP, in the target tissue), or about 0.4 times to about the ATP concentration in the target tissue, and at least 10 times the ATP concentration in non-target tissues.

Demonstration of ATP-induced dimerization of constructs (or their NBDs) may be carried out by size exclusion chromatography in, for example, a buffer such as 20 mM Tris-HCl (pH 8.0), 50 mM NaCl, 5 mM MgCl₂, 10% glycerol buffer, optionally with 0.005% Tween (v/v) (using, e.g., a Superdex 200 10/300 Increase or 16/600 column (depending on total protein quantities)) at 20 to 24° C. The observed retention time of the protein when comparing resulting chromatograms of NBD-containing proteins when run in buffer (as described above) with or without ATP will allow for the determination of monomeric vs dimeric form.

The EC50 value for ATP-induced dimerization of constructs (or their NBDs) may be measured via Biolayer Interferometry (BLI) using an Octet Red 96e instrument (Pall ForteBio LLC, Freemont, CA) by observing the alteration in signal (i.e., the interference pattern of white light reflected from the biological layer constructed on a biosensor tip) in 20 mM Tris-HCl, 150 mM NaCl, 5 mM MgCl₂·6H2O Hexahydrate, 0.005% Tween (v/v) pH 7.0 at 20 to 24° C. at different ATP concentrations (e.g., by titration of ATP). For constructs comprising TSBs EC50 is assessed by immobilizing target TAA to the biosensor tip and observing the signal change for a construct associating with the probe due to the increased avidity of the dimeric form at various ATP concentrations. For other constructs, and particularly where heterodimeric construct pairs are formed, some of the construct (e.g., a first member of a heterodimer) may be immobilized on the surface probe (e.g. using a streptavidin coated probe for constructs with an IgFc or an immobilized antibody to a portion of the construct preferably other than the NBD). Additional construct (e.g., the second member of a heterodimer pair) is exposed to the construct immobilized on the surface probe at various ATP concentrations and the response (alteration in the signal) observed. The midpoint of the ATP induced response of the BLI probe represents the EC50.

As an alternative to measuring ATP to determine if a patient or subject may benefit from treatment with the constructs of this disclosure, the concentration or expression level of CD73 may be assessed in the target tissue. The assessment may be made relative to a control non-target tissue or a peritumoral target tissue. An elevated level of CD73 (e.g., relative to a control tissue) indicates that a patient or subject is likely to benefit from treatment using constructs of the present disclosure because CD73 is understood to correlate with elevated extracellular adenosine/ATP levels. Accordingly, any of the methods of treatment or use of the constructs disclosed herein may comprise assessment of CD73 in a target tissue (a tumor TME tissue), a non-target tissue such as peritumor tissue, or both.

### 3. Methods of Capping or Lattice Formation

Capping (CAP formation), or lattice formation, is the reorganization of molecules on or in a cell's outer membrane from a distributed state (e.g., globally distributed on a cell's surface) into one or more aggregated masses on the cell's surface. The present disclosure includes and provides for methods of inducing capping or lattice formation on the surface of an immune cell (e.g., T cells, macrophages, NK cells, etc.) or a cancer cell. Those methods may comprise administering an NBD-containing construct or complex of the present disclosure to the patient. The method may employ constructs that have a single NBD (e.g., a TAP1 or TAP2 NBD that may engage 2 molecules of ATP) and either a single ICB or a single TSB. Examples of such a construct include a homodimerizing polypeptide (fusion protein/polypeptide) that comprises an NBD, an ICB, and optionally a scaffold sequence (e.g., an IgFc CH2-CH3 aa sequence) comprising a single NBD (e.g., homodimerizing) and an ICB or AD. Examples of such a construct include a homodimerizing polypeptide (fusion protein/polypeptide) that comprises an NBD, a TSB, and optionally a scaffold sequence (e.g., an IgFc CH2-CH3 aa sequence).

The ability of constructs to form a cap or lattice on the surface of a cell may be accessed in vitro by using labeled constructs and suitable microscopic methods (e.g., fluorescently labeled constructs and an optical microscope equipped with a suitable excitation source and visualization optics).

Capping by the constructs of the present disclosure, including the unexpected capping by constructs that are monovalent for a cell surface antigen (even where they become bivalent upon exposure to ATP), may induce a response in the cells. For example, capping proteins on the surface of effector T cells may cause activation (e.g., granule dependent or granule independent responses). In addition, where the constructs comprise IgFc sequences capable of inducing ADCC, ADCP, or CDC, the capping of the target proteins and accordingly the constructs bound to a cell's surface can enhance any one or more of ADCC, ADCP or CDC. Accordingly, constructs having a TSB and comprising IgFc sequences capable of inducing ADCC, ADCP, or CDC that induce capping/lattice formation in the TME due to elevated ATP levels may be utilized to effect any of those process.

### VI. ASPECTS

1. A construct comprised of a polypeptide comprising:
(i) a first nucleotide binding domain (NBD) amino acid (aa) sequence and
(ii) a scaffold (e.g., an immunoglobulin heavy chain constant region such as an scFc or KiHs-s IgFc) aa sequence
that are joined directly or by a linker peptide aa sequence (e.g., as a fusion protein/polypeptide);

wherein
(i) when the scaffold is an immunoglobulin heavy chain constant region (e.g., IgFc such as an scFc) aa sequence, it is capable of stimulating one or more immune cell effector functions (e.g., through engagement of Fcγ receptors), and
(ii) the NBD comprises one or more adenosine triphosphate (ATP) binding sites and can, in the presence of ATP, homodimerize or heterodimerize with a cognate non-identical second NBD optionally linked (e.g., as a fusion protein/polypeptide) to a second IgFc aa sequence by an optional linker. (see, e.g., FIG. 5A at A-E). It is understood that the NBD(s) present may be ATP hydrolysis deficient.

2. The construct of aspect 1, wherein the construct has a structure set forth in FIG. 5A at A or B.

3. The construct of aspect 1, further comprising a second NBD.

4. The construct of aspect 3, wherein the construct has a structure set forth in FIG. 5A at any of C-E.

5. The construct of any of aspects 1-4, wherein the immunoglobulin heavy chain constant region comprises a single chain IgFc that comprises a first immunoglobulin heavy chain constant region aa sequence and a second immunoglobulin heavy chain constant region aa sequence (e.g., a fusion polypeptide comprising a pair of Ig CH2-CH3 aa sequences, whose effector functions are optionally diminished (e.g., substantially or wholly)), wherein the first and second heavy chain aa sequences are optionally linked by one or more disulfide bonds (e.g., corresponding to those of the lower hinge region in a wt. IgG sequence).

6. A construct comprising:
(i) a first polypeptide comprised of a first immunoglobulin heavy chain constant region aa sequence (e.g., comprising an Ig CH2-CH3 aa sequence whose effector functions are optionally diminished (e.g., substantially or wholly)) and optionally an NBD amino acid (aa) sequence joined to the first immunoglobulin heavy chain constant region aa sequence directly or by a linker aa sequence, and
(ii) a second polypeptide comprised of a second immunoglobulin heavy chain constant region aa sequence (e.g., comprising an CH2-CH3 aa sequence whose effector functions are optionally diminished (e.g., substantially or wholly)) and optionally an NBD aa sequence joined to the second immunoglobulin heavy chain constant region aa sequence directly or by a linker aa sequence;

wherein
(i) at least one of the first and second polypeptides comprises an NBD (first NBD),
(ii) each NBD comprises one or more ATP binding sites and can, in the presence of ATP, homodimerize or heterodimerize with a cognate non-identical second NBD,
(iii) the first and second immunoglobulin heavy chain constant region aa sequences dimerize to form an CH2-CH3 dimer (e.g., an IgFc or IgFc-like structure) optionally capable of stimulating one or more effector functions, and
(iv) each linker sequence present is selected independently. (See, e.g., FIG. 5B at A-E.) It is understood that the NBD(s) present may be ATP hydrolysis deficient.

7. The construct of aspect 6, wherein the first polypeptide comprises the first NBD.

8. The construct of aspect 6, wherein the second polypeptide comprises the first NBD.

9. The construct of aspect 6, wherein the first or second construct further comprises a second NBD.

10. The construct of aspect 9, wherein the first polypeptide comprises the second NBD. (The NBDs are constrained so that no two NBDs within the same molecule can interact to form a complex in the presence of ATP. See, e.g., FIG. 5B, structures A-E, where positions 1 and 2 are NBDs, and structures F, H and J.

11. The construct of aspect 9, wherein the second polypeptide comprises the second NBD. (The NBDs are constrained so that no two NBDs within the same molecule can interact to form a complex in the presence of ATP. See, e.g., FIG. 5B, structures A-E, where positions 3 and 4 are NBDs).

12. The construct of any of aspects 6-7, wherein the first polypeptide comprises the first NBD and the second polypeptide comprises a second NBD. (The NBDs are constrained so that no two NBDs within the same molecule can interact to form a complex in the presence of ATP. See, e.g., FIG. 5B, structures A-E, where one of positions 1 and 2 and one of positions 3 and 4 are NBDs, and structures G, I, K, and L).

13. The construct of any of aspects 5-8, wherein the first and second immunoglobulin heavy chain constant region aa sequences form a heterodimer (e.g., are a pair of interspecific aa sequences).

14. The construct of aspect 13 wherein the first and second immunoglobulin heavy chain constant region aa sequences comprise an interspecific aa sequence pair selected from the group consisting of: KiH, KiHs-s, HA-TF, ZW1, 7.8.60, DD-KK, EW-RVT, EW-RVTs-s, and A107 aa sequences.

15. The construct of any of aspects 13 or 14, wherein the first and second immunoglobulin heavy chain constant region aa sequences comprise a KiH or KiHs-s aa sequence pair.

16. The construct of any of aspects 5-14, wherein the first and second immunoglobulin heavy chain constant region aa sequences are joined by one or more (e.g., two or more) disulfide bonds between those sequences.

17. The construct of any of aspects 1-16, wherein each immunoglobulin heavy chain constant region aa sequence comprises an independently selected IgG1, IgG2, IgG3, IgG4 or IgM aa sequence (e.g., SEQ ID NOs: 63-66, or a sequence having greater than 96% or greater than 98% sequence identity to any of SEQ ID NOs: 63-66).

18. The construct of any of aspects 1-17, wherein each immunoglobulin heavy chain constant region aa sequence comprises an IgG1 aa sequence.

19. The construct of any of aspects 1-18, wherein the immunoglobulin heavy chain constant region aa sequence comprises one or more substitutions that result in an enhancement of one or more effector functions relative to the immunoglobulin heavy chain constant regions lacking the substitution.

20. The construct of aspect 19, wherein each immunoglobulin heavy chain constant region aa sequence comprises an IgG1 aa sequence.

21. The construct of aspect 20, wherein one or more (e.g., each) immunoglobulin heavy chain constant region aa sequences comprise a substitution that enhances the ability of the sequence to enhance an effector function selected from the group consisting of: S239D, 1332E, the double substitution S239D and 1332E, and the triple substitution S239D, 1332E, and A330L.

22. The construct of aspect 20, wherein one or more (e.g., each) immunoglobulin heavy chain constant region aa sequences comprises a substitution that enhances the ability of the sequence to enhance an effector function selected from the group consisting of: G236A; E345R; S298A, E333A, and K334A; S239D, A330L, and 1332E; S239D and 1332E; G236A, S239D, A330L, and 1332E; S239D, 1332E, and G236A; L234Y, G236W, and S298A; F243L, R292P, Y300L, V305I, and P396L; K326W and E333S; K326A and E333S; K326M and E333S; C221D and D222C; S267E, H268F, and S324T; and H268F and S324T.

23. The construct of any of aspects 1-22, wherein at least one (e.g., each) NBD is located N-terminal to an immunoglobulin heavy chain constant region aa sequence or C-terminal to an immunoglobulin heavy chain constant region aa sequence.

24. The construct of any of aspects 1-22, wherein, when the construct comprises two or more NBDs on a single polypeptide, each NBD is located N-terminal to an immunoglobulin heavy chain constant region aa sequence or C-terminal to an immunoglobulin heavy chain constant region aa sequence.

25. The construct of any of aspects 1-22, wherein, when the construct comprises two or more NBDs on a single polypeptide, one NBD is located N-terminal to an immunoglobulin heavy chain constant region aa sequence and the second NBD is located C-terminal to an immunoglobulin heavy chain constant region aa sequence.

26. The construct of any of aspects 6-22, wherein, when the construct comprises a first polypeptide and a second polypeptide and two or more NBDs, the first NBD is located on the first polypeptide and the second NBD is located on the second polypeptide (e.g., the NBDs are independently selected to be located N-terminal to an immunoglobulin heavy chain constant region aa sequence or C-terminal to an immunoglobulin heavy chain constant region aa sequence).

27. The constructs of any of aspects 1-26, further comprising one or more (e.g., two or more) independently selected Tumor-Specific Binders (TSBs) (e.g., that bind one or more TAAs independently selected from the group consisting of a mucin (e.g., mucin1, 16 or 18), mesothelial (MSLN), EpCAM, CTLA-4, VISTA, TIM-3, PD-L1, CTLA-4, and LAG-3 protein).

28. The construct of aspect 27, wherein the construct comprises two or more TSBs. For example, the construct may have a structure set forth as in FIG. 5C, structure C or D. The construct may have a structure as set forth in FIG. 5C, structure E.

29. The construct of any of aspects 27-28, wherein at least one TSB is located N-terminal to an immunoglobulin heavy chain constant region aa sequence (e.g., at the N-terminus of one or more peptides of the construct).

30. The construct of any of aspects 27-28, wherein at least one TSB is located C-terminal to an immunoglobulin heavy chain constant region aa sequence (e.g., at the C-terminus of one or more peptides of the construct).

31. The construct of any of aspects 27-28 wherein each polypeptide of the construct comprises a TSB located N-terminal to an immunoglobulin heavy chain constant region aa sequence (e.g., at the N-terminus of each polypeptide of the construct).

32. The construct of any of aspects 27-28 wherein each polypeptide of the construct comprises a TSB located C-terminal to an immunoglobulin heavy chain constant region aa sequence (e.g., at the C-terminus of each polypeptide of the construct).

33. The construct of any of aspects 6-26 further comprising one or more (e.g., two or more) independently selected Tumor-Specific Binders (TSBs) (e.g., that bind one or more TAAs independently selected from the group consisting of a mucin (e.g., mucin1, 16 or 18), mesothelial (MSLN), EpCAM, CTLA-4, VISTA, TIM-3, PD-L1, CTLA-4, and LAG-3 protein wherein the construct comprises:
A (i) a first polypeptide comprising in the N-terminal to C-terminal direction an NBD, a first immunoglobulin heavy chain constant region aa sequence (e.g., comprising an Ig CH2-CH3 aa sequence), and a TSB, each optionally joined by linker aa sequences that are selected independently, and
   (ii) a second polypeptide comprising in the N-terminal to C-terminal direction a second immunoglobulin heavy chain constant region aa sequence (e.g., comprising an Ig CH2-CH3 aa sequence); or
B (i) a first polypeptide comprising in the N-terminal to C-terminal direction a first NBD, a first immunoglobulin heavy chain constant region aa sequence (e.g., comprising an Ig CH2-CH3 aa sequence), and a TSB aa sequence, each optionally joined by linker aa sequence(s) that are selected independently, and
   (ii) a second polypeptide comprising in the N-terminal to C-terminal direction a second NBD, and a second immunoglobulin heavy chain constant region aa sequence (e.g., comprising an Ig CH2-CH3 aa sequence), each optionally joined by linker aa sequence(s) that are selected independently;
wherein the heavy chain constant region aa sequences of the first and second polypeptides are an interspecific pair of scaffold polypeptides and are optionally linked by one or more interpeptide disulfide bonds, the first and second immunoglobulin heavy chain constant region aa sequences form an IgFc or fragment thereof sufficient to stimulate Ig-mediated effector functions and each NBD is optionally capped. See FIG. 5C structures A and B.

34. The construct of any of aspects 6-26 further comprising one or more (e.g., two or more) independently selected TSBs (e.g., TSBs that bind one or more TAAs independently selected from the group consisting of a mucin (e.g., mucin1, 16 or 18), mesothelial (MSLN), EpCAM, CTLA-4, VISTA, TIM-3, PD-L1, CTLA-4, and LAG-3 protein, wherein the construct comprises:
A (i) a first polypeptide comprising in the N-terminal to C-terminal direction a first NBD, a first immunoglobulin heavy chain constant region aa sequence (e.g., comprising an Ig CH2-CH3 sequence), and a first TSB aa sequence optionally joined by linker aa sequence(s) that are selected independently, and
   (ii) a second polypeptide comprising in the N-terminal to C-terminal direction a second immunoglobulin heavy chain constant region aa sequence (e.g., comprising an Ig CH2-CH3 aa sequence), and a second TSB aa sequence optionally joined by linker aa sequence(s) that are selected independently; or
B (i) a first polypeptide comprising in the N-terminal to C-terminal direction a first NBD, a first immunoglobulin heavy chain constant region aa sequence (e.g., comprising an Ig CH2-CH3 aa sequence), and a first TSB aa sequence optionally joined by linker aa sequence(s) that are selected independently, and
   (ii) a second polypeptide comprising in the N-terminal to C-terminal direction a second NBD, a second immunoglobulin heavy chain constant region aa sequence (e.g., comprising an Ig CH2-CH3 aa sequence), and a second TSB aa sequence each optionally joined by linker aa sequence(s) that are selected independently;
wherein the heavy chain constant regions of the first and second polypeptides are optionally a pair of interspecific aa sequences and are optionally linked by one or more interpeptide disulfide bonds, the first and second immunoglobulin heavy chain constant region aa sequences form an IgFc or fragment thereof sufficient to stimulate Ig-mediated effector functions, the first and second TSBs are independently selected, and each NBD is optionally capped. See FIG. 5C, structures C-E.

35. The construct of aspect 33 or 34, wherein the first and second immunoglobulin heavy chain constant region aa sequences are heterodimerizing sequences (e.g., interspecific pair of sequences).

36. The constructs of aspect 35, wherein the immunoglobulin heavy chain constant region aa sequences are a KiH or KiHs-s aa sequence pair. (e.g., wherein the first immunoglobulin heavy chain constant region aa sequence comprises the knob sequence and the second immunoglobulin heavy chain constant region aa sequence comprises the hole sequence).

37. The construct of any of aspects 27-36, wherein at least one of the one or more TSBs comprises an antibody, Fab, Fab′, single chain antibody, scFv, polypeptide aptamer, or nanobody aa sequence.

38. The construct of any of aspects 27-36, wherein at least one of the one or more TSBs comprises an scFv, polypeptide aptamer, or nanobody 39. A construct comprising a polypeptide that comprises:
(i) a first nucleotide binding domain (NBD) amino acid (aa) sequence, and
(ii) one or more immune cell binder (ICB) aa sequences and/or one or more AD aa sequences;

wherein
the NBD comprises one or more adenosine triphosphate (ATP) binding sites and can, in the presence of ATP, homodimerize or heterodimerize with a cognate non-identical second NBD; and
the first NBD, the one or more ICB aa sequences, and/or one or more AD aa sequences are optionally joined by independently selected linker polypeptide sequences. (See, e.g., FIG. 5D at A and G.) It is understood that the NBD(s) present may be ATP hydrolysis deficient.

40. The construct of aspect 39, wherein the construct is organized in the N-terminal to C-terminal direction as (i) an NBD aa sequence, optionally a linker, and one or more ICB and/or one or more AD aa sequences, or (ii) one or more ICB and/or one or more AD aa sequences, optionally a linker, and an NBD aa sequence.

41. The construct of aspect 39 or 40, further comprising a scaffold aa sequence; wherein the scaffold aa sequence is selected from non-dimerizing non-immunoglobulin aa sequences (e.g., leucine zipper proteins), and non-dimerizing immunoglobulin scaffolds (e.g., scFc, or mFc scaffolds), whose ability to stimulate one or more Ig-mediated effector functions is diminished (e.g., substantially or wholly) relative to their wt. Ig sequences. (See, e.g., FIG. 2 at C with a homodimerizing NBD, the structures at D with heterodimerizing NBDs and FIG. 5D at A.)

42. The construct of aspect 41, wherein the construct comprises the elements ordered in the N-terminal to C-terminal direction as either (i) NBD, scaffold, and ICB/AD aa sequences or (ii) NBD, ICB/AD, and scaffold aa sequences; wherein the elements are optionally joined by linker aa sequences that are selected independently.

43. The construct of aspect 41, wherein the construct comprises the elements ordered in the N-terminal to C-terminal direction as either (i) ICB/AD, scaffold, and NBD aa sequences or (ii) ICB/AD, NBD, and scaffold aa sequences; wherein the elements are optionally joined by linker aa sequences that are selected independently.

44. The construct of aspect 41, wherein the construct comprises the elements ordered in the N-terminal to C-terminal direction as either (i) scaffold, ICB/AD, and NBD aa sequences or (ii) scaffold, NBD, and ICB/AD aa sequences; wherein the elements are optionally joined by linker aa sequences that are selected independently.

45. The construct of any one of aspects 39-44, wherein the one or more ICB and/or one or more AD aa sequences comprises two or more (e.g., three or more) independently selected AD and/or ICB aa sequences, wherein the AD aa sequences are optionally placed in tandem.

46. The construct of any of aspects 41-45, wherein the scaffold sequence is a non-dimerizing aa sequence (e.g., an mFc or scFc).

47. A construct comprising:
(i) a first polypeptide comprised of a first scaffold aa sequence (e.g., comprising an Ig heavy chain constant region CH2-CH3 aa sequence optionally with diminished effector function) and optionally a first and/or second NBD aa sequence joined to the first scaffold aa sequence directly or by a linker aa sequence, and
(ii) a second polypeptide comprised of a second scaffold aa sequence (e.g., comprising an Ig heavy chain constant region CH2-CH3 aa sequence optionally with diminished effector function) and optionally a first and/or second NBD aa sequence joined to the second scaffold aa sequence directly or by a linker aa sequence;

wherein
(i) at least one of the first and second polypeptides comprises their first NBD aa sequence,
(ii) each NBD aa sequence comprises one or more ATP binding sites and can, in the presence of ATP, homodimerize or heterodimerize with a cognate non-identical NBD,
(iii) at least one of the first and second polypeptides comprises one or more ICB and/or one or more AD aa sequences,
(iv) the first and second scaffold sequences form a dimer via interactions between the first and second scaffold sequences (e.g., an IgFc), and
(v) each linker sequence present is selected independently. (See, e.g., FIG. 5D at B-F, H and I.)

It is understood that the NBD(s) present may be ATP hydrolysis deficient.

48. The construct of any of aspects 41-47, wherein each scaffold aa sequence comprises an independently selected IgG1, IgG2, IgG3, IgG4 or IgM aa sequence (e.g., SEQ ID NOs: 63-66, or a sequence having greater than 96% or greater than 98% sequence identity to any of SEQ ID NOs: 63-66).

49. The construct of any of aspects 41-48, wherein each scaffold aa sequence comprises an IgG1 aa sequence.

50. The construct of any of aspects 41-49, wherein the scaffold comprises an IgFc with one or more substitutions that can substantially diminish the ability to stimulate one or more Ig-mediated effector functions (e.g., ADCP, CDC, and/or ADCC) relative to the wild type sequences.

51. The construct of aspect 50, wherein the scaffold may comprise an IgG1 aa sequence bearing substitutions at one or more of L234, L235, G236, G237, P238, S239 and/or P331 (appearing as L14, L15, G16, G17, P18, S19 and/or P111 in SEQ ID NO:59 (e.g., a “LALA” substitution alone or in combination with a P331S substitution).

52. The construct of any of aspects 47-51, wherein the first and second scaffold aa sequences form a heterodimer (e.g., are a pair of interspecific aa sequences).

53. The construct of aspect 52, wherein the first and second scaffold aa sequences comprise an interspecific aa sequence pair selected from the group consisting of: KiH, KiHs-s, HA-TF, ZW1, 7.8.60, DD-KK, EW-RVT, EW-RVTs-s, and A107 aa sequences.

54. The construct of any of aspects 52-53, wherein the first and second scaffold aa sequences comprise a KiH or KiHs-s aa sequence pair.

55. The construct of any of aspects 47-54, wherein the first or second construct further comprises a second NBD aa sequence. (The NBDs are constrained so that no two NBDs within the same molecule can interact to form a complex in the presence of ATP.)

56. The construct of aspect 55, wherein the first polypeptide comprises the second NBD aa sequence.

57. The construct of aspect 55, wherein the second polypeptide comprises the second NBD aa sequence.

58. The construct of aspect 57, wherein the first polypeptide comprises the first NBD aa sequence and the second polypeptide comprises a second NBD aa sequence.

59. The construct of any of aspects 47 to 58, wherein at least one (e.g., each) NBD aa sequence is located N-terminal to the first or second scaffold aa sequence or C-terminal to the first or second scaffold aa sequence.

60. The construct of aspect 55, wherein the first and second NBD aa sequences are each located N-terminal to the first or second scaffold aa sequence or both are located C-terminal to the first or second scaffold aa sequence.

61. The construct of aspect 55, wherein the first NBD aa sequence is located N-terminal to a scaffold aa sequence and the second NBD is located C-terminal to a scaffold aa sequence; wherein the first and second NBD aa sequences are optionally both on the same or different ones of the first or second polypeptide.

62. The construct of aspect 61, wherein the first NBD is located on the first polypeptide and the second NBD is located on the second polypeptide, (e.g., the NBDs are independently selected to be located N-terminal to the scaffold aa sequence or C-terminal to the scaffold aa sequence).

63. The construct of any of aspects 47-62, wherein the first and second immunoglobulin heavy chain constant region aa sequences are joined by one or more (e.g., two or more) disulfide bonds between those sequences.

64. The constructs of any of aspects 39-63, further comprising one or more independently selected Tumor Specific Binder aa sequences (TSBs) that bind one or more independently selected TAAs.

65. The construct of aspect 64, wherein the construct comprises two or more independently selected TSBs that are optionally directed to different epitopes.

66. The construct of any of aspects 64-65, wherein one or more independently selected TSBs binds a TAA selected from a mucin (e.g., mucin1, 16 or 18), mesothelial (MSLN), EpCAM, CTLA-4, VISTA, TIM-3, PD-L1, CTLA-4, and LAG-3 protein).

67. The construct of any of aspects 64-66, wherein the one or more independently selected TSBs comprise independently selected antibody, Fab, Fab′, single chain antibody, scFv, polypeptide aptamer, or nanobody aa sequences.

68. The construct of any of aspects 64-67, wherein the one or more independently selected TSBs comprise independently selected scFv, polypeptide aptamer, or nanobody aa sequences.

69. The construct of any of aspects 64-68, wherein at least one TSB is located N-terminal to an immunoglobulin heavy chain constant region aa sequence (e.g., at the N-terminus of one or more peptides of the construct).

70. The construct of any of aspects 64-69, wherein at least one TSB is located C-terminal to an immunoglobulin heavy chain constant region aa sequence (e.g., at the C-terminus of one or more peptides of the construct) of an Ig scaffold.

71. The construct of any of aspects 64-70, wherein each polypeptide of the construct comprises a TSB located N-terminal to an immunoglobulin heavy chain constant region aa sequence of an Ig scaffold (e.g., at the N-terminus of each polypeptide of the construct).

72. The construct of any of aspects 64-70, wherein each polypeptide of the construct comprises a TSB located C-terminal to an immunoglobulin heavy chain constant region aa sequence of an Ig scaffold (e.g., at the C-terminus of each polypeptide of the construct).

73. The construct of any of aspects 64-68, wherein the construct has a structure selected from FIG. 5D, structures A-I (e.g., structures B-F, H and I), and the one or more TSBs are at position 1 and/or position 2 of the structures.

74. The construct of any of aspects 64-68, wherein the construct has a structure selected from FIG. 5D, structures A-I (e.g., structures B-F, H and I), and the one or more TSBs are at position 3 and/or position 4 of the structures.

75. The construct of any of aspects 64-68, wherein the construct has a structure selected from FIG. 5D, structures A-I (e.g., structures B-F, H and I), the one or more TSBs comprise a first and second TSB, and the first TSB is located at position 1 or 2 and the second TSB is located at position 3 or 4.

76. The construct of any of aspects 73-75, wherein each TSB present is directed to the same epitope, or there are at least two TSBs present in the construct directed to non-identical TAA epitopes.

77. The construct of any of aspects 64-68, wherein the construct comprises:
A (i) a first polypeptide comprising in the N-terminal to C-terminal direction an NBD, a first immunoglobulin heavy chain constant region aa sequence (e.g., comprising an Ig CH2-CH3 aa sequence) and a TSB optionally joined by linker aa sequences that are selected independently, and
   (ii) a second polypeptide comprising in the N-terminal to C-terminal direction an AD and a second immunoglobulin heavy chain constant region aa sequence (e.g., comprising an Ig CH2-CH3 aa sequence); or
B (i) a first polypeptide comprising in the N-terminal to C-terminal direction a first NBD, a first immunoglobulin heavy chain constant region aa sequence (e.g., comprising an Ig CH2-CH3 aa sequence), and a TSB aa sequence optionally joined by linker aa sequence(s) that are selected independently, and
   (ii) a second polypeptide comprising in the N-terminal to C-terminal direction a second immunoglobulin heavy chain constant region aa sequence (e.g., comprising an Ig CH2-CH3 aa sequence), and an AD aa sequence optionally joined by linker aa sequence(s) that are selected independently;
wherein the heavy chain constant region aa sequences of the first and second polypeptides are an interspecific pair of scaffold polypeptides optionally linked by one or more interpeptide disulfide bonds, and each NBD is optionally capped. See FIG. 5E, structures A and B.

78. The construct of any of aspects 64-68, wherein the construct comprises:
A (i) a first polypeptide comprising in the N-terminal to C-terminal direction an NBD, an AD, a first immunoglobulin heavy chain constant region aa sequence (e.g., comprising an Ig CH2-CH3 aa sequence), and a TSB optionally joined by linker aa sequences that are selected independently, and
   (ii) a second polypeptide comprising a second immunoglobulin heavy chain constant region aa sequence (e.g., comprising an Ig CH2-CH3 aa sequence); or
B (i) a first polypeptide comprising in the N-terminal to C-terminal direction an NBD, a first immunoglobulin heavy chain constant region aa sequence (e.g., comprising an Ig CH2-CH3 aa sequence), a TSB, and an AD, optionally joined by linker aa sequences that are selected independently, and
   (ii) a second polypeptide comprising a second immunoglobulin heavy chain constant region aa sequence (e.g., comprising an Ig CH2-CH3 aa sequence);
wherein the heavy chain constant region aa sequence of the first and second polypeptides are an interspecific pair of scaffold polypeptides optionally linked by one or more interpeptide disulfide bonds, and each NBD is optionally capped. See FIG. 5E, structures C and D.

79. The construct of any of aspects 77-78, wherein the first and second immunoglobulin heavy chain constant region aa sequences are a heterodimerizing interspecific pair of sequences selected from the group consisting of: KiH, KiHs-s, HA-TF, ZW1, 7.8.60, DD-KK, EW-RVT, EW-RVTs-s, and A107 aa sequences.

80. The construct of aspect 79, wherein the immunoglobulin heavy chain constant region aa sequences are a KiH or KiHs-s aa sequence pair. (e.g., wherein the first immunoglobulin heavy chain constant region aa sequence comprises the knob and the second immunoglobulin heavy chain constant region aa sequence comprises the hole).

81. A construct comprising a polypeptide comprising:
(i) a first nucleotide binding domain (NBD) amino acid (aa) sequence, and
(ii) one or more TSB aa sequences;
wherein
the NBD comprises one or more adenosine triphosphate (ATP) binding sites and can, in the presence of ATP, homodimerize or heterodimerize with a cognate non-identical second NBD; and
the first NBD and the one or more TSB aa sequences are optionally joined by an independently selected linker polypeptide sequence (see, e.g., FIG. 4B, A-D, and FIG. 5D at A, or G substituted as in construct Series 1-10 to 1-12, 2-10 to 2-12, 3-10 to 3-12, or 4-10 to 4-12 where the ICB or TSB is a TSB).

82. The construct of aspect 81, wherein the construct is organized in the N-terminal to C-terminal direction as (i) an NBD aa sequence, an optional linker, and one or more TSB aa sequences, or (ii) one or more TSB aa sequences, an optional linker, and an NBD aa sequence.

83. The construct of aspect 81 or 82 further comprising a scaffold aa sequence; wherein the scaffold aa sequence is selected from non-dimerizing non-immunoglobulin aa sequences (e.g., leucine zipper protein), and non-dimerizing immunoglobulin scaffolds (e.g., scFc, or mFc scaffolds), whose ability to stimulate one or more Ig-mediated effector functions is optionally diminished (substantially or wholly) relative to their wt. immunoglobulin sequences. (See, e.g., FIG. 4B at C and D.)

84. The construct of aspect 83, wherein the construct comprises the elements ordered in the N-terminal to C-terminal direction as either (i) NBD, scaffold, and TSB aa sequences or (ii) NBD, TSB, and scaffold aa sequences; wherein the elements are optionally joined by linker aa sequences that are selected independently.

85. The construct of aspect 83, wherein the construct comprises the elements ordered in the N-terminal to C-terminal direction as either (i) TSB, scaffold, and NBD aa sequences or (ii) TSB, NBD, and scaffold aa sequences; wherein the elements are optionally joined by linker aa sequences that are selected independently.

86. The construct of aspect 83, wherein the construct comprises the elements ordered in the N-terminal to C-terminal direction as either (i) scaffold, TSB, and NBD aa sequences or (ii) scaffold, NBD, and TSB aa sequences; wherein the elements are optionally joined by linker aa sequences that are selected independently.

87. The construct of any one of aspects 81-86, wherein the one or more TSB aa sequences comprises two or more (e.g., three or more) independently selected TSB aa sequences, wherein the TSB aa sequences are optionally placed in tandem.

88. The construct of any of aspects 81-87, wherein the scaffold sequence is a non-dimerizing aa sequence (e.g., a mFc or scFc)

89. A construct comprising:
(i) a first polypeptide comprised of a first scaffold aa sequence (e.g., comprising an Ig heavy chain constant CH2-CH3 aa sequence);
(ii) a second polypeptide comprised of a second scaffold aa sequence (e.g., comprising an Ig heavy chain constant CH2-CH3 aa sequence); and
(iii) a first NBD aa sequence joined to the first or second scaffold aa sequence directly or indirectly by a linker aa sequence (e.g., as a fusion protein);

wherein
(i) each NBD aa sequence comprises one or more ATP binding sites and can, in the presence of ATP, homodimerize or heterodimerize with a cognate non-identical NBD,
(ii) at least one of the first and second polypeptides comprises one or more TSB aa sequences,
(iii) the first and second scaffold sequences form a dimer via interactions between the first and second scaffold sequences (e.g., an IgFc), and
(iv) each linker sequence present is selected independently. (See, e.g., FIG. 5D at B-F, H and I substituted as in construct Series 1-10 to 1-12, 2-10 to 2-12, 3-10 to 3-12, or 4-10 to 4-12 where the ICB or TSB is a TSB).

90. The construct of aspect 89, further comprising a second NBD aa sequence joined to the first or second scaffold aa sequence directly or indirectly by an independently selected linker aa sequence.

91. The construct of any of aspects 83-90, wherein each scaffold aa sequence comprises an independently selected IgG1, IgG2, IgG3, IgG4 or IgM aa sequence (e.g., SEQ ID NOs: 63-66, or a sequence having greater than 96% or greater than 98% sequence identity to any of SEQ ID NOs: 63-66).

92. The construct of any of aspects 83-91 wherein
(i) one or more (e.g., each) scaffold aa sequence comprises an IgG1 aa sequence, or
(ii) one or more (e.g., each) scaffold aa sequence comprises an IgFc with one or more substitutions that can substantially diminish the ability to stimulate one or more Ig-mediated effector functions (e.g., ADCP, CDC, and/or ADCC) relative to the wild type sequences.

93. The construct of aspect 92, wherein the scaffold comprises an IgG1 aa sequence bearing substitutions at one or more of L234, L235, G236, G237, P238, S239 and/or P331 (appearing as L14, L15, G16, G17, P18, S19 and/or P111 in SEQ ID NO:59 (e.g., a “LALA” substitution alone or in combination with a P331S substitution).

94. The construct of any of aspects 89-93, wherein the first and second scaffold aa sequences form a heterodimer (e.g., are a pair of interspecific aa sequences).

95. The construct of aspect 94, wherein the first and second scaffold aa sequences are an interspecific aa sequence pair selected from the group consisting of: KiH, KiHs-s, HA-TF, ZW1, 7.8.60, DD-KK, EW-RVT, EW-RVTs-s, and A107 aa sequence pairs.

96. The construct of any of aspects 94-95, wherein the first and second scaffold aa sequences are a KiH or KiHs-s aa sequence pair.

97. The construct of any of aspects 89-96, wherein the first or second polypeptide further comprises a second NBD aa sequence. (The NBDs are constrained so that no two NBDs within the same molecule can interact to form a complex in the presence of ATP.)

98. The construct of aspect 97, wherein the first polypeptide comprises the second NBD aa sequence.

99. The construct of aspect 97, wherein the second polypeptide comprises the second NBD aa sequence.

100. The construct of aspect 97, wherein the first polypeptide comprises the first NBD aa sequence and the second polypeptide comprises a second NBD aa sequence.

101. The construct of any of aspects 89 to 100, wherein at least one (e.g., each) NBD aa sequence is located N-terminal to the first or second scaffold aa sequence or C-terminal to the first or second scaffold aa sequence.

102. The construct of aspect 97, wherein the first and second NBD aa sequences are each located N-terminal to the first or second scaffold aa sequence or both are located C-terminal to the first or second scaffold aa sequence.

103. The construct of aspect 97, wherein the first NBD aa sequence is located N-terminal to a scaffold aa sequence and the second NBD is located C-terminal to a scaffold aa sequence; wherein the first and second NBD aa sequences are optionally both on the same or different ones of the first or second polypeptide.

104. The construct of aspect 103, wherein the first NBD is located on the first polypeptide and the second NBD is located on the second polypeptide (e.g., the NBDs are independently selected to be located N-terminal to the scaffold aa sequence or C-terminal to the scaffold aa sequence).

105. The construct of any of aspects 89-104, wherein the first and second immunoglobulin heavy chain constant region aa sequences are joined by one or more (e.g., two or more) disulfide bonds between those sequences.

106. The constructs of any of aspects 81-105, further comprising two or more independently selected Tumor Specific Binders (TSBs) that bind one or more independently selected TAAs.

107. The construct of aspect 106, wherein the construct comprises two or more independently selected TSBs directed to different epitopes.

108. The construct of any of aspects 106-107 wherein one or more independently selected TSBs binds a TAA selected from a mucin (e.g., mucin1, 16 or 18), mesothelial (MSLN), EpCAM, CTLA-4, VISTA, TIM-3, PD-L1, CTLA-4, and LAG-3 protein.

109. The construct of any of aspects 106-108, wherein the independently selected TSBs comprise independently selected antibody, Fab, Fab′, single chain antibody, scFv, polypeptide aptamer, or nanobody aa sequences.

110. The construct of any of aspects 106-109, wherein the independently selected TSBs comprise independently selected scFv, polypeptide aptamer, or nanobody aa sequences.

111. The construct of any of aspects 106-110, wherein at least one TSB is located N-terminal to an immunoglobulin heavy chain constant region aa sequence (e.g., at the N-terminus of one or more peptides of the construct).

112. The construct of any of aspects 106-111, wherein at least one TSB is located C-terminal to an immunoglobulin heavy chain constant region aa sequence (e.g., at the C-terminus of one or more peptides of the construct).

113. The construct of any of aspects 106-112, wherein each polypeptide of the construct comprises a TSB located N-terminal to an immunoglobulin heavy chain constant region aa sequence (e.g., at the N-terminus of each polypeptide of the construct).

114. The construct of any of aspects 106-112, wherein each polypeptide of the construct comprises a TSB located C-terminal to an immunoglobulin heavy chain constant region aa sequence (e.g., at the C-terminus of each polypeptide of the construct).

115. The construct of any of aspects 106-110, wherein the construct has a structure selected from FIG. 5D, structures B-F, H and I, and the one or more TSBs are at position 1 and/or position 2 of the structures.

116. The construct of any of aspects 106-110, wherein the construct has a structure selected from FIG. 5D. structures B-F, H and I, and the one or more TSBs are at position 3 and/or position 4 of the structures.

117. The construct of any of aspects 106-110, wherein the construct has a structure selected from FIG. 5D, structures B-F, H and I, the one or more TSBs comprises a first and second TSB, and the first TSB is located at position 1 or 2 and the second TSB is located at position 3 or 4.

118. The construct of any of aspects 115-117, wherein each TSB present is directed to the same epitope, or there are at least two TSBs present in the construct directed to non-identical TAA epitopes.

119. The construct of any of aspects 89-96, wherein the construct comprises:
A (i) a first polypeptide comprising in the N-terminal to C-terminal direction an NBD aa sequence, a first immunoglobulin heavy chain constant region aa sequence (e.g., comprising an Ig CH2-CH3 aa sequence) and a TSB aa sequence optionally joined by linker aa sequences that are selected independently, and
   (ii) a second polypeptide comprising a second immunoglobulin heavy chain constant region aa sequence (e.g., comprising an Ig CH2-CH3 aa sequence); or
B (i) a first polypeptide comprising in the N-terminal to C-terminal direction an NBD aa sequence, a first immunoglobulin heavy chain constant region aa sequence (e.g., comprising an Ig CH2-CH3 aa sequence), and a TSB aa sequence optionally joined by linker aa sequences that are selected independently, and
   (ii) a second polypeptide comprising a second immunoglobulin heavy chain constant region aa sequence (e.g., comprising an Ig CH2-CH3 aa sequence) and a TSB aa sequence;
wherein the heavy chain constant region aa sequences of the first and second polypeptides are an interspecific pair of scaffold polypeptides optionally linked by one or more interpeptide disulfide bonds, and each NBD is optionally capped.

120. The construct of aspect 97, wherein the construct comprises:
A (i) a first polypeptide comprising in the N-terminal to C-terminal direction a first NBD aa sequence, a first immunoglobulin heavy chain constant region aa sequence (e.g., comprising an Ig CH2-CH3 aa sequence), and a TSB aa sequence optionally joined by linker aa sequence(s) that are selected independently, and
   (ii) a second polypeptide comprising in the N-terminal to C-terminal direction a second NBD aa sequence, and a second immunoglobulin heavy chain constant region aa sequence (e.g., comprising an Ig CH2-CH3 aa sequence), optionally joined by linker aa sequence(s) that are selected independently; or
B (i) a first polypeptide comprising in the N-terminal to C-terminal direction an NBD, a first immunoglobulin heavy chain constant region aa sequence (e.g., comprising an Ig CH2-CH3 aa sequence), and a TSB aa sequence, optionally joined by linker aa sequences that are selected independently, and
   (ii) a second polypeptide comprising in the N-terminal to C-terminal direction a second NBD aa sequence, a second immunoglobulin heavy chain constant region aa sequence (e.g., comprising an Ig CH2-CH3 aa sequence), and a TSB aa sequence;
wherein the heavy chain constant region aa sequences of the first and second polypeptides are an interspecific pair of scaffold polypeptides optionally linked by one or more interpeptide disulfide bonds, and each NBD is optionally capped.

121. The construct of any of aspects 119-120, wherein the first and second immunoglobulin heavy chain constant region aa sequences are a heterodimerizing interspecific pair of sequences selected from the group consisting of: KiH, KiHs-s, HA-TF, ZW1, 7.8.60, DD-KK, EW-RVT, EW-RVTs-s, and A107 aa sequences.

122. The construct of aspect 121, wherein the immunoglobulin heavy chain constant region aa sequences are a KiH or KiHs-s aa sequence pair. (e.g., wherein the first immunoglobulin heavy chain constant region aa sequence comprises the knob and the second immunoglobulin heavy chain constant region aa sequence comprises the hole).

123. The construct of any of aspects 119-122 wherein one or more independently selected TSBs binds a TAA selected from a mucin (e.g., mucin1, 16 or 18), mesothelial (MSLN), EpCAM, CTLA-4, VISTA, TIM-3, PD-L1, CTLA-4, and LAG-3 protein.

124. The construct of any of aspects 119-123, wherein the independently selected TSBs comprise independently selected antibody, Fab, Fab′, single chain antibody, scFv, polypeptide aptamer, or nanobody aa sequences.

125. The construct of any of aspects 119-123, wherein the independently selected TSBs comprise independently selected scFv, polypeptide aptamer, or nanobody aa sequences.

It is understood that the NBDs present in any (e.g., all) of constructs of aspects 1-125 may be ATP hydrolysis deficient, Further more any of the NBD in the constructs of any of aspects 1-125, may be capped on their N-terminus by a CAP that comprises both the coupling helix of TAP1 and the coupling helix of TAP2 joined by an intra-CAP linker and/or a stabilizing disulfide bond.

126. A composition comprising:
A) independently selected first and second constructs of any of aspects 1-38, or;
B) (i) a first construct of any one of aspects 1-38, and
   (ii) a second construct of any one of aspects 39-125, wherein one of the first and second constructs comprises a TAP1 NBD aa sequence and the other of the first and second constructs comprises a TAP2 NBD aa sequence;
wherein one or both of the first and second constructs optionally comprises one or more payloads and/or labels; and
wherein, when the composition further comprises ATP (e.g., one or two molecules of ATP per molecule of construct), at least a portion of the first and second constructs may form a complex (e.g., a homodimer, a heterodimer, or higher order complex) comprising one or more (e.g., two or more) ATP molecules.

127. The composition of aspect 126, wherein the NBD aa sequences of the first and second constructs comprise one or more independently selected TAP1 and/or one or more independently selected TAP2 NBD aa sequences.

128. The composition of any of aspects 126-127, wherein: (i) the complex is a homodimer and the first and second constructs each comprise a TAP1 NBD aa sequence or a TAP2 NBD aa sequence; or (ii) the complex is a heterodimer in which the first construct comprises a TAP1 NBD aa sequence, and the second construct comprises a TAP2 NBD aa sequence.

129. The composition of aspect 128, wherein the complex comprises:
A) (i) independently selected first and second constructs of any of aspects 1-5, or
   (ii) independently selected first and second constructs of any of aspects 6-38; or
B) (i) a first construct of any of aspects 1-5 and a second construct of any of aspects 6-38, wherein one of the first and second constructs comprises a TAP1 NBD aa sequence and the other of the first and second constructs comprises a TAP2 NBD aa sequence.

130. A composition comprising independently selected first and second constructs of any of aspects 39-80, wherein, when the composition further comprises ATP (e.g., one or two molecules of ATP per molecule of construct), at least a portion of the first and second constructs may form a complex (e.g., a homodimer, a heterodimer, or higher order complex) comprising one or more (e.g., two or more) ATP molecules.

131. The composition of aspect 130, wherein the NBD aa sequences of the first and second constructs comprise one or more independently selected TAP1 and/or one or more independently selected TAP2 NBD aa sequences.

132. The composition of any of aspects 130-131, wherein: (i) the complex is a homodimer and the first and second constructs each comprise a TAP1 NBD aa sequence or a TAP2 NBD aa sequence; or (ii) the complex is a heterodimer in which the first construct comprises a TAP1 NBD aa sequence, and the second construct comprises a TAP2 NBD aa sequence.

133. The composition of aspect 132, wherein the complex comprises:
(i) independently selected first and second constructs of any of aspects 39-46;
(ii) independently selected first and second constructs of any of aspects 47-80, or
(iii) a first construct of any of aspects 39-46 and a second construct of any of aspects 47-80, wherein one of the first and second constructs comprises a TAP1 NBD aa sequence and the other of the first and second constructs comprises a TAP2 NBD aa sequence.

134. A composition comprising independently selected first and second constructs of any of aspects 81-125, wherein, when the composition further comprises ATP (e.g., one or two molecules of ATP per molecule of construct), at least a portion of the first and second constructs may form a complex (e.g., a homodimer, a heterodimer, or higher order complex) comprising one or more (e.g., two or more) ATP molecules.

135. The composition of aspect 134, wherein the NBD aa sequences of the first and second constructs comprise one or more independently selected TAP1 and/or one or more independently selected TAP2 NBD aa sequences, and the complex optionally comprises at least one or at least two ATP molecules.

136. A composition of any of aspects 134-135, wherein: (i) the complex is a homodimer and the first and second constructs each comprise a TAP1 NBD aa sequence or a TAP2 NBD aa sequence; or (ii) the complex is a heterodimer in which the first construct comprises a TAP1 NBD aa sequence, and the second construct comprises a TAP2 NBD aa sequence.

137. The composition of aspect 136, wherein the complex comprises:
(i) independently selected first and second constructs of any of aspects 81-88;
(ii) independently selected first and second constructs of any of aspects 89-125, or
(iii) a first construct of any of aspects 81-88 and a second construct of any of aspects 89-125, wherein one of the first and second constructs comprises a TAP1 NBD aa sequence and the other of the first and second constructs comprises a TAP2 NBD aa sequence.

138. A composition comprising:
(i) an independently selected first construct of any of aspects 39-46, and
(ii) an independently selected second construct of any of aspects 81-88;
wherein, when the composition further comprises ATP (e.g., one or two molecules of ATP per molecule of construct), at least a portion of the first and second constructs may form a complex (e.g., a heterodimer or higher order complex) comprising one or more (e.g., two or more) ATP molecules.

139. A composition comprising:
(i) an independently selected first construct of any of aspects 39-46, and
(ii) an independently selected second construct of any of aspects 89-125;
wherein, when the composition further comprises ATP (e.g., one or two molecules of ATP per molecule of construct), at least a portion of the first and second constructs may form a complex (e.g., a heterodimer or higher order complex) comprising one or more (e.g., two or more) ATP molecules.

140. A composition comprising:
A (i) an independently selected first construct of any of aspects 47-80, and
   (ii) an independently selected second construct of any of aspects 81-88; or
B (i) an independently selected first construct of any of aspects 47-80, and
   (ii) an independently selected second construct of any of aspects 89-125;
wherein, when the composition further comprises ATP (e.g., one or two molecules of ATP per molecule of construct), at least a portion of the first and second constructs may form a complex (e.g., a heterodimer or higher order complex) comprising one or more (e.g., two or more) ATP molecules.

141. A construct of any one of aspects 1-125 further comprising one or more independently selected payloads and/or one or more independently selected label (e.g., a construct, or one or more of a polypeptides of a construct, comprise one or more labels).

142. A composition according to any one of aspects 126-140, wherein at least one construct or at least one polypeptide thereof comprises one or more independently selected payloads and/or one or more independently selected labels (e.g. the first construct and/or the second construct comprises one or more payloads and/or labels).

143. The construct or composition according to any of aspects 141-142, wherein the one or more independently selected payloads and/or one or more independently selected labels are independently selected from the group consisting of: (i) radiolabels; (ii) radio-opaque labels and other contrast agents, (iii) optical labels, (iv) photoacoustic labels, (v) MRI/NMR labels or contrast agents, (vi) SPECT labels, (vii) positron emission tomography (PET) labels, (ix) paramagnetic labels, (x) radiosensitizers, (xi) photosensitizers, (xii) therapeutic, and (xiii) chemotherapeutic or cytotoxic agents.

144. The construct or composition of any of aspects 141-143, wherein the one or more independently selected payloads and/or labels comprises one or more independently selected radioactive labels and/or payloads.

145. The construct or composition of aspect 144, wherein the one or more independently selected radioactive labels and/or payloads comprises a radionuclide of nitrogen, oxygen, sulfur, and/or hydrogen (e.g., that is incorporated into an amino acid of the construct), or a radionuclide of phosphorous or iodine that is added to the construct (e.g., as a post translational modification).

146. The construct or composition of any one of aspects 141-142, wherein at least one construct or at least one polypeptide thereof comprises:
(i) a chelating group, and the one or more payloads or labels comprises a chelated radionuclide (e.g., chelated metallic radionuclide); and/or
(ii) a radiolabeled moiety (chemical group).

147. The construct or composition of any of aspects 141-143, wherein the one or more payloads or labels comprises one or more radiosensitizers and/or photosensitizers.

148. The construct or composition of any of aspects 141-143, wherein the one or more payloads or labels comprises one or more independently selected therapeutic or chemotherapeutic agents.

149. The construct or composition of aspect 148 wherein the therapeutic or chemotherapeutic agents comprise one or more: microtubule inhibitors (e.g., monomethyl auristatin E (MMAE), monomethyl auristatin F (MMAF), mertansine (also called DM1), and emtansine); DNA binding agents (e.g., calicheamicin); topoisomerase inhibitors (e.g., deruxtecan, SN-38, and exatecan), siRNAs, and cytotoxic agents (e.g., ravtansine (DM4) and pyrrolobenzodiazepine (PBD).

150. The construct or composition of any of aspects 141-149, wherein the one or more payloads or labels is linked to the construct or a polypeptide thereof by a linker.

151. The construct or composition of aspect 150, wherein the linker is cleavable (e.g., in a target TME such as by a protease expressed at higher levels in the TME than in a peritumor tissue).

152. The construct or complex of any of aspects 1-151, wherein each NBD aa sequence comprises an independently selected NBD aa sequence of an ATP-binding cassette (ABC) transporter protein that is optionally capped their N-terminus by a CAP that comprises both the coupling helix of TAP1 and the coupling helix of TAP2 joined by an intra-CAP linker and/or a stabilizing disulfide bond.

153. The construct or composition of aspect 152, wherein the ABC transporter proteins are selected from the group consisting of: ABC1, MDR/TAP, MRP, ALD, OABP, GCN20, and White ABC transporter family members (e.g., human family members).

154. The construct or composition of aspect 152, wherein each independently selected NBD aa sequence comprises an aa sequence independently selected from the group consisting of the NBDs of: ABCA1 (SEQ ID NO: 1), ABCB1 (SEQ ID NO:2), ABCC1 (SEQ ID NO:3), ABCD1 (SEQ ID NO:4), ABCE1 (SEQ ID NO:5), ABCF1 (SEQ ID NO:6), ABCG2 (SEQ ID NO:7), ABCG5 (SEQ ID NOs: 287, 295, or 296), ABCG8 SEQ ID NOs: 289, 290, 297, and 298), TAP1 (SEQ ID NO:9) and TAP2 (SEQ ID NOs: 21, 22), or a sequence having greater than 90% sequence identity to any one of those sequences (see, e.g., FIG. 6).

155. The construct or complex of aspect 154, wherein each independently selected NBD aa sequence comprises an aa sequence having greater than 95% or greater than 98% sequence identity to the NBD of an ABC transporter protein of one of SEQ ID NOs: 1-7, 9, 21, 22, 291-291, ABCG5 (SEQ ID NOs: 286, 287, 295, and 296), and ABCG8 SEQ ID NOs: 289, 290, 297, and 298).

156. The construct or composition of any of aspects 152-155, wherein each NBD comprises a TAP1 and/or TAP2 NBD aa sequence.

157. The construct or composition of any of aspects 152-156, wherein each NBD comprises a TAP1 NBD aa sequence.

158. The construct or composition of aspect 157, wherein each NBD aa sequence comprises an independently selected TAP1 NBD aa sequence selected from the group consisting of SEQ ID NOs: 9, 12-16, 19, 20, and 291-294 or a sequence having greater than 90% aa sequence identity thereto.

159. The construct or composition of aspect 158, wherein each NBD aa sequence comprises an independently selected TAP1 NBD aa sequence having greater than 95% or 98% aa sequence identity to a sequence selected from SEQ ID NOs: 9, 12-16, 19, 20, and 291-294.

160. The construct or composition of aspect 158, wherein each NBD comprises the TAP1 NBD aa sequence of SEQ ID NO: 292 or SEQ ID NO:294, or a sequence having greater than 90% aa sequence identity to SEQ ID NO:292 or SEQ ID NO 294.

161. The construct or composition of aspect 160, wherein each NBD comprises an aa sequence having greater than 95% or 98% aa sequence identity to SEQ ID NO:292 or SEQ ID NO 294.

162. The construct or composition of any of aspects 156-161, wherein each NBD is capable of homodimerization in the presence of ATP.

163. The construct or composition of aspect 162, wherein one or more (e.g., each) NBD aa sequences comprises a TAP1 NBD aa sequence comprising a modified TAP1 D-helix comprising the sequence GNQLRVQRLL (SEQ ID NO: 26).

164. The construct or composition of any of aspects 156-163, wherein each NBD located at the N-terminus of a construct or polypeptide of a construct optionally comprises a CAP (e.g., located N-terminal to the NBD and joined to it by an independently selected linker).

165. The construct or composition of any of aspects 156-164, wherein each NBD located at the N-terminus of a polypeptide comprises a TAP1 aa sequence and a CAP comprising the coupling helix of TAP1 (SEQ ID NO:17) and the coupling helix of TAP2 (SEQ ID NO: 18) joined together by an intra-CAP linker.

166. The construct or composition of any of aspects 156-165, wherein one or more (e.g., each) NBD comprises a TAP1 aa sequence and further comprises a stabilizing disulfide bond formed between a cysteine in the NBD aa sequence and a cysteine substituted in the NBD aa sequence (e.g., a C735: R721C, or C662: Q580C disulfide bond) 167. The construct or composition of any of aspects 156-166, wherein, when one or more (e.g., each) NBDs comprise a TAP1 aa sequence, one or more (e.g. each) TAP1 aa sequence(s) further comprises a stabilizing disulfide bond formed between two cysteines substituted in the NBD aa sequence (e.g., an L493C: E573C disulfide bond).

168. The construct or composition of any of aspects 156-167, wherein, when any one or more (e.g., each) NBD comprises a TAP1 NBD aa sequence, the TAP1 NBD aa sequence comprises a C-terminal deletion comprising, consisting essentially of, or consisting of the C-terminal 5, 6 or 7 aas.

169. The construct or composition of aspect 168, wherein the NBD comprises a C-terminal deletion of the C-terminal 6 aas (i.e. the aa sequence PADAPE (SEQ ID NO:11) see SEQ ID NO:8) of TAP1.

170. The construct or composition of aspect 168, wherein the NBD comprises a C-terminal deletion of the C-terminal 7 aas (i.e. of SEQ ID NO:08) of TAP1.

171. The construct or composition of any of aspects 157-170, wherein, when one or more NBDs present in the construct comprise a TAP1 NBD aa sequence, position 668 (D668) of any one or more (e.g. each) TAP1 NBD aa sequences comprises one or more substitutions (e.g., a D668N, D668A, or D668Q substitution in TAP1) that render the construct ATP hydrolysis deficient or substantially ATP hydrolysis deficient (e.g., when complexed with a second NBD through ATP mediated dimerization.

172. The construct or composition of any of aspects 156-171, wherein, when one or more NBDs comprise a TAP1 aa sequence, position 668 (D668) of any one or more (e.g. each) TAP1 aa sequence is any amino acid other than D or N; or D668 is substituted by A (D668A).

173. The construct or composition of any of aspects 156-172, wherein, when any one or more NBDs comprise a TAP1 NBD aa sequence, the TAP1 NBD aa sequence comprises an S542 substitution with an aa other than serine or threonine (e.g., substituted with an alanine to give an S542A substitution).

174. The construct or composition of any of aspects 153-155, wherein the construct comprises an ABCG5 or ABCG8 NBD and the composition comprises at least one construct comprising an ABCG5 NBD aa sequence and at least one construct comprising an ABCG8 NBD aa sequence.

175. The construct or composition of any of aspects 1-174, wherein, when one or more independently selected ICBs are present in the construct or a construct of a composition, at least one ICB (e.g., at least two ICBs, or each ICB) aa sequence has/have affinity for (e.g., binds to) a protein selected from the group consisting of: CD3, CD2, CD4, CD8, CD13, CD16, CD25, CD28, CD33, CD34, CD66, CD68, CD84, CD137/4-1BB, CD163, CD193, CD206, CXCR1, DR5, FcεR1α, αβTCR, TCRα chain, TCRβ chain, δγ TCR, TCR γ chain, TCR δ chain, and TRGV9.

176. The construct or composition of aspect 175, wherein the at least one ICB comprises at least one aa sequence with affinity for CD3, CD8 or CD16 177. The construct or composition of aspect 175, wherein the at least one ICB comprises at least one aa sequence with affinity for CD3.

178. The construct or composition of any of aspects 175-177, wherein the at least one (e.g., at least two, or each) ICB comprises an scFv, VHH (nanobody), or an anti-human CD3 aa sequence comprising the sequence of any of SEQ ID NOs: 72-79, or a sequence having greater than about 90% or greater than about 95% sequence identity to any of SEQ ID NOs: 72-79 (e.g., an aa sequence having greater than 96% or greater than 98% sequence identity to any of SEQ ID NOs: 72-79).

179. The construct or composition of any of aspects 175-176, wherein the at least one ICB (e.g., at least two, or each) comprises an scFv, VHH (nanobody), or an anti-human CD16 aa sequence comprising the sequence of any of SEQ ID NOs: 80-81, or a sequence having greater than about 90% or greater than about 95% sequence identity to any of SEQ ID NOs: 80-81 (e.g., an aa sequence having greater than 96% or greater than 98% sequence identity to any of SEQ ID NOs: 80-81).

180. The construct or composition of any of aspects 175-179, wherein one or more (each) of the independently selected ICB aa sequences is an antibody, antigen binding fragment(s) of an antibody, Fab, Fab′, scFv, aptamer, or nanobody aa sequence.

181. The construct or composition of any of aspects 1-180, wherein, when one or more ADs are present, at least one (e.g., at least two ADs, or each AD) comprises an independently selected aa sequence of all or part (e.g., comprising all or part of an extracellular domain aa sequence) of a protein selected from the group consisting of: IL-2, IL-7, IL-10, IL-12, IL-15, anti-CD3, anti-CD16, anti-CD28, CD40L, B7 superfamily members, CD80, CD86, anti-CTLA4 IFN-γ, TNF superfamily members, TNFα, lymphotoxin α, lymphotoxin αβ, BAFF (CD257), PD-L1, anti-PDL1, PDL2, anti-PDL2, PD1, and anti-PD1 aa sequences.

182. The construct or composition of any of aspects 1-180, wherein, when one or more independently selected ADs are present, at least one AD (e.g., at least two ADs, or each AD) comprises an aa sequence of all or part (e.g., all or part of an extracellular domain) of a protein selected from the group consisting of: IL-2, IL-7, IL-10, 4-1BBL, anti-CD3, anti-CD16, anti-CD28, CD40L, CD80, CD86, anti-CTLA4, IFN-γ, TNFα, lymphotoxin α, lymphotoxin αβ, BAFF (CD257), PD-L1, anti-PDL1, PDL2, anti-PDL2, PD1, and anti-PD1 aa sequences.

183. The construct or of aspect 181, wherein the at least one AD (e.g., at least two ADs, each AD) comprises an aa sequence having greater than about 90% or greater than about 95% sequence identity to IL-15, IL-12, or IL-7 (e.g., greater than 97% or greater than 99% sequence identity to IL-15, IL-12, or IL-7) aa sequences.

184. The construct or composition of any preceding aspect, wherein, when one or more ADs are present, at least one AD (e.g., at least two ADs, each AD) comprises the (i) single chain IL-12 of SEQ ID NO: 109, or (ii) the IL-12 p40 subunit of SEQ ID NO: 110 and/or the p35 subunit of SEQ ID NO: 111, or an aa sequence having greater than 95% or greater than 98% sequence identity to any of those IL-12 aa sequences.

185. The construct or composition of any preceding aspect, wherein, when one or more ADs are present, at least one AD (e.g., at least two ADs, each AD) comprises an IL-2 aa sequence of SEQ ID NO: 106, or an aa sequence having at least 90% or 95% aa sequence identity to SEQ ID NO: 106.

186. The construct or composition of aspect 185, wherein the IL-2 aa sequence has at least 96% or at least 98% sequence identity to at least 170 or 180 contiguous aas of SEQ ID NO: 106.

187. The construct or composition of aspect 185 or 186, wherein the IL-2 sequence comprises substitutions at H16 and/or F42 (e.g., either H16 and/or F42 may also be replaced by independently selected Ala or Thr).

188. The construct or composition of any preceding aspect, wherein, when one or more ADs are present, at least one AD (e.g., at least two ADs, or each AD) comprises an aa sequence that binds to PD1 (e.g., an anti-PD1 such as an scFv or nanobody aa sequence that may act as an agonist of PD1) or an aa sequence that binds to PDL1 (e.g., an anti-PDL1 scFv or nanobody aa sequence).

189. The construct or composition of any preceding aspect, wherein, when one or more ADs are present, at least one AD (e.g., at least two ADs, each AD) comprises (i) an aa sequence that binds to CD40 (e.g., an anti-CD40 scFv or nanobody aa sequence that may act as an agonist of CD40 signaling) or (ii) an aa sequence that binds to CD40L (e.g., an anti-CD40L scFv or nanobody aa sequence).

190. The construct or composition of any of any preceding aspect, wherein, when one or more ADs are present, at least one AD (e.g., at least two ADs, each AD) comprises the CD40L aa sequence of SEQ ID NO:83, the CD40L trimer aa sequence of SEQ ID NO: 113, or an aa sequence having at least 90% or at least 95% sequence identity to either SEQ ID NO:83 or 113 (e.g., greater than 97% or greater than 99% sequence identity to either SEQ ID NO:83 or 113).

191. The construct or composition of any preceding aspect, wherein, when one or more ADs are present, at least one AD (e.g., at least two ADs, each AD) comprises an aa sequence that binds to CD137/4-1BB (e.g., such as an anti-CD137 scFv or nanobody aa sequence).

192. The construct or composition of any preceding aspect, wherein, when one or more ADs are present, at least one AD (e.g., at least two ADs, each AD) comprises the 4-1BBL sequence of SEQ ID NO: 114 or 115, the 4-1BBL trimer aa sequence of SEQ ID NO:116, or an aa sequence having at least 90% or at least 95% sequence identity to one of SEQ ID NOs: 114-116 (e.g., greater than 97% or greater than 99% sequence identity to one of SEQ ID NOs: 114-116).

193. The construct or composition of any preceding aspect, wherein, when one or more ADs are present, at least one AD (e.g., at least two ADs, each AD) comprises an aa sequence that binds to IL-10 (e.g., an anti-IL-10 scFv or nanobody aa sequence) or an aa sequence that binds to the IL-10 receptor (“IL-10R”) (e.g., an anti-IL-10R scFv or nanobody aa sequence).

194. The construct or composition of any preceding aspect, wherein, when one or more ADs are present, at least one AD (e.g., at least two ADs, each AD) comprises an aa sequence that binds to CTLA-4 (e.g., an anti-CTLA-4 scFv or nanobody aa sequence).

195. The construct or composition of any preceding aspect, wherein, when one or more ADs are present, at least one AD (e.g., at least two ADs, each AD) comprises an aa sequence that binds to CD3 (e.g., an anti-CD3 scFv or nanobody aa sequence).

196. The construct or composition of any preceding aspect, wherein, when one or more ADs are present, at least one AD (e.g., at least two ADs, each AD) comprises an aa sequence that binds to CD28 (e.g., an anti-CD28 scFv or nanobody aa sequence).

197. The construct or composition of any preceding aspect, wherein, when one or more ADs are present, at least one AD (e.g., at least two ADs, each AD) comprises the anti-CD28 scFv sequence of SEQ ID NO:117, or an aa sequence having at least 90% or at least 95% sequence identity to SEQ ID NO:117 (e.g., greater than 96% or greater than 98% sequence identity to to at least 220 or at least 230 contiguous aas of SEQ ID NO:117).

198. The construct or composition of any preceding aspect, wherein, when one or more ADs are present, at least one AD (e.g., at least two ADs, each AD) comprises an aa sequence that binds to IFN-γ receptor (e.g., an anti-IFN-γ receptor scFv or nanobody aa sequence).

199. The construct or composition of any preceding aspect, wherein, when one or more ADs are present, at least one AD (e.g., at least two ADs, each AD) comprises the single chain IFN-γ polypeptide of SEQ ID NO:82, or an aa sequence having at least 90% or at least 95% sequence identity to SEQ ID NO:82 (e.g., greater than 96% or greater than 99% sequence identity to SEQ ID NO:82).

200. The construct or composition of any preceding aspect, wherein, when one or more ADs are present, at least one AD (e.g., at least two ADs, each AD) comprises an aa sequence that binds to CD16 (e.g., an anti-CD16 scFv or nanobody aa sequence).

201. The construct or composition of any preceding aspect, wherein, when one or more ADs are present, at least one AD (e.g., at least two ADs, each AD) comprises the anti-CD16 VHH polypeptide of SEQ ID NO:80 or 81, or an aa sequence having at least 90% or at least 95% sequence identity to either SEQ ID NO:80 or 81 (e.g., greater than 96% or greater than 99% sequence identity to either SEQ ID NO:80 or 81).

202. The construct or composition of any preceding aspect, wherein, when one or more ADs are present, at least one AD (e.g., at least two ADs, each AD) comprises the CD80 aa sequence of SEQ ID NO: 106, or an aa sequence having at least 90% or at least 95% sequence identity to SEQ ID NO: 106 (e.g., greater than 96% or greater than 99% sequence identity to SEQ ID NO:106).

203. The construct or composition of any preceding aspect, wherein, when one or more ADs are present, at least one AD (e.g., at least two ADs, each AD) comprises the CD86 aa sequence of either SEQ ID NO:107 or 108, or an aa sequence having at least 90% or at least 95% sequence identity to either SEQ ID NO: 107 or 108 (e.g., greater than 96% or greater than 99% sequence identity to either SEQ ID NO:107 or 108).

204. The construct or composition of any preceding aspect, wherein when one or more ADs are present, at least one AD (e.g., at least two ADs, each AD) comprises a nucleic acid with CpG repeats (e.g., a CpG oligodeoxynucleotide sequence) or a nucleic acid comprising the sequence TCATCATTTTGTCATTTTGTCATT (SEQ ID NO:87) optionally having 1, 2 or 3 nucleotide substitutions, deletions, or insertions (e.g., greater than 90% or 95% sequence identity).

205. The construct or composition of any preceding aspect, wherein, when one or more TSBs (e.g., two or more, or each TSB) are present in the construct or complex, at least one TSB (e.g., two or more, or each TSB) is independently selected to display affinity for (bind to) a TAA selected from the group consisting of: carcinoembryonic antigen (CEA), mesothelin (MSLN), Erb-B2 Receptor Tyrosine Kinase 2 (HER2 or ERBB2), Epithelial Cell Adhesion Molecule (EPCAM), Vascular Endothelial Growth Factor Receptor (VEGF), Six transmembrane epithelial antigen of the prostate (STEAP), Epidermal Growth Factor Receptor (EGFR), Glypican-3 (GPC3), mucin 17 (MUC17), Prostate-Specific Membrane Antigen (PSMA), mucin 1 (MUC1), mucin 16 (MUC16), trophoblast cell surface antigen (TROP2), fibroblast growth factor receptor 2 (FGFR2b), claudin 6 (CLDN6), CD276 (B7-H3), carbonic anhydrase (CA9), podoplanin (PDPN), alkaline phosphatase, placental-like (ALPP, e.g., ALPPL2), Anthrax toxin receptor 1 (ANTXR1), claudin 18 (CLDN18), folate hydrolase-1 (FOLH1), guanylyl cyclase C (GUCY2C), interleukin-13 receptor 13 subunit alpha-2 (IL13RA2), Podocalyxin (PODXL), prostate stem cell antigen (PSCA), protein Tyrosine Kinase 7 (PTK7), Folate receptor 1 (FOLR1), V-domain Ig suppressor of T cell activation (VISTA), T-cell immunoglobulin and mucin-domain containing-3 (TIM-3), PD-L1, CTLA-4, tissue factor (human), c-Met tyrosine kinase, CD22, CD79b, CD19, CD30, folate receptor alpha (FRα), Nectin-4, B7H3, cMET, and lymphocyte-activation gene 3 (LAG-3).

206. The construct or composition of aspect 205, wherein the at least one TSB (e.g., at least two TSBs, or each TSB) comprises an anti-human mesothelin scFv of SEQ ID NO:88 or an aa sequence having greater than about 90% or greater than about 95% sequence identity to SEQ ID NO:88 (e.g., greater than 96% or greater than 98% sequence identity to SEQ ID NO:88).

207. The construct or composition of any of aspects 205-206, wherein the at least one TSB (e.g., at least two TSBs, or each TSB) comprises an anti-human mesothelin scFv (vH-vL) of SEQ ID NO:89 or an aa sequence having greater than about 90% or greater than about 95% sequence identity to SEQ ID NO:89 (e.g., greater than 96% or greater than 98% sequence identity to SEQ ID NO:89).

208. The construct or composition of any one of aspects 205-207, wherein the at least one TSB (e.g., at least two TSBs, or each TSB) comprises an anti-human mesothelin scFv (vL-vH) of SEQ ID NO:90 or an aa sequence having greater than about 90% or greater than about 95% sequence identity to SEQ ID NO:90 (e.g., greater than 96% or greater than 98% sequence identity to SEQ ID NO:90).

209. The construct or composition of any one of aspects 205-208, wherein the at least one TSB (e.g., at least two TSBs, or each TSB) comprises an anti-human mesothelin scFv (vH-vL) of SEQ ID NO:91 or an aa sequence having greater than about 90% or greater than about 95% sequence identity to SEQ ID NO:91 (e.g., greater than 96% or greater than 98% sequence identity to SEQ ID NO:91).

210. The construct or composition of any one of aspects 205-209, wherein the at least one TSB (e.g., at least two TSBs, or each TSB) comprises an anti-human mesothelin scFv (vL-vH) of any of SEQ ID NOs: 92-96 or an aa sequence having greater than about 90% or greater than about 95% sequence identity to any of SEQ ID NOs: 92-96.

211. The construct or composition of aspect 210, wherein the anti-human mesothelin scFv (vL-vH) has greater than 96% or greater than 98% sequence identity to any of SEQ ID NOs: 92-96.

212. The construct or composition of any one of aspects 205-211, wherein the at least one TSB (e.g., at least two TSBs, or each TSB) comprises an anti-human mesothelin VHH (nanobody) of SEQ ID NO:97, or an aa sequence having greater than about 90% or greater than about 95% sequence identity to SEQ ID NO:97 (e.g., greater than 96% or greater than 98% sequence identity to SEQ ID NO:97).

213. The construct or composition of any one of aspects 205-212, wherein the at least one TSB (e.g., at least two TSBs, or each TSB) comprises an anti-human EpCAM scFv (vH-vL) of SEQ ID NO:98 or an aa sequence having greater than about 90% or greater than about 95% sequence identity to SEQ ID NO:98 (e.g., greater than 96% or greater than 98% sequence identity to SEQ ID NO:98).

214. The construct or composition of any one of aspects 205-213, wherein the at least one TSB (e.g., at least two TSBs, or each TSB) comprises an anti-human EpCAM scFv (vL-vH) of SEQ ID NO:99 or an aa sequence having greater than about 90% or greater than about 95% sequence identity to SEQ ID NO:99 (e.g., greater than 96% or greater than 98% sequence identity to SEQ ID NO:99).

215. The construct or composition of any one of aspects 205-214, wherein the at least one TSB (e.g., at least two TSBs, or each TSB) comprises an anti-human CTLA-4 scFv (vL-vH) of any of SEQ ID NOs: 100-104 or an aa sequence having greater than about 90% or greater than about 95% sequence identity to any of SEQ ID NOs: 100-104.

216. The construct or composition of aspect 215, wherein the anti-human CTLA-4 scFv (vL-vH) has greater than 96% or greater than 98% sequence identity to any of SEQ ID NOs: 100-104.

217. The construct or composition of any of aspects 1-203, wherein, when one or more TSBs are present in the construct composition, at least one TSB (e.g., at least two TSBs, or each TSB) displays affinity for a checkpoint protein.

218. The construct or composition of aspect 217, wherein the checkpoint protein is selected from the group consisting of V-domain Ig suppressor of T cell activation (VISTA), T-cell immunoglobulin and mucin-domain containing-3 (TIM-3), PD-L1, CTLA-4, and lymphocyte-activation gene 3 (LAG-3).

219. The construct or composition of any one of aspects 1-218, wherein the EC50 of ATP for dimer or higher order complex formation is from about 0.05 μM to about 0.5 μM or from about 0.5 μM to about 5.0 μM.

220. The construct or composition of any one of aspects 1-218, wherein the EC50 of ATP for dimer or higher order complex formation is from about 5.0 μM to about 50 μM or from about 50 μM to about 250 μM.

221. A construct comprising an NBD (e.g., an ABC transporter such as TAP1) aa sequence capable of homodimerization or heterodimerization and one or more independently selected payloads and/or labels.

222. A composition comprising a first polypeptide comprising an NBD (e.g., an ABC transporter such as TAP1) aa sequence and a second polypeptide comprising an NBD aa sequence;
wherein at least one of the first and/or second polypeptides comprises one or more independently selected payloads and/or one or more independently selected labels; and
wherein, when the composition further comprises ATP (e.g., one or more or two or more ATP molecules per molecule of first and second construct total), at least a portion of the first and second polypeptides form a homodimer (if the constructs are identical) or a heterodimer (if the constructs are not identical).

223. A composition comprising a first polypeptide comprising an NBD (e.g., an ABC transporter such as TAP1) aa sequence and a second polypeptide comprising an NBD aa sequence, wherein:

the NBDs of the first and second polypeptides heterodimerize;

at least one of the first and/or second polypeptides comprises one or more independently selected payloads and/or one or more independently selected labels; and

wherein, when the composition further comprises ATP (e.g., one or more, or two or more, ATP molecules per molecule of the constructs), at least a portion of the first and second polypeptides form a heterodimer.

224. A method of eliciting a response (e.g., directly or indirectly) from CD4+ T cells, oy T cell, NK cells, neutrophils, mast cells, eosinophils, basophils macrophages, CD8+ T cells, CD4+ T regs, and/or myeloid derived suppressor cells, comprising contacting the cells with a construct or composition of any of aspects 1-220, in the presence of ATP (e.g., at a concentration sufficient for it to induce dimerization such as at least about the EC50 for ATP mediated dimerization).

225. A method of stimulating (e.g., directly, or indirectly) CD4+ T cells, oy T cell, NK cells, neutrophils, mast cells, eosinophils, basophils macrophages, and/or CD8+ T cells, comprising contacting the cells with a construct or composition of any of aspects 1-220, in the presence of sufficient ATP to bind to the NBD (e.g., at a concentration sufficient for it to induce dimerization such as at least about the EC50 for ATP mediated dimerization).

226. A method of stimulating (e.g., directly, or indirectly stimulating) CD4+ T cells, oy T cells, NK cells, and/or CD8+ T cells comprising contacting the cells with a construct or composition of any of aspects 1-220, in the presence of sufficient ATP to bind to the NBD (e.g., at a concentration sufficient for it to induce dimerization such as at least about the EC50 for ATP mediated dimerization).

227. A method of suppressing or depleting (e.g., directly, or indirectly) CD4+ T regs, and/or myeloid derived suppressor cells, the method comprising contacting the cells with a construct or composition of any of aspects 1-220, in the presence of sufficient ATP to bind to the NBD aa sequence (e.g., at a concentration sufficient for it to induce dimerization such as at least about the EC50 for ATP mediated dimerization).

228. The method of any of aspects 224-227, wherein the contacting occurs in vitro (e.g., outside of the body of a mammal or non-mammalian animal such as in isolated a tissue sample).

229. The method of any of aspects 224-227, conducted in vivo or within the body of a mammal (e.g., in a target tissue or TME of a mammalian patient).

230. The method of any of aspects 224-227, wherein the contacting occurs in a TME.

231. A method of treating a patient or subject (e.g., a patient or subject with a cancer), the method comprising administering a construct or composition (e.g. as a pharmaceutically acceptable composition comprising a construct or composition) of any one of aspects 1-223, or one or more molecules comprising one or more nucleic acid sequences encoding construct or composition of any one of aspects 1-223 to a patient or subject.

232. A method of treating a cancer in a mammalian patient or subject comprising administering a construct or composition of any one of aspects 1-223, or one or more molecules comprising one or more nucleic acid sequences encoding construct or composition of any one of aspects 1-223 to a patient or subject.

233. The method of aspect 229 or 232, wherein the mammalian patient or subject is a human.

234. The method of any one of aspects 229-233, wherein prior to said administering or contacting, the concentration of ATP is determined in a target tissue in which the cellular response is to be elicited, cells are to be stimulated or suppressed, or in the target TME.

235. The method of aspect 234, wherein the administering or contacting is conducted using a construct or composition with an EC50 for ATP induced dimerization or higher order complex formation that is less the concentration of ATP in the target tissue or TME, or less than 50% concentration of ATP in the target tissue or TME.

236. The method of aspect 234, wherein the administering or contacting is conducted using a construct or composition with an EC50 for ATP induced dimerization or higher order complex formation that is less than 20% of the concentration of ATP in the target tissue or TME, or less than 5% concentration of ATP in the target tissue or TME.

237. The method of any one of aspects 235-236, wherein the EC50 for ATP induced dimerization is greater than two times the ATP concentration in peritumoral tissue or greater than five times the ATP level in peritumoral tissue.

238. The method of any one of aspects 235-236, wherein the EC50 for ATP induced dimerization is greater than ten times the ATP concentration in peritumoral tissue or greater than twenty-five times the ATP level in peritumoral tissue.

239. The method of any one of aspects 229-233, wherein prior to said administering or contacting, the level of CD73 expression (e.g., relative to peritumoral tissue CD73c expression) by cells in a target tumor is measured as a surrogate for elevated TME ATP levels.

240. The method of any of aspects 231-239, wherein the construct or composition comprises one or more independently selected payloads and/or one or more independently selected labels.

241. The method of aspect 240, wherein the one or more independently selected payloads and/or one or more independently selected labels comprise a radionuclide.

242. The method of any of aspects 240-241, wherein the one or more one or more independently selected payloads and/or one or more independently selected labels comprises a radiosensitizer optionally administering radiation from a source external to the patient or subject.

243. The method of any of aspects 240-241, wherein the patient is scanned or imaged (e.g., by MRI, PET, CT etc.) to detect the distribution of the payload or label (e.g., to detect the location of a tumor).

244. The method of any of aspects 240-243, wherein when the one or more payloads or labels comprises a photosensitizer optionally administering light from a source external to the patient or subject.

245. The use of a construct or composition of any of aspects 1-220, for the preparation of a medicament for the in vivo contacting or stimulating of cells of any of aspects 229 or 230.

246. The use of a construct or composition of any of aspects 1-223, for the preparation of a medicament for the treatment of a cancer (e.g., a solid tumor).

247. A construct or a composition of any of aspects 1-220, for use in the in vivo contacting or stimulating of cells of any of aspects 229 or 230.

248. A construct or a composition of any of aspects 1-223, for use in the preparation of a medicament for the treatment of a cancer.

249. The construct or composition of aspects 246 or 248, wherein the cancer comprises a solid tumor.

250. The method, use, construct or composition of any of aspects 229-249, wherein the target tissue, TME, or cancer is a mesothelioma, melanoma, sarcoma, carcinoma, carcinosarcoma, lymphoma, or germ cell tumor.

251. One or more nucleic acid molecules comprising one or more nucleic acid sequences encoding a construct, or one or more polypeptides of a construct or composition of any of aspects 1-223.

252. A vector comprising the one or more nucleic acid sequences of aspect 251, optionally under the control of a promoter (e.g., a constitutive promoter) or an inducible promoter.

253. A cell comprising the vector or nucleic acid of any of aspects 251-252.

254. The cell of aspect 253, wherein the cell is a mammalian cell, yeast cell, insect cell, or bacterial cell.

255. A method of preparing one or more constructs of any of aspects 1-223, comprising culturing one or more cells of aspect 248 or 249, either individually or collectively, optionally inducing the production of the constructs when their production is under the control of an inducible promoter, collecting the culture media from the cultured one or more cells comprising the one or more constructs, and optionally purifying the one or more constructs individually or collectively (in admixture), such as by affinity chromatography and/or size exclusion chromatography.

256. A method of preparing a composition comprising one or more constructs comprising combining two or more constructs prepared as in aspect 255, optionally adding ATP to form a dimer or higher order complex comprising the two or more constructs.

257. A polypeptide comprising:
A) a human TAP1 NBD aa sequence (e.g., a TAP1 NBD of any of SEQ ID NOs: 9, 12-14, or 291-294) or an NBD having at least 90% amino acid sequence identity thereto; or
B) a human TAP1 NBD aa sequence (e.g., a TAP1 NBD of any of SEQ ID NOs: 9, 12-14, or 291-294), or an NBD having at least 90% amino acid sequence identity thereto), that comprised one or more (e.g., two or more) of:
   (i) a stabilizing disulfide bond formed between a cysteine in the NBD aa sequence and a cysteine substituted in the NBD aa sequence (e.g., a C735: R721C, or C662: Q580C disulfide bond);
   (ii) a stabilizing disulfide bond formed between two cysteines substituted in the NBD aa sequence (e.g., an L493C: E573C disulfide bond);
   (iii) a C-terminal deletion comprising, consisting essentially of, or consisting of the deletion of the C-terminal 5, 6 or 7 aas;
   (iv) a CAP located N-terminal to the NBD and joined to it by an independently selected linker;
   (v) an S542 substitution with an aa other than serine or threonine (e.g., substituted with an alanine), or
   (vi) a payload or label.

258. The polypeptide of aspect 257, wherein the stabilizing disulfide bond formed between a cysteine in the NBD aa sequence and a cysteine substituted in the NBD aa sequence comprises a C735: R721C, and/or a C662: Q580C disulfide bond.

259. The polypeptide of any of aspects 257-258, wherein the stabilizing disulfide bond formed between two cysteines substituted in the NBD aa sequence comprises an L493C: E573C disulfide bond.

260. The polypeptide of any of aspects 257-259, wherein the C-terminal deletion comprises, consists essentially of, or consists of, the deletion of the aa sequence PADAPE (SEQ ID NO: 11) from human TAP1 NBD.

261. The polypeptide of any of aspects 257-260, wherein the CAP located N-terminal to the NBD comprises the aa sequence of the coupling helix of TAP1 (SEQ ID NO: 17) and the aa sequence of the coupling helix of TAP2 (SEQ ID NO: 18) optionally joined together by an intra-CAP linker.

262. The polypeptide of aspect 261, comprising an intra-CAP linker from 1 aa to 5 aas in length or from 6 aas to 10 aas in length.

263. The polypeptide of any of aspects 257-262, comprising a substitution at S542 of the human TAP1 NBD aa sequence with an aa other than serine or threonine.

264. The polypeptide of aspect 263, wherein S542 is substituted with an alanine.

265. The polypeptide of any of aspects 257-264, wherein the TAP1 NBD aa sequence comprises an aa sequence selected from the group consisting of SEQ ID NOs: 9, 12-16, 19, 20 and 291-294, or an aa sequence having greater than 90% aa sequence identity to an aa sequence selected from the group consisting of SEQ ID NOs: 9, 12-16, 19, 20 and 291-294.

266. The polypeptide of any of aspects 257-265, wherein the TAP1 NBD aa sequence comprises an aa sequence having greater than 95% or greater than 98% aa sequence identity to an aa sequence selected from the group consisting of SEQ ID NOs: 9, 12-16, 19, 20 and 291-294.

267. The polypeptide of aspect 265, wherein the TAP1 NBD aa sequence comprises the aa sequence of SEQ ID NOs: 291-294, or an aa sequence having greater than 90% aa sequence identity to SEQ ID NOs: 291-294.

268. The polypeptide of aspect 265, wherein the TAP1 NBD aa sequence comprises an aa sequence having greater than 95% aa or greater than 98% aa sequence identity to SEQ ID NOs: 291-294.

269. The polypeptide of any of aspects 257-265, wherein the TAP1 NBD aa sequence comprises an aa sequence having greater than about 90% or greater than about 95% sequence identity to the aa sequences of SEQ ID NO: 9.

270. The polypeptide of any of aspects 257-265, wherein the TAP1 NBD aa sequence comprises an aa sequence having greater than about 90% or greater than about 95% sequence identity to any one of SEQ ID NOs: 12-15.

271. The polypeptide of any of aspects 257-265, wherein the TAP1 NBD aa sequence comprises an aa sequence having greater than about 90% or greater than about 95% sequence identity to the capped TAP1 aa sequence of SEQ ID NOs: 19, 20, or 293-294.

272. The polypeptide of any of aspects 257-265, wherein the TAP1 NBD aa sequence comprises a substitution that renders the polypeptide substantially ATP hydrolysis deficient.

273. The polypeptide of aspect 272, comprising a D668 substitution that renders the at least one (e.g., each) NBD substantially ATP hydrolysis deficient.

274. The polypeptide of aspect 273, wherein position 668 (e.g., D668 in TAP1) is an aa other than D or N, or D668 is substituted by A or Q.

275. The polypeptide of any of aspects 257-274, wherein the TAP1 NBD aa sequence is substantially non-homodimerizing in the presence of ATP.

276. The polypeptide of any of aspects 257-274, wherein the TAP1 NBD aa sequence comprises a substitution of at least one (e.g., at least two, at least three, or each) of aas N676, S677, Q680, E682, and Q683, and the protein or polypeptide is homodimerizing in the presence of ATP.

277. The polypeptide of any of aspects 257-274, wherein the TAP1 NBD aa sequence comprises a substitution of its D-helix with the rat TAP1 D-helix sequence GNQLRVQRLL (SEQ ID NO:26), and the protein or polypeptide is homodimerizing in the presence of ATP.

278. The polypeptides of any of aspects 257-277, comprising a payload or label.

279. A method of forming a dimer or higher order complex of polypeptides comprising (i) linking independently selected polypeptides comprising ATP binding cassette NBD domains that homodimerize or heterodimerize to the polypeptides to be assembled into the dimer or a higher order complex either covalently (e.g., as a fusion protein or by linking such as by a crosslinker or enzymatically) or noncovalently (e.g., by affinity means such as an antibody or antibody fragment) and (ii) contacting the polypeptides with ATP (e.g., adding ATP or introducing the polypeptides in an environment comprising ATP such as a TME).

280. A method of forming a dimer or higher order complex of polypeptides comprising (i) linking an independently selected polypeptide of any of aspects 257-278 to the polypeptides to be assembled into the dimer or a higher order complex either covalently (e.g., as a fusion protein or by linking such as by a crosslinker or enzymatically) or noncovalently (e.g., by affinity means such as an antibody or antibody fragment) and (ii) contacting the polypeptides with ATP (e.g., adding ATP or introducing the polypeptides in an environment comprising sufficient ATP such as a TME).

281. A polypeptide comprising an aa sequence of any of SEQ ID NOs: 120-173, 188-190, and 192-197, from which N-terminal methionine, if present may optionally be o deleted, and C-terminal His Tag or G4S linker-TEV protease and His tag sequences, if present may be deleted, or a sequence having greater than 95% or greater than 98% aa sequence to any thereof.

282. A polypeptide comprising an aa sequence of any of SEQ ID NOs: 155-169, from which N-terminal methionine and any His tag may be optionally deleted, or a sequence having greater than 95% or greater than 98% aa sequence to any thereof.

283. A protein or polypeptide comprising an aa sequence of any of constructs 436, 473, 476, 533, 571, 586, 587, 590, 647, and 648, or a sequence having greater than 95% or greater than 98% aa sequence to any thereof.

284. A pharmaceutical composition comprising one or more constructs or compositions of any of aspects 1-223, and one or more pharmaceutically acceptable excipients.

285. A pharmaceutical composition comprising one or more polypeptides of any of aspects 257-278, and one or more pharmaceutically acceptable excipients.

286. The pharmaceutical composition of any one of aspects 284-285, wherein the pharmaceutical composition is sterile and is free of detectable pyrogens and/or other toxins, or the detectable pyrogens and/or other toxins are below permissible limits.

### VII. EXAMPLES

### Example 1. TAP1 NBD Homodimerization in Solution

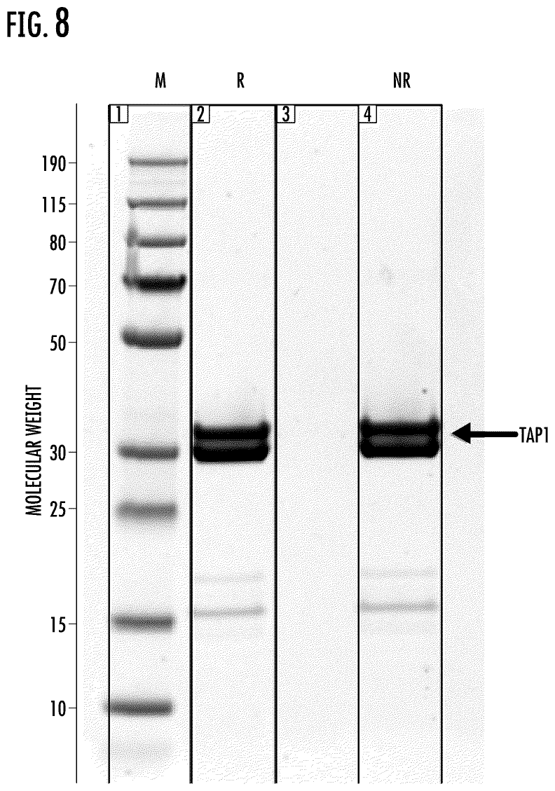

In order to test the functionality of TAP1 NBD to homodimerize in solution, a human TAP1 NBD comprising: (i) N676, S677, Q680, E682, and Q683 (specifically N676G, S677N, Q680R, E682Q, and Q683R substitutions, (ii) substitutions of cysteines present in the sequence with serines (SEQ ID NO:13), and (iii) a C-terminal His tag (construct 24) was expressed in E. coli and subsequently purified. Samples of the molecular weight markers and the purified protein were subject to SDS-PAGE gel analysis under reducing and non-reducing conditions as shown in FIG. 8 (lanes: M-molecular weight markers, R-reduced protein sample, NR non-reduced protein sample).

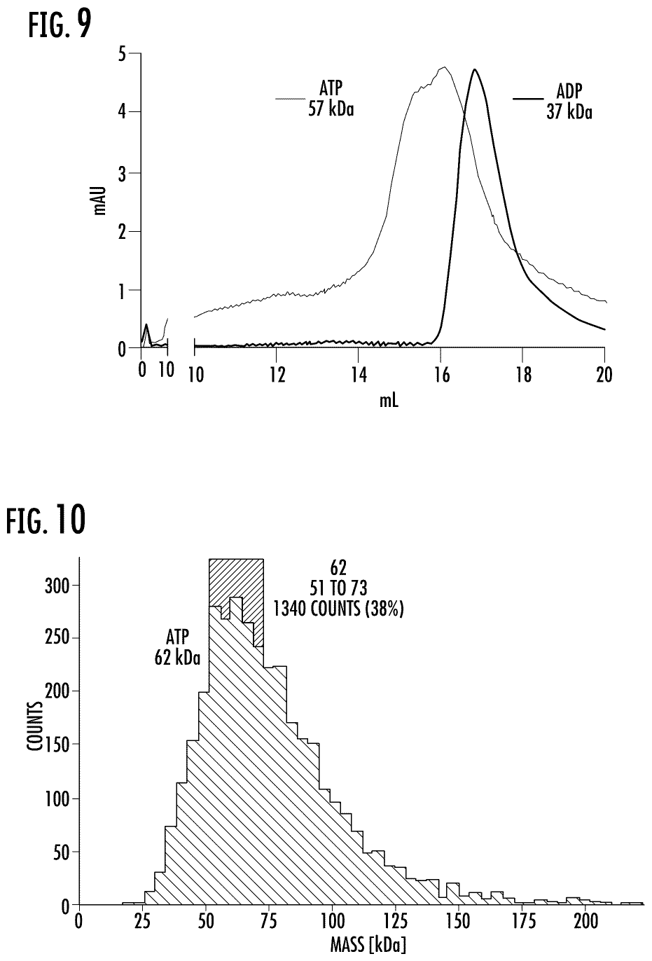

Samples of the purified TAP1 NBD (construct 24) were preincubated in 1 mM ADP or 1 mM ATP in 20 mM Tris (pH 8.0), 50 mM NaCl, 5 mM MgCl₂, 10% glycerol buffer at 4° C. and subject to size exclusion chromatography at 4° C. on a Superdex® 200 column 30 cm×10 mm, 8.6 μm particle (CYTIVA BIOPROCESS R&D AB Corporation) size in the sample's preincubation buffer. The results, shown in FIG. 9, indicate that in the presence of ADP the protein chromatographed at an effective molecular weight of 37 kDa and in the presence of ATP at an effective molecular weight of 57 kDa; indicating that the TAP1 protein dimerizes in the presence of ATP.

The dimerization was confirmed using mass photometry and dynamic light scattering (DLS) to determine molecular weight. Samples for mass photometry were incubated in the presence of 1 mM ADP or ATP for 10 minutes at 4° C. and diluted in the same buffer lacking ADP and ATP immediately prior to analysis to give a final nucleotide concentration of 50 nM. Mass photometry measurements were made on a Refeyn OneMP instrument using glass slides cleaned with isopropanol followed by distilled deionized water and sealed with silicone gaskets. Data was analyzed using the manufacturer's proprietary software calibrated against a standard curve of proteins of known mass from Thermo Fisher Scientific. The experimentally determined effective molecular weight of 62 kDa in the presence of 1 mM ATP (diluted to 50 μM ATP) was in agreement with the chromatographic result (see FIG. 10).

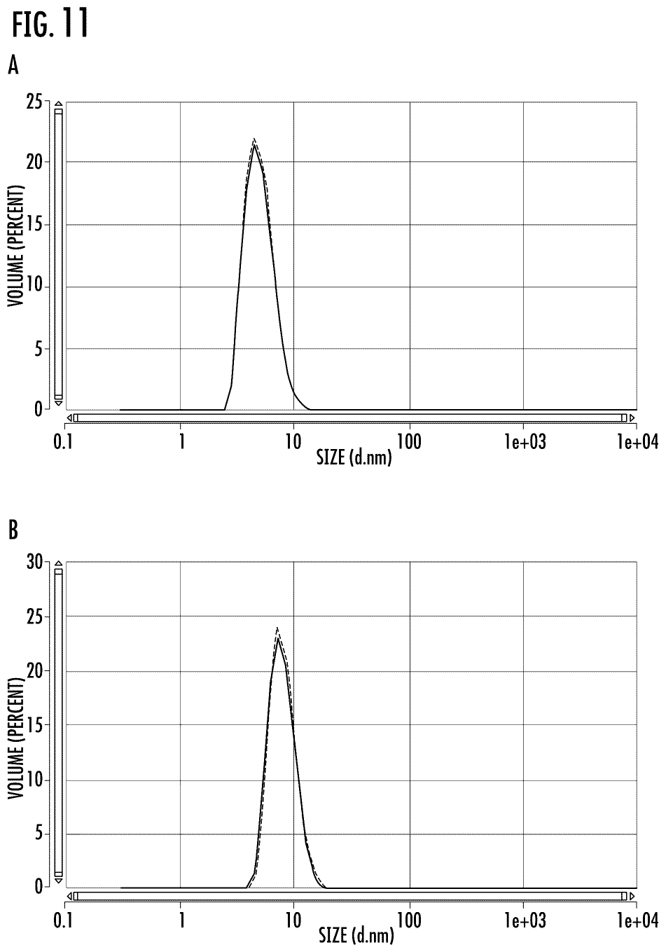

DLS of protein samples that were preincubated in 20 mM Tris (pH 8.0), 50 mM NaCl, 5 mM MgCl₂, 10% glycerol in the presence of 1 mM ADP (FIG. 11 at A) or 1 mM ATP (FIG. 11 at B) yielded a size (volume in nm) of 5.517 nm in the presence of ADP and 8.057 nm in the presence of ATP, again consistent with dimerization of the protein in the presence of 1 mM ATP. DLS analyses were conducted on a Zetasizer® Pro (Model ZSU5800, Malvern Instruments Limited) according to the manufacturer's instructions. DLS results are provided in the table below.

| | Polydispersity | Size distribution |
|---|---|---|
| Sample Name | Index (PI) | by volume (nm) |
| NBD1 in ADP Buffer | 0.3356 | 5.157 |
| NBD1 in ATP Buffer | 0.6278 | 8.057 |

### Example 2

The TAP1 NBD of SEQ ID NO: 118 with a peptide comprising a SpyTag3, TEV protease, and a His tag attached at the C-terminus was expressed in E. coli. and the cells pelleted and frozen until analysis. MPPSGLLTPLHLEGLVQFQDVSFAYPNRPDVLVLQGLTFTLRPGEVTALVGPNGSGKSTVAALLONLYQP TGGQLLLDGKPLPQYEHRYLHRQVAAVGQEPQVFGRSLQENIAYGLTQKPTMEEITAAAVKSGAHSFISG LPQGYDTEVDEAGSQLSGGQRQAVALARALIRKPSVLILDNATSALDAGNQLRVORLLYESPERYSRSVL LITQHLSLVEQADHILFLEGGAIREGGTHQQLMEKKGSYWAMVOAPADAPE (SEQ ID NO:118). The TAP1 NBD fragment was purified by cell lysis followed by chromatograph on a His Trap column. For the analysis cell pellets were thawed and resuspended with 200 ml lysis buffer (20 mM Tris-Cl pH 8.0, 500 mM NaCl, 5 mM MgCl₂, 10% glycerol, 2 mM ATP) supplemented with 100 jug/ml lysozyme, 2 protease inhibitor tablets (Thermo Pierce A32965) and Universal Nuclease (Thermo Pierce 88702) added to 12.5 U/ml. Cells were incubated with stirring at 4° C. for 30 minutes then lysed by sonication at 80% intensity for a total of 4 minutes active time (5 seconds on, 15 seconds rest on ice per cycle). Lysate was then clarified by centrifugation at 12,000×g for 1 hour at 4° C., then 0.22-μm filtered and loaded onto a 1 ml His Trap HP column (Cytiva) at 0.5 ml/min recirculating with a peristaltic pump overnight. The following day, the column was switched to flow-through, and after complete lysate loading at 1 ml/min, the column was washed with 10 column volumes (CV) of lysis buffer supplemented with 7 mM β-mercaptoethanol and 20 mM imidazole. The column was then transferred to an ÄKTAxpress (GE HEALTHCARE BIO-SCIENCES AB) FPLC, and bound proteins were eluted with an imidazole gradient from 0 to 500 mM over 40 CV in binding buffer (20 mM Tris-Cl pH 8.0, 200 mM NaCl, 5 mM MgCl₂, 10% glycerol, 2 mM ATP, 0.5 mM PMSF). Column fractions were assessed by SDS-PAGE, and fractions containing target protein were pooled and concentrated (10 kDa MWCO) to 10 ml before loading as 3 injections (˜3.3 ml per injection) onto a Superdex 200 16/600 column equilibrated in target buffer (20 mM Tris-Cl pH 8.0, 50 mM NaCl, 5 mM MgCl₂, 10% glycerol, 1 mM ATP). Elution peak fractions were assessed by SDS-PAGE, and target protein-containing fractions were pooled and concentrated (10 kDa molecular weight cutoff) to 10 mg/ml before aliquoting (500 μl/tube) and storage at 4° C.

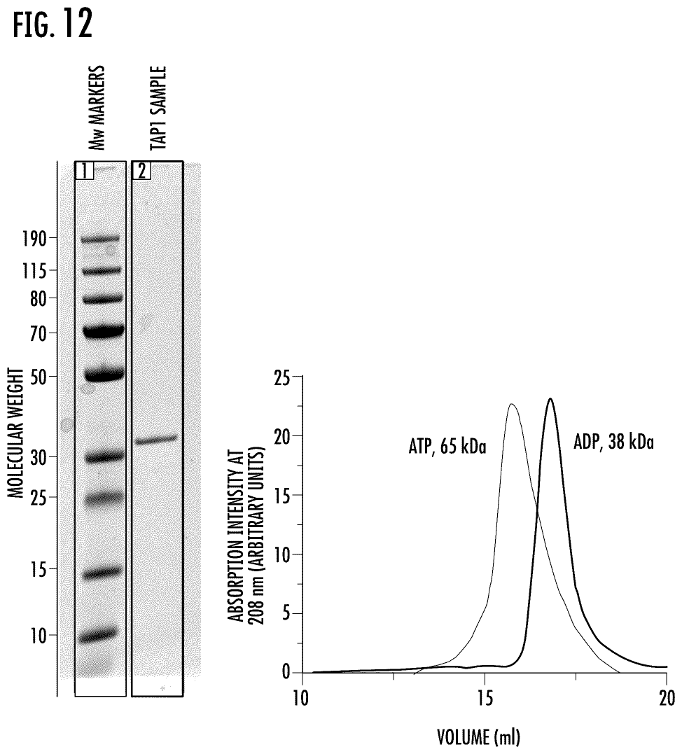

The purified protein was subject to SDS-PAGE along with molecular weight samples. The coomassie blue stained gel is provided in FIG. 12, the leftmost lane is molecular weight samples, and the rightmost lane is the purified protein.

Samples of the purified protein (1 mg each) were buffer-exchanged on PD-10 columns (Cytiva 17085101) according to manufacturer instructions. For the buffer exchange PD-10 desalting columns were equilibrated with target buffer (20 mM Tris-Cl pH 8.0, 50 mM NaCl, 5 mM MgCl₂, 10% glycerol) containing either 1 mM ATP or 1 mM ADP. One milligram (100 μl of 10 mg/ml stock) of protein was added to each column along with 2.4 ml of target buffer, the column was centrifuged at 1000×g for 2 minutes in a 50-ml conical tube, and the eluate was collected. Buffer-exchanged proteins were then concentrated to 0.5 ml (˜60 μM) and loaded onto a Superdex S200 Increase column (Cytiva) equilibrated in the appropriate buffer containing ATP or ADP and eluted at 0.5 ml/min. The ATP-containing sample eluted at a volume of 15.75 ml, corresponding to a molecular weight of approximately 65 kDa, while the ADP sample eluted at 16.81 ml, corresponding to approximately 38 kDa. Traces of the chromatographic analysis are provided in FIG. 12.

For determination of molecular weight by size exclusion chromatography in the presence of ADP and ATP, purified protein samples (1 mg each) were buffer-exchanged on PD-10 columns (Cytiva 17085101) according to manufacturer instructions. Briefly, PD-10 desalting columns were equilibrated with target buffer (20 mM Tris-Cl pH 8.0, 50 mM NaCl, 5 mM MgCl₂, 10% glycerol) containing either 1 mM ATP or ADP. One milligram (100 μl of 10 mg/ml stock) of protein was added to each column along with 2.4 ml of target buffer, the column was centrifuged at 1000×g for 2 minutes in a 50-ml conical tube, and the eluate was collected. Buffer-exchanged proteins were then concentrated to 0.5 ml (˜60 μM), loaded onto a Superdex S200 Increase column (Cytiva), and equilibrated in the appropriate buffer containing ATP or ADP at 0.5 ml/min. The ATP-containing sample eluted at a volume of 15.75 ml, corresponding to a molecular weight of approximately 65 kDa, while the ADP sample eluted at 16.81 ml, corresponding to approximately 38 kDa.

### Example 3

For assessment of the uninduced and induced expression of NBD polypeptide, samples of E. coli BL21 (DE3) were transformed with a vector that permits isopropyl β-d-1-thiogalactopyranoside (IPTG) inducible expression of the NBD of SEQ ID NO: 118 or SEQ ID NO: 119 with a peptide comprising a SpyTag3, TEV protease site, and His tagged at the C-terminus. METEFFQQNQTGGGGSGLQTVRSFGGGGGSGGSGLLTPLHLEGLVQFQDVSFAY PNRPDVLVLQGLTFTLRPGEVTALVGPNGSGKSTVAALLONLYQPTGGQLLLDGKPLPQYEHRYLHRQVA AVGQEPQVFGRSLQENIAYGLTQKPTMEEITAAAVKSGAHSFISGLPQGYDTEVDEAGSQLSGGQRQAVA LARALIRKPSVLILDNAT SALDAGNQLRVORLLYESPERYSRSVLLITQHLSLVEQADHILFLEGGAIRE GGTHQQLMEKKGSYWAMVQAPADAPE (SEQ ID NO:119)

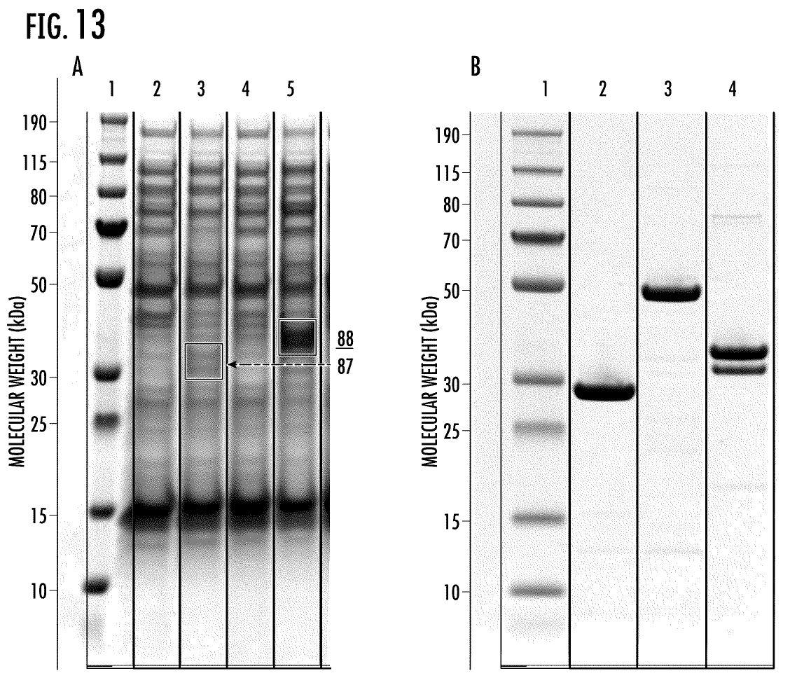

For analysis of the uninduced and induced fractions, the optical density (OD) at 600 nm for each of the cultures was monitored up until induction at 0.4 OD when an aliquot equivalent to 1 OD-ml (e.g. 2.5 ml of 0.4 OD) was taken. The cells in the aliquots were pelleted at 6800× g for 3 minutes, and the pellet was resuspended in 300 μl of 1×PBS, after which the resuspended material was frozen at −80° C. After induction with 80 μM IPTG the cells were grown and a comparable sample of 1 OD-ml (e.g. 0.25 ml of 4 OD) was collected during the final harvest after induction. For analysis of induction the frozen samples were thawed and the cells in all samples were lysed by mixing with an equal volume of 2× lysis buffer (1×PBS with 0.2% Triton X-100, 100 μg/ml lysozyme and 2× concentration of Universal Nuclease from Pierce). Samples were incubated on a rocker at 4° C. for 30 minutes, after which the lysates were spun at 21,000×g for 10 minutes to pellet debris. Samples (30 μl) of the supernatant were mixed with 10 μl of 4× SDS-PAGE loading buffer, heated at 70° C. for about 5 minutes, then loaded on an SDS-PAGE gel. Results are shown in FIG. 13, SDS-PAGE gel A.

For determination expression levels and molecular weight by size exclusion chromatography, samples of the NBD polypeptides of SEQ ID NOs: 118 and 88 were expressed in E. coli and purified for SDS-PAGE and chromatographic analysis as in Example 2. The results of chromatographic molecular weight determinations and protein yield, which is about two orders of magnitude (90-fold) higher for the “capped” NBD of SEQ ID NO:119 relative to that of SEQ ID NO: 118 are provided in the table that follows.

| TAP1 | Yield | | MW in 1 mM | | MW in 1 mM |
|---|---|---|---|---|---|
| polypeptide | from Culture | | ADP buffer | | ATP buffer |
| SEQ ID NO: 118 | 100 | μg/liter | 38 | kDa | 65 kDa |
| SEQ ID NO: 119 | 9 | mg/liter | 38.1 | kDa | 78 kDa |

FIG. 13 provides SDS PAGE analysis of induced and uninduced expression at A and analysis of the purified protein at B. The samples in the lanes of the reducing SDS-PAGE gel images provided in FIG. 13 are indicated in the table that follows.

| Gel (A) Induced vs. | | Gel (B) Evaluation of the purified | |
|---|---|---|---|
| Uninduced Expression | | NBD polypeptide of SEQ ID NO: 119 | |
| Lane | Sample | Lane | Sample |
| 1 | Molecular Weight Markers | 1 | Molecular Weight Markers |
| 2 | Expression of the NBD polypeptide of SEQ ID | 2 | Additional purified NBD polypeptide |
| | NO: 118 prior to induction | | |
| 3 | Expression of the NBD polypeptide of SEQ ID | 3 | Additional purified NBD polypeptide |
| | NO: 118 after induction (see white box and | | |
| | arrow) | | |
| 4 | Expression of the NBD polypeptide of SEQ ID | 4 | The purified NBD polypeptide of |
| | NO: 119 prior to induction | | SEQ ID NO: 119 |
| 5 | Expression of the NBD polypeptide of SEQ ID | | |
| | NO: 119 after induction (see white box) | | |

### Example 4. Enhancement of TAP Construct Functions

To enhance the TAP nucleotide binding domain's dimerization function, dimerization dependence upon ATP (e.g., as opposed to ADP or other nucleotides), and stability, nucleic acids encoding TAP1 constructs were cloned into an antibiotic selectable E. coli expression vector under the control of an IPTG inducible T7 promoter. FIG. 14B provides the aa sequences of the expressed protein constructs by construct number and FIG. 15 provides a table summarizing, among other things, some structural features of various TAP1 constructs, the results of expression testing, dimerization in the presence of ATP, and construct melting points of those constructs.

The constructs comprise a carboxyl terminal His Tag permitting purification by immobilized metal affinity chromatography (IMAC).

For protein production, the expression vectors were transfected into SHuffle® T7 Competent E. coli (New England BioLabs, Inc.). The cells were grown overnight in LB medium with antibiotic selection at 37° C., after which a sample of the overnight culture was diluted into fresh LB with antibiotic. The cells were grown to mid-log phase (A600 0.6-0.8) at 37° C., after which expression was induced by adding IPTG to 1 mM IPTG, and the cells grown for an additional 4-8 hours at 16-20° C. Cells were harvested by centrifugation and the cell pellets frozen for later analysis.

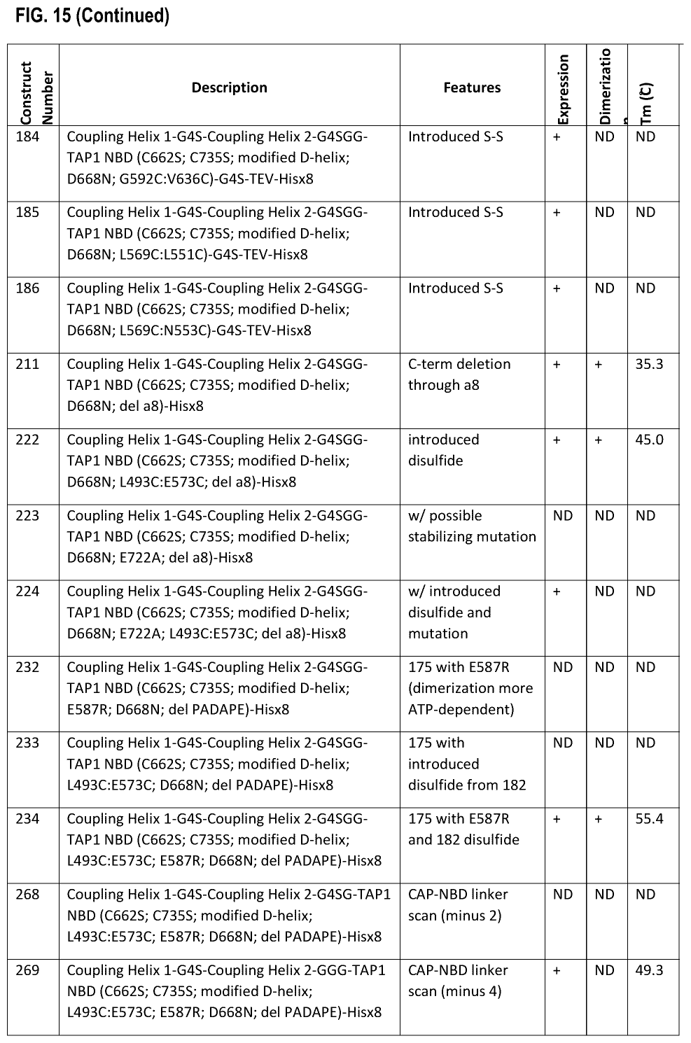

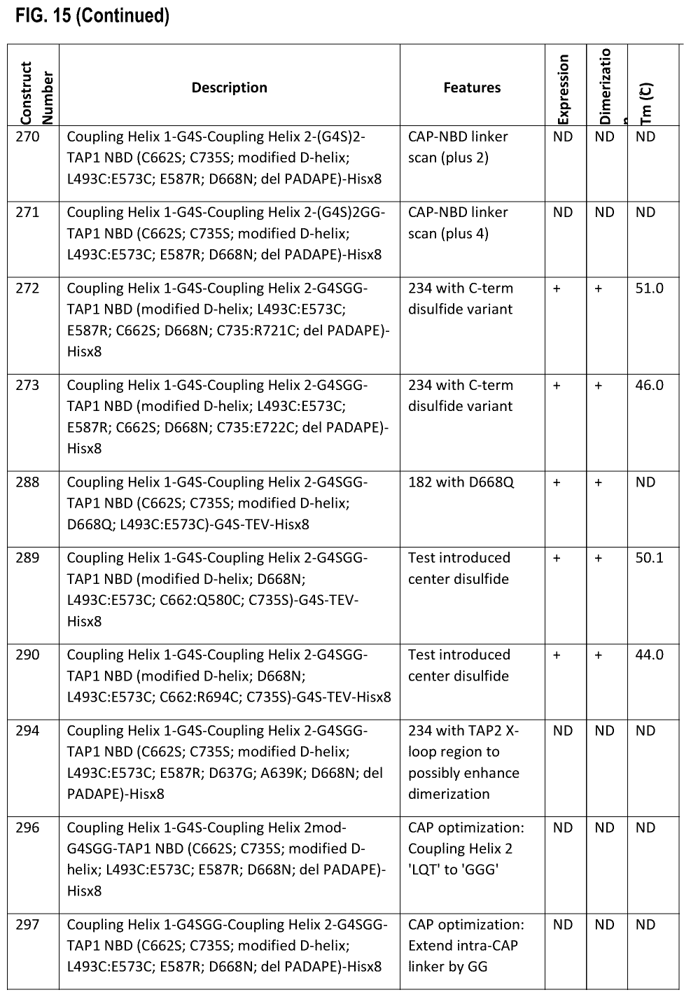

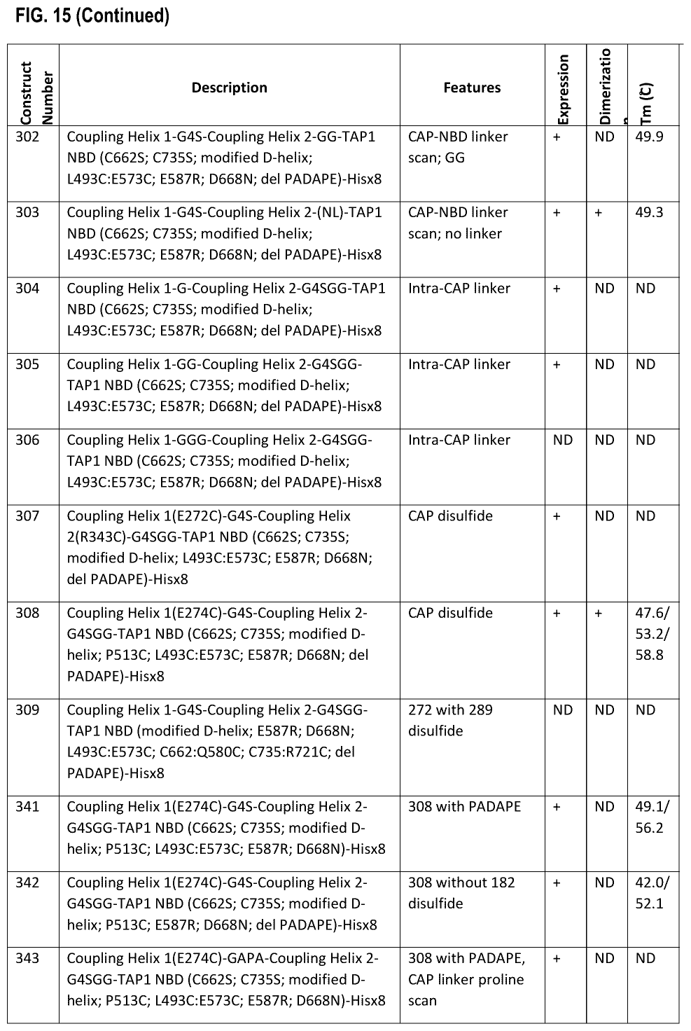

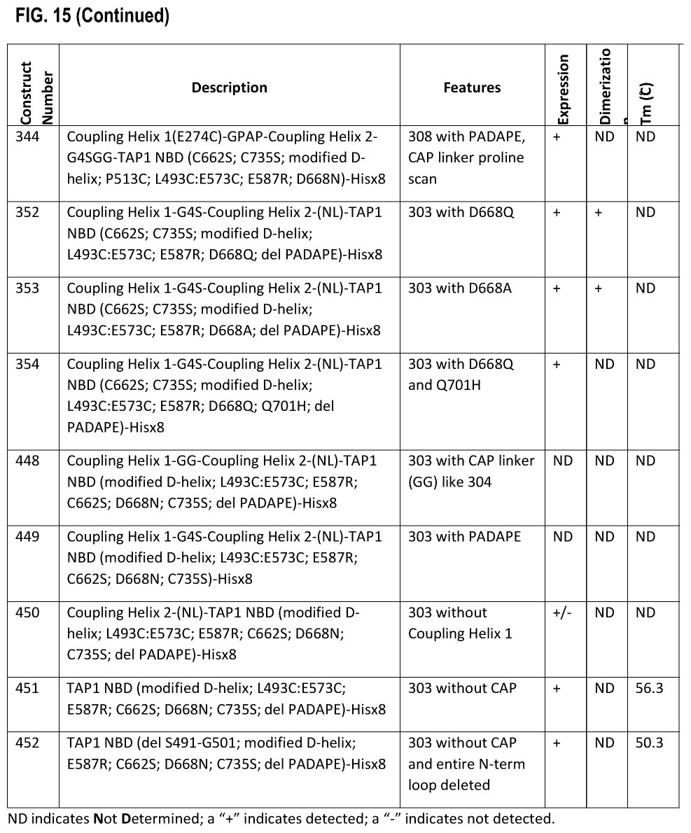

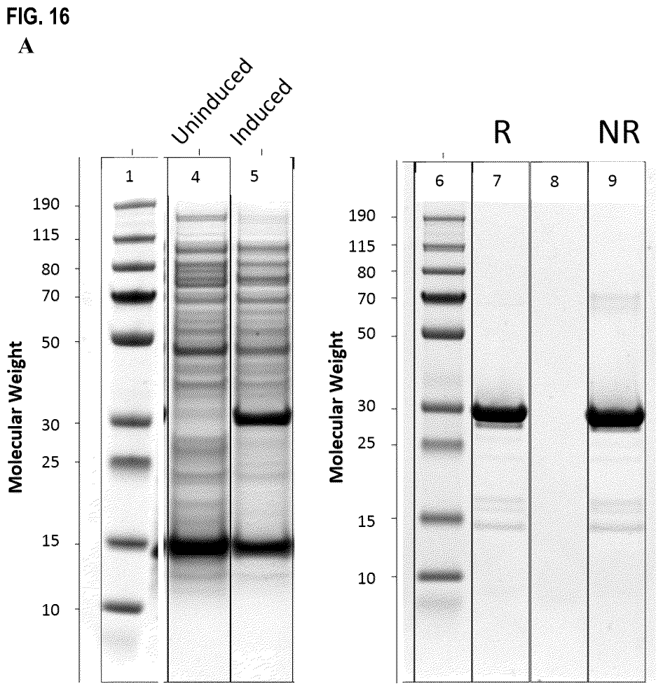

Cell pellets were thawed and resuspended with 200 ml lysis buffer (20 mM Tris-Cl pH 8.0, 500 mM NaCl, 5 mM MgCl₂, 10% glycerol, 2 mM ATP) supplemented with 100 μg/ml lysozyme, 2 protease inhibitor tablets (Thermo Pierce A32965) and Universal Nuclease (Thermo Pierce 88702) added to 12.5 U/ml. Cells were incubated with stirring at 4° C. for 30 minutes then lysed by sonication. Lysate was clarified by centrifugation at 12,000×g for 1 hour at 4° C., then 0.22-μm filtered and loaded onto a 1 ml His Trap HP column (Cytiva) at 0.5 ml/min recirculating with a peristaltic pump overnight. The following day, the column was switched to flow-through and, after complete lysate loading at 1 ml/min, the column was washed with 10 column volumes of lysis buffer supplemented with 7 mM β-mercaptoethanol and 20 mM imidazole. The column was then transferred to an ÄKTAxpress FPLC, and bound proteins were eluted with an imidazole gradient from 0 to 500 mM over 40 column volumes in binding buffer (20 mM Tris-Cl pH 8.0, 200 mM NaCl, 5 mM MgCl₂, 10% glycerol, 2 mM ATP, 0.5 mM PMSF). Column fractions were assessed by SDS-PAGE, and fractions containing target protein were pooled and concentrated (10 kDa MWCO) to 10 ml before loading as 3 injections (˜3.3 ml per injection) onto a Superdex 200 16/600 column equilibrated in target buffer (20 mM Tris-Cl pH 8.0, 50 mM NaCl, 5 mM MgCl₂, 10% glycerol, 1 mM ATP). Elution peak fractions were assessed by SDS-PAGE, and target protein-containing fractions were pooled and concentrated (10 kDa MWCO) to 10 mg/ml before aliquoting (500 μl/tube) and flash-freezing in liquid nitrogen, with storage at −80° C. FIG. 16 at A shows lanes from SDS PAGE analysis of an exemplary TAP1 construct expression and purification. Lanes 1 and 6 are molecular weight standards. Lanes 4 and 5 depict total protein from the E. coli cells before and after induction with IPTG respectively. Lanes 7 and 9 show samples of the purified TAP1 protein construct run under reducing (lane 7) and non-reducing (NR, lane 9) conditions. Lane 8 was unloaded.

The ability of TAP1 protein constructs to dimerize in the presence of 1 mM ADP or 1 mM ATP was assessed by size exclusion chromatography as described in Example 1. An exemplary result for construct 303 is shown in FIG. 16 at B. Melting points for the constructs was determined by differential scanning calorimetry (DSC) using a MicroCal PEAQ-DSC instrument (Malvern Panalytic). All DSC measurements were made in 20 mM Tris (pH 8.0), 50 mM NaCl, 5 mM MgCl₂ and 1 mM ADP present in the buffer. The specific heat capacity (Cp) in kcal per mole per Kelvin degree (kcal/mol/K) is plotted for an exemplary construct 303 at temperatures from about 20° to about 85° C. in FIG. 16 at C. Melting points were determined by using the company's proprietary software.

The effect of C-terminal amino acid deletions on melting points determined by DSC for constructs are plotted in FIG. 17.

The plot provides the melting points of constructs 103, 175-179, and 211 marked with native TAP1 aa sequences (filled circles) and constructs 182, 234, and 222 with added disulfide bonds (filled squares). Also shown is the melting point of construct 451 as an asterisk. The plot indicates that constructs may be prepared with melting points greater 50° C., for example from 50° C. to 50° C. or from 54° C. to 58° C.

### Example 5. Mesothelin and BCMA Targeted Protein Constructs

Two protein constructs comprising anti-CD3 Fab ADs are prepared. The first construct comprises an anti-mesothelin targeting portion and the second construct comprises an anti-B cell maturation antigen (anti-BCMA) targeting portion. The nucleotide binding domain (TAP1) may be part of a fusion protein, or it may be conjugated to the linker of the anti-CD3 (VL-Ck)-linker polypeptide using a Q tag, SpyTag, SpyCatcher, split intern, or sortase conjugation.

The polypeptides of the first construct are arranged from N-terminus to C-terminus as follows:
Anti-CD3 (VH-CH1)-linker-anti-mesothelin, and
Anti-CD3 (VL-Ck)-linker-TAP1.

The polypeptides of the second construct are arranged from N-terminus to C-terminus as follows:
Anti-CD3 (VH-CH1)-linker-anti-BCMA, and
Anti-CD3 (VL-Ck)-linker-TAP1.

The anti-CD3 VH-CH1 amino acid sequence may comprise the aa sequence:
MEPWPLLLLFSLCSAGLVLGQVQLVQSGGGVVQPGRSLRLSCKASGYTFTRYTMHWVRQAPGKGLEWIGYINPSRG YTNYNQKVKDRFTISRDNSKNTAFLQMDSLRPEDTGVYFCARYYDDHYCLDYWGQGTPVTVSSASTKGPSVFPLAP SSKSTSGGTAALGCLVKDYFPEPVTVSWNSGALTSGVHTFPAVLQSSGLYSLSSVVTVPSSSLGTQTYICNVNHKPS NTKVDKKVEPK SC (SEQ ID NO:174), or a sequence having greater than 95% or greater than 98% sequence identity to SEQ ID NO: 174;
and the anti-CD3 V_{L}-Ck amino acid sequence may comprise the aa sequence: MEPWPLLLLFSLCSAGLVLGDIQM TQSPSSLSASVGDRVTITCSASSSVSYMNWYQQTPGKAPKRWIYDTSKLASGVPSRFSGSGSGTDYTFTISSLQPED IATYYCQQWSSNPFTFGQGTKLQITRTVAAPSVFIFPPSDEQLKSGTASVVCLLNNFYPREAKVQWKVDNALQSGNS QESVTEQDSKDSTYSLSSTLTLSKADYEKHKVYACEVTHQGLSSPVTKSFNRGEC (SEQ ID NO:175), or a sequence having greater than 95% or greater than 98% sequence identity to SEQ ID NO: 175.

Alternatively, amino acid sequences for the anti-CD3 VH-CH1 and the anti-CD3 VL-Ck comprising MD13 sequence variations may be employed for the AD. An anti-CD3 VH-CH1 amino acid sequence comprising MD13 substitutions that can be employed may comprise the aa sequence: MEPWPLLLLFSLCSAGLVLGQVQLVQSGGGVVQPGRSLRLSCKASGYTFTRYTMHWVRQAPGKGLEWIGYINPSRG YTNYNQKVKDRFTISRDNSKNTAFLQMDSLRPEDTGVYFCARYYDDHYCLDYWGQGTPVTVSSASTKGPSVFPLAP SSKSTSGGTAALGCLVKDYFPEPVTVSWNSGALTSGVHTFPAVLQSSGLYELVSWVTVPSSSLGTQTYICNVNHKPS NTKVDKKVEPKSC (SEQ ID NO:176) or a sequence having greater than 95% or greater than 98% sequence identity to SEQ ID NO: 176; and an anti-CD3 VL-Ck amino acid sequence that may be employed in the constructs comprising MD13 may comprise the aa sequence: MEPWPLLLLFSLCSAGLVLGDIQMTQSPSSLSASVGDRVTITCSASSSVSYMNWYQQTPGKAPKRWIYDTSKLASGV PSRFSGSGSGTDYTFTISSLQPEDIATYYCQQWSSNPFTFGQGTKLQITRTVAAPSVFIFPPSDEQLKSGTASVVCLLN NFYPREAKVQWKVDNALQSGNSQESVTEQDSKDSTYSLLSSLTLSKADYEKHKVYACEVTHQGLSSPVTKSFNRGE C (SEQ ID NO:177), or a sequence having greater than 95% or greater than 98% sequence identity to SEQ ID NO: 177.

The anti-mesothelin targeting sequence for the preparation of the first construct may comprise an aa sequence selected from:
(i) the anti-mesothelin VHH aa sequence

(SEQ ID NO: 97)
QVQLVQSGGGLVQPGGSLRLSCAASDFDFAAYEMSWVRQAPGQGL
EWVAIISHDGIDKYYTDSVKGRFTISRDNSKNTLYLQMNTLRAED
TATYYCLRLGAVGQGTLVTVSSS;

(ii) the anti-mesothelin scFv aa sequence MEPWPLLLLFSLCSAGLVLGQVQLQQSGPELEKPGASVKISCKASGYSFTGYTMNWVKQSHGKSLEWIGLITPYNGA SSYNQKFRGKATLTVDKSSSTAYMDLLSLTSEDSAVYFCARGGYDGRGFDYWGSGTPVTVSSGVGGSGGGGSGG GGSDIELTQSPAIMSASPGEKVTMTCSASSSVSYMHWYQQKSGTSPKRWIYDTSKLASGVPGRFSGSGSGNSYSLTI SSVEAEDDATYYCQQWSKHPLTFGSGTKVEIK (SEQ ID NO:178); or
(iii) the anti-mesothelin scFv aa sequence

(SEQ ID NO: 179)
MEPWPLLLLFSLCSAGLVLGQQQLEESGGGLVKPEGSLTLTCKAS
GFDLGFYFYACWVRQAPGKGLEWIACIYTAGSGSTYYASWAKGRF
TISKASSTTVTLQMTSLAAADTATYFCARSTANTRSTYLNLWGPG
TLVTVSSGVGGSGGGGSGGGGSDWMTQTPASVSEPVGGTVTIKCQ
ASQRISSYLSWYQQKPGQRPKLLIFGASTLASGVPSRFKGSGSGT
EYTLTISDLECADAATYYCQSYAYFDSNNWHAFGGGTEVVV

The anti-BCMA VHH targeting sequence for the preparation of the second construct may comprise the aa sequence:

(SEQ ID NO: 180)
QVKLEESGGGLVQAGRSLRLSCAASEHTFSSHVMGWFRQAPGKER
ESVAVIGWRDISTSYADSVKGRFTISRDNAKKTLYLQMNSLKPED
TAVYYCAARRIDAADFDSWGQGTQVTVSS

The TAP1 nucleotide binding sequence for the preparation of the constructs may comprise an aa sequence selected from:
(i) the hydrolysis competent (D668) TAP1 aa sequence variant lacking cysteines:

(SEQ ID NO: 181)
PPSGLLTPLHLEGLVQFQDVSFAYPNRPDVLVLQGLTFTLRPGEV
TALVGPNGSGKSTVAALLQNLYQPTGGQLLLDGKPLPQYEHRYLH
RQVAAVGQEPQVFGRSLQENIAYGLTQKPTMEEITAAAVKSGAHS
FISGLPQGYDTEVDEAGSQLSGGQRQAVALARALIRKPSVLILDD
ATSALDAGNQLRVQRLLYESPERYSRSVLLITQHLSLVEQADHIL
FLEGGAIREGGTHQQLMEKKGSYWAMVQAPADAPE;

(ii) the hydrolysis deficient (D668N) TAP1 aa sequence variant lacking cysteines

(SEQ ID NO: 15)
PPSGLLTPLHLEGLVQFQDVSFAYPNRPDVLVLQGLTFTLRPGEV
TALVGPNGSGKSTVAALLQNLYQPTGGQLLLDGKPLPQYEHRYLH
RQVAAVGQEPQVFGRSLQENIAYGLTQKPTMEEITAAAVKSGAHS
FISGLPQGYDTEVDEAGSQLSGGQRQAVALARALIRKPSVLILDN
ATSALDAGNQLRVQRLLYESPERYSRSVLLITQHLSLVEQADHIL
FLEGGAIREGGTHQQLMEKKGSYWAMVQAPADAPE;

(iii) the hydrolysis competent N-terminal CAP stabilized TAP1 aa sequence variant lacking cysteines ETEFFQQNQTGGGGSGLQTVRSFGGGGGSGGSGLLTPLHLEGLVQFQDVSFAYPNRPDVLVLQGLTFTLRPGEVTA LVGPNGSGKSTVAALLQNLYQPTGGQLLLDGKPLPQYEHRYLHRQVAAVGQEPQVFGRSLQENIAYGLTQKPTMEEI TAAAVKSGAHSFISGLPQGYDTEVDEAGSQLSGGQRQAVALARALIRKPSVLILDDATSALDAGNQLRVQRLLYESPE RYSRSVLLITQHLSLVEQADHILFLEGGAIREGGTHQQLMEKKGSYWAMVQAPADAPE (SEQ ID NO:19); or
(iv) the hydrolysis deficient N-terminal CAP stabilized TAP1 aa sequence variant lacking cysteines

(SEQ ID NO: 182)
ETEFFQQNQTGGGGSGLQTVRSFGGGGGSGGSGLLTPLHLEGLVQ
FQDVSFAYPNRPDVLVLQGLTFTLRPGEVTALVGPNGSGKSTVAA
LLQNLYQPTGGQLLLDGKPLPQYEHRYLHRQVAAVGQEPQVFGRS
LQENIAYGLTQKPTMEEITAAAVKSGAHSFISGLPQGYDTEVDEA
GSQLSGGQRQAVALARALIRKPSVLILDNATSALDAGNQLRVQRL
LYESPERYSRSVLLITQHLSLVEQADHILFLEGGAIREGGTHQQL
MEKKGSYWAMVQAPADAPE

When any of the preceding TAP1 aa sequences are located at the N-terminus of a protein construct they may be preceded by a methionine residue.

### Example 6. BCMA-CD3 Bispecific Molecules

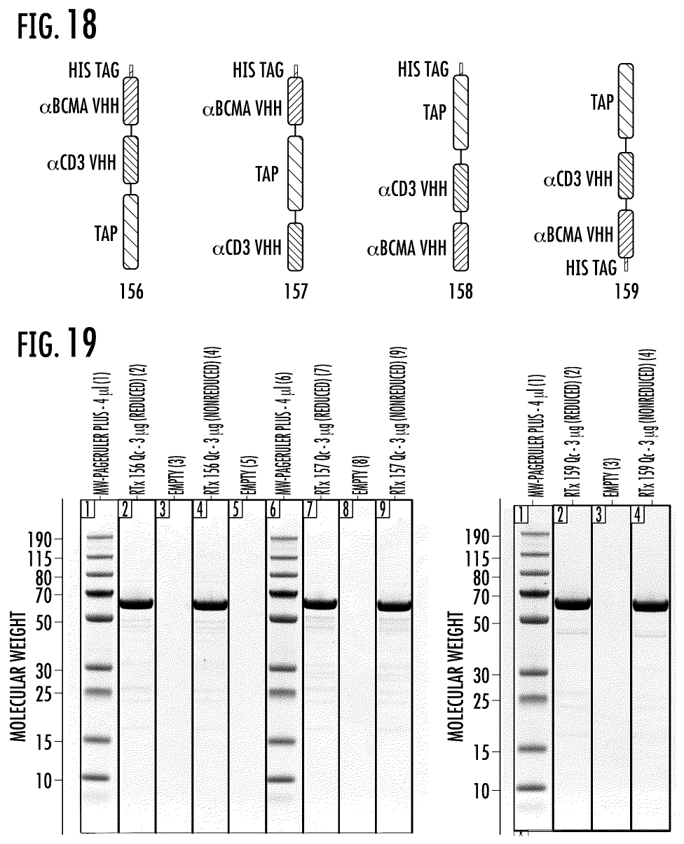

Four nucleic acid constructs (constructs 156-159) encoding polypeptides having as elements an NBD, an anti-BCMA VHH, and an anti-CD3 aa sequences in various orders were prepared. Construct are depicted in FIG. 18, and the order of their elements and sequences are provided in the table that follows.

| Construct | Description |
|---|---|
| 156 | Hisx8-anti-BCMA VHH-(G4S)2-anti- |
| | CD3 VHH-(G4S)2-CH1-CH2-TAP1 |
| | NBD (Cys-less; modified D-helix; |
| | D668N) |
| | MHHHHHHHHEVQLVESGGGLVQAGGSLRLS |
| | CAASGRTFTMGWFRQAPGKEREFVAAISLS |
| | PTLAYYAESVKGRFTISRDNAKNTVVLQMN |
| | SLKPEDTALYYCAADRKSVMSIRPDYWGQG |
| | TQVTVSSGGGGSGGGGSQVQLVESGGGLVQ |
| | PGGSLRLSCAASGSIFSANTMGWYRQAPGK |
| | QRELVAGMNTSGSTVYGDSVKGRFTISRDN |
| | AKNIAYLQMNSLIPEDTAVYYCTLVQRGPN |
| | YWGQGTQVTVSSGGGGSGGGGSETEFFQQN |
| | QTGGGGSGLQTVRSFGGGGGSGGSGLLTPL |
| | HLEGLVQFQDVSFAYPNRPDVLVLQGLTFT |
| | LRPGEVTALVGPNGSGKSTVAALLQNLYQP |
| | TGGQLLLDGKPLPQYEHRYLHRQVAAVGQE |
| | PQVFGRSLQENIAYGLTQKPTMEEITAAAV |
| | KSGAHSFISGLPQGYDTEVDEAGSQLSGGQ |
| | RQAVALARALIRKPSVLILDNATSALDAGN |
| | QLRVQRLLYESPERYSRSVLLITQHLSLVE |
| | QADHILFLEGGAIREGGTHQQLMEKKGSYW |
| | AMVQAPADAPEGGGGS |
| | (SEQ ID NO: 183) |
| 157 | Hisx8-anti-BCMA VHH-(G4S)2-CH1 |
| | -CH2-TAP1 NBD (Cys-less; |
| | modified D-helix; D668N)-(G4S) |
| | 2-anti-CD3 VHH |
| | MHHHHHHHHEVQLVESGGGLVQAGGSLRLS |
| | CAASGRTFTMGWFRQAPGKEREFVAAISLS |
| | PTLAYYAESVKGRFTISRDNAKNTVVLQMN |
| | SLKPEDTALYYCAADRKSVMSIRPDYWGQG |
| | TQVTVSSGGGGSGGGGSETEFFQQNQTGGG |
| | GSGLQTVRSFGGGGGSGGSGLLTPLHLEGL |
| | VQFQDVSFAYPNRPDVLVLQGLTFTLRPGE |
| | VTALVGPNGSGKSTVAALLQNLYQPTGGQL |
| | LLDGKPLPQYEHRYLHRQVAAVGQEPQVFG |
| | RSLQENIAYGLTQKPTMEEITAAAVKSGAH |
| | SFISGLPQGYDTEVDEAGSQLSGGQRQAVA |
| | LARALIRKPSVLILDNATSALDAGNQLRVQ |
| | RLLYESPERYSRSVLLITQHLSLVEQADHI |
| | LFLEGGAIREGGTHQQLMEKKGSYWAMVQA |
| | PADAPEGGGGSGGGGSGGGGSQVQLVESGG |
| | GLVQPGGSLRLSCAASGSIFSANTMGWYRQ |
| | APGKQRELVAGMNTSGSTVYGDSVKGRFTI |
| | SRDNAKNIAYLQMNSLIPEDTAVYYCTLVQ |
| | RGPNYWGQGTQVTVSS |
| | (SEQ ID NO: 184) |
| 158 | Hisx8-CH1-CH2-TAP1 NBD |
| | (Cys-less; modified D-helix; |
| | D668N)-(G4S)2-anti-VHH-(G4S)2 |
| | -anti.BCMAVHH |
| | MHHHHHHHHETEFFQQNQTGGGGSGLQTVR |
| | SFGGGGGSGGSGLLTPLHLEGLVQFQDVSF |
| | AYPNRPDVLVLQGLTFTLRPGEVTALVGPN |
| | GSGKSTVAALLQNLYQPTGGQLLLDGKPLP |
| | QYEHRYLHRQVAAVGQEPQVFGRSLQENIA |
| | YGLTQKPTMEEITAAAVKSGAHSFISGLPQ |
| | GYDTEVDEAGSQLSGGQRQAVALARALIRK |
| | PSVLILDNATSALDAGNQLRVQRLLYESPE |
| | RYSRSVLLITQHLSLVEQADHILFLEGGAI |
| | REGGTHQQLMEKKGSYWAMVQAPADAPEGG |
| | GGSGGGGSGGGGSQVQLVESGGGLVQPGGS |
| | LRLSCAASGSIFSANTMGWYRQAPGKQREL |
| | VAGMNTSGSTVYGDSVKGRFTISRDNAKNI |
| | AYLQMNSLIPEDTAVYYCTLVQRGPNYWGQ |
| | GTQVTVSSGGGGGGGGSEVQLVESGGGLVQ |
| | AGGSLRLSCAASGRTFTMGWFRQAPGKERE |
| | FVAAISLSPTLAYYAESVKGRFTISRDNAK |
| | NTVVLQMNSLKPEDTALYYCAADRKSVMSI |
| | RPDYWGQGTQVTVSS |
| | (SEQ ID NO: 185) |
| 159 | CH1-CH2-TAP1 NBD (Cys-less; mo |
| | dified D-helix; |
| | D668N)-(G4S)2-anti-CD3 VHH- |
| | (G4S)2-anti-BCMAVHH-Hisx8 |
| | METEFFQQNQTGGGGSGLQTVRSFGGGGGS |
| | GGSGLLTPLHLEGLVQFQDVSFAYPNRPDV |
| | LVLQGLTFTLRPGEVTALVGPNGSGKSTVA |
| | ALLQNLYQPTGGQLLLDGKPLPQYEHRYLH |
| | RQVAAVGQEPQVFGRSLQENIAYGLTQKPT |
| | MEEITAAAVKSGAHSFISGLPQGYDTEVDE |
| | AGSQLSGGQRQAVALARALIRKPSVLILDN |
| | ATSALDAGNQLRVQRLLYESPERYSRSVLL |
| | ITQHLSLVEQADHILFLEGGAIREGGTHQQ |
| | LMEKKGSYWAMVQAPADAPEGGGGSGGGGS |
| | GGGGSQVQLVESGGGLVQPGGSLRLSCAAS |
| | GSIFSANTMGWYRQAPGKQRELVAGMNTSG |
| | STVYGDSVKGRFTISRDNAKNIAYLQMNSL |
| | IPEDTAVYYCTLVQRGPNYWGQGTQVTVSS |
| | GGGGSGGGGSEVQLVESGGGLVQAGGSLRL |
| | SCAASGRTFTMGWFRQAPGKEREFVAAISL |
| | SPTLAYYAESVKGRFTISRDNAKNTVVLQM |
| | NSLKPEDTALYYCAADRKSVMSIRPDYWGQ |
| | GTQVTVSSHHHHHHHH |
| | (SEQ ID NO: 186) |

E. coli were transformed with the four nucleic acid constructs and the expressed polypeptides were purified by immobilized metal affinity chromatography (IMAC) followed by size exclusion chromatography. Samples of purified protein from constructs 156, 157, and 159 were subject to SDS polyacrylamide gel electrophoresis (SDS-PAGE). Each construct gave a single band in both the reduced and oxidized states (see, FIG. 19). Construct 158 was expressed at low levels and was not further analyzed.

Melting points for constructs 156, 157, and 159 were determined by differential scanning calorimetry (DSC) using a MicroCal PEAQ-DSC instrument (Malvern Panalytic). All DSC measurements were made in 20 mM Tris (pH 8.0), 50 mM NaCl, 5 mM MgCl₂ and 1 mM ADP present in the buffer. Each construct yielded melting curves with two temperature transitions (Tm-1 and Tm-2). The Tm-1 and Tm-2 values and the temperature (in ° C.) at which melting initially begins (T(onset)) are set forth below.

| | T(onset) | Tm-1 | Tm-2 |
|---|---|---|---|
| Construct | in ° C. | in ° C. | in ° C. |
| 156 | 39.1 | 44.9 | 58.0 |
| 157 | 38.2 | 44.7 | 57.7 |
| 159 | 38.6 | 44.6 | 58.5 |

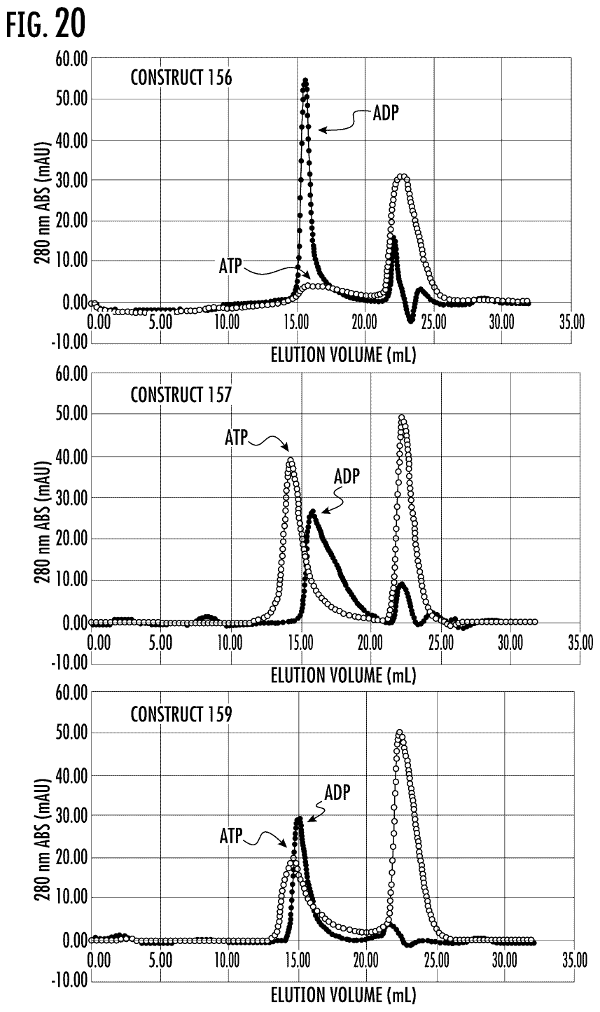

To evaluate the behavior of the constructs in the presence of ATP and ADP samples of the purified protein (1 mg each) were buffer-exchanged on PD-10 columns (Cytiva 17085101) according to manufacturer instructions. For the buffer exchange PD-10 desalting columns were equilibrated with target buffer (20 mM Tris-Cl pH 8.0, 50 mM NaCl, 5 mM MgCl₂, 10% glycerol) containing either 1 mM ATP or 1 mM ADP. One milligram (100 μl of 10 mg/ml stock) of protein was added to each column along with 2.4 ml of target buffer, the column was centrifuged at 1000×g for 2 minutes in a 50-ml conical tube, and the eluate was collected. Buffer-exchanged proteins were then concentrated to 0.5 ml (˜60 μM) and loaded onto a Superdex S200 Increase column (Cytiva) equilibrated in the appropriate buffer containing ATP or ADP and eluted at 0.5 ml/min. The ATP-containing samples were subject to size exclusion chromatography and eluted with a peak at a volume less than about 15 ml, while the ADP containing samples eluted with a peak at a volume greater than about 15 ml. Peaks appearing between about 20 and 25 ml are due to buffer artifacts and/or the presence of unbound nucleotide. Traces of the chromatographic analysis are provided in FIG. 20. Construct 157 displays the most prominent ATP mediated shift in molecular weight due to dimerization of the construct.

### Example 7. ATP Dimerization Set Point and Target Residence Time and Reversibility of Binding

Three constructs comprising a pair of first and second polypeptides that interact through KIH IgFc scaffold sequences were prepared. In each of the constructs the first polypeptide, designated 533 comprised one of the KIH hole sequence and an anti-mesothelin (MSLN) scFv aa sequence. The second polypeptide of each of the three constructs, polypeptides designate 571, 647, and 648, each comprises a KIH IgFc knob aa sequence and a homodimerizing TAP1 NBD with a non-modified TAP1 ATP binding pocket or modified ATP binding pockets.

| Construct | Description and sequence‡ |
|---|---|
| 533 | Human IgG1 (higG1) Fc (wt L234, L235; hole)- |
| | (G₄S)₂ linker bolded and Italicized-SS1 (anti- |
| | mesothelin scFv) |
| | DKTHTCPPCPAPELLGGPSVFLFPPKPKDTLMISRTPEVTCVVVDVSHE |
| | DPEVKFNWYVDGVEVHNAKTKPREEQYNSTYRWSVLTVLHQDWLNGKEY |
| | KCKVSNKALPAPIEKTISKAKGQPREPQVCTLPPSRDELTKNQVSLSCA |
| | VKGFYPSDIAVEWESNGQPENNYKTTPPVLDSDGSFFLVSKLTVDKSRW |
| | QQGNVFSCSVMHEALHNHYTQKSLSLSPGGGGGSGGGGSQVQLQQSGPE |
| | LEKPGASVKISCKASGYSFTGYTMNWVKQSHGKSLEWIGLITPYNGASS |
| | YNQKFRGKATLTVDKSSSTAYMDLLSLTSEDSAVYFCARGGYDGRGFDY |
| | WGSGTPVTVSSGGGGSGGGGSGGGGSDIELTQSPAIMSASPGEKVTMTC |
| | SASSSVSYMHWYQQKSGTSPKRWIYDTSKLASGVPGRFSGSGSGNSYSL |
| | TISSVEAEDDATYYCQQWSKHPLTFGSGTKVEIK |
| | (SEQ ID NO: 187) |
| 571 | Cap (Coupling Helix 1-G₄S-Coupling Helix 2- |
| | underlined) (NL)-TAP1 NBD (modified D-helix; |
| | L493C: E573C (disulfide bond); S542A; C662S; |
| | D668Q; C735S; deletion of PADAPE)-(G₄S)₂ linker- |
| | hIgG1 Fc (wt L234, L235; knob) |
| | ETEFFQQNQTGGGGSGLQTVRSFGSGCLTPLHLEGLVQFQDVSFAYPNR |
| | PDVLVLQGLTFTLRPGEVTALVGPNGAGKSTVAALLQNLYQPTGGQLLL |
| | DGKPLPQYCHRYLHRQVAAVGQEPQVFGRSLQENIAYGLTQKPTMEEIT |
| | AAAVKSGAHSFISGLPQGYDTEVDEAGSQLSGGQRQAVALARALIRKPS |
| | VLILDQATSALDAGNQLRVQRLLYESPERYSRSVLLITQHLSLVEQADH |
| | ILFLEGGAIREGGTHQQLMEKKGSYWAMVQAGGGGSGGGGSDKTHTCPP |
| | CPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNK |
| | ALPAPIEKTISKAKGQPREPQVYTLPPCRDELTKNQVSLWCLVKGFYPS |
| | DIAVEWESNGQPENNYKTTPPVLDSDGSFFLYSKLTVDKSRWQQGNVFS |
| | CSVMHEALHNHYTQKSLSLSPG |
| | (SEQ ID NO: 188) |
| 647 | Cap (Coupling Helix 1-G₄S-Coupling Helix 2- |
| | underlined) (NL)-TAP(D667A)-(G₄S)₂-hIgG1 Fc (wt |
| | L234, L235; knob) |
| | ETEFFQQNQTGGGGSGLQTVRSFGSGCLTPLHLEGLVQFQDVSFAYPNR |
| | PDVLVLQGLTFTLRPGEVTALVGPNGAGKSTVAALLQNLYQPTGGQLLL |
| | DGKPLPQYCHRYLHRQVAAVGQEPQVFGRSLQENIAYGLTQKPTMEEIT |
| | AAAVKSGAHSFISGLPQGYDTEVDEAGSQLSGGQRQAVALARALIRKPS |
| | VLILAQATSALDAGNQLRVQRLLYESPERYSRSVLLITQHLSLVEQADH |
| | ILFLEGGAIREGGTHQQLMEKKGSYWAMVQAGGGGSGGGGSDKTHTCPP |
| | CPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNK |
| | ALPAPIEKTISKAKGQPREPQVYTLPPCRDELTKNQVSLWCLVKGFYPS |
| | DIAVEWESNGQPENNYKTTPPVLDSDGSFFLYSKLTVDKSRWQQGNVFS |
| | CSVMHEALHNHYTQKSLSLSPG |
| | (SEQ ID NO: 189) |
| 648 | Cap (Coupling Helix 1-G₄S-Coupling Helix 2-under |
| | lined) (NL)-TAP(Q586A)-(G₄S)₂-hIgG1 |
| | Fc(wt L234, L235; knob) |
| | ETEFFQQNQTGGGGSGLQTVRSFGSGCLTPLHLEGLVQFQDVSFAYPNR |
| | PDVLVLQGLTFTLRPGEVTALVGPNGAGKSTVAALLQNLYQPTGGQLLL |
| | DGKPLPQYCHRYLHRQVAAVGAEPQVFGRSLQENIAYGLTQKPTMEEIT |
| | AAAVKSGAHSFISGLPQGYDTEVDEAGSQLSGGQRQAVALARALIRKPS |
| | VLILDQATSALDAGNQLRVQRLLYESPERYSRSVLLITQHLSLVEQADH |
| | ILFLEGGAIREGGTHQQLMEKKGSYWAMVQAGGGGSGGGGSDKTHTCPP |
| | CPAPELLGGPSVFLFPPKPKDTLMISRTPEVTCVWVDVSHEDPEVKFNW |
| | YVDGVEVHNAKTKPREEQYNSTYRVVSVLTVLHQDWLNGKEYKCKVSNK |
| | ALPAPIEKTISKAKGQPREPQVYTLPPCRDELTKNQVSLWCLVKGFYPS |
| | DIAVEWESNGQPENNYKTTPPVLDSDGSFFLYSKLTVDKSRWQQGNVFS |
| | CSVMHEALHNHYTQKSLSLSPG |
| | (SEQ ID NO: 190 |

‡Any of the above-mentioned sequence may include a leader sequence for cellular expression, such as the acetylcholine leader sequence: MEPWPLLLLFSLCSAGLVLG (SEQ ID NO: 191)

To demonstrate the ability of constructs to undergo ATP mediated dimerization at different and selectable ATP concentrations biotinylated mesothelin was immobilized onto streptavidin (SAX) biosensors using a Octet Red 96e instrument (Pall ForteBio LLC, Freemont, CA). The EC50 for ATP was determined for each of the three constructs (constructs 533:571, 533:647, and 533:648, see FIG. 21 at A) by measuring the dimerization-induced mesothelin binding constants across a range of ATP concentrations (0.1 to 100 mM) using Biolayer Interferometry (BLI). More specifically, mesothelin was immobilized onto streptavidin (SAX) biosensors in equilibration buffer 20 mM Tris, 150 mM NaCl, 5 mM MgCl₂·6H2O Hexahydrate, 0.005% Tween pH 7.0, and the indicated concentrations of ADP or ATP. After washing away excess mesothelin, with equilibration buffer in the absence of ATP or in the presence of ATP at the concentrations from 0.1 μM to 100 μM, samples of the constructs (at 5, 10, and 20 nM) were then added and the sample allowed to equilibrate with the biosensor. The dissociation rate constants (kdis) at each concentration were the measured by following the rate of signal decline upon replacing the equilibration buffer containing construct with equilibration buffer containing ATP at the indicated concentration but without any construct present. The residence time, which is the inverse of the dissociation constant (i.e., (1/(kdis)) is plotted. The results are provided in FIG. 21 at B and demonstrate that constructs responding to different levels of ATP over four orders of magnitude can be prepared though mutations of amino acid residues having direct interactions with ATP or indirect interactions with ATP, such as mutations affecting the nucleotide-binding cofactor magnesium.

### Example 8. ATP Dimerization, Stability, and Pharmacokinetics of a Construct Interacting with Fcγ Receptors

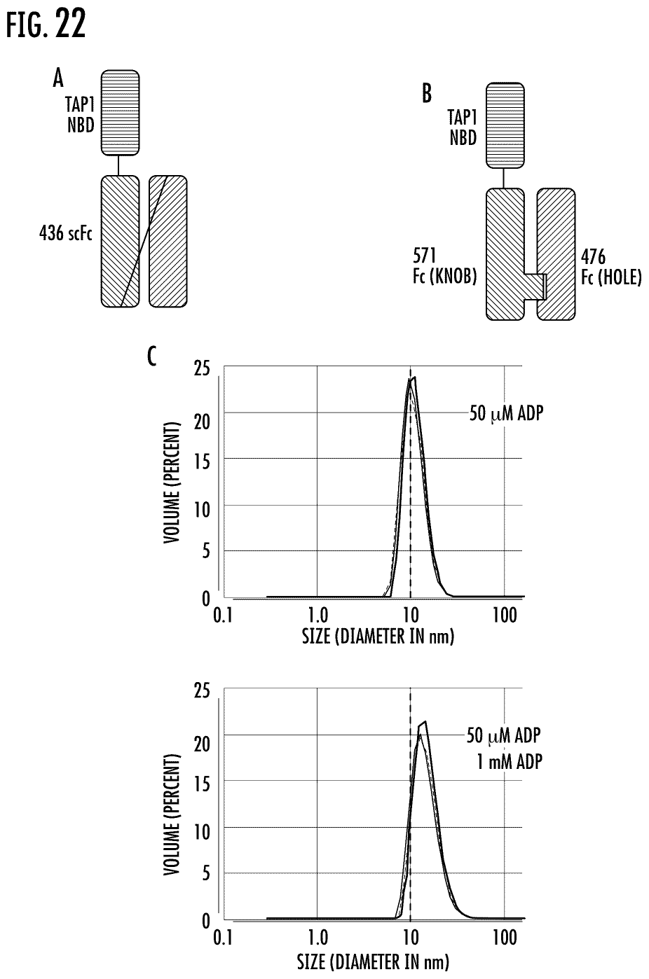

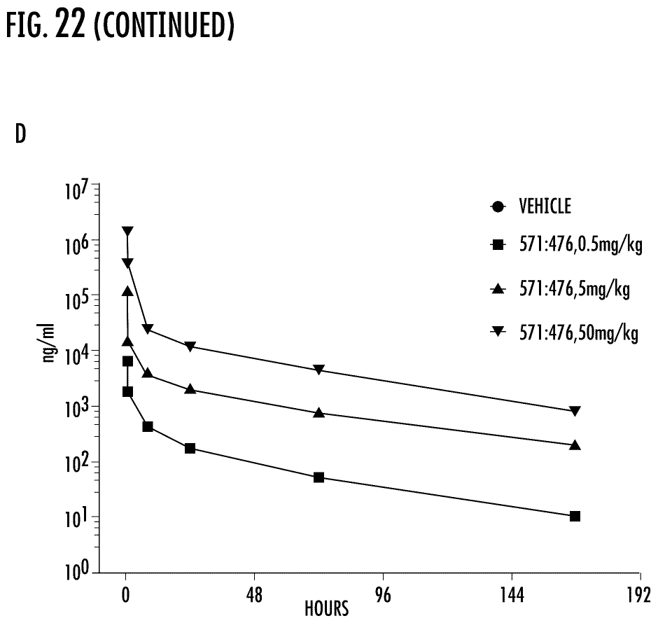

A pair of constructs having IgG scaffolds that are of the type suitable for inducing dimerization of Fc γ receptors in the presence of ATP and stimulating immune cell responses were prepared. The constructs comprised a TAP1 NBD aa sequence and either a scFc (construct 436 FIG. 22 at A) or pair of KiH IgG Fc aa sequences (polypeptides 571 and 476, FIG. 22 at B) were prepared by Expression in ExpiCHO or Expi293 cells.

| Construct | Description and sequence‡ |
|---|---|
| 436 | Cap (Coupling Helix 1-G₄S-Coupling |
| | Helix 2-underlined) (NL)-TAP1 NBD |
| | (modified D-helix; |
| | L493C: E573C; Q580C: C662; E587R; |
| | D668Q; C735S; deletion of the |
| | sequence “PADAPE”)-(G₄S)₂- |
| | scFc(G₄S)₆ |
| | ETEFFQQNQTGGGGSGLQTVRSFGSGCLTPLHLEG |
| | LVQFQDVSFAYPNRPDVLVLQGLTFTLRPGEVTAL |
| | VGPNGSGKSTVAALLQNLYQPTGGQLLLDGKPLPQ |
| | YCHRYLHRCVAAVGQRPQVFGRSLQENIAYGLTQK |
| | PTMEEITAAAVKSGAHSFISGLPQGYDTEVDEAGS |
| | QLSGGQRQAVALARALIRKPCVLILDQATSALDAG |
| | NQLRVQRLLYESPERYSRSVLLITQHLSLVEQADH |
| | ILFLEGGAIREGGTHQQLMEKKGSYWAMVQAGGGG |
| | SGGGGSAPEAAGGPSVFLFPPKPKDTLMISRTPEV |
| | TCVWVDVSHEDPEVKFNWYVDGVEVHNAKTKPREE |
| | QYNSTYRVVSVLTVLHQDWLNGKEYKCKVSNKALP |
| | APIEKTISKAKGQPREPQVYTLPPSRDELTKNQVS |
| | LTCLVKGFYPSDIAVEWESNGQPENNYKTTPPVLD |
| | SDGSFFLYSKLTVDKSRWQQGNVFSCSVMHEALHN |
| | HYTQKSLSLSPGGGGGSGGGGSGGGGSGGGGSGGG |
| | GSGGGGSAPEAAGGPSVFLFPPKPKDTLMISRTPE |
| | VTCVVVDVSHEDPEVKFNWYVDGVEVHNAKTKPRE |
| | EQYNSTYRVVSVLTVLHQDWLNGKEYKCKVSNKAL |
| | PAPIEKTISKAKGQPREPQVYTLPPSRDELTKNQV |
| | SLTCLVKGFYPSDIAVEWESNGQPENNYKTTPPVL |
| | DSDGSFFLYSKLTVDKSRWQQGNVFSCSVMHEALH |
| | NHYTQKSLSLSPG |
| | (SEQ ID NO: 192 |
| 476 | hIgG1 Fc(wt L234, L235; hole) |
| | DKTHTCPPCPAPELLGGPSVFLFPPKPKDTLMISR |
| | TPEVTCVVVDVSHEDPEVKFNWYVDGVEVHNAKTK |
| | PREEQYNSTYRVVSVLTVLHQDWLNGKEYKCKVSN |
| | KALPAPIEKTISKAKGQPREPQVCTLPPSRDELTK |
| | NQVSLSCAVKGFYPSDIAVEWESNGQPENNYKTTP |
| | PVLDSDGSFFLVSKLTVDKSRWQQGNVFSCSVMHE |
| | ALHNHYTQKSLSLSPG |
| | (SEQ ID NO: 193) |

‡Any of the above-mentioned sequence may include a leader sequence for cellular expression, such as the acetylcholine leader sequence: MEPWPLLLLFSLCSAGLVLG (SEQ ID NO: 191)

For expression the cells are transfected with 1 μg of an expression construct DNA per ml total transfection volume (including transfection reagents), according to manufacturer instructions, and cultures are incubated at 37° C. overnight. The following day the cells are fed enhancers are added. Expi293 cultures are continued in incubation at 37° C., while ExpiCHO cultures are transferred to 32° C. ExpiCHO cultures are fed a second time on day 5 post-transfection, per the max titer protocol. Expi293 transfections are harvested on day 4 post-transfection, while ExpiCHO cultures are harvested on day 6. Culture media containing secreted target proteins are collected by centrifugation of the transfection cultures at 12,000×g for 1 hour at 4° C., followed by 0.22 μm filtration of the supernatants, and storage at 4° C. until purification.

Target protein constructs are purified from the filtered supernatants on 1 ml MabSelect PrismA columns (Cytiva 17549852) at 4° C. The columns are equilibrated in MabSelect PrismA binding buffer (20 mM Tris-Cl pH 7.5, 150 mM NaCl, 5 mM MgCl₂, 10% glycerol, 50 μM ADP), then the supernatants are loaded at 0.5-2 ml/min (depending on total supernatant volumes), with unbound proteins removed by washing with 10 column volumes of binding buffer. Bound proteins are eluted in 7 column volumes of elution buffer (100 mM sodium citrate pH 3.3, 150 mM NaCl, 5 mM MgCl₂, 10% glycerol, 50 μM ADP) in 0.9 ml fractions, and the fractions neutralized with 350 μl of 1M Tris pH 9.0 in the fraction block wells. Peak fractions are pooled and concentrated before loading onto either a Superdex 200 10/300 Increase or 16/600 column (depending on total protein quantities), equilibrated in the MabSelect PrismA binding buffer. Peak fractions are assessed by SDS-PAGE, and target protein-containing fractions are pooled and concentrated (10 kDa MW cutoff) to approximately 1 mg/ml before aliquoting and flash-freezing in liquid nitrogen and storage at −80° C.

The proteins were substantially pure and SDS-PAGE analysis showed the expected two band under reducing conditions for the 571:476 construct under reducing conditions, and a single band under non-reducing conditions. LC MS analysis of the denatured, reduced, de-N/O-glycosylated 571:476 complex gave parent peak ions consistent with the expected molecules. In addition, deconvoluted mass spectra were calculated from multiply charged raw data using Bioconfirm software.

The stability of the constructs was assessed using Differential Scanning calorimetry (DSC) and freeze thaw testing. More specifically, the melting temperature (Tm) of the protein samples were measured by differential scanning calorimetry (DSC) using a Malvern MicroCal PEAQ-DSC (MalvernPanalytical, Malvern, UK) instrument in 20 mM Tris-Cl pH 7.5, 150 mM NaCl, 5 mM MgCl2, 10% glycerol, 50 μM AD buffer. A scan rate of 60° C./h was used over the temperature range 25° C.-100° C. DSC thermograms for the 571:476 construct show a melting point onset at 43.82° C., and three distinct Tm transitions at 54.41° C., 67.36° C., and 80.88° C. Construct 436 was subject to size exclusion chromatographic analysis on an Agilent HP1100, 7.8×300 mm TSKgel G3000SWxl column (Agilent Tech. Santa Clara, CA in 20 mM Tris pH 7.0, 150 mM NaCl, 5 mM MgCl2, 0.005% polysorbate20, 50 μM ADP buffer prior to and after each of three freeze-thaw cycles from −80° C. to 22° C. The construct chromatographed as a single peak accounting for more than 99% (approximately 99.5%) of the protein (based on integrated UV absorption) and show no formation of additional peaks following each of the successive freeze thaw cycles. The data suggest that the constructs, and particularly the NBDs are stable to freeze-thaw cycles and have melting points acceptable for administration to a human.

The ability of the constructs to dimerize in solution phase was demonstrated by dimerization of construct 571:476 in solution. Samples of the construct at 1.04 mg/ml were prepared in 20 mM Tris-HCl, pH 7.5, 150 mM NaCl, 5 mM MgCl₂, 10% glycerol with either 50 μM ADP or, after spiking with ATP, 50 μM ADP and 1 mM ATP. The samples were subjected to dynamic light scattering (DLS) using a Malvern Zetasizer Pro ZSU5800. For measurements, 20 μL of protein sample was pipetted into a Malvern ZEN2112 low-capacity quartz cuvette and inserted into the instrument. Temperature was maintained at 25° C. with five scans per sample and 120 seconds of equilibration between scans. Material values were set to protein (refractive index (RI): 1.45, absorption: 0.001) and dispersant values were set to water (RI: 1.33, Viscosity: 0.8872 mPas at 25° C.). Instrument control and data analysis was performed using Malvern ZS XPLORER software. In ADP, samples exhibited a mean hydrodynamic radius of diameter of approximately 11 nm (peak approximately 11.18 nm, see FIG. 22 at C upper panel) and a polydispersity index of about 0.433. Upon spiking 1 mM ATP, the hydrodynamic radius increased by approximately ˜5 nm increase across all constructs to give diameter of about 16 nm (peak approximately 16.25 nm, see FIG. 22 at C lower panel) and a polydispersity index of 0.580, which his indicative of dimerization.

Maximum Tolerated Dose (MTD) and Pharmacokinetic (PK) profile of construct 571:476 was studied in non-tumor bearing C57BL/6 mice relative to a vehicle control. The mice were injected intravenously at 3 dose levels (0.5 mg/kg, 5 mg/kg and 50 mg/kg) and serum concentration of the construct was monitored at 6 time points (5 min, 1 hour, 8 hours, 24 hours, 72 hours, and 168 hours (7 days)). Serum concentrations of the construct were measure by ELISA using polyclonal anti-human TAP1 capture antibody (Proteintech 11114-1-AP) and anti-human IgG1 Fc for detection (Jackson 609-035-213). Body weights and clinical signs were monitored over 7-day observation period to establish tolerability. There were no adverse changes in body weight or other clinical signs during the 7-days of testing. The time course for serum concentrations is provided in FIG. 22 at D (vehicle control is not shown as there is no construct concentrations to report). For the 5 mg/kg cohort the pharmacokinetic parameters were: T½=44.6 hours; Tmax=0.083 hours; Cmax 139,756 (ng/ml); AUC₀₋ₜ=304463 (ng·h/mL); AUC_{0-∞}=317518 (ng·h/mL); Vz (volume of distribution after i.v. administration)=1.01 (L/kg); Cl (plasma clearance)=0.02 (L/hour/kg); and MRTlast (mean residence time)=24.9 hours.

### Example 9. T Cell Agonists that can Selectively Activate T Cells in the Presence Sufficient ATP to Induce Dimerization

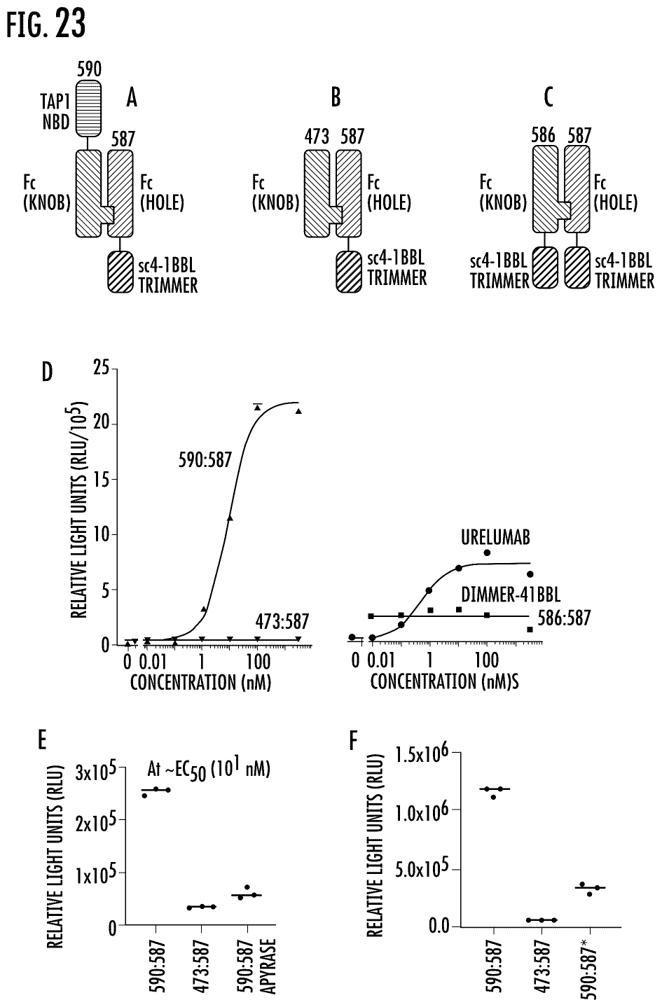

Construct 590:587 comprising polypeptides 590 and 587 having an NBD and a 4-1BBL single chain trimer that alone (in the absence of ATP) is insufficient stimulate T cells was prepared. Construct 590:587 is capable of dimerization in the presence of sufficient ATP, and as shown below, in the dimeric state can stimulate T cell expressing 4-1BB. Construct 473:587 comprising polypeptides 473 and 587 both lacking an NBD, and according incapable of dimerization in the presence of ATP, was prepared as a negative control. A genetic dimer compromising Fc knob-in-hole IgG aa sequences and trimeric 4-1BBL aa sequences are shown in FIG. 23 at C (construct 586:587). See the Table that follows and FIG. 23 at A, B, and C.

| Construct | Description and sequence‡ |
|---|---|
| 586 | hIgG1 Fc(L234A, L235A; knob)- |
| | (G₄S)₃-single chain (sc) trimer |
| | of 41BBL (three 41BBL sequence |
| | separated by (G₄S)₅ linkers) |
| | (“sct41BBL”) |
| | DKTHTCPPCPAPEAAGGPSVFLFPPKPKDTLMISR |
| | TPEVTCVVVDVSHEDPEVKFNWYVDGVEVHNAKTK |
| | PREEQYNSTYRVVSVLTVLHQDWLNGKEYKCKVSN |
| | KALPAPIEKTISKAKGQPREPQVYTLPPCRDELTK |
| | NQVSLWCLVKGFYPSDIAVEWESNGQPENNYKTTP |
| | PVLDSDGSFFLYSKLTVDKSRWQQGNVFSCSVMHE |
| | ALHNHYTQKSLSLSPGGGGGSGGGGSGGGGSDPAG |
| | LLDLRQGMFAQLVAQNVLLIDGPLSWYSDPGLAGV |
| | SLTGGLSYKEDTKELVVAKAGVYYVFFQLELRRVV |
| | AGEGSGSVSLALHLQPLRSAAGAAALALTVDLPPA |
| | SSEARNSAFGFQGRLLHLSAGQRLGVHLHTEARAR |
| | HAWQLTQGATVLGLFRVTPEIPAGGGGSGGGGSGG |
| | GGSGGGGSGGGGSDPAGLLDLRQGMFAQLVAQNVL |
| | LIDGPLSWYSDPGLAGVSLTGGLSYKEDTKELVVA |
| | KAGVYYVFFQLELRRVVAGEGSGSVSLALHLQPLR |
| | SAAGAAALALTVDLPPASSEARNSAFGFQGRLLHL |
| | SAGQRLGVHLHTEARARHAWQLTQGATVLGLFRVT |
| | PEIPAGGGGSGGGGSGGGGSGGGGSGGGGSDPAGL |
| | LDLRQGMFAQLVAQNVLLIDGPLSWYSDPGLAGVS |
| | LTGGLSYKEDTKELVVAKAGVYYVFFQLELRRVVA |
| | GEGSGSVSLALHLQPLRSAAGAAALALTVDLPPAS |
| | SEARNSAFGFQGRLLHLSAGQRLGVHLHTEARARH |
| | AWQLTQGATVLGLFRVTPEIPA |
| | (SEQ ID NO: 194) |
| 587 | hIgG1 Fc(L234A, L235A; hole)- |
| | (G₄S)₃-single chain (sct41BBL |
| | DKTHTCPPCPAPEAAGGPSVFLFPPKPKDTLMISR |
| | TPEVTCVVVDVSHEDPEVKFNWYVDGVEVHNAKTK |
| | PREEQYNSTYRVVSVLTVLHQDWLNGKEYKCKVSN |
| | KALPAPIEKTISKAKGQPREPQVCTLPPSRDELTK |
| | NQVSLSCAVKGFYPSDIAVEWESNGQPENNYKTTP |
| | PVLDSDGSFFLVSKLTVDKSRWQQGNVFSCSVMHE |
| | ALHNHYTQKSLSLSPGGGGGSGGGGSGGGGSDPAG |
| | LLDLRQGMFAQLVAQNVLLIDGPLSWYSDPGLAGV |
| | SLTGGLSYKEDTKELVVAKAGVYYVFFQLELRRVV |
| | AGEGSGSVSLALHLQPLRSAAGAAALALTVDLPPA |
| | SSEARNSAFGFQGRLLHLSAGQRLGVHLHTEARAR |
| | HAWQLTQGATVLGLFRVTPEIPAGGGGSGGGGSGG |
| | GGSGGGGSGGGGSDPAGLLDLRQGMFAQLVAQNVL |
| | LIDGPLSWYSDPGLAGVSLTGGLSYKEDTKELVVA |
| | KAGVYYVFFQLELRRVVAGEGSGSVSLALHLQPLR |
| | SAAGAAALALTVDLPPASSEARNSAFGFQGRLLHL |
| | SAGQRLGVHLHTEARARHAWQLTQGATVLGLFRVT |
| | PEIPAGGGGSGGGGSGGGGSGGGGSGGGGSDPAGL |
| | LDLRQGMFAQLVAQNVLLIDGPLSWYSDPGLAGVS |
| | LTGGLSYKEDTKELVVAKAGVYYVFFQLELRRVVA |
| | GEGSGSVSLALHLQPLRSAAGAAALALTVDLPPAS |
| | SEARNSAFGFQGRLLHLSAGQRLGVHLHTEARARH |
| | AWQLTQGATVLGLFRVTPEIPA |
| | (SEQ ID NO: 195) |
| 590 | Cap (Coupling Helix 1-G₄S-Coupling |
| | Helix 2-underlined) (NL)-TAP1 NBD |
| | (modified D-helix; |
| | L493C: E573C; S542A; C662S; D668Q; |
| | C735S; deletion of the sequence |
| | “PADAPE”)-(G₄S)₂-hIgG1 |
| | Fc(L234A, L235A; knob) |
| | ETEFFQQNQTGGGGSGLQTVRSFGSGCLTPLHLEG |
| | LVQFQDVSFAYPNRPDVLVLQGLTFTLRPGEVTAL |
| | VGPNGAGKSTVAALLQNLYQPTGGQLLLDGKPLPQ |
| | YCHRYLHRQVAAVGQEPQVFGRSLQENIAYGLTQK |
| | PTMEEITAAAVKSGAHSFISGLPQGYDTEVDEAGS |
| | QLSGGQRQAVALARALIRKPSVLILDQATSALDAG |
| | NQLRVQRLLYESPERYSRSVLLITQHLSLVEQADH |
| | ILFLEGGAIREGGTHQQLMEKKGSYWAMVQAGGGG |
| | SGGGGSDKTHTCPPCPAPEAAGGPSVFLFPPKPKD |
| | TLMISRTPEVTCVVVDVSHEDPEVKFNWYVDGVEV |
| | HNAKTKPREEQYNSTYRVVSVLTVLHQDWLNGKEY |
| | KCKVSNKALPAPIEKTISKAKGQPREPQVYTLPPC |
| | RDELTKNQVSLWCLVKGFYPSDIAVEWESNGQPEN |
| | NYKTTPPVLDSDGSFFLYSKLTVDKSRWQQGNVFS |
| | CSVMHEALHNHYTQKSLSLSPG |
| | (SEQ ID NO: 196) |
| 473 | hIgG1 Fc(L234A, L235A; knob) |
| | DKTHTCPPCPAPEAAGGPSVFLFPPKPKDTLMISR |
| | TPEVTCVVVDVSHEDPEVKFNWYVDGVEVHNAKTK |
| | PREEQYNSTYRVVSVLTVLHQDWLNGKEYKCKVSN |
| | KALPAPIEKTISKAKGQPREPQVYTLPPCRDELTK |
| | NQVSLWCLVKGFYPSDIAVEWESNGQPENNYKTTP |
| | PVLDSDGSFFLYSKLTVDKSRWQQGNVFSCSVMHE |
| | ALHNHYTQKSLSLSPG |
| | (SEQ ID NO: 197) |

‡Any of the above-mentioned sequence may include a leader sequence for cellular expression, such as the acetylcholine leader sequence: MEPWPLLLLFSLCSAGLVLG (SEQ ID NO: 191)

Cellular expression and purification was conducted in the manner described in Example 8. Constructs 590:587 and 473:587 showed a single band on non-reducing SDS-PAGE gels and two bands on reducing SDS PAGE. Size exclusion chromatograph showed 590:587 chromatographed principally as a single peak accounting for greater than 98% of the protein based on integration of the chromatogram's absorption profile. Chromatograph of a sample subject to a cycle of freezing and thawing again gave a single peak of the same molecular weight accounting for greater than 98% of the total protein.

To confirm the ability of the 590:587 construct to stimulate T cells a genetically engineered Jurkat T cell line expressing human 4-1BB and a luciferase reporter driven by a response element that can respond to 4-1BB ligand/agonist antibody stimulation was obtained from Promega (4-1BB Effector Cells JA2355). Constructs were examined in a ten-fold dilution series FIG. 23 at D (left side). Titration of the genetic dimer construct and positive control anti-4-1BB Ab (Urelumab) is sown in FIG. 23 at D (right side). All antibody concentrations and untreated controls were examined in the presence or absence of 10 U/ml apyrase, which degrades ATP, as Jurkat T cells produce sufficient ATP in in vitro cultures to permit dimerization of 590:587 constructs. All conditions were examined in triplicate. Constructs and control antibodies were prepared and kept at ambient temperature (22-25° C.) in the presence or absence of 10 U/mL apyrase. 4-1BB reporter cells were added to all wells according to manufacturer's recommendations. Samples were incubated at 37° C., 5% CO2, for 6 hours prior to analysis. Results from triplicate control tests of 590:587, 590:587+Apyrase, 473:587 and a 590:587 variant attenuate activity (marked 590:587*) are shown in FIG. 23 at E and F.

1. A construct comprising:
(i) a first polypeptide comprised of a first immunoglobulin heavy chain constant region amino acid sequence, and
(ii) a second polypeptide comprised of a second immunoglobulin heavy chain constant region amino acid sequence;
wherein
(i) the first polypeptide comprises a nucleotide binding domain (NBD) amino acid sequence joined to the first immunoglobulin heavy chain constant region amino acid sequence directly or indirectly by a linker amino acid sequence, the NBD amino acid sequence having at least 90% amino acid sequence identity to the capped NBD amino acid sequence of SEQ ID NO:294, or a sequence having at least 90% sequence identity to the aa sequence of SEQ ID NO:16, that can homodimerize in the presence of adenosine triphosphate (ATP),
(ii) the first and second immunoglobulin heavy chain constant region amino acid sequences form an IgFc whose effector functions are optionally diminished, and
(iii) each linker sequence present is selected independently.

2. The construct of claim 1, wherein the first and second immunoglobulin heavy chain constant region amino acid sequences comprise an interspecific amino acid sequence pair selected from the group consisting of: KiH, KiHs-s, HA-TF, ZW1, 7.8.60, DD-KK, EW-RVT, EW-RVTs-s, and A107 amino acid sequences.

3. The construct of claim 2, wherein the interspecific amino acid sequence pair is a KiH or KiHs-s sequence pair.

4. The construct of claim 3, wherein at least one of the first or second polypeptide of the construct comprises a payload or a label, wherein:
(i) the payload or label comprises one or more independently selected therapeutic or chemotherapeutic agents, and/or
(ii) the payload or label comprises one or more independently selected radioactive payloads or labels.

5. The construct of claim 4, further comprising one or more independently selected Tumor-Specific Binder amino acid sequences (TSBs).

6. The construct of claim 5, wherein the amino acid sequences of the one or more TSBs are selected from: nanobody, scFv, and VH (heavy chain only variable fragment) amino acid sequences.

7. The construct of claim 5, wherein the TSBs bind one or more Tumor Associated Antigens (TAAs) independently selected from the group consisting of a mucin, mesothelial (MSLN), EpCAM, CTLA-4, VISTA, TIM-3, PD-L1, and LAG-3 protein.

8. A construct comprising:
(i) a first polypeptide comprised of a first scaffold amino acid sequence comprising a first Ig heavy chain constant region amino acid sequence and a first nucleotide binding domain (NBD) amino acid sequence joined to the first scaffold amino acid sequence directly or by a linker amino acid sequence, and
(ii) a second polypeptide comprised of a second scaffold amino acid sequence comprising a second Ig heavy chain constant region amino acid sequence and optionally a first and/or a second NBD amino acid sequence joined to the second scaffold amino acid sequence directly or by a linker amino acid sequence;
wherein
(i) at least one of the first and second polypeptides comprises their first NBD amino acid sequence each of which has at least 90% amino acid sequence identity to the capped NBD amino acid sequence of SEQ ID NO:294, or a sequence having at least 90% sequence identity to the aa sequence of SEQ ID NO:16,
(ii) each NBD amino acid sequence comprises one or more ATP binding sites and can, in the presence of adenosine triphosphate (ATP), homodimerize,
(iii) at least one of the first and second polypeptides comprises one or more immune cell binder (ICB) and/or one or more activating domain (AD) amino acid sequences,
(iv) the first and second scaffold sequences form a dimer via interactions between the first and second scaffold sequences, and
(v) each linker sequence present is selected independently.

9. The construct of claim 8, wherein the only NBD amino acid sequence in the construct is the first NBD of the first polypeptide, and the second polypeptide comprises only one ICB or AD amino acid sequence in the construct.

10. The construct of claim 9, wherein the first and second immunoglobulin heavy chain constant region amino acid sequences comprise an interspecific amino acid sequence pair selected from the group consisting of: KiH, KiHs-s, HA-TF, ZW1, 7.8.60, DD-KK, EW-RVT, EW-RVTs-s, and A107 amino acid sequences.

11. The construct of claim 10, wherein at least one of the first or second polypeptide of the construct comprises a payload or a label, wherein:
(i) the payload or label comprises one or more independently selected therapeutic or chemotherapeutic agents, and/or
(ii) the payload or label comprises one or more independently selected radioactive payloads or labels.

12. The construct of claim 11, wherein the interspecific amino acid sequence pair is a KiH or KiHs-s sequence pair.

13. The construct of claim 12, wherein the ICB and/or AD amino acid sequence has affinity for a protein selected from the group consisting of: CD3, CD2, CD4, CD8, CD13, CD16, CD25, CD28, CD33, CD34, CD66, CD68, CD84, CD137/4-1BB, CD163, CD193, CD206, CXCR1, DR5, FcεR1α, αβTCR, TCRα chain, TCRβ chain, δγ TCR, TCR γ chain, TCR δ chain, and TRGV9.

14. The construct of claim 13, wherein the ICB and/or AD amino acid sequence has affinity for CD28 or CD137/4-1BB.

15. The construct of claim 14, wherein the ICB and/or AD is an anti-CD28 antibody, an anti-CD137 antibody, the 4-1BBL sequence of SEQ ID NO:114 or 115, the 4-1BBL trimer aa sequence of SEQ ID NO:116, or an amino acid sequence having at least 90% sequence identity to any of SEQ ID NOs: 114-116.

16. A complex comprising the construct of claim 1 and one or more molecules of ATP.

17. A pharmaceutical composition comprising a construct of claim 1 and at least one pharmaceutically acceptable excipient, wherein the composition is sterile and free of detectable pyrogens, or the pyrogens are below an acceptable limit.

18. A method of treating cancer in a human patient comprising administering to the patient a pharmaceutical composition of claim 17.

19-21. (canceled)

22. A pharmaceutical composition comprising a construct of claim 8 and at least one pharmaceutically acceptable excipient, wherein the composition is sterile and free of detectable pyrogens, or the pyrogens are below an acceptable limit.

23. A method of treating cancer in a human patient comprising administering to the patient a pharmaceutical composition of claim 22.
